SEQUENCE LISTING (155 sequences)

SEQ ID NO: 1 - protein, 10 aa, Homo sapiens
GVYDGEEHSV

SEQ ID NO: 2 - protein, 6 aa, Synthetic construct
DASSTY

SEQ ID NO: 3 - protein, 9 aa, Synthetic construct
AVKDNARLL

SEQ ID NO: 4 - protein, 9 aa, Synthetic construct
AVKDNARLM

SEQ ID NO: 5 - protein, 5 aa, Synthetic construct
RPVTM

SEQ ID NO: 6 - protein, 9 aa, Synthetic construct
SVLGAYGYT

SEQ ID NO: 7 - protein, 7 aa, Synthetic construct
IYSNMDM

SEQ ID NO: 8 - protein, 7 aa, Synthetic construct
IFSNMDM

SEQ ID NO: 9 - protein, 7 aa, Synthetic construct
AYDEGSA

SEQ ID NO: 10 - protein, 7 aa, Synthetic construct
AYDVGSA

SEQ ID NO: 11 - protein, 108 aa, Synthetic construct
EDVEQSSFLSVREGDSAVINCTYTDASSTYFYWYKQEPGAGLQLLTYIYSNMDMKQDQRLTVLLNKKDKHLSLRIADTQTGDSAIYFCAVKDNARLLFGDVTQLVVKP

SEQ ID NO: 12 - protein, 108 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDVTQLVVKP

SEQ ID NO: 13 - protein, 108 aa, Synthetic construct
EDVEQSSFLSVREGDSAVINCTYTDASSTYFYWYKQEPGAGLQLLTYIYSNMDMKQDQRLTVLLNKKDKHLSLRIADTQTGDSAIYFCAVKDNARLLFGDGTQLVVKP

SEQ ID NO: 14 - protein, 108 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKP

SEQ ID NO: 15 - protein, 111 aa, Synthetic construct
AVISQKPSRVIVQRGTSVTIQCQVDRPVTMMYWYRQQPGQSLTLIATAYDEGSATYESGFDIDKFPISRPNLTFSTLTVSNVEPEDSSIYLCSVLGAYGYTFGSGTRLTVV

SEQ ID NO: 16 - protein, 111 aa, Synthetic construct
AVISQKPSRYIVQRGTSVTIQCQVDRPVTMMYWYRQQPGQSLTLIATAYDVGSATYESGFVIDKFPISRPNLTFSTLTVSNVEPEDSSIYLCSVLGAYGYTFGSGTRLTVV

SEQ ID NO: 17 - protein, 111 aa, Synthetic construct
AVISQKPSRYIVQRGTSVTIQCQVDRPVTMMYWYRQQPGQSLTLIATAYDVGSATYESGFDIDKFPISRPNLTFSTLTVSNMEPEDSSIYLCSVLGAYGYTFGSGTRLTVV

SEQ ID NO: 18 - protein, 111 aa, Synthetic construct
AVISQKPSRVIVQRGTSVTIQCQVDRPVTMMYWYRQQPGQSLTLIATAYDVGSATYESGFVIDKFPISRPNLTFSTLTVSNVEPEDSSIYLCSVLGAYGYTFGSGTRLTVV

SEQ ID NO: 19 - protein, 111 aa, Synthetic construct
AVISQKPSRYIVQRGTSVTIQCQVDRPVTMMYWYRQQPGQSLTLIATAYDVGSATYESGFDIDKFPISRPNLTFSTLTVSNVEPEDSSIYLCSVLGAYGYTFGSGTRLTVV

SEQ ID NO: 20 - protein, 111 aa, Synthetic construct
AVISQKPSRYIVQRGNSVTIQCQVDRPVTMMFWYRQQPGQSPTLIATAYDVGSATYESGFVIDKFPISRPNLTFSTLTVSNMEPEDSSIYLCSVLGAYGYTFGSGTRLTVV

SEQ ID NO: 21 - protein, 111 aa, Synthetic construct
AVISQKPSRYIVQRGTSVTIQCQVDRPVTMMFWYRQQPGQSLTLIATAYDVGSATYESGFDIDKFPISRPNLTFSTLTVSNMEPEDSSIYLCSVLGAYGYTFGSGTRLTVV

SEQ ID NO: 22 - protein, 111 aa, Synthetic construct
AVISQKPSRDIVQRGTSVTIQCQVDRPVTMMYWYRQQPGQSLTLIATAYDEGSATYESGFDIDKFPISRPNLTFSTLTVSNMEPEDSSIYLCSVLGAYGYTFGSGTRLTVV

SEQ ID NO: 23 - protein, 111 aa, Synthetic construct
AVISQKPSRDIVQRGNSVTIQCQVDRPVTMMFWYRQQPGQSLTLIATAYDEGSATYESGFVIDKFPISRPNLTFSTLTVSNMSPEDSSIYLCSVLGAYGYTFGSGTRLTVV

SEQ ID NO: 24 - protein, 229 aa, Synthetic construct
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 25 - protein, 229 aa, Synthetic construct
EPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 26 - protein, 8 aa, Synthetic construct
GGGSGGGG

SEQ ID NO: 27 - protein, 465 aa, Synthetic construct
EDVEQSSFLSVREGDSAVINCTYTDASSTYFYWYKQEPGAGLQLLTYIYSNMDMKQDQRLTVLLNKKDKHLSLRIADTQTGDSAIYFCAVKDNARLLFGDVTQLVVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 28 - protein, 465 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDVTQLVVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 29 - protein, 465 aa, Synthetic construct
EDVEQSSFLSVREGDSAVINCTYTDASSTYFYWYKQEPGAGLQLLTYIYSNMDMKQDQRLTVLLNKKDKHLSLRIADTQTGDSAIYFCAVKDNARLLFGDGTQLVVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 30 - protein, 465 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 31 - protein, 451 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPGGGSGGGGQIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 32 - protein, 451 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGEDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 33 - protein, 465 aa, Synthetic construct
EVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSGGGSGGGGEDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 34 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRVIVQRGTSVTIQCQVDRPVTMMYWYRQQPGQSLTLIATAYDEGSATYESGFDIDKFPISRPNLTFSTLTVSNVEPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 35 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRYIVQRGTSVTIQCQVDRPVTMMYWYRQQPGQSLTLIATAYDVGSATYESGFVIDKFPISRPNLTFSTLTVSNVEPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 36 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRYIVQRGTSVTIQCQVDRPVTMMYWYRQQPGQSLTLIATAYDVGSATYESGFDIDKFPISRPNLTFSTLTVSNMEPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 37 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRVIVQRGTSVTIQCQVDRPVTMMYWYRQQPGQSLTLIATAYDVGSATYESGFVIDKFPISRPNLTFSTLTVSNVEPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 38 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRYIVQRGTSVTIQCQVDRPVTMMYWYRQQPGQSLTLIATAYDVGSATYESGFDIDKFPISRPNLTFSTLTVSNVEPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 39 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRYIVQRGNSVTIQCQVDRPVTMMFWYRQQPGQSPTLIATAYDVGSATYESGFVIDKFPISRPNLTFSTLTVSNMEPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 40 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRYIVQRGTSVTIQCQVDRPVTMMFWYRQQPGQSLTLIATAYDVGSATYESGFDIDKFPISRPNLTFSTLTVSNMEPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 41 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRDIVQRGTSVTIQCQVDRPVTMMYWYRQQPGQSLTLIATAYDEGSATYESGFDIDKFPISRPNLTFSTLTVSNMEPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 42 - protein, 454 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKGGGSGGGGAVISQKPSRDIVQRGNSVTIQCQVDRPVTMMFWYRQQPGQSLTLIATAYDEGSATYESGFVIDKFPISRPNLTFSTLTVSNMSPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 43 - protein, 468 aa, Synthetic construct
EVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSGGGSGGGGAVISQKPSRDIVQRGNSVTIQCQVDRPVTMMFWYRQQPGQSLTLIATAYDEGSATYESGFVIDKFPISRPNLTFSTLTVSNMSPEDSSIYLCSVLGAYGYTFGSGTRLTVVEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 44 - protein, 468 aa, Synthetic construct
AVISQKPSRDIVQRGNSVTIQCQVDRPVTMMFWYRQQPGQSLTLIATAYDEGSATYESGFVIDKFPISRPNLTFSTLTVSNMSPEDSSIYLCSVLGAYGYTFGSGTRLTVVGGGSGGGGEVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 45 - protein, 454 aa, Synthetic construct
AVISQKPSRDIVQRGNSVTIQCQVDRPVTMMFWYRQQPGQSLTLIATAYDEGSATYESGFVIDKFPISRPNLTFSTLTVSNMSPEDSSIYLCSVLGAYGYTFGSGTRLTVVGGGSGGGGQIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIKEPKSSDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 46 - protein, 120 aa, Synthetic construct
EVQLVQSGAEVKKPGASVKVSCKASGYKFTSYVMHWVRQAPGQGLEWMGYINPRNDVTKYAEKFQGRVTLTSDTSTSTAYMELSSLRSEDTAVYYCARGSYYDYEGFVYWGQGTLVTVSS

SEQ ID NO: 47 - protein, 106 aa, Synthetic construct
QIQMTQSPSSLSASVGDRVTITCSATSSVSYMHWYQQKPGKAPKRWIYDTSKLASGVPSRFSGSGSGTDYTLTISSLQPEDAATYYCQQWSSNPLTFGGGTKVEIK

SEQ ID NO: 48 - protein, 5 aa, Synthetic construct
SYVMH

SEQ ID NO: 49 - protein, 17 aa, Synthetic construct
YINPRNDVTKYAEKFQG

SEQ ID NO: 50 - protein, 11 aa, Synthetic construct
GSYYDYEGFVY

SEQ ID NO: 51 - protein, 10 aa, Synthetic construct
SATSSVSYMH

SEQ ID NO: 52 - protein, 7 aa, Synthetic construct
DTSKLAS

SEQ ID NO: 53 - protein, 9 aa, Synthetic construct
QQWSSNPLT

SEQ ID NO: 54 - protein, 120 aa, Synthetic construct
EVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYDGFVYWGQGTLVTVSS

SEQ ID NO: 55 - protein, 120 aa, Synthetic construct
EVQLVQSGAEVKKPGASVKVSCKASGYKFTSYVMHWVRQAPGQGLEWMGYINPYNDVTKYAEKFQGRVTLTSDTSTSTAYMELSSLRSEDTAVHYCARGSYYDYEGFVYWGQGTLVTVSS

SEQ ID NO: 56 - protein, 120 aa, Synthetic construct
EVQLVQSGAEVKKPGASVKVSCKASGYKFTSYVMHWVRQAPGQGLEWMGYINPRNDVTKYAEKFQGRVTLTSDTSTSTAYMELSSLRSEDTAVHYCARGSYYDYEGFVYWGQGTLVTVSS

SEQ ID NO: 57 - protein, 17 aa, Synthetic construct
YINPYNDVTKYAEKFQG

SEQ ID NO: 58 - protein, 11 aa, Synthetic construct
GSYYDYDGFVY

SEQ ID NO: 59 - protein, 10 aa, Homo sapiens
FIVDGVEVSV

SEQ ID NO: 60 - protein, 10 aa, Homo sapiens
FVYDEPGHAV

SEQ ID NO: 61 - protein, 10 aa, Homo sapiens
GLSDGEWQLV

SEQ ID NO: 62 - protein, 10 aa, Homo sapiens
GLYDGPVHEV

SEQ ID NO: 63 - protein, 10 aa, Homo sapiens
GVIDGHIYAV

SEQ ID NO: 64 - protein, 10 aa, Homo sapiens
GVMAGDIYSV

SEQ ID NO: 65 - protein, 10 aa, Homo sapiens
GVYGGSVHEA

SEQ ID NO: 66 - protein, 10 aa, Homo sapiens
TVYGGYLCSV

SEQ ID NO: 67 - protein, 10 aa, Homo sapiens
YTYDEAIHSV

SEQ ID NO: 68 - protein, 10 aa, Synthetic construct
AVYDGEEHSV

SEQ ID NO: 69 - protein, 10 aa, Synthetic construct
GVADGEEHSV

SEQ ID NO: 70 - protein, 10 aa, Synthetic construct
GVYAGEEHSV

SEQ ID NO: 71 - protein, 10 aa, Synthetic construct
GVYDAEEHSV

SEQ ID NO: 72 - protein, 10 aa, Synthetic construct
GVYDGAEHSV

SEQ ID NO: 73 - protein, 10 aa, Synthetic construct
GVYDGEAHSV

SEQ ID NO: 74 - protein, 10 aa, Synthetic construct
GVYDGEEASV

SEQ ID NO: 75 - protein, 10 aa, Synthetic construct
GVYDGEEHAV

SEQ ID NO: 76 - protein, 249 aa, Homo sapiens
EDVEQSLFLSVREGDSSVINCTYTDSSSTYLYWYKQEPGAGLQLLTYIFSNMDMKQDQRLTVLLNKKDKHLSLRIADTQTGDSAIYFCAEKDNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 77 - protein, 288 aa, Homo sapiens
AVISQKPSRDICQRGTSLTIQCQVDSQVTMMFWYRQQPGQSLTLIATANQGSEATYESGFVIDKFPISRPNLTFSTLTVSNMSPEDSSIYLCSVLGAYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 78 - protein, 108 aa, Homo sapiens
EDVEQSLFLSVREGDSSVINCTYTDSSSTYLYWYKQEPGAGLQLLTYIFSNMDMKQDQRLTVLLNKKDKHLSLRIADTQTGDSAIYFCAEKDNARLMFGDGTQLVVKP

SEQ ID NO: 79 - protein, 111 aa, Homo sapiens
AVISQKPSRDICQRGTSLTIQCQVDSQVTMMFWYRQQPGQSLTLIATANQGSEATYESGFVIDKFPISRPNLTFSTLTVSNMSPEDSSIYLCSVLGAYGYTFGSGTRLTVV

SEQ ID NO: 80 - protein, 6 aa, Homo sapiens
DSSSTY

SEQ ID NO: 81 - protein, 9 aa, Homo sapiens
AEKDNARLM

SEQ ID NO: 82 - protein, 5 aa, Homo sapiens
SQVTM

SEQ ID NO: 83 - protein, 7 aa, Homo sapiens
ANQGSEA

SEQ ID NO: 84 - protein, 87 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVF

SEQ ID NO: 85 - protein, 25 aa, Synthetic construct
GGGGSGGGGSGGGGSGGGGSGGGGS

SEQ ID NO: 86 - protein, 8 aa, Synthetic construct
DYKDDDDK

SEQ ID NO: 87 - protein, 10 aa, Synthetic construct
EQKLISEEDL

SEQ ID NO: 88 - protein, 368 aa, Synthetic construct
MQLLRCFSIFSVIASVLAQELTTICEQIPSPTLESTPYSLSTTTILANGKAMQGVFEYYKSVTFVSNCGSHPSTTSKGSPINTQYVFGGGGSDYKDDDDKGGGASEDVEQSLFLSVREGDSAVINCTYTDSSSTYLYWYKQEPGAGLQLLTYIFSNMDMKQDQRLTVLLNKKDKHLSLRIADTQTGDSAIYFCAEKDNARLMFGDGTQLVVKPGGGGSGGGGSGGGGSGGGGSGGGGSGAVISQKPSRDICQRGTSLTIQCQVDSQVTMMFWYRQQPGQSLTLIATANQGSEATYESGFVIDKFPISRPNLTFSTLTVSNMSPEDSSIYLCSVLGAYGYTFGSGTRLTVVAAAGGSGGEQKLISEEDL

SEQ ID NO: 89 - protein, 245 aa, Synthetic construct
EDVEQSLFLSVREGDSSVINCTYTDSSSTYLYWYKQEPGAGLQLLTYIFSNMDMKQDQRLTVLLNKKDKHLSLRIADTQTGDSAIYFCAEKDNARLMFGDGTQLVVKPGGGGSGGGGSGGGGSGGGGSGGGGSGAVISQKPSRDICQRGTSLTIQCQVDSQVTMMFWYRQQPGQSLTLIATANQGSEATYESGFVIDKFPISRPNLTFSTLTVSNMSPEDSSIYLCSVLGAYGYTFGSGTRLTVV

SEQ ID NO: 90 - protein, 245 aa, Synthetic construct
EDVEQSLFLSVREGDSAVINCTYTDSSSTYLYWYKQEPGAGLQLLTYIFSNMDMKQDQRLTVLLNKKDKHLSLRIADTQTGDSAIYFCAEKDNARLMFGDGTQLVVKPGGGGSGGGGSGGGGSGGGGSGGGGSGAVISQKPSRDIVQRGTSLTIQCQVDSQVTMMFWYRQQPGQSLTLIATANQGSEATYESGFVIDKFPISRPNLTFSTLTVSNMSPEDSSIYLCSVLGAYGYTFGSGTRLTVV

SEQ ID NO: 91 - protein, 245 aa, Synthetic construct
EDVEQSSFLSVREGDSAVINCTYTDSSSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSGAVISQKPSRDIVQRGNSVTIQCQVDSQVTMMFWYRQQPGQSLTLIATANQGSEATYESGFVIDKFPISRPNLTFSTLTVSNMSPEDSSIYLCCVLGAYGYTFGSGTRLTVV

SEQ ID NO: 92 - protein, 9 aa, Synthetic construct
CVLGAYGYT

SEQ ID NO: 93 - protein, 245 aa, Synthetic construct
EDVEQSSFLSVREGDSAVINCTYTDSSSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSGAVISQKPSRDIVQRGNSVTIQCQVDSQVTMMFWYRQQPGQSLTLIATANQGSEATYESGFVIDKFPISRPNLTFSTLTVSNMSPEDSSIYLCSVLGAYGYTFGSGTRLTVV

SEQ ID NO: 94 - protein, 245 aa, Synthetic construct
EDVEQSSFLSVREGDSAVINCTYTDSSSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSGAVISQKPSRDIVQRGNSVTIQCQVDRPVTMMFWYRQQPGQSLTLIATAYDDGSATYESGFVIDKFPISRPNLTFSTLTVSNMSPEDSSIYLCSVLGAYGYTFGSGTRLTVV

SEQ ID NO: 95 - protein, 245 aa, Synthetic construct
EDVEQSSFLSVREGDSAVINCTYTDSSSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSGAVISQKPSRDIVQRGNSVTIQCQVDRPVTMMFWYRQQPGQSLTLIATAYSDGTATYESGFVIDKFPISRPNLTFSTLTVSNMSPEDSSIYLCSVLGAYGYTFGSGTRLTVV

SEQ ID NO: 96 - protein, 245 aa, Synthetic construct
EDVEQSSFLSVREGDSAVINCTYTDSSSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSGAVISQKPSRDIVQRGNSVTIQCQVDRPVTMMFWYRQQPGQSLTLIATAYDDNTTTYESGFVIDKFPISRPNLTFSTLTVSNMSPEDSSIYLCSVLGAYGYTFGSGTRLTVV

SEQ ID NO: 97 - protein, 244 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSAVISQKPSRDIVQRGNSVTIQCQVDRPVTMMFWYRQQPGQSLTLIATAYDEGSATYESGFVIDKFPISRPNLTFSTLTVSNMSPEDSSIYLCSVLGAYGYTFGSGTRLTVV

SEQ ID NO: 98 - protein, 244 aa, Synthetic construct
EDVEQSSFLSVREGDSAVINCTYTDDSSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSAVISQKPSRDIVQRGNSVTIQCQVDRPVTMMFWYRQQPGQSPTLIATAYDEGSATYESGFVIDKFPISRPNLTFSTLTVSNMSPEDSSIYLCCVLGAYGYTFGSGTRLTVV

SEQ ID NO: 99 - protein, 244 aa, Synthetic construct
EDVEQSSFLSVREGDSAVINCTYTDASSTYFYWYKQEPGAGLQLLTYIYSNMDMKQDQRLTVLLNKKDKHLSLRIADTQTGDSAIYFCAVKDNARLLFGDGTQLVVKPGGGGSRGGGSGGGGSGGGGSGGGDSAVISQKPSRDIVQRGNSVTIQCQVDRPVTMMFWYRQQPGQSLTLIATAYDEGSATYESGFDIDKFPISRPNLTFSTLTVSNMSPEDSSIYLCSVLGAYGYTFGSGTRLTVV

SEQ ID NO: 100 - protein, 244 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSAVISQKPSRDIVQRGNSVTIQCQVDRPVTMMYWYRQQPGQSLTLIATAYDEGSATYESGFDIDKFPISRPNLTFSTLTVSNMSPEDSSIYFCSVLGAYGYTFGSGTRLTVV

SEQ ID NO: 101 - protein, 244 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSAVISQKPSRYIVQRGNSVTIQCQVDRPVTMMFWYRQQPGQSPTLIATAYDVGSATYESGFVIDKFPISRPNLTFSTLTVSNMSPEDSSIYLCSVLGAYGYTFGSGTRLTVV

SEQ ID NO: 102 - protein, 244 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDVTQLVVKPGGGGSGGGGSGGGGSGGGGSGGGGSAVISQKPSRVIVQRGNSVTIQCQVDRPVTMMYWYRQQPGQSLTLIATAYDEGSATYESGFVIDKFPISRPNLTFSTLTVSNVSPEDSSIYLCSVLGAYGYTFGSGTRLTVV

SEQ ID NO: 103 - protein, 244 aa, Synthetic construct
EDVEQSSFLSVREGDSAVINCTYTDSSSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSAVISQKPSRDIVQRGTSVTIQCQVDRPVTMMFWYRQQPGQSLTLIATAYDEGSATYESGFVIDKFPISRPNLTFSTLTVSNLEPEDSSIYLCSVLGAYGYTFGSGTRLTVV

SEQ ID NO: 104 - protein, 244 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSAVISQKPSRDIVQRGTSVTIQCQVDRPVTMMFWYRQQPGQSLTLIATAYDEGSATYESGFVIDKFPISRPNLTFSTLTVSNMEPEDSSIYLCSVLGAYGYTFGSGTRLTVV

SEQ ID NO: 105 - protein, 244 aa, Synthetic construct
EDVEQSSFLSVREGDSAVINCTYTDASSTYFYWYKQEPGAGLQLLTYIFSNMDMKQDQRLTVLLNKKDKHLSLRIADTQTGDSAIYFCAVKDNARLMFGDGTQLVVKPGGGGSGGGGSGGGGSGGGGSGGGGSAVISQKPSRDIVQRGTSVTIQCQVDRPVTMMFWYRQQPGQSLTLIATAYDEGSATYESGFVIDKFPISRPNLTFSTLTVSSMEPEDSSIYLCSVLGAYGYTFGSGTRLTVV

SEQ ID NO: 106 - protein, 10 aa, Homo sapiens
RLYDGLFKVI

SEQ ID NO: 107 - protein, 7 aa, Synthetic construct
AYDNNRT

SEQ ID NO: 108 - protein, 7 aa, Synthetic construct
AFDDNTT

SEQ ID NO: 109 - protein, 7 aa, Synthetic construct
AYDDGSA

SEQ ID NO: 110 - protein, 7 aa, Synthetic construct
AYSDGTA

SEQ ID NO: 111 - protein, 5 aa, Synthetic construct
  VARIANT 1: note = S, R, K or H
XYVMH

SEQ ID NO: 112 - protein, 17 aa, Synthetic construct
  VARIANT 5: note = Y, R, K, or H
  VARIANT 6: note = N, R, or K
  VARIANT 12: note = A or N
  VARIANT 16: note = Q or K
YINPXXDVTKYXZKFXG

SEQ ID NO: 113 - protein, 10 aa, Synthetic construct
  VARIANT 7: note = S, R, or K
SATSSVXYMH

SEQ ID NO: 114 - protein, 7 aa, Synthetic construct
  VARIANT 7: note = S, R, or K
DTSKLAX

SEQ ID NO: 115 - protein, 121 aa, unidentified
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 116 - protein, 112 aa, unidentified
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 117 - protein, 121 aa, unidentified
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSS

SEQ ID NO: 118 - protein, 112 aa, unidentified
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIK

SEQ ID NO: 119 - protein, 119 aa, unidentified
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 120 - protein, 112 aa, unidentified
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 121 - protein, 122 aa, unidentified
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 122 - protein, 112 aa, unidentified
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 123 - protein, 121 aa, unidentified
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSS

SEQ ID NO: 124 - protein, 106 aa, unidentified
DIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIK

SEQ ID NO: 125 - protein, 123 aa, unidentified
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 126 - protein, 107 aa, unidentified
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 127 - protein, 123 aa, unidentified
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 128 - protein, 107 aa, unidentified
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 129 - protein, 118 aa, unidentified
QVQLVQSGAEVKKPGASVKVSCKASGYTFTKYVIHWVRQAPGQGLEWMGYINPYNDVSKYNEKFRGRVTMTTDTSTSTAYMELRSLRSDDTAVYYCARNKDYYPMDYWGQGTTVTVSS

SEQ ID NO: 130 - protein, 106 aa, unidentified
EIVLTQSPATLSLSPGERATLSCSASSSVSYMHWYQQKPGQAPRLLIYDTSKLASGIPARFSGSGSGTDFTLTISSLEPEDFAVYYCQQWSSNPLTFGGGTKVEIK

SEQ ID NO: 131 - protein, 118 aa, unidentified
QVQLVQSGGGVVQPGRSLRLSCVASGFTFSSYGMHWVRQAPGKGLEWVAAIWYNARKQDYADSVKGRFTISRDNSKNTLYLQMNSLRAEDTAVYYCTRGTGYNWFDPWGQGTLVTVSS

SEQ ID NO: 132 - protein, 107 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 133 - protein, 9 aa, Homo sapiens
YLLPAIVHI

SEQ ID NO: 134 - protein, 10 aa, Homo sapiens
GMYDGPVYEV

SEQ ID NO: 135 - protein, 10 aa, Homo sapiens
FIYDNTEFTL

SEQ ID NO: 136 - protein, 10 aa, Homo sapiens
NQWDGTQHGV

SEQ ID NO: 137 - protein, 10 aa, Homo sapiens
SQYSGQLHEV

SEQ ID NO: 138 - protein, 10 aa, Homo sapiens
VLYEGPVRGL

SEQ ID NO: 139 - protein, 10 aa, Homo sapiens
GLYEGEFTLA

SEQ ID NO: 140 - protein, 10 aa, Homo sapiens
GMYDGPVFDL

SEQ ID NO: 141 - protein, 10 aa, Homo sapiens
GLYDGPVCEV

SEQ ID NO: 142 - protein, 10 aa, Homo sapiens
GLYEGLDWLA

SEQ ID NO: 143 - protein, 10 aa, Homo sapiens
GLYEGLDWLS

SEQ ID NO: 144 - protein, 10 aa, Homo sapiens
FLHDGQEYTL

SEQ ID NO: 145 - protein, 10 aa, Homo sapiens
ALYPGTDYTV

SEQ ID NO: 146 - protein, 10 aa, Homo sapiens
SLYPFQIHSI

SEQ ID NO: 147 - protein, 10 aa, Homo sapiens
RMYPFQIHSI

SEQ ID NO: 148 - protein, 10 aa, Homo sapiens
LLYDGKLSSA

SEQ ID NO: 149 - protein, 10 aa, Homo sapiens
ALYPGTNYTV

SEQ ID NO: 150 - protein, 10 aa, Homo sapiens
ALENSEEHSA

SEQ ID NO: 151 - protein, 10 aa, Homo sapiens
KFYDGVVQTV

SEQ ID NO: 152 - protein, 108 aa, synthetic construct
EDVEQSSFLSVREGDSAVINCTYTDASSTYFYWYKQEPGAGLQLLTYIXSNMDMKQDQRLTVLLNKKDKHLSLRIADTQTGDSAIYFCAVKDNARLXFGDXTQLVVKP

SEQ ID NO: 153 - protein, 108 aa, synthetic construct; identical to SEQ ID NO: 152
  VARIANT 49: note = Y or F
  VARIANT 97: note = L or M
  VARIANT 101: note = V or G

SEQ ID NO: 154 - protein, 111 aa, synthetic construct
AVISQKPSRXIVQRGXSVTIQCQVDRPVTMMXWYRQQPGQSXTLIATAYDXGSATYESGFXIDKFPISRPNLTFSTLTVSNXXPEDSSIYLCSVLGAYGYTFGSGTRLTVV

SEQ ID NO: 155 - protein, 111 aa, synthetic construct; identical to SEQ ID NO: 154
  VARIANT 10: note = V or Y or D
  VARIANT 16: note = T or N
  VARIANT 32: note = Y or F
  VARIANT 42: note = L or P
  VARIANT 51: note = E or V
  VARIANT 61: note = D or V
  VARIANT 82: note = V or M
  VARIANT 83: note = E or S

(19) United States
(12) Patent Application Publication
(10) Pub. No.: US 2025/0034227 A1
(43) Pub. Date: Jan. 30, 2025

(54) ANTIGEN BINDING PROTEINS AGAINST MAGEB2

(71) Applicant: Immatics Biotechnologies GmbH, Tuebingen (DE)

(72) Inventors: Felix UNVERDORBEN, Tuebingen (DE); Sebastian BUNK, Tuebingen (DE); Martin HOFMANN, Tuebingen (DE); Meike HUTT, Tuebingen (DE); Timo MANZ, Tuebingen (DE); Nadine ASCHMONEIT, Tuebingen (DE); Maike JAWORSKI, Tuebingen (DE); Lena SCHMOHL, Tuebingen (DE); Claudia WAGNER, Tuebingen (DE); Dominik MAURER, Tuebingen (DE); Heiko SCHUSTER, Tuebingen (DE); Nora TREIBER, Tuebingen (DE)

(21) Appl. No.: 18/785,378

(22) Filed: Jul. 26, 2024

Related U.S. Application Data
(60) Provisional application No. 63/516,005, filed on Jul. 27, 2023.

(51) Int. Cl. C07K 14/725; A61K 38/00; A61P 35/00

(52) U.S. Cl. CPC C07K 14/7051; A61P 35/00; A61K 38/00

(57) ABSTRACT

The present invention relates to antigen binding proteins that specifically bind to a tumor expressed melanoma-associated antigen (MAGE) B2 antigenic peptide in a complex with MHC. The antigen binding proteins are provided for use in the treatment of MAGEB2-expressing cancers. Further provided are nucleic acids encoding the antigen binding proteins, vectors comprising the nucleic acids, recombinant cells expressing the antigen binding proteins and pharmaceutical compositions comprising the antigen binding proteins.

### CROSS-REFERENCE TO RELATED APPLICATIONS

This application claims priority to U.S. Provisional Patent Application No. 63/516,005, filed 27 Jul. 2023, the entire contents of which are incorporated herein by reference.

### REFERENCE TO SEQUENCE LISTING SUBMITTED AS AN XML FILE (.xml)

A Sequence Listing is submitted herewith as an XML file named “3000058-027001_Sequence_Listing” created on 25 Jul. 2024 and having a size of 159,649 bytes as permitted under 37 C.F.R. § 1.821 (c). The material in the aforementioned file is hereby incorporated by reference in its entirety.

### FIELD

The present invention relates to antigen binding proteins that specifically bind to a tumor expressed melanoma-associated antigen (MAGE) B2 antigenic peptide in a complex with MHC. The antigen binding proteins are provided for use in the treatment of MAGEB2-expressing cancers. Further provided are nucleic acids encoding the antigen binding proteins, vectors comprising the nucleic acids, recombinant cells expressing the antigen binding proteins and pharmaceutical compositions comprising the antigen binding proteins.

### BACKGROUND

TCR based immunotherapy targets peptide epitopes derived from tumor-associated or tumor-specific proteins, which are presented by molecules of the major histocompatibility complex (MHC). These tumor associated antigens (TAAs) can be peptides derived from all protein classes, such as enzymes, receptors, transcription factors, etc., which are specifically expressed by cancer cells, and/or upregulated in cancer cells. Unlike chimeric antigen receptor (CAR)-T cell therapy and current antibody-based approaches, which can only target cell surface proteins, TCR based immunotherapy enables the targeting of otherwise inaccessible intracellular proteins and thus significantly increases the number and diversity of targets derived from tumor-associated or tumor-specific proteins.

Cancer-testis antigens (CTA) are attractive targets for cancer immunotherapy due to their restricted expression in germ cells and aberrant reactivation in various cancers, and their immunogenic properties. The melanoma antigen (MAGE) gene family includes intracellular cancer-testis antigens, such as MAGEB2. MAGEB2 is typically only expressed in normal testis. MAGEB2, which may function to enhance ubiquitin ligase activity of RING-type zinc finger containing E3 ubiquitin protein ligases, has been found to be aberrantly expressed in a variety of human tumors such as lung carcinoma, breast carcinoma, melanoma, and others. Targeting MAGEB2 for immunotherapy was previously disclosed in WO 2016/102272 A1, WO 2017/097602 A1, WO 2017/148888, WO 2017/174822 A1 and WO 2021/236638 A1. Many types of cancers still have a high unmet medical need, with patients needing improved, effective, and specific therapeutics. Accordingly, there exists a need to develop new anti-cancer agents that specifically target intracellular proteins highly specific to cancer cells. The present invention addresses that need by providing novel antigen binding proteins specifically binding to MAGEB2.

### SUMMARY

The invention provides an antigen binding protein specifically binding to a MAGEB2 antigenic peptide that is in a complex with a major histocompatibility complex (MHC) protein, wherein the MAGEB2 antigenic peptide comprises or consists of the amino acid sequence GVYDGEEHSV (SEQ ID NO: 1),
wherein the antigen binding protein comprises a variable domain V_{A} comprising complementarity determining regions (CDRs) CDRa1, CDRa2, and CDRa3, and a variable domain V_{B} comprising CDRb1, CDRb2, and CDRb3, wherein CDRa1, CDRa2, CDRa3, CDRb1, CDRb2 and CDRb3 form an antigen binding domain A;
preferably wherein the variable domain V_{A} comprises or consists of the amino acid sequence according to SEQ ID NO: 152, wherein X may be any amino acid; or an amino acid sequence having at least 85%, 90%, 95%, 98% or 99% identity to SEQ ID NO: 152, wherein X may be any amino acid;
preferably wherein the variable domain V_{B} comprises or consists of the amino acid sequence according to SEQ ID NO: 154, wherein X may be any amino acid; or an amino acid sequence having at least 85%, 90%, 95%, 98% or 99% identity to SEQ ID NO: 154, wherein X may be any amino acid;
preferably wherein
(1) CDRa1 comprises SEQ ID NO: 2, CDRa3 comprises SEQ ID NO: 3, CDRb1 comprises SEQ ID NO: 5, and CDRb3 comprises SEQ ID NO: 6; or
(2) CDRa1 comprises SEQ ID NO: 2, CDRa3 comprises SEQ ID NO: 4, CDRb1 comprises SEQ ID NO: 5, and CDRb3 comprises SEQ ID NO: 6;
wherein the CDRa1, CDRa3, CDRb1 and/or CDRb3 sequence(s) may comprise one, two or three amino acid mutations.

The antigen binding proteins of the invention are characterized by a high stability, high affinity, high functional avidity, high efficacy and high specificity. The antigen binding proteins of the invention are thus both more effective and safer than prior art antigen binding proteins as demonstrated in the exemplary part below. Furthermore, these new antigen binding proteins, in particular in the form of soluble antigen binding proteins (e.g., TCER® molecules), show a high cytotoxicity against tumor cells.

In a second aspect, the invention provides (a) nucleic acid(s) comprising a sequence encoding the antigen binding protein of the first aspect of the invention.

In a third aspect, the invention provides (a) vector(s) comprising the nucleic acid(s) of the second aspect of the invention.

In a fourth aspect, the invention provides a host cell comprising the antigen binding protein of the first aspect of the invention, the nucleic acid(s) of the second aspect of the invention, or the vector(s) of the third aspect of the invention.

In a fifth aspect, the invention provides a method of making the antigen binding protein according to the first aspect of the invention.

In a sixth aspect, the invention provides a pharmaceutical composition comprising the antigen binding protein of the first aspect of the invention, the nucleic acid(s) of the second aspect of the invention, the vector(s) of the third aspect of the invention or the host cell of the fourth aspect of the invention, and optionally a pharmaceutically acceptable carrier.

In a seventh aspect, the invention provides the antigen binding protein of the first aspect of the invention, the nucleic acid(s) of the second aspect of the invention, the vector(s) of the third aspect of the invention, the host cell of the fourth aspect of the invention, or the pharmaceutical composition of the fourth aspect of the invention for use in the treatment of cancer, in particular a MAGEB2-expressing cancer.

In an eighth aspect, the invention provides a kit comprising the antigen binding protein of the first aspect of the invention, the nucleic acid(s) of the second aspect of the invention, and/or the vector(s) of the third aspect of the invention.

### BRIEF DESCRIPTION OF THE FIGURES

FIG. 1. Target tetramer titration for stabilized scTCR.

HLA-A*02/MAGEB2-001 tetramer was titrated on the selected clone R76P1E1C from the scTCR stabilization library.

FIGS. 2A-2C. Target binding and specificity analysis on affinity matured scTCRs

Selected yeast clones with scTCRs from CDRb1 and CDRb2 libraries were analyzed for target and similar peptide binding. The parental library clone R76P1E1S was included in the analysis. CDRb1 clones were stained with HLA-A*02/MAGEB2-001 tetramers (FIG. 2A), while CDRb2 clones were stained with HLA-A*02/MAGEB2-001 monomers (FIG. 2B). Specificity was addressed using a mix of 9 similar peptides together with HLA-A*02 as peptide-HLA tetramer at a concentration of 25 nM each (FIG. 2C).

FIGS. 3A and 3B. Target binding and specificity analysis on scTCRs after CDR combination

Binding of scTCR variants on yeast cells to the target HLA-A*02/MAGEB2-001 was investigated as a titration series in a monomeric format (FIG. 3A), while 9 similar peptides together with HLA-A*02 were addressed individually as peptide-HLA tetramer at a concentration of 10 nM each (FIG. 3B).

FIG. 4. scTCR motif determination

MAGEB2-001 peptide or single amino acid substitutions thereof in the context of HLA-A*02 were investigated for binding to scTCR-displaying yeast cells. Four concentrations (31.6 nM, 10 nM, 3.2 nM, 1 nM, respectively) of HLA-A*02 monomers with MAGEB2-001 or the substituted peptides were used to stain the high affinity scTCR-bearing yeast cells, as indicated with the respective four bars per peptide.

FIG. 5. Target titration after bio-optimization

HLA-A*02/MAGEB2-001 monomer was titrated on yeast clones selected from the bio-optimization libraries and compared to their parental molecule CL-21753. Closed symbol clones originate from the error-prone PCR-based library, open symbol clones from the library with directed amino acid substitutions.

FIGS. 6A-6C. Results of an LDH-release assay with the bispecific TCR/mAb diabody

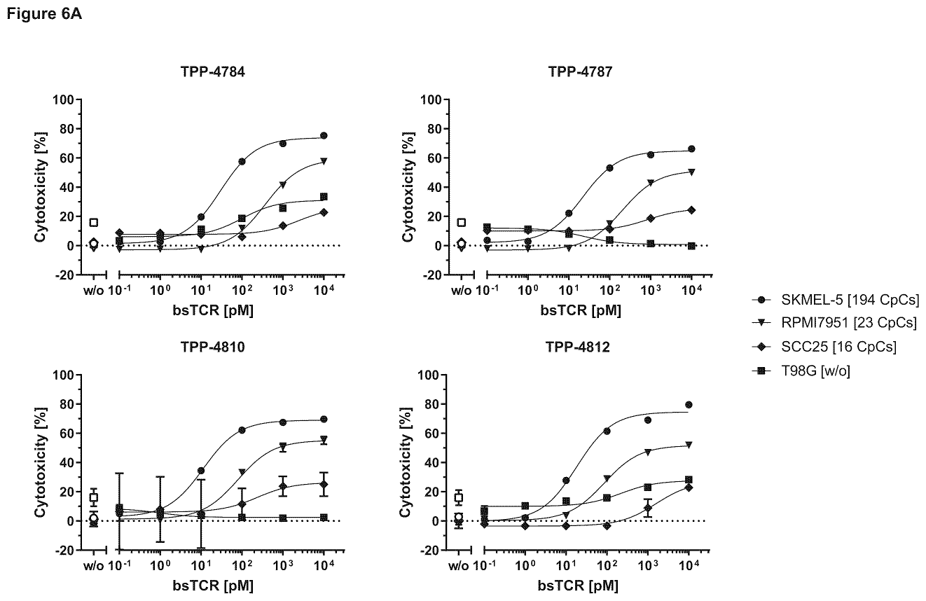

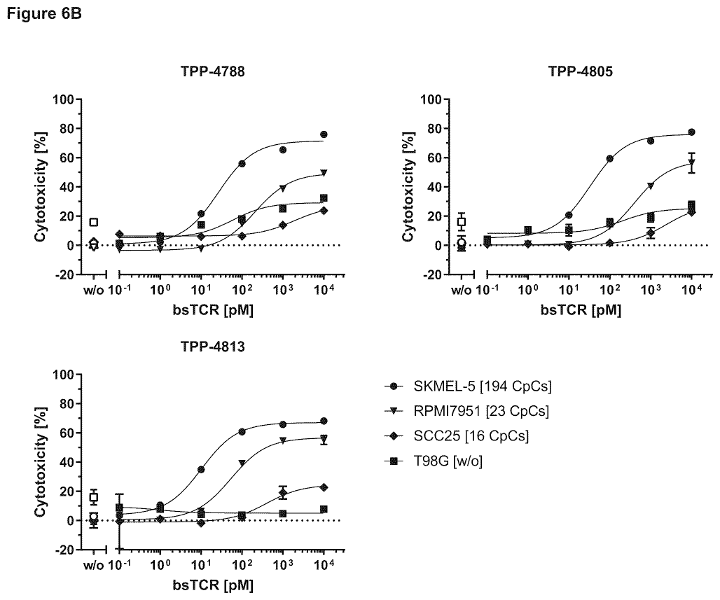

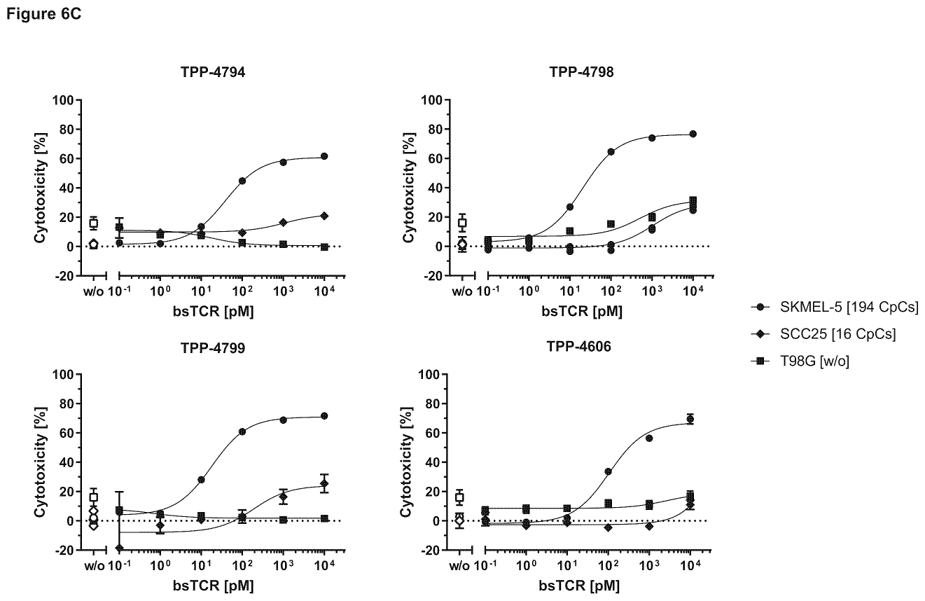

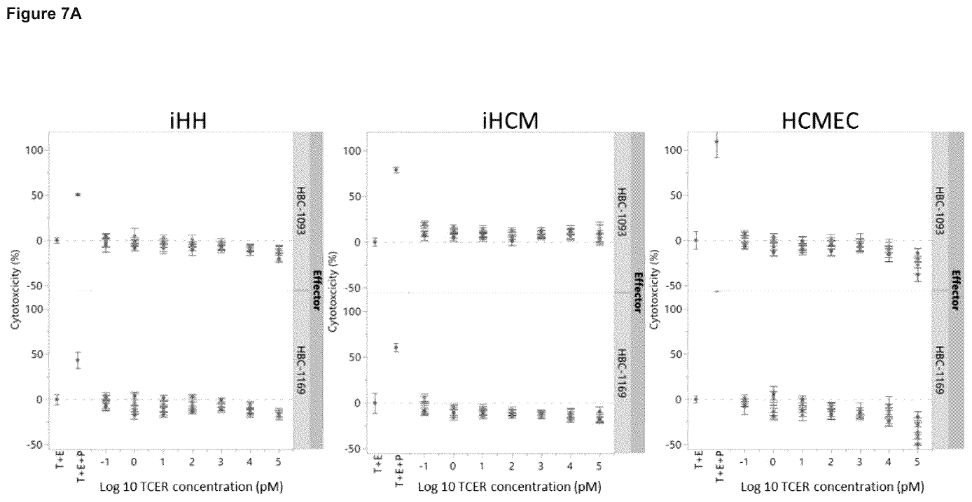

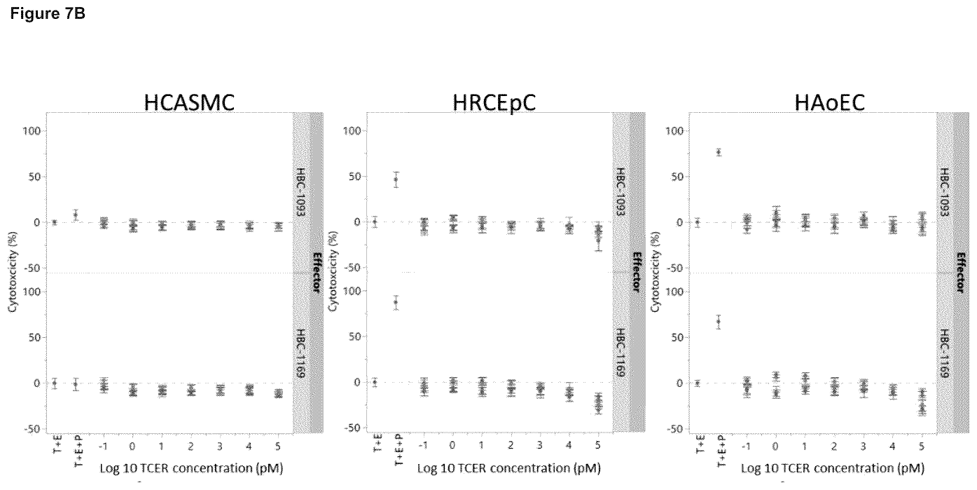

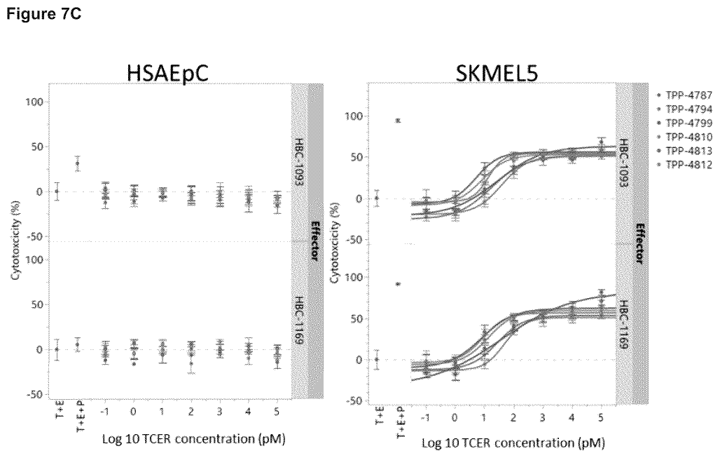

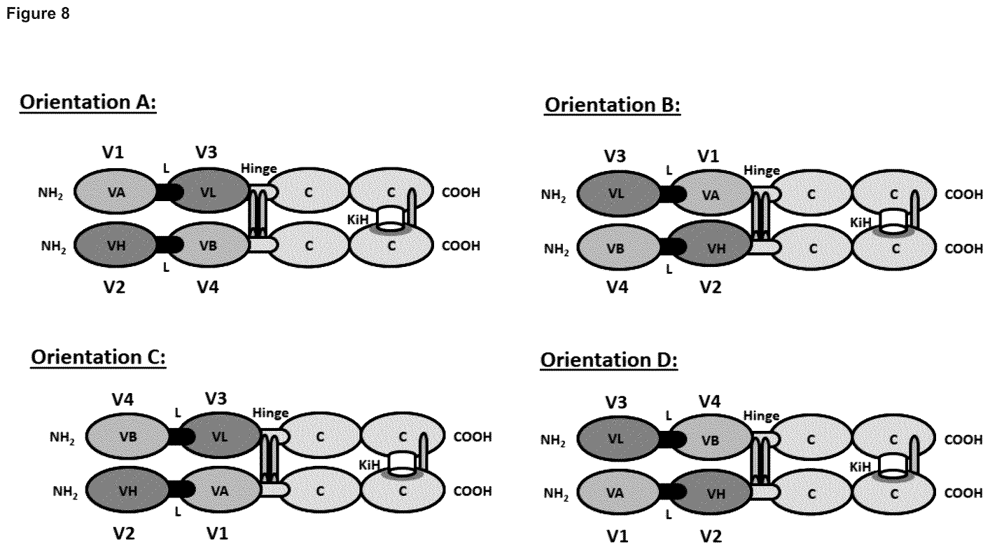

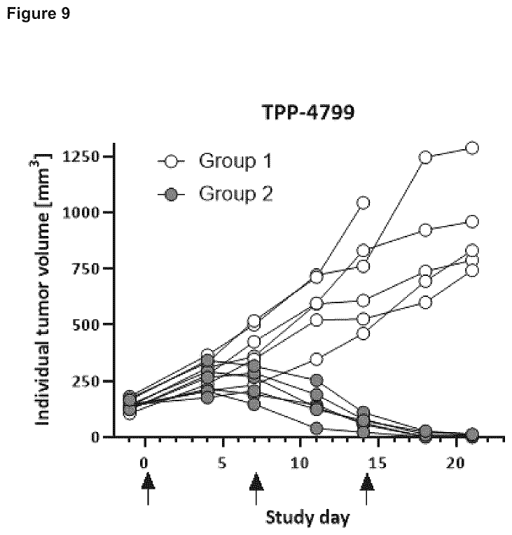

An LDH-release assay with the bispecific TCR/mAb diabody constructs targeting tumor-associated peptide MAGEB2-001 (SEQ ID NO. 1) presented on HLA-A*02 was performed. PBMCs isolated from a healthy donor were co-incubated with cancer cell lines SKMEL-5, RPMI7951, and SCC25 presenting differing amounts of MAGB2-001:HLA-A*02 complexes on the cell surface (194 copies per cell for SKMEL-5, 23 copies per cell for RPMI7951 and 16 copies per cell for SCC25, as determined by M/S analysis) at an effector:target ratio of 10:1, in the presence of increasing concentrations of TCR/mAb diabody molecules, e.g., TPP-4784, TPP-4787, TPP-4810, and TPP-4812 (FIG. 6A); TPP-4788, TPP-4805, and TPP-4813 (FIG. 6B); and TPP-4794, TPP-4798, TPP-4799, and TPP-4606 (FIG. 6C). After 48 hours of co-culture, target cell lysis was quantified utilizing LDH-release assays according to the manufacturer's instructions (Promega).

FIGS. 7A-7C. Safety of bispecific molecules

The reactivity of several MAGEB2-001-targeting bispecific molecules was tested towards the target-positive cell line SKMEL5 (194 cpc) and several primary and iPSC-derived cell types in a cytotoxicity assay with PBMCs from healthy donors as effector cells (FIG. 7A to 7C).

FIG. 8. Distinct orientations of variable domains in bispecific molecules

Arranging the variable domains VA, VB, V_{L} and V_{H} in different positions across the first and/or second polypeptides of a bispecific molecule as disclosed herein results in different “orientations” A to D. In each case, V_{A} and V_{B} are able to form a first binding site (e.g., binding domain A), and V_{L} and V_{H} are able to form a second binding site (e.g., binding domain B). L denotes linker, C denotes constant domain, KiH denotes “knob-into-hole”. An exemplary denotation of V1 to V4 is indicated.

FIG. 9. In vivo efficacy in tumor xenograft bearing NOG mice TCER-mediated in vivo efficacy in human PBMC-engrafted, immunodeficient, female NOG mice against tumor xenografts of BCPAP cells, a tumor cell line with detectable presentation of HLA-A*02/MAGEB2-001. Group 1 represents the vehicle-treated control group and group 2 represents mice treated with TCER TPP-4799 [0.25 mg/kg body weight], once weekly, three times. Each symbol connected by a line represents an individual animal and treatment days are indicated with an arrow.

### DETAILED DESCRIPTION

In order for the present invention to be readily understood, several definitions of terms used in the course of the invention are set forth below.

As used herein, the term “amino acid” refers to one of the 20 naturally occurring amino acids or any non-natural analogues. Preferably, the term “amino acid” refers to one of the 20 naturally occurring amino acids.

As used herein, the terms “polypeptide” or “protein” mean a macromolecule composed of one or more sequence(s) of amino acids. A protein can be a native protein, that is, a protein produced by a naturally-occurring and non-recombinant cell; or it can be produced by a genetically-engineered or recombinant cell, and comprise molecules having the amino acid sequence of the native protein, or molecules having deletions from, additions to, and/or substitutions of one or more amino acids of the native sequence.

As used herein, a “domain” may be any region of a protein, generally defined on the basis of sequence homologies and often referring to a specific structural or functional entity. In the context of variable domains of an antibody or a TCR, the domain comprises the antibody or TCR-derived CDR sequences as also disclosed below.

The term “antigen” or “target antigen” as used herein refers to a molecule or a portion of a molecule or complex that is capable of being bound by an antigen binding site, wherein said antigen binding site is present in an antigen binding protein, preferably an antigen binding protein of the present invention. The antigen in the context of the present invention is melanoma-associated antigen (MAGE) B2 antigenic peptide, more particularly a MAGEB2 antigenic peptide that comprises or consists of the amino acid sequence GVYDGEEHSV (SEQ ID NO:1), in a complex with a MHC protein, such as an HLA protein, for instance HLA-A*02. The peptide GVYDGEEHSV (SEQ ID NO: 1) corresponds to amino acid residue numbers 231-241 of the known MAGEB2 protein. In instances where the MAGEB2 antigenic peptide comprises further amino acids in addition to the amino acid sequence GVYDGEEHSV (SEQ ID NO: 1), it is preferred that the overall length of the MAGEB2 antigenic peptide does not exceed 30 or 20 amino acids, more preferably does not exceed 15 amino acids, even more preferably does not exceed 12 amino acids. In instances where the MAGEB2 antigenic peptide comprises further amino acids in addition to SEQ ID NO: 1, the amino acids of SEQ ID NO: 1 are preferably located within the peptide binding groove of the MHC protein when the antigenic peptide is in a complex with an MHC protein. The person skilled in the art is aware that antigenic peptides presented on MHC I are usually no longer than 12 amino acids.

A “MAGEB2:MHC complex presenting cell” herein refers to a cell that presents on its surface the MAGEB2 antigenic peptide in a complex with an MHC molecule. In preferred embodiments, the MAGEB2:MHC complex presenting cell is a tumor cell, wherein the tumor is preferably a cancer as defined herein below in the section ‘Therapeutic Methods and Uses’. In the context of the present invention, the MAGEB2:MHC complex is over-presented on the cell surface of a MAGEB2:MHC complex presenting cell, compared to levels of said complex on the surface of cells in normal (healthy) tissue (also referred to as “healthy cells”) or on the surface of control cells loaded with a different antigen presenting peptide or no peptide. By “over-presented” is meant that the MAGEB2:MHC complex is present at a level at least 2-fold, preferably between 5-fold to 10-fold of the level present in healthy tissue or control cells. For example, GVYDGEEHSV (SEQ ID NO: 1) was found over-presented in cancers, as disclosed in US20160250307, US20170165337, and US20170253633, respectively; the contents of which are hereby incorporated by reference in their entireties.

The term “immunoglobulin (Ig) domain” in the context of the present invention refers to a protein domain that consists of a 2-layer sandwich of 7-9 antiparallel β-strands arranged in two β-sheets with a Greek key topology. Proteins containing Ig domains are subsumed into the immunoglobulin superfamily, including e.g. antibodies, T cell receptors (TCRs) and cell adhesion molecules. Examples of Ig domains are the variable and constant domains of antibodies and TCRs.

The term “antigen binding protein” herein (occasionally abbreviated to “ABP”) refers to a polypeptide or a complex of two or more polypeptides comprising an antigen binding site that specifically binds to a MAGEB2 antigenic peptide that is in a complex with a major histocompatibility complex (MHC) protein, wherein the MAGEB2 antigenic peptide comprises or consists of the amino acid sequence GVYDGEEHSV (SEQ ID NO: 1), and that polypeptide or the two or more polypeptides comprise(s) the CDRs as herein provided, such as CDRa1, CDRa3, and optionally CDRa2, and CDRb1, CDRb3, and optionally CDRb2. The two or more polypeptides of the antigen binding protein may be covalently or non-covalently linked together. As used in the context of the present specification, the term antigen binding protein includes antigen binding proteins of multiple different formats as described below, including soluble antigen binding proteins, monovalent, bivalent and multivalent antigen binding proteins, monospecific, bispecific and multispecific antigen binding proteins, single chain antigen binding proteins and antigen binding proteins comprising two or more chains, fusion proteins and chimeric proteins. The skilled person is aware that the CDRs provided herein can be included in various formats as disclosed in the prior art, e.g., in Brinkmann U, Kontermann R E. The making of bispecific antibodies (MAbs. 2017 February/March; 9 (2): 182-212, doi: 10.1080/19420862.2016.1268307) or WO 2019/012138.

The term includes antigen binding proteins having the overall structure of a TCR, an antibody and/or a chimeric antigen receptor (CAR). The antigen binding protein of the present invention comprises the TCR-derived CDRs, in particular a variable domain V_{A} comprising TCR-derived CDRa1, CDRa3, and optionally CDRa2, and a variable domain V_{B} comprising TCR-derived CDRb1, CDRb3, and optionally CDRb2. Antigen binding proteins of the invention comprise a variable domain V_{A} comprising complementarity determining regions (CDRs) CDRa1, CDRa2, and CDRa3, e.g. on a first polypeptide, and a variable domain V_{B} comprising CDRb1, CDRb2, and CDRb3, e.g. on a second polypeptide, wherein CDRa1, CDRa2, CDRa3, CDRb1, CDRb2 and CDRb3 form an “antigen binding domain A”. “Antigen binding domain A” denotes a binding domain that binds to the antigenic peptide (MAGEB2 antigenic peptide in the context of the present invention) that is in a complex with a major histocompatibility complex (MHC) protein. In a particular embodiment, the entire V_{A} domain and/or the entire V_{B} domain are TCR-derived and are thus TCR alpha, beta, gamma or delta variable domains (V_{α}, V_{β}, V_{γ} or V_{δ}). In preferred embodiments, the antigen binding protein is a TCR or functional fragment(s) thereof, e.g. the variable domains V_{A} and V_{B} of the TCR. In particular embodiments, the antigen binding protein of the present invention comprises CDRs and optionally the V_{A} and V_{B} as herein provided, and comprises further (an) additional domain(s) fused directly or indirectly to V_{A} or V_{B}. The further domains may form (an) additional binding domain(s) or (a) binding site(s). For example and in particular embodiments, the additional binding domains may form antigen binding domain B. Further binding domains may be comprised that, e.g., form further antigen binding domains, e.g. antigen binding domain C, etc. The additional/further domains comprised in the antigen binding protein may also be a further protein.

Such an antigen binding protein can also be referred to as “fusion protein”. Examples of additional domains comprised in an antigen binding protein of the invention that is a fusion protein are listed below.

If the antigen binding protein is a bispecific or multispecific antigen binding protein, it comprises—in addition to V_{A} and V_{B} as herein defined—at least one more binding domain/site, e.g. a variable domain, preferably two variable domains, and optionally a further domain, such as constant domain(s), wherein the variable and/or constant domains may be derived from an antibody or TCR. In this aspect, the antigen binding protein may thus comprise two different antigen binding sites (one formed by V_{A} and V_{B} and one formed by the additional at least one, preferably two, variable domains) and is able to specifically bind to two different antigens. In particular embodiments, the antigen binding protein comprises TCR-derived V_{A} and V_{B} and in addition two antibody-derived variable domains, in particular V_{L} and VH. Such constructs comprising elements of both antibodies and TCRs represent hybrid formats and may be e.g. referred to as bispecific TCR-antibody fusion protein or bispecific TCR molecules/proteins as herein used in the exemplary part. In the bispecific fusion proteins, the variable domains may be included in different formats and may be arranged in various orientations. Techniques to produce such bispecific fusion proteins are known to the skilled in the art as also disclosed herein below, who can thus use the variable domains as herein defined to generate and produce bispecific antigen binding proteins in various formats. The skilled person is capable of selecting suitable linkers to ensure folding in the desired conformation. Antigen binding proteins described herein may take the form of a TCR, an antibody, including engineered TCRs and antibodies, such as single chain fragment variable (scFv), disulfide-stabilized Fv (dsFv), scTVs, Fab, Fab′, F(ab′)2, nanobody, DARPin, Knottin, diabody, single-chain diabody or oligomers thereof.

The term “bispecific” in the context of the present invention refers to antigen binding proteins with at least two valences and binding specificities for at least two different antigens (preferably two different antigens) and thus comprises at least two antigen-binding sites. The term “valence” refers to the number of binding sites of an antigen binding protein, e.g. a bivalent antigen binding protein relates to an antigen binding protein that has two binding sites. The binding sites may bind to the same or different targets, i.e. a bivalent antigen binding protein may be monospecific, e.g. binding one target, or bispecific, e.g. binding two different targets. The bispecific antigen-binding molecules of the present invention comprise at least one antigen binding domain A comprising TCR-derived CDRs. In preferred embodiments, the antigen-binding molecules of the present invention comprise at least one TCR-derived antigen-binding site. The bispecific antigen binding proteins of the invention may be referred to as a bispecific TCR, or as a bispecific TCR/mAb diabody. The antigen binding protein of this embodiment may also be referred to as a T cell engaging receptor, or TCER®.

Bispecific antigen binding proteins of the invention, such as TCER®, may comprise a VH comprising a CDRH1, a CDRH2 and a CDRH3, and a V_{L} comprising a CDRL1, a CDRL2, and a CDRL3, wherein CDRH1, CDRH2, CDRH3, CDRL1, CDRL2 and CDRL3 form an antigen binding domain B. The antigen binding domain B denotes a binding domain that specifically binds to immune cells, preferably T cells or natural killer (NK) cells.

“TCER®” are soluble antigen binding proteins comprising two antigen binding domains, a first variable domain (VA) and second variable domain (VB) as defined in the context of the invention and a further antigen binding domain that is formed by the heavy and light chain variable domains of an antibody, also referred to as “recruiter”, such as variable heavy (VH) and variable light (VL) domains directed against a T cell antigen (such as CD3) or directed against TCRα/β or a combination thereof.

“V_{A}” or Va in the context of the present invention refers to a TCR variable domain comprising TCR-derived CDR sequences and TCR-derived framework sequences. The CDR and framework sequences may be derived from a variable domain of a TCR α-chain (V_{α}), β-chain (V_{β}), γ-chain (V_{γ}) or δ-chain (V_{δ}), preferably from a Vα. The sequences surrounding the CDRs, i.e. the framework sequences, may be derived from a variable domain of a TCR, i.e. a variable domain of a TCR α-chain, β-chain, γ-chain or δ-chain, or from a variable domain of an antibody, preferably from a variable domain of a TCR α-chain. In the examples, various framework and CDR mutations/substitutions are shown.

The CDR and framework sequences of the V_{A} domain in context of the present invention may not necessarily be derived from the same TCR chain. For example, the CDRs derived from one TCR variable domain (of the donor TCR) could also be grafted onto another TCR variable domain (of the acceptor TCR). For example, the donor TCR may comprise a V_{A} encoded by e.g. TRAV5*01 and TRAJ31*01, and the acceptor TCR may comprise a V_{A} encoded by e.g. TRAV14 and TRAJ33.

“V_{B}” or Vb in the context of the present invention refers to a variable domain comprising TCR-derived CDR sequences and TCR-derived framework sequences. The CDR and framework sequences may be derived from a variable domain of a TCR α-chain (V_{α}), β-chain (V_{β}), γ-chain (V_{γ}) or δ-chain (V_{δ}), preferably from a Vβ. The sequences surrounding the CDRs, i.e. the framework sequences, may be derived from a variable domain of a TCR, i.e. a variable domain of a TCR α-chain, β-chain, γ-chain or δ-chain, or from a variable domain of an antibody, preferably from a variable domain of a TCR β-chain. In the examples, various framework and CDR mutations/substitutions are shown.

The CDR and framework sequences of the V_{B} domain in context of the present invention may not necessarily be derived from the same TCR. For example, CDRs derived from one TCR variable domain (of the donor TCR) could be grafted onto another TCR variable domain (of the acceptor TCR). For example, the donor TCR may comprise a V_{B} encoded by e.g. TRBV29-1*01 and TRBJ1-2*01, and the acceptor TCR may comprise a V_{B} encoded by e.g. TRBV27 and TRBJ1-5.
V_{α} in the context of the present invention refers to a variable domain of a TCR α-chain.
V_{β} in the context of the present invention refers to a variable domain of a TCR β-chain.
V_{γ} in the context of the present invention refers to a variable domain of a TCR γ-chain.
V_{δ} in the context of the present invention refers to a variable domain of a TCR δ-chain.
V_{L} in the context of the present invention refers to a variable domain of an antibody light chain.
V_{H} in the context of the present invention refers to a variable domain of an antibody heavy chain.
C_{L} in the context of the present invention may refer to a constant domain of an antibody light chain.
C_{H1}, C_{H2} and C_{H3} in the context of the present invention may refer to constant domains of an antibody heavy chain, in particular an IgG heavy chain.

The constant domains as herein disclosed may be comprised in the antigen binding protein and may further improve certain characters of the antigen binding protein and thus can be present or absent. For example, the stability or half life of the antigen binding protein may be increased or the purification of the antigen binding protein may be eased. The constant domains may also comprise a further binding domain. Exemplary constant domains may be constant domains, e.g. of an antibody, for example, CL or CH, and/or F_{C} domains/portions. The constant domains may also be albumin, unstructured polypeptides, and/or Leu-Zipper etc.

The term “epitope”, also known as antigenic determinant, is the part of an antigen that is recognized by the immune system. As used herein, the term epitope comprises the terms “structural epitope” and “functional epitope”. The “structural epitope” are those amino acids of the antigen, e.g. peptide-MHC complex, that are covered by the antigen binding protein when bound to the antigen. Typically, all amino acids of the antigen are considered covered that are within 5 Å of any atom of an amino acid of the antigen binding protein. The structural epitope of an antigen may be determined by art known methods including X-ray crystallography or NMR analysis. The structural epitope of an antibody typically comprises 20 to 30 amino acids. The structural epitope of a TCR typically comprises 20 to 30 amino acids. The “functional epitope” as herein defined is a subset of those amino acids forming the structural epitope and comprises the amino acids of the antigen that are critical for formation of the interface with the antigen binding protein of the invention or functional fragment thereof, either by directly forming non-covalent interactions such as H-bonds, salt bridges, aromatic stacking or hydrophobic interactions or by indirectly stabilizing the binding conformation of the antigen and is, for instance, determined by mutational scanning. In the context of the present invention, the functional epitope is also referred to as “binding motif”. Typically, the functional epitope of an antigen bound by an antibody comprises between 4 and 6 amino acids (see Example 2 herein). Typically, the functional epitope of a peptide-MHC complex comprises between 2 to 6 or 7 amino acids of the peptide and 2 to 7 amino acids of the MHC molecule. Since MHC I presented peptides typically have a length between 8 to 10 amino acids only a subset of amino acids of each given peptide is part of the functional epitope of a peptide-MHC complex. The epitope, in particular the functional epitope, bound by the antigen binding proteins of the present invention comprises or consists of the amino acids of the antigen that are required for formation of the binding interface.

The “Major Histocompatibility Complex” (MHC) is a set of cell surface proteins essential for the acquired immune system to recognize foreign molecules in vertebrates, which in turn determines histocompatibility. The main function of MHC molecules is to bind to antigens derived from pathogens and display them on the cell surface for recognition by the appropriate T cells. The human MHC is also called the HLA (human leukocyte antigen) complex (or just HLA). Thus, in a preferred embodiment, MHC is HLA. The MHC gene family is divided into three subgroups: class I, class II, and class III. Complexes of peptide and MHC class I molecules (MHC I) are usually recognized by CD8-positive T cells (CD8+ T cells) bearing the appropriate T cell receptor (TCR), whereas complexes of peptide and MHC class II molecules (MHC II) are usually recognized by CD4-positive helper-T cells (CD4+ T cells) bearing the appropriate TCR. CD4 and CD8 usually function as co-receptors of a TCR in binding to MHC I and MHC II, respectively. In some exceptional cases, complexes of peptide and MHC I are recognized by CD8-negative (in particular CD8-negative, CD4-positive) T cells (Soto et al., 2013, Cancer Immunol Immunother. 2013 February; 62 (2): 359-369). Since the responses of CD8-positive and CD4-positive T cells contribute jointly and synergistically to the anti-tumor effect, the identification and characterization of tumor-associated antigens and corresponding T cell receptors is important in the development of cancer immunotherapies such as vaccines and cell therapies. The HLA-A gene is located on the short arm of chromosome 6 and encodes the larger, α-chain, constituent of HLA-A. Variation of HLA-A α-chain is key to HLA function. This variation promotes genetic diversity in the population. Since each HLA has a different affinity for peptides of certain structures, greater variety of HLAs means greater variety of antigens to be ‘presented’ on the cell surface. The MHC class I HLA protein in the context of the present disclosure may be an HLA-A, HLA-B or HLA-C protein, suitably HLA-A protein, for example HLA-A*02. In the MHC class I dependent immune reaction, peptides not only have to be able to bind to certain MHC class I molecules expressed by tumor cells, they subsequently also have to be recognized by T cells bearing specific T cell receptors (TCR).

“Antigenic peptide in a complex with an MHC protein”, herein refers to an antigenic peptide that is non-covalently bound to an MHC molecule. In particular, the antigenic peptide is located to a “peptide-binding groove” formed by the MHC molecule. A complex of an MHC molecule and an antigenic peptide is herein also referred to as “peptide-MHC complex” or “pMHC complex”. In the case of the MAGEB2 antigenic peptide, the complex is also referred to as “MAGEB2 antigenic peptide-MHC complex” or “MAGEB2:MHC complex”.

The term “HLA-A*02” signifies a specific HLA allele, wherein the letter A signifies the allele and “*02” indicates the A2 serotype.

“At least one” herein refers to one or more of the specified objects such as 1, 2, 3, 4, 5 or 6 or more of the specified objects. For example, at least one binding site herein refers to 1, 2, 3, 4, 5 or 6 or more binding sites.

The term “TCR” as used herein includes engineered TCRs.

A “native TCR” refers to a wildtype TCR that can be isolated from nature. Native TCRs are heterodimeric cell surface proteins of the immunoglobulin super-family, which are associated with invariant proteins of the CD3 complex involved in mediating signal transduction. Native heterodimeric TCRs exist in αβ and γδ forms, which are structurally similar but have distinct locations and probably functions. Native, full-length αβ heterodimeric TCRs consist of an α-chain and a β-chain. The α-chain comprises a variable region (V region) encoded by a TRAV gene, a joining region (J region) encoded by a TRAJ gene, and a constant region (C region) encoded by a TRAC gene. The β-chain comprises a variable region (V region) encoded by a TRBV gene, a joining region (J region) encoded by a TRBJ gene and a constant region (C region) encoded by a TRBC gene, and usually a short diversity region (D region) encoded by a TRBD gene between the V and J regions, although this D region is often considered as part of the J region (Lefranc, (2001), Curr Protoc Immunol Appendix 1: Appendix 10). The genes encoding different α-chain and β-chain variable, joining and constant regions are referred to in IMGT nomenclature by unique numbers (Folch and Lefranc, (2000), Exp Clin Immunogenet 17 (1): 42-54; Scaviner and Lefranc, (2000), Exp Clin Immunogenet 17 (2): 83-96; LeFranc and LeFranc, (2001), “T cell Receptor Factsbook”, Academic Press). Further information on TCR genes can be found in the international ImMunoGeneTics information System®, Lefranc M P et al., (Nucleic Acids Res. 2015 January; 43 (Database issue): D413-22; and http://www.imgt.org/).

The alpha chain TRAC constant domain sequence and the beta chain TRBC1 or TRBC2 constant domain are in the following, also referred to as TCR constant domain sequences. In one embodiment, the TCR constant domain sequences may be derived from any suitable species, such as any mammal, e.g., human, rat, monkey, rabbit, donkey, or mouse, preferably human. In some preferred embodiments, the TCR constant domain sequences may be slightly modified, for example, by the introduction of heterologous sequences, preferably mouse sequences, which may increase TCR expression and stability. Also, further stabilizing mutations as known from the state of the art (e.g. WO 2018/104407, PCT/EP2018/069151, WO 2011/044186, WO 2014/018863) may be introduced, such as replacement of unfavorable amino acids in the variable regions and/or the introduction of a disulfide bridge between the TCR C domains and the removal of unpaired cysteine.

On the protein level, TCR α-, β-, γ- and δ-chains comprise two immunoglobulin domains, the variable domain and the constant domain. The variable domain corresponds to the V(D)J region. The constant domain corresponds to the C region. The constant domain is the membrane-proximal domain and in the context of the present invention also includes the transmembrane (TM) domain and a short cytoplasmic tail. Each of the constant and variable domains include an intra-chain disulfide bond. The variable domains (V_{α} and V_{β} in αβ TCRs and V_{γ} and V_{δ} in γδ TCRs) contain highly polymorphic loops comprising the complementarity determining regions (CDRs).

Each TCR variable domain comprises three “TCR complementarity determining regions (CDRs)” embedded in a framework sequence, one being the hypervariable region named CDR3. In the context of the present invention, CDRa1, CDRa2 and CDRa3 denote α-chain CDRs, and CDRb1, CDRb2 and CDRb3 denote β-chain CDRs. The sequences encoding CDRa1 and CDRa2 are comprised in TRAV, the sequences encoding CDRa3 are comprised in TRAV and TRAJ, the sequences encoding CDRb1 and CDRb2 are comprised in TRBV, and the sequences encoding CDRb3 are comprised in TRBV, TRBD and TRBJ. In TCRs, the CDR1 and CDR3 amino acid residues make contact with the antigenic peptide, while the CDR2 amino acid residues mainly contact the HLA molecule (Stadinski et al., J Immunol. 2014 Jun. 15; 192 (12): 6071-6082; Cole et al., J Biol Chem. 2014 Jan. 10; 289 (2): 628-38). The antigen specificity of a TCR is thus defined by the CDR3 and CDR1 sequences. The CDR2 sequences are not required for the determination of antigen specificity, but may play a role in the overall affinity of a TCR towards a peptide:MHC complex.

“TCR framework regions” (FRs) refer to amino acid sequences interposed between the CDRs, i.e. to those portions of the variable domains that are to some extent conserved among different TCRs. The α-, β-, γ- and δ-chain variable domains each have four FRs, herein designated FR1-a, FR2-a, FR3-a, FR4-a (for an α- or γ-chain), and FR1-b, FR2-b, FR3-b, FR4-b (for a β- or δ-chain), respectively. Accordingly, an α-chain or γ-chain variable domain may be described as (FR1-a)-(CDRa1)-(FR2-a)-(CDRa2)-(FR3-a)-(CDRa3)-(FR4-a) and a β- or δ-chain variable domain may be described as (FR1-b)-(CDRb1)-(FR2-b)-(CDRb2)-(FR3-b)-(CDRb3)-(FR4-b). In the context of the present invention, the CDR/FR sequences in an α-, β-, γ- or δ-chain variable domain is determined based on IMGT definition (Lefranc et al., Dev. Comp. Immunol., 2003, 27 (1): 55-77; www.imgt.org). Accordingly, CDR/FR amino acid positions when related to TCR or TCR-derived domains are indicated according to said IMGT definition. Preferably, the IMGT position of the CDR/FR amino acid positions of the variable domain Vα is given in analogy to the IMGT numbering of TRAV5*01 and/or the IMGT position of the CDR/FR amino acid positions of the variable domain V_{B} is given in analogy to the IMGT numbering of TRBV29-1*01.

An “engineered TCR” may be a protein resembling a native TCR, but comprising modifications in the variable and/or constant domains, e.g. a humanized TCR or a TCR with improved/altered characteristics (e.g. improved binding, heterodimerization or expression level), or may be a soluble TCR and/or a single-chain TCR, a monovalent, bivalent or multivalent TCR, a monospecific, bispecific or multispecific TCR, and/or a functional fragment of a TCR, or functional fragments of one or more TCRs, or a fusion protein and/or chimeric protein comprising a functional fragment of a TCR or functional fragments of one or more TCRs.

The term “F_{C} domain” as used in the context of the present invention encompasses native F_{C} domains and F_{C} domain variants and sequences as also further defined herein below. As with F_{C} variants and native F_{C} molecules, the term “F_{C} domain” includes molecules in monomeric or multimeric form, whether digested from whole antibody or produced by other means.

The term “native F_{C}” as used herein refers to a molecule comprising the sequence of a non-antigen binding fragment resulting from digestion of an antibody or produced by other means, whether in monomeric or multimeric form, and may contain the hinge region. The original immunoglobulin source of the native F_{C} is, in particular, of human origin and can be any of the immunoglobulins, preferably IgG1 or IgG2, most preferably IgG1. Native F_{C} molecules are made up of monomeric polypeptides that can be linked into dimeric or multimeric forms by covalent (i.e., disulfide bonds) and non-covalent association. The number of intermolecular disulfide bonds between monomeric subunits of native F_{C} molecules ranges from 1 to 4 depending on class (e.g., IgG, IgA, and IgE) or subclass (e.g., IgG1, IgG2, IgG3, IgA1, and IgGA2). One example of a native F_{C} is a disulfide-bonded dimer resulting from papain digestion of an IgG. The term “native Fc” as used herein is generic to the monomeric, dimeric, and multimeric forms. An example of a native F_{C} amino acid sequence is provided in WO2021023658 (A1).

The “hinge” or “hinge region” or “hinge domain” refers typically to the flexible portion of a heavy chain located between the C_{H1} domain and the C_{H2} domain. It is approximately 25 amino acids long, and is divided into an “upper hinge,” a “middle hinge” or “core hinge,” and a “lower hinge.” A “hinge subdomain” refers to the upper hinge, middle (or core) hinge or the lower hinge. The amino acids sequences of the hinges of an IgG1, IgG2, IgG3 and IgG4 molecule are provided, for example, in WO2021023658 (A1).

In the context of the present invention, in case reference is made to amino acid positions in the F_{C} domain, these amino acid positions or residues are indicated according to the EU numbering system as described, for example in Edelman, G. M. et al., Proc. Natl. Acad. USA, 63, 78-85 (1969).

The term “F_{C} variant” as used herein refers to a molecule or sequence that is modified from a native F_{C} but still comprises a binding site for the salvage receptor, F_{C}Rn (neonatal Fc receptor). Exemplary F_{C} variants, and their interaction with the salvage receptor, are known in the art. Thus, the term “F_{C} variant” can comprise a molecule or sequence that is humanized from a non-human native Fc. Furthermore, a native F_{C} comprises regions that can be removed because they provide structural features or biological activity that are not required for the antigen binding proteins of the invention. Thus, the term “F_{C} variant” comprises a molecule or sequence that lacks one or more native F_{C} sites or residues, or in which one or more F_{C} sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

Accordingly, in one embodiment the F_{C}-domain, such as F_{C1} and/or F_{C2}, comprises a hinge domain.

In one embodiment, the F_{C}-domain is a human IgG F_{C} domain, preferably derived from human IgG1, IgG2, IgG3 or IgG4, preferably IgG1 or IgG2, more preferably IgG1.

In some embodiments, in particular, when the antigen binding protein comprises two F_{C} domains, e.g. in the TCER® format described herein below (such as F_{C1} and F_{C2}), the two Fc domains may be of the same immunoglobulin isotype or isotype subclass or of different immunoglobulin isotype or isotype subclass, preferably of the same. Accordingly, in some embodiments F_{C1} and F_{C2}, are of the IgG1 subclass, or of the IgG2 subclass, or of the IgG3 subclass, or of the IgG4 subclass, preferably of the IgG1 subclass, or of the IgG2 subclass, more preferably of the IgG1 subclass.

In some embodiments, the F_{C} domain is a variant F_{C} domain and thus comprises one or more of the amino acid substitutions described herein below.

In some embodiments, the F_{C} domain comprises or further comprise the “RF” and/or “Knob-into-hole” mutation, preferably the “Knob-into-hole”.

The “RF mutation” generally refers to the amino acid substitutions of the amino acids HY into RF in the C_{H3} domain of F_{C} domains, such as the amino acid substitution H435R and Y436F in C_{H3} domain as described by Jendeberg, L. et al. (1997, J. Immunological Meth., 201:25-34) and is described as advantageous for purification purposes as it abolishes binding to protein A. In case the antigen binding protein comprises two F_{C}-domains, the RF mutation may be in one or both, preferably in one F_{C}-domain.

The “Knob-into-Hole” or also called “Knob-into-Hole”-technology refers to amino acid substitutions T366S, L368A and Y407V (Hole) and T366W (Knob) both in the C_{H3}-C_{H3} interface to promote heteromultimer formation. Those knob-into-hole mutations can be further stabilized by the introduction of additional cysteine amino acid substitutions Y349C and S354C. The “Knob-into-Hole” technology together with the stabilizing cysteine amino acid substitutions has been described in patents U.S. Pat. Nos. 5,731,168 and 8,216,805.

In the context of the present invention, the “Knob” mutation together with the cysteine amino acid substitution S354C is, for example, present in the F_{C} domain comprising or consisting of the amino acid sequence of SEQ ID NO: 24 and the “Hole” mutation together with the cysteine amino acid substitutions Y349C is present in the F_{C} domain comprising or consisting amino acid sequence of SEQ ID NO: 25.

In some embodiments, the F_{C} domain of one of the polypeptides, for example F_{C1}, comprises the amino acid substitution T366W (Knob) in its C_{H3} domain and the F_{C} domain of the other polypeptide, for example F_{C2}, comprises the amino acid substitution T366S, L368A and Y407V (Hole) in its C_{H3} domain, or vice versa.

In some embodiments, the F_{C} domain of one of the polypeptides, for example F_{C1}, comprises or further comprises the amino acid substitution S354C in its C_{H3} domain and the Fc domain of the other polypeptide, for example F_{C2}, comprises or further comprises the amino acid substitution Y349C in its C_{H3} domain, or vice versa.

Accordingly, in some embodiments, the F_{C} domain of one of the polypeptides, for example F_{C1}, comprises the amino acid substitutions S354C and T366W (Knob) in its C_{H3} domain and the F_{C} domain of the other polypeptide, for example F_{C2}, comprises the amino acid substitution Y349C, T366S, L368A and Y407V (Hole) in its C_{H3} domain, or vice versa.

This set of amino acid substitutions can be further extended by inclusion of the amino acid substitutions K409A on one polypeptide and F405K in the other polypeptide as described by Wei et al. (Structural basis of a novel heterodimeric F_{C} for bispecific antibody production, Oncotarget. 2017). Accordingly, in some embodiments, the F_{C} domain of one of the polypeptides, for example F_{C1}, comprises or further comprises the amino acid substitution K409A in its C_{H3} domain and the F_{C} domain of the other polypeptide, for example F_{C2}, comprises or further the amino acid substitution F405K in its C_{H3} domain, or vice versa.

In some cases, artificially introduced cysteine bridges may improve the stability of the antigen binding proteins, optimally without interfering with the binding characteristics of the antigen binding proteins. Such cysteine bridges can further improve heterodimerization.

Further amino acid substitutions, such as charged pair substitutions, have been described in the art, for example in EP 2 970 484 to improve the heterodimerization of the resulting proteins.

Accordingly in one embodiment, the F_{C} domain of one of the polypeptides, for example F_{C1}, comprises or further comprises the charge pair substitutions E356K, E356R, D356R, or D356K and D399K or D399R, and the F_{C} domain of the other polypeptide, for example F_{C2}, comprises or further comprises the charge pair substitutions R409D, R409E, K409E, or K409D and N392D, N392E, K392E, or K392D, or vice versa.

In a further embodiment, the F_{C} domain on one or both, preferably both polypeptide chains can comprise one or more alterations that inhibit F_{C} gamma receptor (FcyR) binding. Such alterations can include L234A, L235A.

With the inclusion of Fc-parts consisting of Hinges, C_{H2} and C_{H3} domains, or parts thereof, into antigen binding proteins, more particularly into bispecific antigen binding proteins the problem of unspecific immobilization of these molecules, induced by Fc:Fc-gamma receptor (FcgR) interactions arose. FcgRs are composed of different cell surface molecules (FcgRI, FcgRIIa, FcgRIIb, FcgRIII) binding with differing affinities to epitopes displayed by Fc-parts of IgG-molecules. As such an unspecific (i.e. not induced by either of the two binding domains of a bispecific molecule) immobilization is unfavorable due to i) influence on pharmacokinetics of a molecule and ii) off-target activation of immune effector cells various Fc-variants and mutations to ablate FcgR-binding have been identified. In this context, Morgan et al. 1995, Immunology (The N-terminal end of the C_{H2} domain of chimeric human IgG1 anti-HLA-DR is necessary for C1q, FcyRI and FcyRIII binding) disclose the exchange of the residues 233-236 of human IgG1 with the corresponding sequence derived from human IgG2, i.e., the residues 233P, 234V and 235A and wherein no amino acid is present at position 236, resulting in abolished FcgRI binding, abolished C1q binding and diminished FcgRIII binding. EP1075496 discloses antibodies and other Fc-containing molecules with variations in the F_{C} domain (such as one or more of 233P, 234V, 235A and no residue or G in position 236 and 327G, 330S and 331S) wherein the recombinant antibody is capable of binding the target molecule without triggering significant complement dependent lysis, or cell mediated destruction of the target.

Accordingly, in some embodiments, the F_{C} domain comprises or further comprises one or more of the amino acids or deletions selected from the group consisting of 233P, 234V, 235A, 236 (no residue) or G, 327G, 330S, 331S, preferably, the F_{C} domain comprises or further comprises the amino acids 233P, 234V, 235A, 236 (no residue) or G and one or more amino acids selected from the group consisting of 327G, 330S, 331S, most preferably, the F_{C} domain comprises or further comprises the amino acids 233P, 234V, 235A, 236 (no residue) and 331S.

In one further embodiment, the F_{C} domain comprises or further comprises the amino acid substitution N297Q, N297G or N297A, preferably N297Q.

The amino acid substitution “N297Q”, “N297G” or “N297A” refer to amino acid substitutions at position 297 that abrogate the native N-Glycosylation site within the F_{C}-domain. This amino acid substitution further prevents Fc-gamma-receptor interaction and decreases the variability of the final protein products, i.e. the antigen binding proteins of the present invention, due to sugar residues as described for example in Tao, M H and Morrison, SL (J Immunol. 1989 Oct. 15; 143 (8): 2595-601).

In one further embodiment, in particular when no light chain, the F_{C} domain comprises or further comprises the amino acid substitution S220C. The amino acid substitution “S220C” deletes the cysteine forming the C_{H1}-C_{L} disulfide-bridge.

In some embodiments, the F_{C} domain comprises or further comprises at least two additional cysteine residues, for example S354C and Y349C or L242C and K334C, wherein S354C is in the F_{C}-domain of one polypeptide, such as F_{C1}, and Y349C is in the F_{C} domain of the other polypeptide, such as F_{C2}, to form a heterodimer and/or wherein L242C and K334C are located in the same F_{C}-domain, either in the F_{C1} or F_{C2} of one or both polypeptides to form a intradomain C—C bridge.

Additional substitutions and description may be found in U.S. patent application No. 20180162922 the contents of which is incorporated by reference in its entirety.

It may be also desirable to modify the antigen binding protein of the present invention with respect to effector function, e.g. so as to enhance or reduce antigen-dependent cell-mediated cytotoxicity (ADCC) and/or complement dependent cytotoxicity (CDC) of the antigen binding protein. This may be achieved by introducing one or more amino acid substitutions in an F_{C} domain of the antigen binding protein, herein also called Fc-variants in the context with the antigen binding proteins of the present invention. Alternatively, or additionally, cysteine residue(s) may be introduced in the F_{C} domain, thereby allowing inter-chain disulfide bond formation in this region. The heterodimeric antigen binding protein thus generated may have improved or reduced internalization capability and/or increased complement-mediated cell killing and/or antibody-dependent cellular cytotoxicity (ADCC) (Caron P C. et al. 1992; and Shopes B. 1992).

Another type of amino acid modification of the antigen binding protein of the invention may be useful for altering the original glycosylation pattern of the antigen binding protein, i.e. by deleting one or more carbohydrate moieties found in the antigen binding protein, and/or adding one or more glycosylation sites that are not present in the antigen binding protein. The presence of either of the tripeptide sequences asparagine-X-serine, and asparagine-X-threonine, where X is any amino acid except proline, creates a potential glycosylation site. Addition or deletion of glycosylation sites to the antigen binding protein is conveniently accomplished by altering the amino acid sequence such that it contains one or more of the above-described tripeptide sequences (for N-linked glycosylation sites).

Another type of modification involves the removal of sequences identified, either in silico or experimentally, as potentially resulting in degradation products or heterogeneity of antigen binding protein preparations. As examples, deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in an antigen binding protein of the invention, it may therefore be desirable to remove the site, typically by conservative substitution to remove one of the implicated residues. Such substitutions in a sequence to remove one or more of the implicated residues are also intended to be encompassed by the present invention.

Another type of covalent modification involves chemically or enzymatically coupling glycosides to the antigen binding protein. These procedures are advantageous in that they do not require production of antigen binding protein in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. For example, such methods are described in WO 87/05330.

Removal of any carbohydrate moieties present on the antigen binding protein may be accomplished chemically or enzymatically. Chemical deglycosylation requires exposure of the antigen binding protein to the compound trifluoromethanesulfonic acid, or an equivalent compound. This treatment results in the cleavage of most or all sugars except the linking sugar (N-acetylglucosamine or N-acetylgalactosamine), while leaving the antigen binding protein intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Another type of covalent modification of the antigen binding protein comprises linking the antigen binding protein to one of a variety of non-proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

“Functional fragment of a TCR” refers to a fragment of a TCR that retains or substantially retains the affinity, functional avidity and/or specificity of the TCR from which it is derived for a target antigen. Exemplary functional fragments, e.g. V_{A} and V_{B}, are herein demonstrated in the examples below. As binding to the target antigenic peptide is defined by the CDR1 and CDR3 sequences, and binding to the target antigenic peptide MHC complex is defined by CDR1, CDR2 and CDR3, antigen binding proteins comprising the CDR1 and CDR3 and optionally CDR2 sequences of a TCR retain the affinity, functional avidity and/or specificity of the parental TCR for a target antigen. The person skilled in the art is aware that the CDRs are usually interspersed with framework regions (FRs), however the specific amino acid sequences of the framework regions may not directly be involved in target antigen specificity. Examples of functional TCR fragments include the variable domains, such as TCR alpha, beta, gamma or delta variable domains, or fragments of the α, β, δ or γ chain, such as an α, β, δ or γ chain without transmembrane domain and short cytoplasmic tail. The term “fragment” as used herein refers to naturally occurring fragments (e.g. splice variants or peptide fragments) as well as artificially constructed fragments, in particular to those obtained by gene-technological means. The functional fragments of the TCRs may be comprised in various formats in the antigen binding protein of the invention.

A functional fragment of a TCR may have retained or substantially retained the affinity for a target antigen, if, for example, the KD for binding to the target antigen measured as outlined below is identical to the KD of the TCR or is increased or reduced, preferably reduced, no more than 10×, 5×, 3×, or 2×. The functional fragments as herein provided in the examples have improved affinity for the target antigen, e.g. by at least 100×, at least 500× or at least 1000× fold, compared to the variable domains as comprised in the naturally occurring TCR from which they were derived.

A functional fragment of a TCR is considered to have retained or substantially retained the functional avidity for a target antigen, if, for example, the functional avidity for the target antigen is identical to that of the TCR or is increased or reduced, preferably reduced, no more than 50%, 40%, 30%, 20%, 15%, 10%, 8%, 5%, 3%, 2% or 1%. In particular, a functional fragment of a TCR is considered to have retained or substantially retained the functional avidity for a target antigen, if, for example, its cytotoxic activity in response to the target of the parent protein measured in a cytotoxicity assay is identical to the cytotoxic activity of the TCR or is increased or reduced, preferably reduced, no more than 50%, 40%, 30%, 20%, 15%, 10%, 8%, 5%, 3%, 2% or 1%, preferably 10%, 8%, 5%, 3%, 2% or 1%.

A functional fragment of a TCR is considered to have retained or substantially retained the specificity for a target antigen (i.e. the ability to specifically bind to a target antigen), if it does not significantly bind to peptides other than the target antigenic peptide of the TCR.

“Does not significantly bind” in the context of antigenic peptide variants and in the context of antigen binding proteins of the invention, in particular soluble antigen binding proteins of the invention, denotes, typically in a binding assay, for example biolayer interferometry, a relative response signal was determined for antigenic peptide variants that is not higher than 30%, not higher than 25%, not higher than 20%, not higher than 15%, preferably not higher than 20% of the signal obtained for binding to the MAGEB2 peptide consisting of the amino acid sequence of SEQ ID NO: 1, preferably in the same experimental conditions. For example, the relative response signal obtained for the similar peptide SP-02-1640 consisting of the amino acid sequence ‘RLYDGLFKVI’ of SEQ ID NO: 106 is not higher than 30% of the signal obtained in the same experimental conditions for binding to the MAGEB2 peptide consisting of the amino acid sequence of SEQ ID NO: 1.

The terms “α/β TCR” or a “γ/δ TCR” refer to a TCR comprising an α-chain and a β-chain as described above, or a γ-chain and a δ-chain, respectively. Such a TCR may also be described as “full length TCR” or “conventional TCR”. An α/β TCR or a γ/δ TCR may be a native TCR or may be an engineered TCR that retains the structure of a native TCR, i.e. an engineered TCR comprising minor modifications in the variable and/or constant domains as described above, such as a humanized TCR.

“Single chain TCR (scTCR)” as used herein denotes a TCR in which the variable domains of the TCR are located on a single polypeptide. Typically, the variable domains in scTCRs are separated by a linker, wherein said linker typically comprises 10 to 30 amino acids, such as 25 amino acids.

A “chimeric protein” herein refers to a protein comprising sequences from multiple species. A “chimeric TCR” herein refers to a TCR comprising sequences from multiple species. Preferably, a chimeric TCR in the context of the invention may comprise an α-chain comprising at least one domain from a human and one domain from mouse. More preferably, a chimeric TCR in the context of the invention may comprise an α-chain comprising a variable domain of a human α-chain and, for example, a constant domain of a murine TCR α-chain.

The term “antibody” as used herein is meant to include native and engineered antibodies. The term “engineered antibody includes functional antibody fragments, single chain antibodies, single domain antibodies, bispecific or multispecific antibodies.

A “native antibody” comprises two heavy and two light chains, wherein the heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct domains (also referred to as regions). The light chain includes two domains, a variable domain (V_{L}) and a constant domain (C_{L}). The heavy chain includes four or five domains depending on the antibody isotype; a variable domain (VH) and three or four constant domains (C_{H1}, C_{H2} and C_{H3}, and optionally C_{H4}, collectively referred to as C_{H}). The variable domains of both light (V_{L}) and heavy (V_{H}) chains determine binding recognition and specificity to the antigen. The constant domains of the light (C_{L}) and heavy (C_{H}) chains confer important biological properties such as antibody chain association, secretion, trans-placental mobility, complement binding, and binding to Fc receptors (FcR).

The specificity of the antibody resides in the structural complementarity between the antibody binding site and the antigenic determinant. Antibody binding sites are made up of residues that are primarily from the “antibody complementarity determining regions” (CDRs) or hypervariable regions. Occasionally, residues from non-hypervariable or framework regions (FR) influence the overall domain structure and hence the binding site. CDRs refer to amino acid sequences that together define the binding affinity and specificity of the natural Fv region of a native antibody binding site. The light and heavy chains of an antibody each have three CDRs, designated CDR1-L, CDR2-L, CDR3-L and CDR1-H, CDR2-H, CDR3-H, respectively. An antibody antigen-binding site, therefore, includes six CDRs, comprising the CDR set from each of a heavy and a light chain V region. “Antibody framework regions” (FRs) refer to amino acid sequences interposed between CDRs, i.e. to those portions of antibody light and heavy chain variable regions that are relatively conserved among different antibodies in a single species. The light and heavy chains of an antibody each have four FRs, designated FR1-L, FR2-L, FR3-L, FR4-L, and FR1-H, FR2-H, FR3-H, FR4-H, respectively. Accordingly, the light chain variable domain may be described as (FR1-L)-(CDR1-L)-(FR2-L)-(CDR2-L)-(FR3-L)-(CDR3-L)-(FR4-L) and the heavy chain variable domain may be described as (FR1-H)-(CDR1-H)-(FR2-H)-(CDR2-H)-(FR3-H)-(CDR3-H)-(FR4-H). As used herein, a “human framework region” is a framework region that is substantially identical (about 85%, or more, in particular 90%, 95%, 97%, 99% or 100%) to the framework region of a naturally occurring human antibody. In the context of the invention, CDR/FR definition in an antibody light or heavy chain variable domain is determined based on Kabat definition (Kabat et al., U.S. Dept. of Health and Human Services, “Sequences of proteins of immunological interest”, 1991). Accordingly, amino acid sequences of the CDR1, CDR2 and CDR3 of a given variable chain and the amino acid sequences of the framework regions (e.g. FR1, FR2, FR3, and FR4) are indicated according to said Kabat definition in the herein provided disclosure

Knowing the amino acid sequence of the CDRs of an antibody, a TCR or an antigen binding protein of the invention, one skilled in the art can easily determine the framework regions, such as the TCR framework regions or antibody framework regions. In cases where the CDRs are not indicated, the skilled in the art can first determine the CDR amino acid sequences based on the IMGT definition for TCRs or the Kabat definition for antibodies and then determine the amino acid sequences of the framework regions.

Engineered antibody formats include functional antibody fragments, single chain antibodies, single domain antibodies, and Fc- and/or hinge-engineered, effector function-silenced, -augmented or -altered antibodies, chimeric, humanized, bispecific or multispecific antibodies. Engineered antibody formats further include constructs in which the light chain variable domain of an antibody may be replaced with the α-chain variable domain of a TCR and the heavy chain variable domain may be replaced with the β-chain variable domain of a TCR, or vice versa. A “functional antibody fragment” refers to a portion of a full-length antibody that retains the ability to bind to its target antigen, in particular the affinity and/or specificity for its target antigen. Preferably, a functional antibody fragment comprises the antigen binding region or variable region of the full-length antibody. Examples of functional antibody fragments include Fv, Fab, F(ab′)2, Fab′, dsFv, (dsFv)2, scFv, sc (Fv)2, nanobodies, DARPins, Knottins and diabodies. A functional antibody fragment may also be a single domain antibody, such as a heavy chain antibody. The term “Fab” denotes an antibody fragment having a molecular weight of about 50,000 Daltons and antigen binding activity, in which about a half of the N-terminal side of H chain and the entire L chain, among fragments obtained by treating IgG with a protease, e.g. papain, are bound together through a disulfide bond. The Fv fragment is the N-terminal part of the Fab fragment of an antibody and consists of the variable portions of one light chain and one heavy chain.

The antigen binding protein and the CDRs as provided herein can be incorporated in various formats as herein disclosed above. Further exemplary antigen binding proteins comprising the inventive CDRs in different formats are herein provided below.

The antigen binding protein may be a “diabody” or the herein provided binding sites may be comprised in the “diabody format”. These terms refer to bivalent molecules composed of two polypeptide chains, each comprising two variable domains connected by a linker (e.g. L_{Db1} and L_{Db2}), wherein two of the domains may be first and second variable domains as defined herein (e.g. V1 and V2) and the other two domains may be TCR derived or antibody derived variable domains (e.g. V3 and V4). Particularly, the V1 and V2 domains may be located on two different polypeptides and the V3 and V4 domains may be located on two different polypeptides and the domains dimerize in a cross-over confirmation. The domains may also be located on further polypeptide chains, e.g. four polypeptide chains. The L_{Db1} and L_{Db1}, can be identical or different and may be short linkers. A short linker is a linker that is typically between 2 to 12, 3 to 13, such as 3, 4, 5, 6, 7, 8, 9 amino acids long, for example 4, 5 (Brinkmann U. and Kontermann R. E. (MAbs. 2017 February-March; 9 (2): 182-212) or 8 amino acids long, such as ‘GGGSGGGG’ of SEQ ID NO: 26.

A further format of the antigen binding protein may be the “dual-variable-domain immunoglobulin (DVD-Ig™)” format as described in 2007 by Wu C. et al. (Nat Biotechnol. 2007 November; 25(11):1290-7). The DVD-Ig™ as described in the art, is thus typically composed of two polypeptide chains, one heavy chain comprising V-L-V-C_{H1}-C_{H2}-C_{H3} and one light chain comprising V-L-V-CL. The domain pairs V/V and V/V are thus pairing in parallel. The herein provided CDRs and variable domains may be incorporated into this exemplary format.

The “dual-variable-domain Ig format” (or “DVD-Ig format”) refers to a protein comprising two polypeptide chains, each comprising two variable domains connected by a linker (L1, L3), wherein two of the variable domains are first and second variable domains as defined in the context of the present invention (V_{A} and V_{B}) and the other two domains are antibody or TCR derived.

As used herein, a “format” of an antigen binding protein specifies a defined spatial arrangement of domains, in particular of variable and optionally constant domains. Characteristics of such antigen binding protein formats are the number of polypeptide chains (single chain, double chain or multiple chains), the type and length of linkers connecting different domains, the number of variable domains (and thus the number of valences), the number of different variable domains (and thus the number of specificities for different antigens, e.g. bispecific, multispecific), and the order and orientation of variable domains (e.g. cross-over, parallel). As indicated herein above and as shown in the examples, the inventive antigen binding domains, e.g. specified by the CDRs as provided herein, may be comprised in various formats.

The term “humanized” in the context of an antigen binding protein or antibody refers to an antibody which is completely or partially of non-human origin and which has been modified by replacing certain amino acids, in particular in the framework regions of the heavy and light chains, in order to avoid or minimize an immune response in humans. The constant domains of a humanized antibody are mainly human CH and CL domains. Numerous methods for humanization of an antibody sequence are known in the art. For example, a “humanized” antibody can be made by the introduction of conservative substitutions, consensus sequence substitutions, germline substitutions and/or back mutations, see, e.g., Teng et al., Proc. Natl. Acad. Sci. U.S.A., 80:7308-7312, 1983; Kozbor et al., Immunology Today, 4:7279, 1983; Olsson et al., Meth. Enzymol., 92:3-16, 1982, and the review by Almagro & Fransson (2008) Front Biosci. 13:1619-1633. One commonly used method is CDR grafting, or antibody reshaping, which involves grafting of the CDR sequences of a donor antibody, generally a mouse antibody, into the framework scaffold of a human antibody of different specificity. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. An alternative humanization technique to CDR grafting and back mutation is resurfacing, in which non-surface exposed residues of non-human origin are retained, while surface residues are altered to human residues. Another alternative technique is known as “guided selection” (Jespers et al. (1994) Biotechnology 12, 899) and can be used to derive from for example a murine or rat antibody a fully human antibody conserving the epitope and binding characteristics of the parental antibody. A further method of humanization is the so-called 4D humanization. The 4D humanization protocol is described in the patent application US20110027266 A1 (WO2009032661A1) and is exemplified in the following applying the 4D humanization to humanize the rat antibody variable light (V_{L}) and heavy (V_{H}) domains.

For chimeric antibodies, humanization typically involves modification of the framework regions of the variable region sequences.

The term “percentage of identity” indicates a quantitative measure of the degree of homology between two sequences, which in the context of the present invention are amino acid sequences. If the two sequences to be compared are not of equal length, they must be aligned to give the best possible fit, allowing the insertion of gaps or alternatively, truncation at the ends of the nucleic acid sequences or amino acid sequences. The skilled person will acknowledge that various means for comparing sequence identity are available (see below).

For example, in the context of the present application, a sequence that is “at least 85% identical to a reference sequence” is a sequence having, over its entire length, 85%, or more, in particular 90%, 91%, 92%, 93%, 94%, 95%, 96%, 97%, 98% or 99% sequence identity with the entire length of a reference sequence (e.g., a variable domain disclosed herein). Proteins consisting of an amino acid sequence “at least 80%, 85%, 90%, 95%, 96%, 97%, 98% or 99% Identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

In the context of the present application, the “percentage of identity” can be calculated using a global pairwise alignment (i.e. the two sequences are compared over their entire length). Methods for comparing the identity of two or more sequences are well known in the art. For example, the “needle” program, which uses the Needleman-Wunsch global alignment algorithm (Needleman and Wunsch, 1970 J. Mol. Biol. 48:443-453) to find the optimum alignment (including gaps) of two sequences when considering their entire length, may be used. The needle program is for example available on the ebi.ac.uk World Wide Web site and is further described in the following publication (EMBOSS: The European Molecular Biology Open Software Suite (2000) Rice, P. Longden, I. and Bleasby, A. Trends in Genetics 16, (6) pp. 276-277). The percentage of identity between two polypeptides, in accordance with the invention, is calculated using the EMBOSS: needle (global) program with a “Gap Open” parameter equal to 10.0, a “Gap Extend” parameter equal to 0.5, and a Blosum62 matrix.

“Amino acid mutations” may be deletions, insertions or substitutions. Mutations may also be post-translational modification(s), e.g. at the N-terminus or the C-terminus of the polypeptide chains. Post-translational modification may also be comprised in the antigen binding protein upon formulation of the antigen binding protein in a pharmaceutical composition.

“Amino acid substitutions” may be conservative or non-conservative. In an embodiment, substitutions are conservative substitutions, in which one amino acid is substituted for another amino acid with similar structural and/or chemical properties. Amino acid substitutions may also be caused by post-translational modifications. Thus, amino acid substitutions may also be comprised in the antigen binding protein upon formulation of the antigen binding protein in a pharmaceutical composition.

In one embodiment, a conservative amino acid substitution may include the substitution of an amino acid by another amino acid of the same class, for example, non-polar amino acids substituted by other non-polar amino acids.

In another embodiment, conservative substitutions may be made in accordance with Table 1. Methods for predicting tolerance to protein modification may be found in, for example, Guo et al., Proc. Natl. Acad. Sci., USA, 101(25):9205-9210 (2004), the contents of which are incorporated by reference in their entirety.

TABLE 1
Conservative Amino Acid substitutions

| Amino acid | Substitutions (others are known in the art) |
|---|---|
| Ala | Ser, Gly, Cys |
| Arg | Lys, Gln, His |
| Asn | Gln, His, Glu, Asp |
| Asp | Glu, Asn, Gln |
| Cys | Ser, Met, Thr |
| Gln | Asn, Lys, Glu, Asp, Arg |
| Glu | Asp, Asn, Gln |
| Gly | Pro, Ala, Ser |
| His | Asn, Gln, Lys |
| Ile | Leu, Val, Met, Ala |
| Leu | Ile, Val, Met, Ala |
| Lys | Arg, Gln, His |
| Met | Leu, Ile, Val, Ala, Phe |
| Phe | Met, Leu, Tyr, Trp, His |
| Ser | Thr, Cys, Ala |
| Thr | Ser, Val, Ala |
| Trp | Tyr, Phe |
| Tyr | Trp, Phe, His |
| Val | Ile, Leu, Met, Ala, Thr |

The antigen binding proteins of the present invention can comprise synthetic amino acids in place of one or more naturally-occurring amino acids. Such synthetic amino acids are known in the art, and may include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α, γ-diaminobutyric acid, α,β-diaminopropionic acid, homophenylalanine, and α-tert-butylglycine.

In one embodiment, the antigen binding protein or the amino acid(s) of the antigen binding protein of the present invention can be glycosylated, amidated, carboxylated, phosphorylated, esterified, N-acylated, cyclized, e.g., via a disulfide bridge, or converted into an acid addition salt and/or optionally dimerized or polymerized, or conjugated. In a preferred embodiment, a glutamine of the antigen binding protein is cyclized to pyroglutamate. Further, the N-terminal glutamine of the antigen binding protein may be cyclized to pyroglutamate.

A “covalent link” herein refers for example to a disulfide bridge or a peptide link or a covalent link via a linker or a linker sequence, such as a polypeptide linker.

The term “linker” as used herein refers to one or more amino acid residues inserted between domains or a domain and an agent to provide sufficient mobility for the domains or elements, for example the variable domains of bispecific antigen binding to fold correctly to form the antigen binding sites.

In some embodiments, a linker consists of 0 amino acid meaning that the linker is absent. A linker is inserted at the transition between variable domains or between variable domains and constant domains (or dimerization domains), respectively, at the amino acid sequence level. The transition between domains can be identified because the approximate size of the antibody domains as well as of the TCR domains is well understood. The precise location of a domain transition can be determined by locating peptide stretches that do not form secondary structural elements such as beta-sheets or alpha-helices as demonstrated by experimental data or as can be assumed by techniques of modelling or secondary structure prediction.

A linker, as long as it is not specified otherwise in the respective context, can be from at least 1 to 30 amino acids in length. In some embodiments, a linker can be 2-25, 2-20, or 3-18 amino acids long. In some embodiments, a linker can be a peptide of a length of no more than 14, 13, 12, 11, 10, 9, 8, 7, 6, or 5 amino acids. In other embodiments, a linker can be 5-25, 5-15, 4-11, 10-20, or 20-30 amino acids long. In other embodiments, a linker can be about, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids long. In a particular embodiment, a linker can be less than 24, less than 20, less than 16, is less than 12, less than 10, for example from 5 to 24, 10 to 24 or 5-10 amino acid residues in length. In some embodiments, said linker is equal to 1 or more amino acid residues in length, such as more than 1, more than 2, more than 5, more than 10, more than 20 amino acid residues in length, more than 22 amino acid residues in length. In preferred embodiments, the linker is a glycine/serine linker, i.e. a linker consisting of or essentially consisting of glycine and serine residues.

The antigen binding protein of the present disclosure can be synthetic, recombinant, isolated, engineered and/or purified. In particular aspects, the antigen binding protein is a non-naturally occurring antigen binding protein, e.g. if the antigen binding protein is a soluble bispecific antigen binding protein, such as a TCER®.

An “engineered” antigen binding protein, in particular an engineered TCR in the context of the present invention refers to a protein that is not naturally occurring or that has been modified by biotechnological methods, in particular by introducing amino acid mutations/substitutions into the native protein sequence. Such biotechnological methods are well known to the skilled in the art.

By “purified” is meant, when referring to a polypeptide, e.g. to the antigen binding protein of the invention) or a nucleotide sequence, e.g. encoding antigen binding proteins or functional fragment thereof described herein, that the indicated molecule is present in the substantial absence of other biological macromolecules of the same type. The term “purified” as used herein in particular means that at least 75%, 85%, 95%, or 98% by weight, of biological macromolecules of the same type are present. The term “purified” as used herein may further indicate that the antigen binding protein is free from DNA, RNA, proteins, polypeptides or cells that would interfere with its therapeutic, diagnostic, prophylactic, research or other use.

A purified nucleic acid molecule that encodes a particular polypeptide refers to a nucleic acid molecule that is substantially free of other nucleic acid molecules that do not encode the subject polypeptide; however, the molecule may include some additional bases or moieties, which do not deleteriously affect the basic characteristics of the composition.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated”. An isolated nucleic acid or protein can exist in substantially purified form, or can exist in a non-native environment such as, for example, a host cell. An isolated antigen binding protein is substantially free of other antigen binding proteins having different antigenic specificities (e.g., an antigen binding protein that specifically binds MAGEB2 is substantially free of antigen binding proteins that specifically bind antigens other than MAGEB2). Moreover, an isolated antigen binding protein may be substantially free of other cellular material and/or chemicals. Accordingly, “isolated” may refer to a protein that is removed from cell culture and separated from cell culture components, e.g., it may have been separated from at least 90%, preferably at least 95%, of cell culture components.

A “recombinant” molecule is one that has been prepared, expressed, created, or isolated by recombinant means. Recombinant molecules do not exist in nature. Accordingly, “recombinant” refers to a polypeptide or protein molecule which is made using recombinant techniques, i.e., which is not naturally occurring. Methods and techniques for the production of recombinant nucleic acids and polypeptides are well known in the art.

The term “gene” means a DNA sequence that codes for, or corresponds to, a particular sequence of amino acids which comprises all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

“Affinity” is defined in the context of the present invention by the equilibrium binding between the antigen binding protein and its antigen, namely the MAGEB2 peptide in a complex with a MHC protein. Affinity is usually expressed as equilibrium dissociation constant (K_{D}).

“K_{D}” is the equilibrium dissociation constant, a ratio of K_{off}/kₒₙ, between the antigen binding protein and its antigen. K_{D} and affinity are inversely related. The K_{D} value relates to the concentration of the antigen binding protein and the lower the K_{D} value, the higher the affinity of the antigen binding protein. The K_{D} value can be experimentally assessed by a variety of known methods, such as measuring association and dissociation rates with surface plasmon resonance (SPR) or biolayer interferometry (BLI). Binding interactions can be measured at a broad range of settings, including, but not limited to, a temperature range of 25° C. to 37° C. and a shake speed range of 500 rpm to 1500 rpm using a suitable buffer that minimizes nonspecific binding and maintains protein stability. Examples of such buffers are phosphate buffered saline (PBS), Tris buffered saline (TBS), HEPES buffered saline (HBS), or other physiological buffers, with or without additives such as Tween, BSA, DMSO, or EDTA. The analyte can be immobilized on various sensors including, but not limited to HIS1K-, NTA-, SA-, ProA-, ProG-, AHC-, FAB2G-sensors, at a concentration range, including, but not limited to, 1 μg/ml to 100 μg/ml for a duration range of 30 s to 300 s. K_{D} determination can be measured at various molarities of the analyte sample, including, but not limited to, a range from 25 nM to 500 nM, or up to 10 UM for detecting potential off-target reactivities with high sensitivity. Exemplary determination of the KD is herein provided in the Examples. For example, the KD may be determined using BLI at 30° C. and 1000 rpm shake speed using PBS, 0.05% Tween, 0.1% BSA as buffer, with HLA-A*02/MAGEB2-001 immobilized on HIS1K-sensors (at a concentration of 10 μg/ml for 120 s) prior to analyzing TCR variants at concentrations ranging from 25 nM to 200 nM.

“Efficacy” is defined, in the context of the present invention, as a parameter that describes the capability of an antigen binding protein to activate an effector cell, preferably a T cell or a NK cell, to kill a cancer cell presenting on its surface the target antigenic peptide in a complex with MHC. The efficacy can be determined in a functional assay, in particular a cytotoxicity assay as described below.

In a “functional assay”, an antigen binding protein is co-cultured with “effector cells (E)” and “target cells (T)”, i.e. with cancer cells presenting a peptide:MHC complex. Functional assays can thus also be described as “co-culture assays”. For all cell culture assays described herein, the cell culture temperature preferably is at about 37° C. The target cells may be cells that are artificially loaded with the antigenic peptide (e.g. T2 cells) or may be cells that endogenously present the target antigenic peptide on their surface (e.g. cancer cells expressing MAGEB2). Binding of the antigen binding protein to the peptide:MHC complex on the target cell and binding to the effector cell leads to activation of the effector cell. Depending of the type of functional assay, there are different readouts for measuring the degree of activation. In a cytotoxicity assay, the killing of target cells by the effector cells is determined, e.g. by measuring a decline in proliferation of target cells, in particular cancer cells or by measuring the release of intracellular proteins from the target cells. Suitable intracellular proteins to be measured in a cytotoxicity assay can be endogenous proteins, e.g. LDH.

The term “E:T ratio” refers to the ratio of effector cells (i.e. immune cells) to target cells. In some embodiments, the E:T ratio corresponds to the seeding ratio, i.e. the ratio of the total number of immune cells to target cells.

In an example of a lactate dehydrogenase (LDH)-release assay, the effector cells are immune cells. These effector cells are co-cultured with tumor cells endogenously expressing and presenting the MAGEB2 antigenic peptide and optionally additionally loaded with the MAGEB2 antigenic peptide. In some embodiments, the tumor cells are SKMEL-5 cells, RPMI7951 cells or SCC25 cells. In some embodiments of the LDH-release assay, the seeding ratio of total immune cells and target cells is 10:1. The efficacy of an antigen binding protein is considered high if in a LDH-release assay as defined above, killing of tumor cells (as determined LDH release) is observed at an E:T ratio of 10:1. Alternatively, the efficacy of an antigen binding protein is considered high, if in a cytotoxicity assay, preferably a LDH-release assay as defined above, the cytotoxic activity of the effector cells against the target cells at the highest concentration of the antigen binding protein tested is at least 50%, at least 60%, at least 70%, at least 75%, preferably at least 80%, at least 85%, at least 90%, or at least 95% of the cytotoxic activity of a control toxic reagent. The skilled in the art is aware that the cytotoxic activity can be higher than 100%. This is due to the fact that 100% cytotoxic activity is defined by a “maximum lysis control”, which refers to incubation of the target cells with the toxic reagent. In some embodiments, the toxic reagent is a detergent, e.g. Triton-X100, Tween-20, Tween-80 or NP-40, that effects lysis of the target cells. In some specific examples, the maximum lysis control comprises adding a 0.9% Triton-X100 solution to the target cell culture. The cytotoxic activity of the toxic reagent, i.e. the number of target cells killed by the toxic reagent is defined as 100%. Since the target cells can still proliferate during the co-culture, the effector cells may eventually kill an even higher number of target cells during the cytotoxicity assay than the toxic reagent killed during the maximum lysis control. In such instances, the calculated cytotoxic activity will be higher than 100%.

“Half maximal effective concentration” also called “EC₅₀”, typically refers to the concentration of a molecule which induces a response halfway between the baseline and maximum after a specified exposure time. EC50 and affinity are inversely related, the lower the EC50 value the higher the affinity of the molecule. In one example, the “EC₅₀” refers to the concentration of the antigen binding protein of the invention which induces a response halfway between the baseline and maximum after a specified exposure time, more particularly, refers to the concentration of the antigen binding protein of the invention which induces a response halfway between the baseline and maximum after a specified exposure time. EC50 values can be experimentally assessed by a variety of known methods, using for example binding assays such as ELISA or flow cytometry, or functional assays such as cytokine release assay or lactate dehydrogenase (LDH) release assay. In particular embodiments, the “EC₅₀” refers to the concentration of the antigen binding protein, which induces a response halfway between the baseline and maximum, when target cells are co-cultured with effector cells in a LDH-release assay as defined above. The functional avidity of an antigen binding protein is considered high, if the EC₅₀ determined in a cytotoxicity assay, preferably a LDH-release assay as defined above, is less than about 500 pM, preferably less than about 200 pM, more preferably less than about 100 pM, or most preferably less than about 30 pM, determined using a co-culture assays with MAGEB2-expressing tumor cells having MAGEB2 copy number per cell of less than 200.

The term “specificity” in the context of the present invention denotes the capacity of an antigen binding protein to discriminate its target peptide from peptides having a different amino acid sequence, e.g. similar peptides as defined below. An antigen binding protein is considered specific for a target peptide, if binding to the target peptide presented in an MHC molecule occurs with a significantly higher affinity and/or higher functional avidity than the binding to similar peptides. The specificity of the antigen binding protein is determined by the amino acid sequences CDRa1, CDRa3, CDRb1 and CDRb3. The amino acid sequences of CDRa2 and CDRb2 contact the MHC molecule and are not required for antigen specificity.

In the context of the present invention “similar peptides” herein refers to potential off-target peptides, i.e. peptides that may potentially be bound by the antigen binding proteins of the invention based on their biochemical/biophysical characteristics, including but not limited to a homologous sequence or a similar motif. Similar peptides comprise typically 8 to 12 amino acids in length. The similar peptides in the context of the present invention are typically MHC, in particular MHC I, presented. Furthermore, similar peptides in the context of the present invention include peptides that comprise or consist of an amino acid sequence that is similar to the amino acid sequence of the MAGEB2 antigenic peptide, more particular, peptides that, in comparison to the epitope of the MAGEB2 antigenic peptide, comprise an epitope wherein some or all amino acids have identical and/or similar biochemical/biophysical characteristics as the amino acids that constitute the epitope of the MAGEB2 antigenic peptide. In some examples, similar peptides investigated in the context of the present invention were selected from a database of tumor and normal tissue-presented HLA-A*02 bound peptides (XPRESIDENT® database) using a similarity scoring within the binding-relevant positions of MAGEB2 and the requirement of at least one detection on normal tissues. Binding of an antigen binding protein to a similar peptide presented by an MHC protein may lead to adverse reactions. Such adverse reactions may be “off-tumor” side effects, such as cross-reactivity of a specific TCR with a similar peptide in healthy tissues as reported in Lowdell et al., Cytotherapy, published on Dec. 4, 2018).

In particular, the following peptides are similar peptides in the context of the present invention: SEQ ID NOs: 59 to 67, 106, and 133 to 151.

The skilled person is aware that among the similar peptides, there are some that are not bound by the antigen binding proteins of the invention to a detectable degree, e.g. peptides for which no binding signal during affinity determination or no response in a functional assay beyond the background level is detectable. “Background level” in this context refers to a response in a functional assay observed for the co-culture of target cells and effector cells at the respective E:T ratio without the addition of bispecific TCR-antibody fusion protein.

For other similar peptides, a low, but non-significant binding may be detectable. These latter similar peptides may also be described as “potentially relevant” similar peptides. An antigen binding protein is considered to not significantly bind to a similar peptide and to be specific for its target antigenic peptide if at least one of the following applies when binding to the similar peptide and the target antigenic peptide is compared under similar, preferably identical experimental conditions:

The cytotoxic activity in response to the similar peptide, determined in a cytotoxicity assay as described above, is 25% or less, 20% or less, 15% or less, 10% or less of the cytotoxic activity in response to the target antigenic peptide MAGEB2.

The EC₅₀ of the similar peptide, determined in a functional assay, preferably a cytotoxicity assay, as described above, is increased by a factor of at least 50, at least 100, at least 200 or at least 500, compared to the EC₅₀ of the target antigenic peptide MAGEB2.

The K_{D} for the similar peptide is increased by a factor at least 25, at least 30, at least 40, at least 50, at least 75, or at least 100, compared to the K_{D} for the target antigenic peptide MAGEB2.

The relative response signal for the similar peptide is not higher than 30%, not higher than 25%, not higher than 20%, or not higher than 15%, compared to the response signal to the target antigenic peptide.

“CD3” is a protein complex and is composed of four distinct chains. In mammals, the complex contains a CD3γ chain, a CD3δ chain, and two CD3ε chains. These chains associate with a molecule known as the T cell receptor (TCR) and the ζ-chain to generate an activation signal in T lymphocytes.

“CD28” is also expressed on T cells and can provide co-stimulatory signals, which are required for T cell activation. CD28 plays important roles in T cell proliferation and survival, cytokine production, and T-helper type-2 development.

“CD134” is also termed OX40. CD134/OX40 is being expressed after 24 to 72 hours following activation and can be taken to define a secondary costimulatory molecule.

“4-1BB” is capable of binding to 4-1BB-Ligand on antigen presenting cells (APCs), whereby a costimulatory signal for the T cell is generated.

“CD5” is another example of a receptor predominantly found on T cells, CD5 is also found on B cells at low levels.

“CD95” is a further example of a receptor modifying T cell functions and is also known as the Fas receptor, which mediates apoptotic signaling by Fas-ligand expressed on the surface of other cells. CD95 has been reported to modulate TCR/CD3-driven signaling pathways in resting T lymphocytes.

A “NK cell specific receptor molecule” is, for example, CD16, a low affinity Fc receptor and NKG2D.

“Copy number” herein refers to the number of MAGEB2/MHC complexes as defined in the context of the present invention that are present on the cell surface of a cell, such as a MAGEB2/MHC presenting cell, for example a cancer cell, or a healthy cell. Copy numbers of a protein can be determined by a variety of art known methods including FACS analysis of diseased cells with fluorescently labelled antigen binding proteins.

“Safety profile” herein refers to the capacity to distinguish tumor cells from healthy tissue cells and this is often determined by determining the safety window.

The “safety window” or “therapeutic window” herein refers to a factor that compares the half maximal concentration of a compound that is required for inducing 100% cytotoxicity in a tumor cell line in comparison to the half maximal concentration of a compound that is required for inducing 100% cytotoxicity healthy tissue cells. If for an antigen binding protein of interest the EC50 determined for a tumor cell line is 1 pM and the EC50 value determined for, for instance, primary cells is 1000 pM then the safety window is 1000 since the EC50 for the tumor cell line is 1000 times smaller than the EC50 for the primary cells.

The term “nucleic acid” refers in the context of this invention to single or double-stranded oligo- or polymers of deoxyribonucleotide or ribonucleotide bases or both. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers, In the context of the present invention, the term nucleic acid includes but is not limited to ribonucleic acid (RNA) and deoxyribonucleic acid (DNA) molecules but also includes synthetic forms of nucleic acids comprising other linkages (e.g., peptide nucleic acids as described in Nielsen et al. (Science 254:1497-1500, 1991). Typically, nucleic acids are single- or double-stranded molecules and are composed of naturally occurring nucleotides. The depiction of a single strand of a nucleic acid also defines (at least partially) the sequence of the complementary strand. The nucleic acid may be single or double stranded or may contain portions of both double and single stranded sequences. Exemplified, double-stranded nucleic acid molecules can have 3′ or 5′ overhangs and as such are not required or assumed to be completely double-stranded over their entire length. The term nucleic acid comprises chromosomes or chromosomal segments, vectors (e.g., expression vectors), expression cassettes, naked DNA or RNA polymer, primers, probes, cDNA, genomic DNA, recombinant DNA, cRNA, mRNA, tRNA, microRNA (miRNA) or small interfering RNA (SIRNA). A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques.

The terms “vector”, “cloning vector” and “expression vector” refers to a vehicle by which a DNA or RNA sequence (e.g. a foreign gene) can be introduced into a host cell, so as to transform the host and promote expression (e.g. transcription and translation) of the introduced sequence.

The term “viral vector” refers to a nucleic acid vector construct that includes at least one element of viral origin and has the capacity to be packaged into a viral vector particle and encodes at least an exogenous nucleic acid. The vector and/or particle can be utilized for the purpose of transferring a nucleic acid of interest into cells either in vitro or in vivo. Numerous forms of viral vectors are known in the art. Useful viral vectors include vectors based on retroviruses, lentiviruses, adenoviruses, adeno-associated viruses, herpes viruses, vectors based on SV40, papilloma virus, Epstein Barr virus, vaccinia virus vectors, and Semliki Forest virus (SFV). Recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The term “transformation” means the introduction of a “foreign” (i.e. extrinsic) gene, DNA or RNA sequence to a host cell, so that the host cell will express the introduced gene or sequence to produce a desired substance, typically the antigen-binding protein or functional fragment thereof described herein. A host cell that receives and expresses introduced DNA or RNA has been “transformed”.

The term “expression system” means a host cell and compatible vector under suitable conditions, e.g. for the expression of a protein coded for by foreign DNA carried by the vector and introduced to the host cell.

A “host cell” is a cell that can be used to express a nucleic acid, e.g., a nucleic acid disclosed herein.

The terms “pharmaceutical composition” or “therapeutic composition” as used herein refer to a compound or composition capable of inducing a desired therapeutic effect when properly administered to a subject.

The terms “subject” or “individual” are used interchangeably and include all mammals, preferably humans. In some embodiments, the subject to be treated may also be referred to as “patient”.

A “therapeutic agent” herein refers to an agent that has a therapeutic effect. In one embodiment, such a therapeutic agent may be a growth inhibitory agent, such as a cytotoxic agent or a radioactive isotope.

A “growth inhibitory agent”, or “anti-proliferative agent”, which can be used indifferently, refers to a compound or composition which inhibits growth of a cell, especially a tumor cell, either in vitro or in vivo.

The term “cytotoxic agent” as used herein refers to a substance that inhibits or prevents the function of cells and/or causes destruction of cells. The term “cytotoxic agent” is intended to include chemotherapeutic agents, enzymes, antibiotics, and toxins such as small molecule toxins or enzymatically active toxins of bacterial, fungal, plant or animal origin, including fragments and/or variants thereof, and the various antitumor or anticancer agents disclosed below. In some embodiments, the cytotoxic agent is a taxoid, vincas, taxanes, a maytansinoid or maytansinoid analog such as DM1 or DM4, a small drug, a tomaymycin or pyrrolobenzodiazepine derivative, a cryptophycin derivative, a leptomycin derivative, an auristatin or dolastatin analog, a prodrug, topoisomerase II inhibitors, a DNA alkylating agent, an anti-tubulin agent, a CC-1065 or CC-1065 analog.

The term “radioactive isotope” is intended to include radioactive isotopes suitable for treating cancer, such as At²¹¹, Bi²¹², Er¹⁶⁹, I¹³¹, I¹²⁵, Y90, In¹¹¹, P³², Re¹⁸⁶, Re¹⁸⁸, Sm¹⁵³, Sr⁸⁹, and radioactive isotopes of Lu. Such radioisotopes generally emit mainly beta-radiation. In an embodiment the radioactive isotope is alpha-emitter isotope, more precisely Thorium 227 which emits alpha-radiation.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier refers to a non-toxic solid, semi-solid or liquid filler, diluent, encapsulating material or formulation auxiliary of any type.

A “pharmaceutically acceptable carrier” and may include solvents, bulking agents, stabilizing agents, dispersion media, coatings, antibacterial and antifungal agents, isotonic and absorption delaying agents, and the like which are physiologically compatible. In one embodiment the carrier is an aqueous carrier. In some embodiments, the aqueous carrier is capable of imparting improved properties when combined with an antigen binding protein described herein, for example, improved solubility, efficacy, and/or improved immunotherapy.

In the context of the invention, the terms “treating” or “treatment”, include both therapeutic treatment (i.e. on a subject having a given disease) and/or preventive or prophylactic treatment (i.e. on a subject susceptible of developing a given disease). Therapeutic treatment and means reversing, alleviating and/or inhibiting the progress of one or more symptoms of a disorder or condition. Prophylactic treatment means preventing the occurrence of one or more symptoms of a disorder or condition. Therefore, treatment does not only refer to a treatment that leads to a complete cure of the disease, but also to treatments that slow down the progression of the disease, prevent or delay the occurrence of the disease and/or prolong the survival of the subject.

By a “therapeutically effective amount” of the antigen binding protein or pharmaceutical composition thereof is meant a sufficient amount of the antigen binding protein to treat said proliferative disease, at a reasonable benefit/risk ratio applicable to any medical treatment. It will be understood, however, that the total daily usage of the antigen binding proteins, the nucleic acid or vector, the host cell or the pharmaceutical composition of the present invention will be decided by the attending physician within the scope of sound medical judgment. The specific therapeutically effective dose level for any particular patient will depend upon a variety of factors including the disorder being treated and the severity of the disorder; activity of the specific antigen binding protein employed; the specific composition employed, the age, body weight, general health, sex and diet of the patient; the time of administration, route of administration, and rate of excretion of the specific polypeptide employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved.

In one embodiment, a “disease” or “disorder” is any condition that would benefit from treatment with the antigen binding protein of the invention. In one embodiment, this includes chronic and acute disorders or diseases including those pathological conditions which predisposes the subject to the disorder in question. The term “in need of treatment” refers to a subject already having the disorder as well as a subject in which the disorder is to be prevented.

“Proliferative diseases”, such as cancer, involve the unregulated and/or inappropriate proliferation of cells.

A cancer is considered to be a “MAGEB2-expressing cancer” (also referred to as MAGEB2 “positive” cancer), if the related peptide, such as, for example the MAGEB2 antigenic peptide of SEQ ID NO: 1, is over-presented in patient cancer cells as defined herein. In all other indications named here a biopsy can be performed as it is standard in the treatment of these cancers and the peptide can be identified according to the XPresident® and related methods (according to WO 03/100432; WO 2005/076009; WO 2011/128448; WO 2016/107740, U.S. Pat. Nos. 7,811,828, 9,791,444, and US 2016/0187351, the contents of each are hereby incorporated by reference in their entirety). In one embodiment, the cancer is readily assayed (i.e. diagnosed) for instance by using an antigen binding protein of the invention. Methods to identify an antigen expressing cancer using an antigen binding protein are known to the skilled in the art. It is to be understood that the terms “cancer” and “carcinoma” are not used interchangeably herein since a carcinoma is a specific type of cancer emerging in the skin or in tissues that line or cover body organs.

The terms “first container” and “second container” refer to the chambers of a multi-chambered pre-filled syringe (e.g., lyosyringes).

In the context of the present specification, the term “about” or “approximately” when referring to a specific value is meant to indicate that the value may deviate by +10%, +9%, +8%, +7%, +6%, +5%, +4%, +3%, +2% or #1%. It also includes the concrete value, e.g., “about 50” includes the value “50”.

Throughout the instant application, the term “and/or” is a grammatical conjunction that is to be interpreted as encompassing that one or more of the cases it connects may occur.

Furthermore, throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”). Accordingly, both meanings are specifically intended, and hence individually disclosed, embodiments according to the present invention.

Furthermore, the indefinite article “a” or “an” does not exclude a plurality. The mere fact that certain measures are recited in mutually different dependent claims does not indicate that a combination of these measures cannot be used to advantage. Accordingly, the articles “a” and “an” preceding an element or component are intended to be non-restrictive regarding the number of instances (i.e., occurrences) of the element or component. Therefore, “a” or “an” is to be read to include one or at least one, and the singular word form of the element or component also includes the plural unless the number is obviously meant to be singular.

The terms “of the invention” or “according to the invention” as used herein are intended to refer to all aspects and embodiments of the invention disclosed and/or claimed herein. Any aspects, items or embodiments referred to herein as being “disclosed herein” or “described herein” are to be understood as being aspects, items or embodiments “of the invention” or “according to the invention”.

The invention will now be described in more details with reference to the following figures and examples. All literature and patent documents cited herein are hereby incorporated by reference. While the invention has been illustrated and described in detail in the foregoing description, the examples are to be considered illustrative or exemplary and not restrictive.

### Antigen Binding Proteins

Exemplary antigen binding proteins are shown in Table 7. Thus, the invention also relates to the sequence(s) as herein provided in Table 7. The residues marked in bold (italic and underlined) in Table 7 are exemplary residues which may undergo N-terminal post-translational modification, as specified elsewhere herein. Said post-translational N-terminal modification may for example results in a pyroglutamate residue, as specified elsewhere herein.

In a first aspect, the invention provides an antigen binding protein specifically binding to a MAGEB2 antigenic peptide that is in a complex with a major histocompatibility complex (MHC) protein, wherein the MAGEB2 antigenic peptide comprises or consists of the amino acid sequence GVYDGEEHSV (SEQ ID NO: 1),
wherein the antigen binding protein comprises the complementarity determining regions (CDRs) CDRa1, CDRa2, and CDRa3 as provided herein, and comprises the CDRb1, CDRb2, and CDRb3 as provided herein, and wherein CDRa1, CDRa2, CDRa3, CDRb1, CDRb2 and CDRb3 form an antigen-binding domain A. For example, in one embodiment of the invention, the antigen binding protein comprises

CDRa1 comprising an amino acid sequence of SEQ ID NO: 2, CDRa3 comprising an amino acid sequence selected from the group consisting of: SEQ ID NOs: 3 and 4, CDRb1 comprising an amino acid sequence of SEQ ID NO: 5, and CDRb3 comprising an amino acid sequence of SEQ ID NO: 6;
wherein the CDRa1, CDRa3, CDRb1 and/or CDRb3 sequence(s) may comprise one, two or three amino acid mutations. In another embodiment, the CDRa1, CDRa3, CDRb1 and/or CDRb3 sequence(s) may each comprise at most one, at most two or at most three amino acid mutations.

In some embodiments of the antigen binding protein, CDRa2 comprises an amino acid sequence selected from the group consisting of: SEQ ID NOs: 7 and 8, and CDRb2 comprises an amino acid sequence selected from the group consisting of: SEQ ID NOs: 9 and 10;
wherein the CDRa2 and/or CDRb2 sequence(s) may comprise one, two or three amino acid mutations. In another embodiment, the CDRa2 and/or CDRb2 sequence(s) may each comprise at most one, at most two or at most three amino acid mutations.

In another aspect, the invention provides an antigen binding protein specifically binding to a MAGEB2 antigenic peptide that is in a complex with an MHC protein, wherein the MAGEB2 antigenic peptide comprises or consists of the amino acid sequence GVYDGEEHSV (SEQ ID NO: 1),
wherein the antigen binding protein comprises a first polypeptide comprising a variable domain V_{A} comprising complementarity determining regions (CDRs) CDRa1, CDRa2, and CDRa3, and a second polypeptide comprising a variable domain V_{B} comprising CDRb1, CDRb2, and CDRb3, wherein CDRa1, CDRa2, CDRa3, CDRb1, CDRb2 and CDRb3 form an antigen-binding domain A; wherein
(1) CDRa1 comprises SEQ ID NO: 2, CDRa3 comprises SEQ ID NO: 3, CDRb1 comprises SEQ ID NO: 5, and CDRb3 comprises SEQ ID NO: 6; or
(2) CDRa1 comprises SEQ ID NO: 2, CDRa3 comprises SEQ ID NO: 4, CDRb1 comprises SEQ ID NO: 5, and CDRb3 comprises SEQ ID NO: 6;
wherein the CDRa1, CDRa3, CDRb1 and/or CDRb3 sequence(s) may comprise one, two or three amino acid mutations.

In another aspect, the invention provides an antigen binding protein comprising a variable domain V_{A} comprising complementarity determining regions (CDRs) CDRa1, CDRa2, and CDRa3, and comprising a variable domain V_{B} comprising CDRb1, CDRb2, and CDRb3, wherein CDRa1, CDRa2, CDRa3, CDRb1, CDRb2 and CDRb3 form an antigen-binding domain A; wherein
(1) CDRa1 comprises SEQ ID NO: 2, CDRa3 comprises SEQ ID NO: 3, CDRb1 comprises SEQ ID NO: 5, and CDRb3 comprises SEQ ID NO: 6; or
(2) CDRa1 comprises SEQ ID NO: 2, CDRa3 comprises SEQ ID NO: 4, CDRb1 comprises SEQ ID NO: 5, and CDRb3 comprises SEQ ID NO: 6.

In some embodiments of the antigen binding protein, the CDRa1, CDRa3, CDRb1 and CDRb3 sequence(s) comprise up to three amino acid mutations in each of CDRa1, CDRa3, CDRb1 and CDRb3.

In some embodiments of the antigen binding protein,
(1) CDRa1 comprises SEQ ID NO: 2, CDRa2 comprises SEQ ID NO: 7, CDRa3 comprises SEQ ID NO: 3, CDRb1 comprises SEQ ID NO: 5, CDRb2 comprises SEQ ID NO: 9, and CDRb3 comprises SEQ ID NO: 6;
(2) CDRa1 comprises SEQ ID NO: 2, CDRa2 comprises SEQ ID NO: 8, CDRa3 comprises SEQ ID NO: 4, CDRb1 comprises SEQ ID NO: 5, CDRb2 comprises SEQ ID NO: 10, and CDRb3 comprises SEQ ID NO: 6;
(3) CDRa1 comprises SEQ ID NO: 2, CDRa2 comprises SEQ ID NO: 7, CDRa3 comprises SEQ ID NO: 3, CDRb1 comprises SEQ ID NO: 5, CDRb2 comprises SEQ ID NO: 10, and CDRb3 comprises SEQ ID NO: 6; or
(4) CDRa1 comprises SEQ ID NO: 2, CDRa2 comprises SEQ ID NO: 8, CDRa3 comprises SEQ ID NO: 4, CDRb1 comprises SEQ ID NO: 5, CDRb2 comprises SEQ ID NO: 9, and CDRb3 comprises SEQ ID NO: 6;
wherein the CDRa1, CDRa2, CDRa3, CDRb1, CDRb2 and/or CDRb3 sequence(s) may comprise one, two or three amino acid mutations. In another embodiment, the CDRa1, CDRa2, CDRa3, CDRb1, CDRb2 and/or CDRb3 sequence(s) may each comprise at most one, at most two or at most three amino acid mutations.

In some embodiments of the antigen binding protein,
(1) CDRa1 includes SEQ ID NO: 2, CDRa2 includes SEQ ID NO: 7, CDRa3 includes SEQ ID NO: 3, CDRb1 includes SEQ ID NO: 5, CDRb2 includes SEQ ID NO: 9, and CDRb3 includes SEQ ID NO: 6;
(2) CDRa1 includes SEQ ID NO: 2, CDRa2 includes SEQ ID NO: 8, CDRa3 includes SEQ ID NO: 4, CDRb1 includes SEQ ID NO: 5, CDRb2 includes SEQ ID NO: 10, and CDRb3 includes SEQ ID NO: 6;
(3) CDRa1 includes SEQ ID NO: 2, CDRa2 includes SEQ ID NO: 7, CDRa3 includes SEQ ID NO: 3, CDRb1 includes SEQ ID NO: 5, CDRb2 includes SEQ ID NO: 10, and CDRb3 includes SEQ ID NO: 6; or
(4) CDRa1 includes SEQ ID NO: 2, CDRa2 includes SEQ ID NO: 8, CDRa3 includes SEQ ID NO: 4, CDRb1 includes SEQ ID NO: 5, CDRb2 includes SEQ ID NO: 9, and CDRb3 includes SEQ ID NO: 6;
wherein the CDRa1, CDRa2, CDRa3, CDRb1, CDRb2 and/or CDRb3 sequence(s) may have one, two or three amino acid mutations.

In some embodiments of the antigen binding protein, the CDRa1, CDRa2, CDRa3, CDRb1, CDRb2 and CDRb3 sequence(s) comprise up to three amino acid mutations in each of CDRa1, CDRa2, CDRa3, CDRb1, CDRb2 and CDRb3.

In some embodiments of the antigen binding protein, the V_{A} comprises an amino acid sequence selected from the group consisting of SEQ ID NOs: 11, 12, 13, and 14, or
an amino acid sequence having at least 85%, 90%, 95%, 98% or 99% identity to SEQ ID NO: 11, and comprising the CDRa1, CDRa2, and CDRa3 of SEQ ID NOs: 2, 7, and 3, respectively,
an amino acid sequence having at least 85%, 90%, 95%, 98% or 99% identity to SEQ ID NO: 12, and comprising the CDRa1, CDRa2, and CDRa3 of SEQ ID NOs: 2, 8, and 4, respectively,
an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, or
an amino acid sequence having at least 85%, 90%, 95%, 98% or 99% identity to SEQ ID NO: 14, and comprising the CDRa1, CDRa2, and CDRa3 of SEQ ID NOs: 2, 8, and 4, respectively; and
the V_{B} comprises an amino acid sequence selected from the group consisting of SEQ ID NOs: 15, 16, 17, 18, 19, 20, 21, 22, and 23, or
an amino acid sequence having at least 85%, 90%, 95%, 98% or 99% identity to SEQ ID NO: 15, and comprising the CDRb1, CDRb2, and CDRb3 of SEQ ID NOs: 5, 9, and 6, respectively,
an amino acid sequence having at least 85%, 90%, 95%, 98% or 99% identity to SEQ ID NO: 16, and comprising the CDRb1, CDRb2, and CDRb3 of SEQ ID NOs: 5, 10, and 6, respectively,
an amino acid sequence having at least 85%, 90%, 95%, 98% or 99% identity to SEQ ID NO: 17, and comprising the CDRb1, CDRb2, and CDRb3 of SEQ ID NOs: 5, 10, and 6, respectively,
an amino acid sequence having at least 85%, 90%, 95%, 98% or 99% identity to SEQ ID NO: 18, and comprising the CDRb1, CDRb2, and CDRb3 of SEQ ID NOs: 5, 10, and 6, respectively,
an amino acid sequence having at least 85%, 90%, 95%, 98% or 99% identity to SEQ ID NO: 19, and comprising the CDRb1, CDRb2, and CDRb3 of SEQ ID NOs: 5, 10, and 6, respectively,
an amino acid sequence having at least 85%, 90%, 95%, 98% or 99% identity to SEQ ID NO: 20, and comprising the CDRb1, CDRb2, and CDRb3 of SEQ ID NOs: 5, 10, and 6, respectively,
an amino acid sequence having at least 85%, 90%, 95%, 98% or 99% identity to SEQ ID NO: 21, and comprising the CDRb1, CDRb2, and CDRb3 of SEQ ID NOs: 5, 10, and 6, respectively,
an amino acid sequence having at least 85%, 90%, 95%, 98% or 99% identity to SEQ ID NO: 22, and comprising the CDRb1, CDRb2, and CDRb3 of SEQ ID NOs: 5, 9, and 6, respectively, or
an amino acid sequence having at least 85%, 90%, 95%, 98% or 99% identity to SEQ ID NO: 23, and comprising the CDRb1, CDRb2, and CDRb3 of SEQ ID NOs: 5, 9, and 6, respectively,
wherein optionally the CDRa1, CDRa2, CDRa3, CDRb1, CDRb2, and/or CDRb3 sequences may comprise one, two or three amino acid mutations, preferably amino acid substitutions.

In some embodiments of the antigen binding protein, the CDRa1, CDRa2, CDRa3, CDRb1, CDRb2 and CDRb3 sequence(s) comprise up to three amino acid mutations in each of CDRa1, CDRa2, CDRa3, CDRb1, CDRb2 and CDRb3.

In some embodiments of the antigen binding protein,
the V_{A} comprises an amino acid sequence of SEQ ID NO: 11 or an amino acid sequence having at least 85%, 90%, 95%, 98% or 99% identity to SEQ ID NO: 11, and comprising the CDRa1, CDRa2, and CDRa3 of SEQ ID NOS: 2, 7, and 3, respectively, and
the V_{B} comprises an amino acid sequence of SEQ ID NO: 15 or an amino acid sequence having at least 85%, 90%, 95%, 98% or 99% identity to SEQ ID NO: 15, and comprising the CDRb1, CDRb2, and CDRb3 of SEQ ID NOs: 5, 9, and 6, respectively;
the V_{A} comprises an amino acid sequence of SEQ ID NO: 12 or an amino acid sequence having at least 85%, 90%, 95%, 98% or 99% identity to SEQ ID NO: 12, and comprising the CDRa1, CDRa2, and CDRa3 of SEQ ID NOs: 2, 8, and 4, respectively, and
the V_{B} comprises an amino acid sequence of SEQ ID NO: 16 or an amino acid sequence having at least 85%, 90%, 95%, 98% or 99% identity to SEQ ID NO: 16, and comprising the CDRb1, CDRb2, and CDRb3 of SEQ ID NOs: 5, 10, and 6, respectively;
the V_{A} comprises an amino acid sequence of SEQ ID NO: 13 or an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, and
the V_{B} comprises an amino acid sequence of SEQ ID NO: 17 or an amino acid sequence having at least 85%, 90%, 95%, 98% or 99% identity to SEQ ID NO: 17, and comprising the CDRb1, CDRb2, and CDRb3 of SEQ ID NOs: 5, 10, and 6, respectively;
the V_{A} comprises an amino acid sequence of SEQ ID NO: 14 or an amino acid sequence having at least 85%, 90%, 95%, 98% or 99% identity to SEQ ID NO: 14, and comprising the CDRa1, CDRa2, and CDRa3 of SEQ ID NOs: 2, 8, and 4, respectively, and
the V_{B} comprises an amino acid sequence of SEQ ID NO: 18 or an amino acid sequence having at least 85%, 90%, 95%, 98% or 99% identity to SEQ ID NO: 18, and comprising the CDRb1, CDRb2, and CDRb3 of SEQ ID NOs: 5, 10, and 6, respectively;
the V_{A} comprises an amino acid sequence of SEQ ID NO: 13 or an amino acid sequence having at least 85%, 90%, 95%, 98% or 99% identity to SEQ ID NO: 13, and comprising the CDRa1, CDRa2, and CDRa3 of SEQ ID NOS: 2, 7, and 3, respectively, and
the V_{B} comprises an amino acid sequence of SEQ ID NO: 19 or an amino acid sequence having at least 85%, 90%, 95%, 98% or 99% identity to SEQ ID NO: 19, and comprising the CDRb1, CDRb2, and CDRb3 of SEQ ID NOs: 5, 10, and 6, respectively;
the V_{A} comprises an amino acid sequence of SEQ ID NO: 11 or an amino acid sequence having at least 85%, 90%, 95%, 98% or 99% identity to SEQ ID NO: 11, and comprising the CDRa1, CDRa2, and CDRa3 of SEQ ID NOS: 2, 7, and 3, respectively, and
the V_{B} comprises an amino acid sequence of SEQ ID NO: 19 or an amino acid sequence having at least 85%, 90%, 95%, 98% or 99% identity to SEQ ID NO: 19, and comprising the CDRb1, CDRb2, and CDRb3 of SEQ ID NOs: 5, 10, and 6, respectively;
the V_{A} comprises an amino acid sequence of SEQ ID NO: 14 or an amino acid sequence having at least 85%, 90%, 95%, 98% or 99% identity to SEQ ID NO: 14, and comprising the CDRa1, CDRa2, and CDRa3 of SEQ ID NOs: 2, 8, and 4, respectively, and
the V_{B} comprises an amino acid sequence of SEQ ID NO: 20 or an amino acid sequence having at least 85%, 90%, 95%, 98% or 99% identity to SEQ ID NO: 20, and comprising the CDRb1, CDRb2, and CDRb3 of SEQ ID NOs: 5, 10, and 6, respectively;
the V_{A} comprises an amino acid sequence of SEQ ID NO: 13 or an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, and
the V_{B} comprises an amino acid sequence of SEQ ID NO: 21 or an amino acid sequence having at least 85%, 90%, 95%, 98% or 99% identity to SEQ ID NO: 21, and comprising the CDRb1, CDRb2, and CDRb3 of SEQ ID NOs: 5, 10, and 6, respectively;
the V_{A} comprises an amino acid sequence of SEQ ID NO: 11 or an amino acid sequence having at least 85%, 90%, 95%, 98% or 99% identity to SEQ ID NO: 11, and comprising the CDRa1, CDRa2, and CDRa3 of SEQ ID NOs: 2, 7, and 3, respectively, and
the V_{B} comprises an amino acid sequence of SEQ ID NO: 21 or an amino acid sequence having at least 85%, 90%, 95%, 98% or 99% identity to SEQ ID NO: 21, and comprising the CDRb1, CDRb2, and CDRb3 of SEQ ID NOs: 5, 10, and 6, respectively;
the V_{A} comprises an amino acid sequence of SEQ ID NO: 12 or an amino acid sequence having at least 85%, 90%, 95%, 98% or 99% identity to SEQ ID NO: 12, and comprising the CDRa1, CDRa2, and CDRa3 of SEQ ID NOs: 2, 8, and 4, respectively, and
the V_{B} comprises an amino acid sequence of SEQ ID NO: 18 or an amino acid sequence having at least 85%, 90%, 95%, 98% or 99% identity to SEQ ID NO: 18, and comprising the CDRb1, CDRb2, and CDRb3 of SEQ ID NOs: 5, 10, and 6, respectively;
the V_{A} comprises an amino acid sequence of SEQ ID NO: 13 or an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, and
the V_{B} comprises an amino acid sequence of SEQ ID NO: 22 or an amino acid sequence having at least 85%, 90%, 95%, 98% or 99% identity to SEQ ID NO: 22, and comprising the CDRb1, CDRb2, and CDRb3 of SEQ ID NOs: 5, 9, and 6, respectively; or
the V_{A} comprises an amino acid sequence of SEQ ID NO: 14 or an amino acid sequence having at least 85%, 90%, 95%, 98% or 99% identity to SEQ ID NO: 14, and comprising the CDRa1, CDRa2, and CDRa3 of SEQ ID NOs: 2, 8, and 4, respectively, and
the V_{B} comprises an amino acid sequence of SEQ ID NO: 23 or an amino acid sequence having at least 85%, 90%, 95%, 98% or 99% identity to SEQ ID NO: 23, and comprising the CDRb1, CDRb2, and CDRb3 of SEQ ID NOs: 5, 9, and 6, respectively;
wherein optionally said CDRa1, CDRa2, CDRa3, CDRb1, CDRb2, and/or CDRb3 sequences may comprise one, two or three amino acid mutations, preferably amino acid substitutions.

In some embodiments of the antigen binding protein, the CDRa1, CDRa2, CDRa3, CDRb1, CDRb2 and CDRb3 sequence(s) comprise up to three amino acid mutations in each of CDRa1, CDRa2, CDRa3, CDRb1, CDRb2 and CDRb3.

In all embodiments of the antigen binding protein of the invention, amino acid mutations within the CDRa1, CDRa2, CDRa3, CDRb1, CDRb2 and CDRb3 sequences—if present—are preferably amino acid substitutions, more preferably conservative amino acid substitutions (see Table 1). It is preferred that the CDR sequences comprise not more than two, preferably not more than one, amino acid mutation(s). It is further preferred that the amino acid mutation(s)—if present—are at the first or last position of the respective CDR sequence. In most preferred embodiments, the CDR sequences do not comprise any amino acid mutation.

In some embodiments of the antigen binding protein, the first and the last two positions of the CDR1, CDR2 and/or CDR3 of the V_{A} and/or the V_{B} comprise a conservative substitution, and preferably when the CDRa1, CDRa3, CDRb1 and/or CDRb3 may comprise one, two or three amino acid mutations. In particular embodiments of the antigen binding protein, the first and the last two positions of the CDR3 of the V_{A} and/or the V_{B} domain comprise a conservative substitution, and preferably when the CDRa3 and/or CDRb3 may comprise one, two or three amino acid mutations.

Introducing a mutation into a known amino acid sequence is standard procedure well-known in the art and routine work for the skilled person. Respective methods are known in the field (e.g. Stratagene's QuikChange Site Directed Mutagenesis Kit since 2007). The skilled person is thus very well capable of introducing specific mutations such as substitutions into an amino acid sequence in general and into a CDR sequence in particular.

Screening of variants of a given CDR for binding to its target is also a procedure applied by the skilled person. The present application describes functional assays, including cytokine production assays to determine binding of an antigen binding protein of the invention to the MAGEB2 antigenic peptide. Binding of an antigen binding protein of the invention to the MAGEB2 antigenic peptide can also be determined by peptide MHC multimer staining or biolayer interferometry.

While the outcome of an amino acid mutation in a CDR may not be readily predictable, the skilled person would be well capable of generating and screening multiple mutants without undue burden. The skilled person would thus be able to generate antigen binding proteins carrying one, two or three amino acid mutations within their CDRs and subsequently identify antigen binding proteins having the same binding characteristics as an antigen binding protein comprising the CDR sequences of Table 7.

In some embodiments of the antigen binding protein, the V_{A} comprises an amino acid sequence selected from the group consisting of SEQ ID NOS: 11, 12, 13, and 14; and the V_{B} comprises an amino acid sequence selected from the group consisting of SEQ ID NOs: 15, 16, 17, 18, 19, 20, 21, 22, and 23.

In preferred embodiments of the antigen binding protein,
the V_{A} comprises an amino acid sequence of SEQ ID NO: 11 and the V_{B} comprises an amino acid sequence of SEQ ID NO: 15;
the V_{A} comprises an amino acid sequence of SEQ ID NO: 12 and the V_{B} comprises an amino acid sequence of SEQ ID NO: 16;
the V_{A} comprises an amino acid sequence of SEQ ID NO: 13 and the V_{B} comprises an amino acid sequence of SEQ ID NO: 17;
the V_{A} comprises an amino acid sequence of SEQ ID NO: 14 and the V_{B} comprises an amino acid sequence of SEQ ID NO: 18;
the V_{A} comprises an amino acid sequence of SEQ ID NO: 13 and the V_{B} comprises an amino acid sequence of SEQ ID NO: 19;
the V_{A} comprises an amino acid sequence of SEQ ID NO: 11 and the V_{B} comprises an amino acid sequence of SEQ ID NO: 19;
the V_{A} comprises an amino acid sequence of SEQ ID NO: 14 and the V_{B} comprises an amino acid sequence of SEQ ID NO: 20;
the V_{A} comprises an amino acid sequence of SEQ ID NO: 13 and the V_{B} comprises an amino acid sequence of SEQ ID NO: 21;
the V_{A} comprises an amino acid sequence of SEQ ID NO: 11 and the V_{B} comprises an amino acid sequence of SEQ ID NO: 21;
the V_{A} comprises an amino acid sequence of SEQ ID NO: 12 and the V_{B} comprises an amino acid sequence of SEQ ID NO: 18;
the V_{A} comprises an amino acid sequence of SEQ ID NO: 13 and the V_{B} comprises an amino acid sequence of SEQ ID NO: 22; or the V_{A} comprises an amino acid sequence of SEQ ID NO: 14 and the V_{B} comprises an amino acid sequence of SEQ ID NO: 23;
optionally wherein the framework region(s) comprise(s) at least one amino acid substitution selected from the group of amino acid substitutions consisting of:
   an amino acid substitution at position 19 in FR1-a, wherein said amino acid substitution is a hydrophobic amino acid selected from the group consisting of A, G, F, L, I, Y, W, V, M and P, preferably A, F, I, L, V and M, such as S19A, S191, S19L, or S19V, more preferably S19A, or S19V, even more preferably S19A,
   an amino acid substitution at position 39 in FR2-a, wherein said amino acid substitution is L39F, L39I, or L39V, most preferably L39F,
   an amino acid substitution at position 13 in FR1-b, wherein said amino acid substitution is C13V, C13G, C13T, C13S, or C13A, preferably C13V,
   amino acid substitutions at positions 94 and/or 95 in FR3-b, wherein said amino acid substitution at position 94 is M94V, and/or said amino acid substitution at position 95 is S95E, or S95D, preferably S95E, respectively;
and wherein the positions of the substitutions are given according to the IMGT nomenclature.

In some embodiments, the antigen binding protein further comprises a constant domain, wherein the constant domain comprises an amino acid sequence selected from the group consisting of SEQ ID NOs: 24 and 25, or an amino acid sequence having at least 85%, 90%, 95%, 98% or 99% identity to SEQ ID NOs: 24 and 25; and/or comprises a linker, wherein the linker comprises an amino acid sequence of SEQ ID NO: 26, or an amino acid sequence having at least 85%, 90%, 95%, 98% or 99% identity to SEQ ID NO: 26.

In some embodiments of the antigen binding protein,
the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively;
the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively;
the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively;
the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively;
the first polypeptide comprises an amino acid sequence of SEQ ID NO: 31, or an amino acid sequence having at least 85%, 90%, 95%, 98% or 99% identity to SEQ ID NO: 31 and comprising the CDRa1, CDRa2, and CDRa3 of SEQ ID NO: 2, 8, and 4, respectively;
the first polypeptide comprises an amino acid sequence of SEQ ID NO: 32, or an amino acid sequence having at least 85%, 90%, 95%, 98% or 99% identity to SEQ ID NO: 32 and comprising the CDRa1, CDRa2, and CDRa3 of SEQ ID NO: 2, 8, and 4, respectively; or
the first polypeptide comprises an amino acid sequence of SEQ ID NO: 33, or an amino acid sequence having at least 85%, 90%, 95%, 98% or 99% identity to SEQ ID NO: 33 and comprising the CDRa1, CDRa2, and CDRa3 of SEQ ID NO: 2, 8, and 4, respectively; and
the second polypeptide comprises an amino acid sequence of SEQ ID NO: 34, or an amino acid sequence having at least 85%, 90%, 95%, 98% or 99% identity to SEQ ID NO: 34 and comprising the CDRb1, CDRb2, and CDRb3 of SEQ ID NO: 5, 9, and 6, respectively;
the second polypeptide comprises an amino acid sequence of SEQ ID NO: 35, or an amino acid sequence having at least 85%, 90%, 95%, 98% or 99% identity to SEQ ID NO: 35 and comprising the CDRb1, CDRb2, and CDRb3 of SEQ ID NO: 5, 10, and 6, respectively;
the second polypeptide comprises an amino acid sequence of SEQ ID NO: 36, or an amino acid sequence having at least 85%, 90%, 95%, 98% or 99% identity to SEQ ID NO: 36 and comprising the CDRb1, CDRb2, and CDRb3 of SEQ ID NO: 5, 10, and 6, respectively;
the second polypeptide comprises an amino acid sequence of SEQ ID NO: 37, or an amino acid sequence having at least 85%, 90%, 95%, 98% or 99% identity to SEQ ID NO: 37 and comprising the CDRb1, CDRb2, and CDRb3 of SEQ ID NO: 5, 10, and 6, respectively;
the second polypeptide comprises an amino acid sequence of SEQ ID NO: 38, or an amino acid sequence having at least 85%, 90%, 95%, 98% or 99% identity to SEQ ID NO: 38 and comprising the CDRb1, CDRb2, and CDRb3 of SEQ ID NO: 5, 10, and 6, respectively;
the second polypeptide comprises an amino acid sequence of SEQ ID NO: 39, or an amino acid sequence having at least 85%, 90%, 95%, 98% or 99% identity to SEQ ID NO: 39 and comprising the CDRb1, CDRb2, and CDRb3 of SEQ ID NO: 5, 10, and 6, respectively;
the second polypeptide comprises an amino acid sequence of SEQ ID NO: 40, or an amino acid sequence having at least 85%, 90%, 95%, 98% or 99% identity to SEQ ID NO: 40 and comprising the CDRb1, CDRb2, and CDRb3 of SEQ ID NO: 5, 10, and 6, respectively;
the second polypeptide comprises an amino acid sequence of SEQ ID NO: 41, or an amino acid sequence having at least 85%, 90%, 95%, 98% or 99% identity to SEQ ID NO: 41 and comprising the CDRb1, CDRb2, and CDRb3 of SEQ ID NO: 5, 9, and 6, respectively;
the second polypeptide comprises an amino acid sequence of SEQ ID NO: 42, or an amino acid sequence having at least 85%, 90%, 95%, 98% or 99% identity to SEQ ID NO: 42 and comprising the CDRb1, CDRb2, and CDRb3 of SEQ ID NO: 5, 9, and 6, respectively;
the second polypeptide comprises an amino acid sequence of SEQ ID NO: 43, or an amino acid sequence having at least 85%, 90%, 95%, 98% or 99% identity to SEQ ID NO: 43 and comprising the CDRb1, CDRb2, and CDRb3 of SEQ ID NO: 5, 9, and 6, respectively;
the second polypeptide comprises an amino acid sequence of SEQ ID NO: 44, or an amino acid sequence having at least 85%, 90%, 95%, 98% or 99% identity to SEQ ID NO: 44 and comprising the CDRb1, CDRb2, and CDRb3 of SEQ ID NO: 5, 9, and 6, respectively; or
the second polypeptide comprises an amino acid sequence of SEQ ID NO: 45, or an amino acid sequence having at least 85%, 90%, 95%, 98% or 99% identity to SEQ ID NO: 45 and comprising the CDRb1, CDRb2, and CDRb3 of SEQ ID NO: 5, 9, and 6, respectively;
wherein optionally said CDRa1, CDRa2, CDRa3, CDRb1, CDRb2 and/or CDRb3 sequences may comprise one, two or three amino acid mutations, preferably amino acid substitutions.

In some embodiments of the antigen binding protein, the CDRa1, CDRa2, CDRa3, CDRb1, CDRb2 and CDRb3 sequence(s) comprise up to three amino acid mutations in each of CDRa1, CDRa2, CDRa3, CDRb1, CDRb2 and CDRb3.

In some embodiments of the antigen binding protein,
the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
the second polypeptide comprises an amino acid sequence of SEQ ID NO: 34, or an amino acid sequence having at least 85%, 90%, 95%, 98% or 99% identity to SEQ ID NO: 34 and comprising the CDRb1, CDRb2, and CDRb3 of SEQ ID NO: 5, 9, and 6, respectively;
the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively, and
the second polypeptide comprises an amino acid sequence of SEQ ID NO: 35, or an amino acid sequence having at least 85%, 90%, 95%, 98% or 99% identity to SEQ ID NO: 35 and comprising the CDRb1, CDRb2, and CDRb3 of SEQ ID NO: 5, 10, and 6, respectively;
the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
the second polypeptide comprises an amino acid sequence of SEQ ID NO: 36, or an amino acid sequence having at least 85%, 90%, 95%, 98% or 99% identity to SEQ ID NO: 36 and comprising the CDRb1, CDRb2, and CDRb3 of SEQ ID NO: 5, 10, and 6, respectively;
the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
the second polypeptide comprises an amino acid sequence of SEQ ID NO: 37, or an amino acid sequence having at least 85%, 90%, 95%, 98% or 99% identity to SEQ ID NO: 37 and comprising the CDRb1, CDRb2, and CDRb3 of SEQ ID NO: 5, 10, and 6, respectively;
the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
the second polypeptide comprises an amino acid sequence of SEQ ID NO: 38, or an amino acid sequence having at least 85%, 90%, 95%, 98% or 99% identity to SEQ ID NO: 38 and comprising the CDRb1, CDRb2, and CDRb3 of SEQ ID NO: 5, 10, and 6, respectively;
the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
the second polypeptide comprises an amino acid sequence of SEQ ID NO: 38, or an amino acid sequence having at least 85%, 90%, 95%, 98% or 99% identity to SEQ ID NO: 38 and comprising the CDRb1, CDRb2, and CDRb3 of SEQ ID NO: 5, 10, and 6, respectively;
the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
the second polypeptide comprises an amino acid sequence of SEQ ID NO: 39, or an amino acid sequence having at least 85%, 90%, 95%, 98% or 99% identity to SEQ ID NO: 39 and comprising the CDRb1, CDRb2, and CDRb3 of SEQ ID NO: 5, 10, and 6, respectively;
the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
the second polypeptide comprises an amino acid sequence of SEQ ID NO: 40, or an amino acid sequence having at least 85%, 90%, 95%, 98% or 99% identity to SEQ ID NO: 40 and comprising the CDRb1, CDRb2, and CDRb3 of SEQ ID NO: 5, 10, and 6, respectively;
the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
the second polypeptide comprises an amino acid sequence of SEQ ID NO: 40, or an amino acid sequence having at least 85%, 90%, 95%, 98% or 99% identity to SEQ ID NO: 40 and comprising the CDRb1, CDRb2, and CDRb3 of SEQ ID NO: 5, 10, and 6, respectively;
the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively, and
the second polypeptide comprises an amino acid sequence of SEQ ID NO: 37, or an amino acid sequence having at least 85%, 90%, 95%, 98% or 99% identity to SEQ ID NO: 37 and comprising the CDRb1, CDRb2, and CDRb3 of SEQ ID NO: 5, 10, and 6, respectively;
the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
the second polypeptide comprises an amino acid sequence of SEQ ID NO: 41, or an amino acid sequence having at least 85%, 90%, 95%, 98% or 99% identity to SEQ ID NO: 41 and comprising the CDRb1, CDRb2, and CDRb3 of SEQ ID NO: 5, 9, and 6, respectively;
the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
the second polypeptide comprises an amino acid sequence of SEQ ID NO: 42, or an amino acid sequence having at least 85%, 90%, 95%, 98% or 99% identity to SEQ ID NO: 42 and comprising the CDRb1, CDRb2, and CDRb3 of SEQ ID NO: 5, 9, and 6, respectively;
the first polypeptide comprises an amino acid sequence of SEQ ID NO: 31, or an amino acid sequence having at least 85%, 90%, 95%, 98% or 99% identity to SEQ ID NO: 31 and comprising the CDRa1, CDRa2, and CDRa3 of SEQ ID NO: 2, 8, and 4, respectively, and
the second polypeptide comprises an amino acid sequence of SEQ ID NO: 43, or an amino acid sequence having at least 85%, 90%, 95%, 98% or 99% identity to SEQ ID NO: 43 and comprising the CDRb1, CDRb2, and CDRb3 of SEQ ID NO: 5, 9, and 6, respectively;
the first polypeptide comprises an amino acid sequence of SEQ ID NO: 32, or an amino acid sequence having at least 85%, 90%, 95%, 98% or 99% identity to SEQ ID NO: 32 and comprising the CDRa1, CDRa2, and CDRa3 of SEQ ID NO: 2, 8, and 4, respectively, and
the second polypeptide comprises an amino acid sequence of SEQ ID NO: 44, or an amino acid sequence having at least 85%, 90%, 95%, 98% or 99% identity to SEQ ID NO: 44 and comprising the CDRb1, CDRb2, and CDRb3 of SEQ ID NO: 5, 9, and 6, respectively; or
the first polypeptide comprises an amino acid sequence of SEQ ID NO: 33, or an amino acid sequence having at least 85%, 90%, 95%, 98% or 99% identity to SEQ ID NO: 33 and comprising the CDRa1, CDRa2, and CDRa3 of SEQ ID NO: 2, 8, and 4, respectively, and
the second polypeptide comprises an amino acid sequence of SEQ ID NO: 45, or an amino acid sequence having at least 85%, 90%, 95%, 98% or 99% identity to SEQ ID NO: 45 and comprising the CDRb1, CDRb2, and CDRb3 of SEQ ID NO: 5, 9, and 6, respectively;
wherein optionally said CDRa1, CDRa2, CDRa3, CDRb1, CDRb2 and/or CDRb3 sequences may comprise one, two or three amino acid mutations, preferably amino acid substitutions.

In particular embodiments of the antigen binding protein,
the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
the second polypeptide comprises an amino acid sequence of SEQ ID NO: 34, or an amino acid sequence having at least 85%, 90%, 95%, 98% or 99% identity to SEQ ID NO: 34 and comprising the CDRb1, CDRb2, and CDRb3 of SEQ ID NO: 5, 9, and 6, respectively; or
the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively, and
the second polypeptide comprises an amino acid sequence of SEQ ID NO: 35, or an amino acid sequence having at least 85%, 90%, 95%, 98% or 99% identity to SEQ ID NO: 35 and comprising the CDRb1, CDRb2, and CDRb3 of SEQ ID NO: 5, 10, and 6, respectively.

In some embodiments of the antigen binding protein, the CDRa1, CDRa2, CDRa3, CDRb1, CDRb2 and CDRb3 sequence(s) comprise up to three amino acid mutations in each of CDRa1, CDRa2, CDRa3, CDRb1, CDRb2 and CDRb3.

In some embodiments of the antigen binding protein,
the first polypeptide comprises an amino acid sequence of SEQ ID NO: 27 and the second polypeptide comprises an amino acid sequence of SEQ ID NO: 34;
the first polypeptide comprises an amino acid sequence of SEQ ID NO: 28 and the second polypeptide comprises an amino acid sequence of SEQ ID NO: 35;
the first polypeptide comprises an amino acid sequence of SEQ ID NO: 29 and the second polypeptide comprises an amino acid sequence of SEQ ID NO: 36;
the first polypeptide comprises an amino acid sequence of SEQ ID NO: 30 and the second polypeptide comprises an amino acid sequence of SEQ ID NO: 37;
the first polypeptide comprises an amino acid sequence of SEQ ID NO: 29 and the second polypeptide comprises an amino acid sequence of SEQ ID NO: 38;
the first polypeptide comprises an amino acid sequence of SEQ ID NO: 27 and the second polypeptide comprises an amino acid sequence of SEQ ID NO: 38;
the first polypeptide comprises an amino acid sequence of SEQ ID NO: 30 and the second polypeptide comprises an amino acid sequence of SEQ ID NO: 39;
the first polypeptide comprises an amino acid sequence of SEQ ID NO: 29 and the second polypeptide comprises an amino acid sequence of SEQ ID NO: 40;
the first polypeptide comprises an amino acid sequence of SEQ ID NO: 27 and the second polypeptide comprises an amino acid sequence of SEQ ID NO: 40;
the first polypeptide comprises an amino acid sequence of SEQ ID NO: 28 and the second polypeptide comprises an amino acid sequence of SEQ ID NO: 37;
the first polypeptide comprises an amino acid sequence of SEQ ID NO: 29 and the second polypeptide comprises an amino acid sequence of SEQ ID NO: 41;
the first polypeptide comprises an amino acid sequence of SEQ ID NO: 30 and the second polypeptide comprises an amino acid sequence of SEQ ID NO: 42;
the first polypeptide comprises an amino acid sequence of SEQ ID NO: 31 and the second polypeptide comprises an amino acid sequence of SEQ ID NO: 43;
the first polypeptide comprises an amino acid sequence of SEQ ID NO: 32 and the second polypeptide comprises an amino acid sequence of SEQ ID NO: 44; or
the first polypeptide comprises an amino acid sequence of SEQ ID NO: 33 and the second polypeptide comprises an amino acid sequence of SEQ ID NO: 45.

In some particular aspects, the antigen binding protein comprises,
the first polypeptide comprises an amino acid sequence of SEQ ID NO: 27 and the second polypeptide comprises an amino acid sequence of SEQ ID NO: 34; or
the first polypeptide comprises an amino acid sequence of SEQ ID NO: 28 and the second polypeptide comprises an amino acid sequence of SEQ ID NO: 35.

In some embodiments, the antigen-binding domain A is derived from a TCR, or is a TCR or fragment(s) thereof.

In a preferred embodiment, the TCR is selected from the group consisting of an α/β TCR, a γ/δ TCR, functional fragments of a TCR, and a fusion protein or chimeric protein comprising a functional fragment of a TCR. In some embodiments, V_{A} and V_{B} are TCR variable domains, in particular TCR alpha, beta, gamma or delta variable domains. In some embodiments, V_{A} is a TCR alpha, gamma or delta variable domain and V_{B} is a TCR beta, gamma or delta variable domain. Preferably, V_{A} is a TCR alpha variable domain and V_{B} is a TCR beta variable domain, or V_{A} is a TCR gamma variable domain and V_{B} is a TCR delta variable domain, or V_{A} is a TCR alpha variable domain and V_{B} is a TCR gamma variable domain, or V_{A} is a TCR delta variable domain and V_{B} is a TCR beta variable domain. In preferred embodiments, V_{A} and V_{B} are TCRα and TCRβ domains, respectively. In some embodiments, V_{A} is a TCR gamma variable domain comprising CDR1 and CDR3 and optionally CDR2 derived from a TCR alpha variable domain, and/or V_{B} is a TCR delta variable domain comprising CDR1 and CDR3 and optionally CDR2 derived from a TCR beta variable domain.

In some embodiments, the MHC protein is an HLA protein, preferably HLA-A, more preferably HLA-A*02.

The antigen binding proteins of the invention have a high specificity for the MAGEB2 antigenic peptide (SEQ ID NO: 1), in particular an increased specificity in comparison to a reference protein when measured under similar, preferably identical experimental conditions. The inventors demonstrate in Example 6 that the antigen binding proteins of the present invention bind the target antigen, i.e. the MAGEB2 antigenic peptide, in a complex with a MHC protein, with high specificity.

The inventors identified potential off-target peptides that are, for example, similar to the sequence and/or motif of MAGEB2, and thus have an increased risk of being bound by an antigen binding protein binding to MAGEB2. However, the antigen binding proteins of the invention do not specifically bind to these peptides.

In some embodiments, the antigen binding protein does not significantly bind to at least 1, at least 2, at least 3, at least 4, at least 5, or all similar peptides selected from the group consisting of SEQ ID NO: 59 (SP-02-1621), SEQ ID NO: 60 (SP-02-1622), SEQ ID NO: 61 (SP-02-1623), SEQ ID NO: 62 (SP-02-1624), SEQ ID NO: 63 (SP-02-1625), SEQ ID NO: 64 (SP-02-1626), SEQ ID NO: 65 (SP-02-1627), SEQ ID NO: 66 (SP-02-1628), SEQ ID NO: 67 (SP-02-1629), SEQ ID NO: 106 (SP-02-1640), SEQ ID NO: 133 (SP-01-0010), SEQ ID NO: 134 (SP-02-1631), SEQ ID NO: 135 (SP-02-1635), SEQ ID NO: 136 (SP-02-1641), SEQ ID NO: 137 (SP-02-1642), SEQ ID NO: 138 (SP-02-1643), SEQ ID NO: 139 (SP-02-1644), SEQ ID NO: 140 (SP-02-1647), SEQ ID NO: 141 (SP-02-1648), SEQ ID NO: 142 (SP-02-1650), SEQ ID NO: 143 (SP-02-1651), SEQ ID NO: 144 (SP-02-1652), SEQ ID NO: 145 (SP-02-1653), SEQ ID NO: 146 (SP-02-1655), SEQ ID NO: 147 (SP-02-1656), SEQ ID NO: 148 (SP-02-1664), SEQ ID NO: 149 (SP-02-1673), SEQ ID NO: 150 (SP-02-1722) and SEQ ID NO: 151 (SP-02-1724).

In preferred embodiments, the antigen binding protein is a soluble protein.

In some embodiments, the antigen binding protein is a multispecific antigen binding protein, in particular a bispecific antigen binding protein.

In particular embodiments, the antigen binding protein comprises a further antigen-binding domain or site B. In further embodiments, the antigen binding protein may comprise further antigen binding sites/domains, e.g. C, D, E, etc. The further binding domain may for example be a recruiter that concentrates or recruits further binding moieties in spatial proximity to the antigen binding protein, e.g. T cells.

In some embodiments, the antigen binding protein further comprises a variable domain V_{H} comprising a CDRH1, a CDRH2 and a CDRH3, and a V_{L} comprising a CDRL1, a CDRL2, and a CDRL3, wherein CDRH1, CDRH2, CDRH3, CDRL1, CDRL2 and CDRL3 form an antigen-binding domain B. The V_{H} and VL may be derived from an antibody. The antigen binding domain B may also be a further TCR derived binding domain, or be a further binding domain, such as an Fc domain.

In particular embodiments, the antigen-binding domain B specifically binds to immune cells, preferably T cells or natural killer (NK) cells.

In some embodiments, the antigen-binding domain B specifically binds to an antigen selected from the group consisting of CD2, CD3, in particular CD3γ, CD3δ, and/or CD3ε, CD4, CD5, CD7, CD8, CD10, CD11b, CD11c, CD14, CD16, CD18, CD22, CD25, CD28, CD32a, CD32b, CD33, CD41, CD41b, CD42a, CD42b, CD44, CD45RA, CD49, CD55, CD56, CD61, CD64, CD68, CD69, CD89, CD90, CD94, CD95, CD117, CD123, CD125, CD134, CD137, CD152, CD163, CD193, CD203c, CD235a, CD278, CD279, CD287, Ly-6.2C, Mel14, Nkp46, NKG2D, GITR, F_{C}εRI, TCRα/β, TCRγ/δ, HLA-DR and 4-1 BB, or combinations thereof.

An example of a receptor molecule that is present on the surface of both T cells and natural killer (NK) cells is CD2 and further members of the CD2-superfamily. CD2 is able to act as a co-stimulatory molecule on T and NK cells.

In preferred embodiments, the antigen-binding domain B specifically binds to the antigen CD3. In more preferred embodiments, the antigen-binding domain B specifically binds to an α/β T cell receptor (TCR)/CD3 complex.

In some embodiments, the CDRH3 comprises the amino acid sequence of GSYYDYEGFVY [SEQ ID NO: 50] or GSYYDYDGFVY [SEQ ID NO: 58], and optionally CDRH3 comprises up to three amino acid mutations, preferably amino acid substitutions.

In preferred embodiments of an antibody-binding protein comprising a binding domain B, the V_{H} comprises (a) a CDRH1 comprising the amino acid sequence of X₁YVMH, wherein X₁ is S, R, K or H [SEQ ID NO: 111], (b) a CDRH2 comprising the amino acid sequence of YINPX₁X₂DVTKYX₃X₄KFX₅G, wherein X1 is Y, R, K, or H, preferably Y or R; X₂ is N, R, or K, preferably N or R; X₃ is A or N; X₄ is E or Q, preferably E; and X5 is Q or K [SEQ ID NO: 112], and (c) a CDRH3 comprising the amino acid sequence of GSYYDYEGFVY [SEQ ID NO: 50] or GSYYDYDGFVY [SEQ ID NO: 58], and the V_{L} comprises (a) a CDRL1 comprising the amino acid sequence of SATSSVXYMH, wherein X is S, R, or K [SEQ ID NO: 113], (b) a CDRL2 comprising the amino acid sequence of DTSKLAX, wherein X is S, R or K [SEQ ID NO: 114], and (c) a CDRL3 comprising the amino acid sequence of QQWSSNPLT [SEQ ID NO: 53]; and optionally wherein each of the CDRH1, the CDRH2 and the CDRH3, and the CDRL1, CDRL2, and CDRL3 comprises up to three amino acid substitutions.

More preferably, the V_{H} comprises (a) a CDRH1 comprising the amino acid sequence of SYVMH [SEQ ID NO: 48], (b) a CDRH2 comprising the amino acid sequence of YINPRNDVTKYAEKFQG [SEQ ID NO: 49] or YINPYNDVTKYAEKFQG [SEQ ID NO: 57], and (c) a CDRH3 comprising the amino acid sequence of GSYYDYEGFVY [SEQ ID NO: 50] or GSYYDYDGFVY [SEQ ID NO: 58], and the V_{L} comprises (a) a CDRL1 comprising the amino acid sequence of SATSSVSYMH [SEQ ID NO: 51], (b) a CDRL2 comprising the amino acid sequence of DTSKLAS [SEQ ID NO: 52], and (c) a CDRL3 comprising the amino acid sequence of QQWSSNPLT [SEQ ID NO: 53]; and optionally wherein each of the CDRH1, the CDRH2 and the CDRH3, and the CDRL1, CDRL2, and CDRL3 comprises up to three amino acid substitutions.

In some embodiments, the ABP comprises a variable domain V_{H} comprising a CDRH1, a CDRH2 and a CDRH3 of SEQ ID NOs: 48, 57, 50, respectively, and a V_{L} comprising a CDRL1, a CDRL2, and a CDRL3 of SEQ ID NOs: 51 to 53, respectively, wherein CDRH1, CDRH2, CDRH3, CDRL1, CDRL2 and CDRL3 form an antigen binding domain B.

In some embodiments, the ABP comprises a variable domain V_{H} comprising a CDRH1, a CDRH2 and a CDRH3 of SEQ ID NOs: 48, 49, 58, respectively, and a V_{L} comprising a CDRL1, a CDRL2, and a CDRL3 of SEQ ID NOs: 51 to 53, respectively, wherein CDRH1, CDRH2, CDRH3, CDRL1, CDRL2 and CDRL3 form an antigen binding domain B.

In some embodiments, the ABP comprises a variable domain V_{H} comprising a CDRH1, a CDRH2 and a CDRH3 of SEQ ID NOs: 48, 57, 58, respectively, and a V_{L} comprising a CDRL1, a CDRL2, and a CDRL3 of SEQ ID NOs: 51 to 53, respectively, wherein CDRH1, CDRH2, CDRH3, CDRL1, CDRL2 and CDRL3 form an antigen binding domain B.

In preferred embodiments, the ABP comprises a variable domain V_{H} comprising a CDRH1, a CDRH2 and a CDRH3 of SEQ ID NOs: 48, 49, 50, respectively, and a V_{L} comprising a CDRL1, a CDRL2, and a CDRL3 of SEQ ID NOs: 51 to 53, respectively, wherein CDRH1, CDRH2, CDRH3, CDRL1, CDRL2 and CDRL3 form an antigen binding domain B.

In some embodiments of the antigen binding protein, the V_{H} comprises an amino acid sequence selected from the group consisting of SEQ ID NOs: 46, 54, 55 and 56, or an amino acid sequence having at least 85%, 90%, 95%, 98% or 99% identity to SEQ ID NOs: 46, 54, 55 or 56 and preferably comprising the CDRH1, CDRH2, and CDRH3 as disclosed hereinabove; and the V_{L} comprises an amino acid sequence of SEQ ID NO: 47, or an amino acid sequence having at least 85%, 90%, 95%, 98% or 99% identity to SEQ ID NO: 47, and preferably comprising the CDRL1, CDRL2, and CDRL3 as disclosed hereinabove; and optionally wherein each of said CDRH1, CDRH2 and CDRH3, and said CDRL1, CDRL2 and CDRL3 comprises up to three amino acid substitutions.

In preferred embodiments, the antigen-binding site B comprises (i) the V_{H} that comprises an amino acid sequence selected from the group consisting of SEQ ID NOs: 46, 54, 55 and 56, or (an) antigen-binding fragment(s) thereof, and (ii) the V_{L} that comprises an amino acid sequence of SEQ ID NO: 47, or (an) antigen-binding fragment(s) thereof.

In some embodiments, the ABP comprises a variable domain V_{H} comprising an amino acid sequence of SEQ ID NO: 46 and a variable domain V_{L} comprising an amino acid sequence of SEQ ID NO: 47.

In some embodiments, the ABP comprises a variable domain V_{H} comprising an amino acid sequence of SEQ ID NO: 54 and a variable domain V_{L} comprising an amino acid sequence of SEQ ID NO: 47.

In some embodiments, the ABP comprises a variable domain V_{H} comprising an amino acid sequence of SEQ ID NO: 55 and a variable domain V_{L} comprising an amino acid sequence of SEQ ID NO: 47.

In some embodiments, the ABP comprises a variable domain V_{H} comprising an amino acid sequence of SEQ ID NO: 56 and a variable domain V_{L} comprising an amino acid sequence of SEQ ID NO: 47.

In some embodiments, the ABP comprises a variable domain V_{H} comprising an amino acid sequence of SEQ ID NO: 115 and a variable domain V_{L} comprising an amino acid sequence of SEQ ID NO: 116.

In some embodiments, the ABP comprises a variable domain V_{H} comprising an amino acid sequence of SEQ ID NO: 117 and a variable domain V_{L} comprising an amino acid sequence of SEQ ID NO: 118.

In some embodiments, the ABP comprises a variable domain V_{H} comprising an amino acid sequence of SEQ ID NO: 119 and a variable domain V_{L} comprising an amino acid sequence of SEQ ID NO: 120.

In some embodiments, the ABP comprises a variable domain V_{H} comprising an amino acid sequence of SEQ ID NO: 121 and a variable domain V_{L} comprising an amino acid sequence of SEQ ID NO: 122.

In some embodiments, the ABP comprises a variable domain V_{H} comprising an amino acid sequence of SEQ ID NO: 123 and a variable domain V_{L} comprising an amino acid sequence of SEQ ID NO: 124.

In some embodiments, the ABP comprises a variable domain V_{H} comprising an amino acid sequence of SEQ ID NO: 125 and a variable domain V_{L} comprising an amino acid sequence of SEQ ID NO: 126.

In some embodiments, the ABP comprises a variable domain V_{H} comprising an amino acid sequence of SEQ ID NO: 127 and a variable domain V_{L} comprising an amino acid sequence of SEQ ID NO: 128.

In some embodiments, the ABP comprises a variable domain V_{H} comprising an amino acid sequence of SEQ ID NO: 129 and a variable domain V_{L} comprising an amino acid sequence of SEQ ID NO: 130.

In some embodiments, the ABP comprises a variable domain V_{H} comprising an amino acid sequence of SEQ ID NO: 131 and a variable domain V_{L} comprising an amino acid sequence of SEQ ID NO: 132.

Other recruiters may also be used in the antigen binding proteins of the invention as e.g. disclosed in WO 2022/233957, Zhu et al. (J Immunol, 1995, 155, 1903-1 0 1910), Shearman et al. (J Immunol, 1991, 147, 4366-73), WO 2021/023657, Liu et al. (mAbs, 2023 Structure-based engineering of a novel CD38-targeting antibody for reduced polyreactivity, mAbs, 15:1, 2189974).

In some embodiments, the antigen binding protein comprises
a V_{A} comprising:
CDRa1 comprising or consisting of SEQ ID NO: 2,
CDRa2 comprising or consisting of SEQ ID NO: 7 and
CDRa3 comprising or consisting of SEQ ID NO: 3,
a V_{B} comprising:
CDRb1 comprising or consisting of SEQ ID NO: 5,
CDRb2 comprising or consisting of SEQ ID NO: 9 and/or
CDRb3 comprising or consisting of SEQ ID NO: 6.
a V_{H} comprising:
CDRH1 comprising or consisting of SEQ ID NO: 48,
CDRH2 comprising or consisting of SEQ ID NO: 49, and
CDRH3 comprising or consisting of SEQ ID NO: 50,
and a V_{L} comprising:
CDRL1 comprising or consisting of SEQ ID NO: 51,
CDRL2 comprising or consisting of SEQ ID NO: 52, and
CDRL3 comprising or consisting of SEQ ID NO: 53.

In some embodiments, the antigen binding protein comprises
a V_{A} comprising:
CDRa1 comprising or consisting of SEQ ID NO: 2,
CDRa2 comprising or consisting of SEQ ID NO: 8,
CDRa3 comprising or consisting of SEQ ID NO: 4,
a V_{B} comprising:
CDRb1 comprising or consisting of SEQ ID NO: 5,
CDRb2 comprising or consisting of SEQ ID NO: 10,
CDRb3 comprising or consisting of SEQ ID NO: 6,
a V_{H} comprising:
CDRH1 comprising or consisting of SEQ ID NO: 48,
CDRH2 comprising or consisting of SEQ ID NO: 49,
CDRH3 comprising or consisting of SEQ ID NO: 50,
and a V_{L} comprising
CDRL1 comprising or consisting of SEQ ID NO: 51,
CDRL2 comprising or consisting of SEQ ID NO: 52, and
CDRL3 comprising or consisting of SEQ ID NO: 53.

In some embodiments, the antigen binding protein comprises
a V_{A} comprising:
CDRa1 comprising or consisting of SEQ ID NO: 2,
CDRa2 comprising or consisting of SEQ ID NO: 7,
CDRa3 comprising or consisting of SEQ ID NO: 3,
a V_{B} comprising:
CDRb1 comprising or consisting of SEQ ID NO: 5,
CDRb2 comprising or consisting of SEQ ID NO: 10,
CDRb3 comprising or consisting of SEQ ID NO: 6,
a V_{H} comprising:
CDRH1 comprising or consisting of SEQ ID NO: 48,
CDRH2 comprising or consisting of SEQ ID NO: 49,
CDRH3 comprising or consisting of SEQ ID NO: 50,
and a V_{L} comprising
CDRL1 comprising or consisting of SEQ ID NO: 51,
CDRL2 comprising or consisting of SEQ ID NO: 52, and
CDRL3 comprising or consisting of SEQ ID NO: 53.

In these embodiments, the CDRa1, CDRa3, CDRb1, CDRb3, CDRH1, CDRH2, CDRH3, CDRL1, CDRL2, and/or CDRL3 sequence(s) may comprise one, two or three amino acid mutations.

In some embodiments, the first polypeptide and the second polypeptide of the antigen binding protein are covalently linked together, e.g., directly or via a cleavable or non-cleavable linker. In some embodiments, the first polypeptide and the second polypeptide of the antigen binding protein are non-covalently linked together.

In some embodiments, the binding domains A and/or B are in the format selected from the group consisting of an antibody, a single chain fragment variable (scFv), disulfide-stabilized Fv (dsFv), Fab, Fab′, F(ab′)2, a nanobody, a DARPin, a Knottin, a diabody or oligomers thereof. The single chain fragment variable (scFv) may also include single chain fragment(s) of the TCR, e.g. scTvs.

In some embodiments, the binding domains A and B are in the format of an scFv. The antigen binding protein may also comprise the binding domains A and B on two scFvs, e.g. on two polypeptide chains.

In some embodiments, the antigen binding protein comprises a single chain TCR (scTCR) and/or a single-chain bispecific antibody.

In the following, further particular formats of the antigen binding protein are exemplified wherein the antigen binding site A and antigen binding site B are in a particular configuration, e.g., on a particular polypeptide chain. Such formats/configuration only have an exemplary character and the antigen binding sites (e.g. A and B) may for example be on one, two, three, four or more polypeptide chains. For example, the variable domains V1 to V4 are each present on one polypeptide chain, i.e. on four polypeptide chains; or V1 and V2 are present on a polypeptide chain and V3 and V4 are present on a polypeptide chain, e.g. two polypeptide chains; or V1, V2, and V3 are present on a polypeptide chain, and V4 is present on a polypeptide chain, e.g. two polypeptide chains.

As a further example, VB, VA and VH may be on a polypeptide chain and VL may be on a further polypeptide chain (or any other further configuration) as long as VA and VB are able to form antigen binding site A and VH and VL are able to form antigen binding site B, or any further binding site that may be comprised in the antigen binding protein. Alternatively, VB, VA and VH and a dimerizing protein may be on a polypeptide chain and VL may be on a further polypeptide chain, and the other part of the dimerizing protein may be on a third polypeptide chain. The herein provided examples exemplify further antigen binding proteins, such as the TCER format. Thus, the further indicated formulae have no limiting character but are merely examples.

The indicated formulae V1-V4 comprise the herein provided inventive CDR regions. For example, Vα comprise the herein provided CDRs of the alpha variable domain and Vβ comprise the herein provided CDRs of the beta variable domain.

In some embodiments, the antigen binding protein comprises two antigen-binding sites A and B, wherein a polypeptide chain comprises a structure represented by formula [I]:

V1-L1-V2 [I]
wherein V1 is a first variable domain; V2 is a second variable domain; L1 is a linker domain, that is present or absent;
and wherein a further polypeptide chain comprises a structure represented by formula [II]:

V3-L3-V4 [II]
wherein V3 is a third variable domain; V4 is a fourth variable domain; L3 is a linker, that is present or absent;
optionally wherein
V1 and V2 form the antigen-binding site A and V3 and V4 form the antigen-binding site B, or
V1 and V2 form the antigen-binding site B and V3 and V4 form the antigen-binding site A, or
V1 and V3 form the antigen-binding site A and V2 and V4 form the antigen-binding site B, or
V1 and V3 form the antigen-binding site B and V2 and V4 form the antigen-binding site A, or
V1 and V4 form the antigen-binding site A and V2 and V3 form the antigen-binding site B, or
V1 and V4 form the antigen-binding site B and V2 and V3 form the antigen-binding site A, and
optionally, wherein the first polypeptide and the second polypeptide are covalently or non-covalently linked together.

As also disclosed above, V1 and V2 do not necessarily have be on the same polypeptide chain, or V2 and V3 do not necessarily have be on the same polypeptide chain. For example, in some embodiments, the antigen binding protein comprises two antigen-binding sites A and B, wherein a polypeptide chain comprises a structure represented by formula [I]:

V1-L1-V2-L2-V3 [I]
wherein V1 is a first variable domain; V2 is a second variable domain; V3 is a third variable domain; L1 and L2 are (a) linker domain(s) that is present or absent;
and wherein a further polypeptide chain comprises a structure represented by formula [II]:

V4 [II]
wherein V4 is a fourth variable domain;
optionally wherein
V1 and V2 form the antigen-binding site A and V3 and V4 form the antigen-binding site B, or
V1 and V2 form the antigen-binding site B and V3 and V4 form the antigen-binding site A, or
V1 and V3 form the antigen-binding site A and V2 and V4 form the antigen-binding site B, or
V1 and V3 form the antigen-binding site B and V2 and V4 form the antigen-binding site A, or
V1 and V4 form the antigen-binding site A and V2 and V3 form the antigen-binding site B, or
V1 and V4 form the antigen-binding site B and V2 and V3 form the antigen-binding site A, and
optionally, wherein the first polypeptide and the second polypeptide are covalently or non-covalently linked together

Such antigen binding proteins, or the polypeptide chains of the antigen binding protein may further be covalently or non-covalently linked to further proteins/domains, e.g. constant domains of antibodies or TCRs, e.g., F_{C} domains, or albumin, dimerizing proteins or fragments thereof, or further binding domains, e.g. to further improve stability or include further binding sites.

In some embodiments, the antigen binding protein comprises one polypeptide chain that forms the antigen-binding sites A and B, wherein for example, this polypeptide chain comprises a structure represented by any of the formulae [I] to [VIII]:

V1-L1-V4-L2-V2-L3-V3-C [I],

V4-L1-V1-L2-V2-L3-V3-C [II],

V1-L1-V4-L2-V3-L3-V2-C [III],

V4-L1-V1-L2-V3-L3-V2-C [IV],

V2-L1-V3-L2-V1-L3-V4-C [V],

V3-L1-V2-L2-V1-L3-V4-C [VI],

V2-L1-V3-L2-V4-L3-V1-C [VII],

V3-L1-V2-L2-V4-L3-V1-C [VIII],
wherein V1 is a first variable domain; V2 is a second variable domain; V3 is a third variable domain; V4 is a fourth variable domain; L1, L2 and L3 are linkers; C is a constant domain, which might be present or absent.

In one preferable embodiment, V1 and V4 may form the antigen-binding site A and V2 and V3 may form the antigen-binding site B.

In some embodiments, the antigen binding protein comprises one polypeptide chain that forms the antigen-binding sites A and B, wherein this polypeptide chain comprises a structure represented by any of the formulae [I] to [VIII]:

V1-L1-V2-L2-V3-L3-V4-C [I],

V1-L1-V3-L2-V2-L3-V4-C [II],

V2-L1-V1-L2-V4-L3-V3-C [III],

V2-L1-V4-L2-V1-L3-V3-C [IV],

V3-L1-V1-L2-V4-L3-V2-C [V],

V3-L1-V4-L2-V1-L3-V2-C [VI],

V4-L1-V2-L2-V3-L3-V1-C [VII],

V4-L1-V3-L2-V2-L3-V1-C [VIII],
wherein V1 is a first variable domain; V2 is a second variable domain; V3 is a third variable domain; V4 is a fourth variable domain; L1, L2 and L3 are linker domains; C is a constant domain, which might be present or absent.

As indicated above, in a preferable embodiment, V1 and V4 may form the antigen-binding site A and V2 and V3 may form the antigen-binding site B.

In some embodiments, the antigen binding protein comprises two polypeptide chains that form the antigen-binding sites A and B, wherein a polypeptide chain comprises a structure represented by one of the formulae [I] to [IV]:

V2-L1-V3-L2-V1-C1 [I]

V2-L1-V3-L2-V4-C1 [II]

V3-L1-V2-L2-V1-C1 [III]

V3-L1-V2-L2-V4-C1 [IV]
wherein V1 is a first variable domain; V2 is a second variable domain; V3 is a third variable domain; V4 is a fourth variable domain; C1 and C2 are constant domains; L1 and L2 are linker domains;
and wherein a further polypeptide chain comprises a structure represented by formulae [V] and [VI]:

V4-C2 [V]

V1-C2 [VI]
wherein [I] or [III] bind to [VI] and [II] or [IV] bind to [VI].

In a preferred embodiment, V1 and V4 may form the antigen-binding site A and V2 and V3 may form the antigen-binding site B.

In some embodiments, the antigen binding protein comprises two polypeptide chains that together form the antigen-binding sites A and B, wherein a first polypeptide chain comprises a structure represented by formula [III]:

V1-L1-V2-L2-C1 [III]
wherein V1 is a first variable domain; V2 is a second variable domain; L1 and L2 are linkers; L2 may be present or absent; C1 may be a constant domain or a dimerizing portion thereof (e.g. CL) or another dimerizing protein or fragment thereof and present or absent, or in particular a light chain constant domain or a dimerizing portion thereof and present or absent;
and wherein a second polypeptide chain comprises a structure represented by formula [IV]:

V3-L3-V4-L4-C2 [IV]
wherein V3 is a third variable domain; V4 is a fourth variable domain; L3 and L4 are linkers; L4 may be present or absent; C2 may be a constant domain or a dimerizing portion thereof (e.g. CH) or another dimerizing protein or fragment thereof and present or absent, or in particular a heavy chain constant domain 1 or a dimerizing portion thereof and is present or absent; and wherein
V1 and V2 form the antigen-binding site A and V3 and V4 form the antigen-binding site B, or
V1 and V2 form the antigen-binding site B and V3 and V4 form the antigen-binding site A, or
V1 and V3 form the antigen-binding site A and V2 and V4 form the antigen-binding site B, or
V1 and V3 form the antigen-binding site B and V2 and V4 form the antigen-binding site A, or
V1 and V4 form the antigen-binding site A and V2 and V3 form the antigen-binding site B, or
V1 and V4 form the antigen-binding site B and V2 and V3 form the antigen-binding site A, and
optionally, wherein the first polypeptide and the second polypeptide are covalently or non-covalently linked together.

In a particular aspect, the antigen binding comprises
a first polypeptide of formula V₁-L₁-V₂-L₂-C₁-L₅-F_{C1}
wherein L₂, C₁, L₅ may be absent, and
a second polypeptide of formula V₃-L₃-V₄-L₄-C₂-L₆-F_{C2}
wherein L₄, C₂, L₆ may be absent,
wherein L2, L4, L5 and L6 are linkers,
C1 may be CL a light chain constant domain or a dimerizing portion thereof, or CH a heavy chain constant domain or a dimerizing portion thereof; and C2 may be a CH heavy chain constant domain or a dimerizing portion thereof if C1 is CL, and C2 may be a CL or dimerizing portion thereof if C1 is CH.

In some embodiments, the first polypeptide chain has a structure represented by the formula [V]:

V1-L1-V2-L2-C1-L5-F_{C1} [V]

and the second polypeptide chain has a structure represented by the formula [VI]:

V3-L3-V4-L4-C2-L6-F_{C2} [VI]
wherein V1-V4, L1-L4, C1 and C2 are as defined hereinabove, and wherein L2, C1, L5, L4, C2, and C6 may be present or absent and F_{C1}, and F_{C2} may be Fc-domains and wherein F_{C1} and F_{C2} are the same or different.

In an exemplary embodiment, the antigen binding protein of the invention has the variable domain orientations for VA, VB, VL and VH according to orientation D of FIG. 8.

In particular embodiments as herein provided above,
V₁ comprises CDRa1, CDRa2 and CDRa3 as defined hereinabove,
V₂ comprises CDRH1, CDRH2, and CDRH3 as defined hereinabove,
V₃ comprises CDRL1, CDRL2, and CDRL3 as defined hereinabove,
V₄ comprises CDRb1, CDRb2 and/or CDRb3 as defined hereinabove.

For example, the antigen binding protein may comprise
V₁ comprising:
CDRa1 comprising or consisting of SEQ ID NO: 2,
CDRa2 comprising or consisting of SEQ ID NO: 7 and
CDRa3 comprising or consisting of SEQ ID NO: 3,
V₂ comprising:
CDRH1 comprising or consisting of SEQ ID NO: 48,
CDRH2 comprising or consisting of SEQ ID NO: 49, and
CDRH3 comprising or consisting of SEQ ID NO: 50,
V₃ comprising:
CDRL1 comprising or consisting of SEQ ID NO: 51,
CDRL2 comprising or consisting of SEQ ID NO: 52, and
CDRL3 comprising or consisting of SEQ ID NO: 53,
V₄ comprising:
CDRb1 comprising or consisting of SEQ ID NO: 5,
CDRb2 comprising or consisting of SEQ ID NO: 9 and/or
CDRb3 comprising or consisting of SEQ ID NO: 6.

In a particular aspect, the antigen binding protein comprises
a polypeptide of formula V₁-L₁-V₂, and
a further polypeptide of formula V₃-L₃-V₄ wherein V1 is a first variable domain; V2 is a second variable domain; V3 is a third variable domain; V4 is a fourth variable domain, and L1 and L3 are linkers, and wherein
V1 is Vα, V2 is VH, V3 is VL, V4 is Vβ;
V1 is VL, V2 is Vα, V3 is Vβ, V4 is VH;
V1 is Vβ, V2 is VL, V3 is VH, V4 is Vα;
V1 is VH, V2 is Vα, V3 is Vβ, V4 is VL;
V1 is Vα, V2 is VL, V3 is VH, V4 is Vβ;
V1 is VL, V2 is Vβ, V3 is Vα, V4 is VH;
V1 is Vβ, V2 is VH, V3 is VL, V4 is Vα; or
V1 is VH, V2 is Vβ, V3 is Vα, V4 is VL.

In more preferred aspects, V1 is Vα, V2 is VH, V3 is VL, V4 is Vβ.

In an even more preferred aspect, the antigen binding is a TCER as exemplified in the Examples and comprises
a polypeptide of formula V₁-L₁-V₂-F_{C1}, and
a polypeptide of formula V₃-L₃-V₄-F_{C2}
   wherein V1 is a first variable domain; V2 is a second variable domain; V3 is a third variable domain; V4 is a fourth variable domain, and L₁ and L₃ are linkers, and wherein F_{C1} and F_{C2} are F_{C}-domains and wherein F_{C1} and F_{C2} are the same or preferably different, and wherein
V1 and V4 form the antigen-binding site A and V2 and V3 form the antigen-binding site B, or
V1 and V4 form the antigen-binding site B and V2 and V3 form the antigen-binding site A. In this preferred aspect,
V1 is Vα, V2 is VH, V3 is VL, V4 is Vβ;
V1 is VL, V2 is Vα, V3 is Vβ, V4 is VH;
V1 is Vβ, V2 is VL, V3 is VH, V4 is Vα;
V1 is VH, V2 is Vα, V3 is Vβ, V4 is VL;
V1 is Vα, V2 is VL, V3 is VH, V4 is Vβ;
V1 is VL, V2 is Vβ, V3 is Vα, V4 is VH;
V1 is Vβ, V2 is VH, V3 is VL, V4 is Vα; or
V1 is VH, V2 is Vβ, V3 is Vα, V4 is VL.

In more preferred aspects, V1 is Vα, V2 is VH, V3 is VL, V4 is Vβ.

In other exemplary embodiments, the antigen binding protein comprises
a polypeptide of formula V₁-L₁-V₂, and
a further polypeptide of formula V₃-L₃-V₄, and
wherein
   V₁ comprises CDRa1, CDRa2 and CDRa3 as defined hereinabove,
   V₂ comprises CDRH1, CDRH2, and CDRH3 as defined hereinabove,
   V₃ comprises CDRL1, CDRL2, and CDRL3 as defined hereinabove,
V₄ comprises CDRb1, CDRb2 and/or CDRb3 as defined hereinabove.
For example, the antigen binding protein may comprise
V₁ comprising:
CDRa1 comprising or consisting of SEQ ID NO: 2,
CDRa2 comprising or consisting of SEQ ID NO: 7 and
CDRa3 comprising or consisting of SEQ ID NO: 3,
V₂ comprising:
CDRH1 comprising or consisting of SEQ ID NO: 48,
CDRH2 comprising or consisting of SEQ ID NO: 49, and
CDRH3 comprising or consisting of SEQ ID NO: 50,
V₃ comprising:
CDRL1 comprising or consisting of SEQ ID NO: 51,
CDRL2 comprising or consisting of SEQ ID NO: 52, and
CDRL3 comprising or consisting of SEQ ID NO: 53,
V₄ comprising:
CDRb1 comprising or consisting of SEQ ID NO: 5,
CDRb2 comprising or consisting of SEQ ID NO: 9 and/or
CDRb3 comprising or consisting of SEQ ID NO: 6.

In preferred aspects of the invention, F_{C1} and/or F_{C2} comprises or consists of the amino acid sequence SEQ ID NO: 24 [Fc knob] and/or SEQ ID NO: 25 [F_{C} hole]; and/or L₁ and L₃ comprise or consist of the amino acid sequence GGGSGGGG of SEQ ID NO: 26.

In these embodiments, optionally the following may further be comprised:
L₁ and L₃ comprise or consist of the amino acid sequence GGGSGGGG of SEQ ID NO: 26, and L₂, L₄, L₅ and L₆ are absent, and optionally wherein
F_{C1} comprises or consists of the amino acid sequence SEQ ID NO: 25 [F_{C} hole], and, F_{C2} comprises or consists of the amino acid sequence SEQ ID NO: 24 [Fc knob], or
F_{C1} comprises or consists of the amino acid sequence SEQ ID NO: 24 [Fc knob], and F_{C2} comprises or consists of the amino acid sequence SEQ ID NO: 25 [F_{C} hole].

Preferably, the antigen binding protein of the invention has the variable domain orientations for Vα, Vβ, VL and VH according to orientation D of FIG. 8.

In a preferred embodiment, the antigen binding protein of the invention comprises the following CDR amino acid sequences:
CDRa1 comprising or consisting of SEQ ID NO: 2,
CDRa2 comprising or consisting of SEQ ID NO: 7,
CDRa3 comprising or consisting of SEQ ID NO: 3,
CDRb1 comprising or consisting of SEQ ID NO: 5,
CDRb2 comprising or consisting of SEQ ID NO: 9,
CDRb3 comprising or consisting of SEQ ID NO: 6,
CDRH1 comprising or consisting of SEQ ID NO: 48,
CDRH2 comprising or consisting of SEQ ID NO: 49,
CDRH3 comprising or consisting of SEQ ID NO: 50,
CDRL1 comprising or consisting of SEQ ID NO: 51,
CDRL2 comprising or consisting of SEQ ID NO: 52, and
CDRL3 comprising or consisting of SEQ ID NO: 53;
optionally wherein the CDRa1, CDRa3, CDRb1, CDRb3, CDRH1, CDRH2, CDRH3, CDRL1, CDRL2, and/or CDRL3 sequence(s) may comprise one, two or three amino acid mutations.

In another preferred embodiment, the antigen binding protein of the invention comprises the following CDR amino acid sequences:
CDRa1 comprising or consisting of SEQ ID NO: 2,
CDRa2 comprising or consisting of SEQ ID NO: 8,
CDRa3 comprising or consisting of SEQ ID NO: 4,
CDRb1 comprising or consisting of SEQ ID NO: 5,
CDRb2 comprising or consisting of SEQ ID NO: 10,
CDRb3 comprising or consisting of SEQ ID NO: 6,
CDRH1 comprising or consisting of SEQ ID NO: 48,
CDRH2 comprising or consisting of SEQ ID NO: 49,
CDRH3 comprising or consisting of SEQ ID NO: 50,
CDRL1 comprising or consisting of SEQ ID NO: 51,
CDRL2 comprising or consisting of SEQ ID NO: 52, and
CDRL3 comprising or consisting of SEQ ID NO: 53;
optionally wherein the CDRa1, CDRa3, CDRb1, CDRb3, CDRH1, CDRH2, CDRH3, CDRL1, CDRL2, and/or CDRL3 sequence(s) may comprise one, two or three amino acid mutations.

In further preferred embodiment, the antigen binding protein of the invention comprises the following CDR amino acid sequences:
CDRa1 comprising or consisting of SEQ ID NO: 2,
CDRa2 comprising or consisting of SEQ ID NO: 7,
CDRa3 comprising or consisting of SEQ ID NO: 3,
CDRb1 comprising or consisting of SEQ ID NO: 5,
CDRb2 comprising or consisting of SEQ ID NO: 10,
CDRb3 comprising or consisting of SEQ ID NO: 6,
CDRH1 comprising or consisting of SEQ ID NO: 48,
CDRH2 comprising or consisting of SEQ ID NO: 49,
CDRH3 comprising or consisting of SEQ ID NO: 50,
CDRL1 comprising or consisting of SEQ ID NO: 51,
CDRL2 comprising or consisting of SEQ ID NO: 52, and
CDRL3 comprising or consisting of SEQ ID NO: 53;
optionally wherein the CDRa1, CDRa3, CDRb1, CDRb3, CDRH1, CDRH2, CDRH3, CDRL1, CDRL2, and/or CDRL3 sequence(s) may comprise one, two or three amino acid mutations.

In the following, the formats of the exemplary provided sequences are explained. The skilled person is well aware that the binding domains may also be included in other formats.

In a preferred embodiment, the antigen binding protein of the invention comprises:
a first polypeptide of formula V₁-L₁-V₂-L₂-C₁-L₅-F_{C1} [V] of the amino acid sequence (SEQ ID NO: 27):

EDVEQSSFLSVREGDSAVINCTYTDASSTYFYWYKQEPGAGLQLLTYIYS
NMDMKQDQRLTVLLNKKDKHLSLRIADTQTGDSAIYFCAVKDNARLLFGD
VTQLVVKPGGGSGGGGEVQLVQSGAEVKKPGASVKVSCKASGYKFTSYVM
HWVRQAPGQGLEWMGYINPRNDVTKYAEKFQGRVTLTSDTSTSTAYMELS
SLRSEDTAVYYCARGSYYDYEGFVYWGQGTLVTVSSEPKSSDKTHTCPPC
PAPPVAGPSVFLFPPKPKDTLMISRTPEVTCVVVDVSHEDPEVKFNWYVD
GVEVHNAKTKPREEQYQSTYRVVSVLTVLHQDWLNGKEYKCKVSNKALPA
SIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSP

wherein L₂, C₁, L₅ are absent and which comprises V₁ (maturated TCR derived alpha variable domain) of sequence SEQ ID NO: 11 (with the CDRa1, CDRa2, CDRa3 of SEQ ID NO: 2, SEQ ID NO: 7 and SEQ ID NO: 3, respectively; in bold), L₁ (TCER linker; underlined) of sequence SEQ ID NO: 26, V₂ (BMhanced T cell Recruiter V_{H} with the CDRH1, CDRH2, CDRH3 of SEQ ID NO: 48, SEQ ID NO: 49 and SEQ ID NO: 50, respectively; in bold) of sequence SEQ ID NO: 46, and F_{C1} (fc hole; in italic and underlined) of sequence SEQ ID NO: 25; and
a second polypeptide of formula V₃-L₃-V₄-L₄-C₂-L₆-F_{C2} [VI] of the amino acid sequence (SEQ ID NO: 34):

QIQMTQSPSSLSASVGDRVTITCSATSSVSYMHWYQQKPGKAPKRWIYDT
SKLASGVPSRFSGSGSGTDYTLTISSLQPEDAATYYCQQWSSNPLTFGGG
TKVEIKGGGSGGGGAVISQKPSRVIVQRGTSVTIQCQVDRPVTMMYWYRQ
QPGQSLTLIATAYDEGSATYESGFDIDKFPISRPNLTFSTLTVSNVEPED
SSIYLCSVLGAYGYTFGSGTRLTVVEPKSSDKTHTCPPCPAPPVAGPSVF
LFPPKPKDTLMISRTPEVTCVVVDVSHEDPEVKFNWYVDGVEVHNAKTKP
REEQYQSTYRVVSVLTVLHQDWLNGKEYKCKVSNKALPASIEKTISKAKG
QPREPQVYTLPPCRDELTKNQVSLWCLVKGFYPSDIAVEWESNGQPENNY
KTTPPVLDSDGSFFLYSKLTVDKSRWQQGNVESCSVMHEALHNHYTQKSL
SLSP

wherein L₄, C₂, L₆ are absent and which comprises V₃ (BMhanced T cell recruiter VL with the CDRL1, CDRL2, CDRL3 of SEQ ID NO: 51, SEQ ID NO: 52 and SEQ ID NO: 53, respectively; in bold) of sequence SEQ ID NO: 47, L₃ (TCER linker; underlined) of sequence SEQ ID NO: 26, V₄ (TCR derived beta variable domain) of sequence SEQ ID NO: 15 (with the CDRb1, CDRb2, CDRb3 of SEQ ID NO: 5, SEQ ID NO: 9 and SEQ ID NO: 6, respectively in bold), and F_{C2} (fc knob; in italic and underlined) of sequence SEQ ID NO: 24, wherein the F_{C1} and F_{C2} sequence comprise a cysteine to serine amino acid substitution (S indicated in bold) since no light chain needs to be connected via said cysteine, amino acid substitutions of the IgG1 hinge region to resemble the IgG2 hinge to abrogate F_{c}-gamma receptor interaction (PVA indicated in bold in positions 233 to 235 and 331S, and the removal of G236), a N297Q amino acid substitution (Q in bold) to remove the N-Glycosylation site within the F_{c}-part to abrogate F_{c}-gamma-receptor interaction and in addition the hole mutation (Y349C, T366S, L368A and Y407V in bold) in case of F_{C1} and the knob mutation (S354C and T366W in bold) in case of F_{C2}. Furthermore, the C-terminal glycine-lysine (G446, K447) residues of human IgG1 heavy chains may be deleted to decrease C-terminal heterogeneity.

In another preferred embodiment, the antigen-binding protein of the invention comprises:
a first polypeptide of formula V₁-L₁-V₂-L₂-C₁-L₅-F_{C1} [V] of the amino acid sequence (SEQ ID NO: 28):

EDVEQSSFLSVREGDSAVINCTYTDASSTYFYWYKQEPGAGLQLLTYIFS
NMDMKQDQRLTVLLNKKDKHLSLRIADTQTGDSAIYFCAVKDNARLMFGD
VTQLVVKPGGGSGGGGEVQLVQSGAEVKKPGASVKVSCKASGYKFTSYVM
HWVRQAPGQGLEWMGYINPRNDVTKYAEKFQGRVTLTSDTSTSTAYMELS
SLRSEDTAVYYCARGSYYDYEGFVYWGQGTLVTVSSEPKSSDKTHTCPPC
PAPPVAGPSVFLFPPKPKDTLMISRTPEVTCVVVDVSHEDPEVKFNWYVD
GVEVHNAKTKPREEQYQSTYRVVSVLTVLHQDWLNGKEYKCKVSNKALPA
SIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSP

wherein L₂, C₁, L₅ are absent and which comprises V₁ (maturated TCR derived alpha variable domain) of sequence SEQ ID NO: 12 (with the CDRa1, CDRa2, CDRa3 of SEQ ID NO: 2, SEQ ID NO: 8 and SEQ ID NO: 4, respectively; in bold), L₁ (TCER linker; underlined) of sequence SEQ ID NO: 26, V₂ (BMhanced T cell Recruiter V_{H} with the CDRH1, CDRH2, CDRH3 of SEQ ID NO: 48, SEQ ID NO: 49 and SEQ ID NO: 50, respectively; in bold) of sequence SEQ ID NO: 46, and F_{C1} (fc hole; in italic and underlined) of sequence SEQ ID NO: 25; and
a second polypeptide of formula V₃-L₃-V₄-L₄-C₂-L₆-F_{C2} [VI] of the amino acid sequence (SEQ ID NO: 35):

QIQMTQSPSSLSASVGDRVTITCSATSSVSYMHWYQQKPGKAPKRWIYDT
SKLASGVPSRFSGSGSGTDYTLTISSLQPEDAATYYCQQWSSNPLTFGGG
TKVEIKGGGSGGGGAVISQKPSRYIVQRGTSVTIQCQVDRPVTMMYWYRQ
QPGQSLTLIATAYDVGSATYESGFVIDKFPISRPNLTFSTLTVSNVEPED
SSIYLCSVLGAYGYTFGSGTRLTVVEPKSSDKTHTCPPCPAPPVAGPSVF
LFPPKPKDTLMISRTPEVTCVVVDVSHEDPEVKFNWYVDGVEVHNAKTKP
REEQYQSTYRVVSVLTVLHQDWLNGKEYKCKVSNKALPASIEKTISKAKG
QPREPQVYTLPPCRDELTKNQVSLWCLVKGFYPSDIAVEWESNGQPENNY
KTTPPVLDSDGSFFLYSKLTVDKSRWQQGNVFSCSVMHEALHNHYTQKSL
SLSP

wherein L₄, C₂, L₆ are absent and which comprises V₃ (BMhanced T cell recruiter VL with the CDRL1, CDRL2, CDRL3 of SEQ ID NO: 51, SEQ ID NO: 52 and SEQ ID NO: 53, respectively; in bold) of sequence SEQ ID NO: 47, L₃ (TCER linker; underlined) of sequence SEQ ID NO: 26, V₄ (TCR derived beta variable domain) of sequence SEQ ID NO: 16 (with the CDRb1, CDRb2, CDRb3 of SEQ ID NO: 5, SEQ ID NO: 10 and SEQ ID NO: 6 in bold), and F_{C2} (fc knob; in italic and underlined) of sequence SEQ ID NO: 24,
wherein the F_{C1} and F_{C2} sequence comprise a cysteine to serine amino acid substitution (S indicated in bold) since no light chain needs to be connected via said cysteine, amino acid substitutions of the IgG1 hinge region to resemble the IgG2 hinge to abrogate F_{c}-gamma receptor interaction (PVA indicated in bold in positions 233 to 235 and 331S, and the removal of G236), a N297Q amino acid substitution (Q in bold) to remove the N-Glycosylation site within the Fc-part to abrogate Fc-gamma-receptor interaction and in addition the hole mutation (Y349C, T366S, L368A and Y407V in bold) in case of F_{C1} and the knob mutation (S354C and T366W in bold) in case of F_{C2}. Furthermore, the C-terminal glycine-lysine (G446, K447) residues of human IgG1 heavy chains may be deleted to decrease C-terminal heterogeneity.

In a further preferred embodiment, the antigen binding protein of the invention comprises:
a first polypeptide of formula V₁-L₁-V₂-L₂-C₁-L₅-F_{C1} [V] of the amino acid sequence (SEQ ID NO: 29):

EDVEQSSFLSVREGDSAVINCTYTDASSTYFYWYKQEPGAGLQLLTYIYS
NMDMKQDQRLTVLLNKKDKHLSLRIADTQTGDSAIYFCAVKDNARLLFGD
GTQLVVKPGGGSGGGGEVQLVQSGAEVKKPGASVKVSCKASGYKFTSYVM
HWVRQAPGQGLEWMGYINPRNDVTKYAEKFQGRVTLTSDTSTSTAYMELS
SLRSEDTAVYYCARGSYYDYEGFVYWGQGTLVTVSSEPKSSDKTHTCPPC
PAPPVAGPSVFLFPPKPKDTLMISRTPEVTCVVVDVSHEDPEVKENWYVD
GVEVHNAKTKPREEQYQSTYRVVSVLTVLHQDWLNGKEYKCKVSNKALPA
SIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHE
ALHNHYTQKSLSLSP

wherein L₂, C₁, L₅ are absent and which comprises V₁ (maturated TCR derived alpha variable domain) of sequence SEQ ID NO: 13 (with the CDRa1, CDRa2, CDRa3 of SEQ ID NO: 2, SEQ ID NO: 7 and SEQ ID NO: 3, respectively; in bold), L₁ (TCER linker; underlined) of sequence SEQ ID NO: 26, V₂ (BMhanced T cell Recruiter V_{H} with the CDRH1, CDRH2, CDRH3 of SEQ ID NO: 48, SEQ ID NO: 49 and SEQ ID NO: 50, respectively; in bold) of sequence SEQ ID NO: 46, and F_{C} (fc hole; in italic and underlined) of sequence SEQ ID NO: 25; and
a second polypeptide of formula V₃-L₃-V₄-L₄-C₂-L₆-F_{C2} [VI] of the amino acid sequence (SEQ ID NO: 36):

QIQMTQSPSSLSASVGDRVTITCSATSSVSYMHWYQQKPGKAPKRWIYDT
SKLASGVPSRFSGSGSGTDYTLTISSLQPEDAATYYCQQWSSNPLTFGGG
TKVEIKGGGSGGGGAVISQKPSRYIVQRGTSVTIQCQVDRPVTMMYWYRQ
QPGQSLTLIATAYDVGSATYESGFDIDKFPISRPNLTFSTLTVSNMEPED
SSIYLCSVLGAYGYTFGSGTRLTVVEPKSSDKTHTCPPCPAPPVAGPSVF
LFPPKPKDTLMISRTPEVTCVVVDVSHEDPEVKENWYVDGVEVHNAKTKP
REEQYQSTYRVVSVLTVLHQDWLNGKEYKCKVSNKALPASIEKTISKAKG
QPREPQVYTLPPCRDELTKNQVSLWCLVKGFYPSDIAVEWESNGQPENNY
KTTPPVLDSDGSFFLYSKLTVDKSRWQQGNVFSCSVMHEALHNHYTQKSL
SLSP

wherein L₄, C₂, L₆ are absent and which comprises V₃ (BMhanced T cell recruiter VL with the CDRL1, CDRL2, CDRL3 of SEQ ID NO: 51, SEQ ID NO: 52 and SEQ ID NO: 53, respectively; in bold) of sequence SEQ ID NO: 47, L₃ (TCER linker; underlined) of sequence SEQ ID NO: 26, V₄ (TCR derived beta variable domain) of sequence SEQ ID NO: 17 (with the CDRb1, CDRb2, CDRb3 of SEQ ID NO: 5, SEQ ID NO: 10 and SEQ ID NO: 6, respectively in bold), and F_{C2} (fc knob; in italic and underlined) of sequence SEQ ID NO: 24,
wherein the F_{C1} and F_{C2} sequence comprise a cysteine to serine amino acid substitution (S indicated in bold) since no light chain needs to be connected via said cysteine, amino acid substitutions of the IgG1 hinge region to resemble the IgG2 hinge to abrogate Fc-gamma receptor interaction (PVA indicated in bold in positions 233 to 235 and 331S, and the removal of G236), a N297Q amino acid substitution (Q in bold) to remove the N-Glycosylation site within the Fc-part to abrogate Fc-gamma-receptor interaction and in addition the hole mutation (Y349C, T366S, L368A and Y407V in bold) in case of F_{C1} and the knob mutation (S354C and T366W in bold) in case of F_{C2}. Furthermore, the C-terminal glycine-lysine (G446, K447) residues of human IgG1 heavy chains may be deleted to decrease C-terminal heterogeneity.

In some embodiments, the amino acid sequences of the binding domains A and B are chimeric, humanized or human.

In some embodiments, the N-terminal or the C-terminal amino acid(s) of the polypeptide chains of the antigen binding protein comprise(s) (a) modification(s), e.g. a post-translational modification. Preferably, the N-terminal amino acid(s) of the polypeptide chains of the antigen binding protein comprise(s) a modification.

In one embodiment, the N-terminal or the C-terminal amino acid(s) of the polypeptide chains of the antigen binding protein comprise(s) (a) modification(s), such as a naturally-occurring modification in aqueous solution, preferably a pyro-glutamate modification.

In one embodiment, the N-terminal amino acid(s) of the polypeptide chains, e.g. in the VL, of the antigen binding protein comprise a modification, preferably a pyro-glutamate modification.

In some embodiments, a soluble antigen binding protein according to the present invention is capable of activating a CD4+ T cell, preferably a CD4+ CD8-T cell, and/or a CD8+ T cell, preferably a CD8+ CD4-T cell.

In some embodiments, the antigen binding protein binds the MAGEB2 antigenic peptide of SEQ ID NO: 1 with a K_{D} of less than 50 nM, preferably less than 20 nM, more preferably less than 5 nM, as measured by biolayer interferometry.

In one embodiment, the antigen binding protein of the invention specifically binds to the MAGEB2 antigenic peptide comprising or consisting of the amino acid sequence of SEQ ID NO: 1 and a HLA molecule, preferably HLA-A*02, with a K_{D} which is ≤100 μM, ≤50 μM, ≤30 μM, ≤25 μM, ≤1 μM, ≤500 nM, ≤100 nM, ≤50 nM, ≤10 nM, preferably ≤5 nM, for example, 50 pM to 100 μM, 50 pM to 10 μM, 50 pM to 1 μM, more particularly, 50 pM to 500 nM, 50 PM to 100 nM, 50 pM to 50 nM, 50 pM to 10 nM, 50 pM to 5 nM. “K_{D}” and “affinity” are as defined herein above in the section “Definitions”.

Accordingly, in one example, the antigen binding proteins of the invention are expressed, for instance, as soluble TCER® described herein above and are analyzed for their binding affinity towards the HLA-A*02/MAGEB2 antigenic peptide complex. General conditions for biolayer interferometry are provided above. Typically, measurements are performed, for instance, on an Octet RED384 system using, typically, settings recommended by the manufacturer. Briefly, binding kinetics are, typically, measured at 30° C. and, for instance, 1000 rpm shake speed using, for example, PBS, 0.05% Tween-20, 0.1% BSA as buffer. The antigen binding proteins can be either loaded onto biosensors, such as FAB2G or AHC, or analyzed in solution.

In some embodiments, the antigen binding protein is capable of killing MAGEB2-expressing tumor cells in an in vitro cytotoxicity assay, wherein the MAGEB2-expressing tumor cells have a MAGEB2 copy number per cell of less than 200, preferably less than 100, more preferably less than 50. In a preferred embodiment, the MAGEB2-expressing tumor cells have a MAGEB2 copy number per cell is determined by AbsQuant® (e.g. as disclosed in PCT/EP2015/079873).

In some embodiments, the antigen binding protein is capable of killing MAGEB2-expressing tumor cells in an in vitro cytotoxicity assay, wherein the MAGEB2-expressing tumor cells have a MAGEB2 copy number per cell of less than 200, with an EC50 of less than 200 pM, preferably less than 100 pM, more preferably less than 30 pM.

### Nucleic Acids, Vectors, Host Cells

The polypeptides of the antigen binding proteins of the invention can be encoded by nucleic acids and expressed in vivo, ex vivo or in vitro. Thus, in a second aspect, the invention provides a nucleic acid or nucleic acids encoding the antigen binding protein of the first aspect of the invention. For example, one, two, three, or four or more nucleic acids may encode any antigen binding protein as defined herein.

Nucleic acid molecules of the disclosure can be obtained using standard molecular biology techniques, including but not limited to methods of amplification, and reverse transcription of RNA. Once DNA fragments encoding, for example, variable chains are obtained, these DNA fragments can be further manipulated by standard recombinant DNA techniques, for example to convert the variable region genes to full-length chain genes. In these manipulations, a variant-encoding DNA fragment is operatively linked to another DNA molecule, or to a fragment encoding another protein, such as a constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter. The isolated DNA encoding the variable region, e.g. the variable alpha region and/or variable beta region, can be converted to a full-length chain gene by operatively linking the variable-encoding DNA to another DNA molecule encoding constant regions. The sequences of human constant region genes, e.g. for TCRs or antibodies, are known in the art and DNA fragments encompassing these regions can be obtained by standard PCR amplification.

Typically, said nucleic acid is a DNA or RNA molecule, which may be included in a suitable vector.

Accordingly, also provided herein are expression vectors and host cells for producing the antigen binding proteins or functional fragments thereof described herein.

In a third aspect, the invention relates to a vector comprising the nucleic acid of the second aspect of the invention.

Various expression vectors can be employed to express the polynucleotides encoding the antigen binding proteins or functional fragments thereof. Both viral-based and non-viral expression vectors can be used to produce the antigen binding proteins or functional fragments thereof described herein in a mammalian host cell. Non-viral vectors and systems include plasmids, plasmid, cosmid, episome, artificial chromosome, phage or a viral vector.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of antibody heavy chain and the like.

For example, non-viral vectors useful for expression of polynucleotides and polypeptides described herein in mammalian (e.g. human or non-human) cells include all suitable vectors known in the art for expressing proteins Other examples of plasmids and include replicating plasmids comprising an origin of replication, or integrative plasmids, such as for instance pUC, pcDNA, pBR, and the like.

The first polypeptide and the second polypeptide described herein can be present in the same vector or separate vectors.

In a fourth aspect, the invention relates to a host cell comprising the antigen binding protein of the first aspect, the nucleic acid of the second aspect or the vector of the third aspect of the invention. The host cell may have been transfected, infected or transformed with a nucleic acid and/or a vector according to the invention.

The nucleic acids of the invention may be used to produce a recombinant antigen binding protein of the invention in a suitable expression system.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, HEK cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene is defective (Urlaub G et al; 1980), rat YB2/3HL.P2.G11.16Ag.20 cell (ATCC CRL1662), and the like. In some embodiments, the YB2/0 cell may be preferred, since ADCC activity of chimeric or humanized antibodies is enhanced when expressed in this cell.

According to the above, in one embodiment, the invention refers to a host cell comprising the antigen binding protein of the invention which is defined herein above, or the nucleic acid encoding the antigen binding protein of the invention, or the vector encoding the antigen binding protein of the invention, wherein said host cell may be a) a lymphocyte, such as a T lymphocyte or T lymphocyte progenitor cell, for example a CD4 or CD8 positive T cell or b) a cell for recombinant expression, such as a Chinese Hamster Ovary (CHO) cell.

In particular, for expression of some of the antigen binding proteins of the invention, in particular the antigen binding proteins comprising two polypeptides that are not linked, the expression vector may be either of a type in which a gene encoding an antibody heavy chain and a gene encoding an antibody light chain exists on separate vectors or of a type in which both genes exist on the same vector (tandem type). In respect of ease of construction of antigen binding protein expression vector, ease of introduction into animal cells, and balance between the expression levels of antibody Hand L chains in animal cells, humanized antibody expression vector of the tandem type is preferred (Shitara K et al. J Immunol Methods. 1994 Jan. 3; 167 (1-2): 271-8). Examples of tandem type humanized antibody expression vector include pKANTEX93 (WO 97/10354), pEE18 and the like.

In one embodiment, such recombinant host cells can be used for the production of at least one antigen binding protein of the invention.

For purposes of producing a recombinant antigen binding protein, for example a TCR, polypeptide, or protein, the host cell is preferably a mammalian cell. In preferred embodiments, the host cell is a cell for recombinant expression, such as a Chinese Hamster Ovary (CHO) cell.

The present invention further provides a composition comprising or consisting of the antigen binding protein of the first aspect of the invention, a nucleic acid or nucleic acids of the second aspect of the invention or a vector or vectors of the third aspect of the invention.

### Methods of Producing Antigen Binding Proteins

The recombinant host cells of the fourth aspect can be used for the production of at least one antigen binding protein of the first aspect of the invention. Thus, in a fifth aspect, the present invention provides a method of making any of the antigen binding proteins of the first aspect of the invention, comprising culturing the host cell of the fourth aspect of the invention under suitable conditions and, optionally, isolating the antigen binding protein produced by the host cells.

Accordingly, an antigen binding protein of the invention can be produced by a method of the fifth aspect.

In one embodiment, the method comprises comprising the steps of (a) providing a host cell according to the fourth aspect of the invention, (b) providing a vector of the third aspect comprising a coding sequence encoding the antigen binding protein of first aspect, (c) introducing the vector into the host cell, and (d) expressing the vector by the host cell.

In one embodiment, the method further comprises the isolation and purification of the antigen binding protein from the host cell.

An antigen binding protein of the invention may be produced by any technique known in the art, such as, without limitation, any chemical, biological, genetic or enzymatic technique, either alone or in combination.

Antigen binding proteins of the invention are suitably isolated and purified from the culture medium by antibody purification procedures such as, for example, protein A-sepharose, hydroxylapatite chromatography, gel electrophoresis, dialysis, or affinity chromatography.

In one embodiment, recovering the expressed antigen binding proteins or polypeptides herein refers to performing a protein A chromatography, a Kappa select chromatography, and/or a size exclusion chromatography, preferably a protein A chromatography and/or a size exclusion chromatography, more preferably a protein A chromatography and a size exclusion chromatography.

Knowing the amino acid sequence of the desired sequence, one skilled in the art can produce the antigen binding proteins of the present invention, by standard techniques for production of polypeptides. For instance, they can be synthesized using well-known solid phase method, in particular using a commercially available peptide synthesis apparatus (such as that made by Applied Biosystems, Foster City, California) and following the manufacturer's instructions. Alternatively, antibodies, and antigen binding proteins of the invention can be produced by recombinant DNA and gene transfection techniques well known in the art (see Morrison S L. et al. (1984) and patent documents U.S. Pat. Nos. 5,202,238; and 5,204,244). For example, fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

Methods for producing humanized antibodies based on conventional recombinant DNA and gene transfection techniques are well known in the art (See, e.g., Riechmann L. et al. 1988; Neuberger M S. et al. 1985) and can be easily applied to the production of the antigen binding proteins of the invention.

In one example, vectors for the expression of the recombinant antigen binding proteins of the invention were designed as monocistronic, for instance, controlled by HCMV-derived promoter elements, pUC19-derivatives. Plasmid DNA can be amplified, for example, in E. coli according to standard culture methods and subsequently purified using commercial-available kits (Macherey & Nagel). Purified plasmid DNA can be used for transient transfection of, for example, CHO-S cells according to instructions of the manufacturer (ExpiCHO™ system; Thermo Fisher Scientific). Transfected CHO-cells can be cultured, for instance, for 6-14 days at, for example, 32° C. to 37° C. and receive one to two feeds of ExpiCHO™ Feed solution.

Conditioned cell supernatant can be cleared by, for example, filtration (0.22 μm) utilizing, for instance, Sartoclear Dynamics® Lab Filter Aid (Sartorius). Bispecific antigen binding proteins can be purified using, for example, an Äkta Pure 25 L FPLC system (GE Lifesciences) equipped to perform affinity and size-exclusion chromatography in line. Affinity chromatography can be performed on, for example, protein A or L columns (GE Lifesciences) following standard affinity chromatographic protocols. For instance, size exclusion chromatography can be performed directly after elution (pH 2.8) from the affinity column to obtain highly pure monomeric protein using, for example, Superdex 200 μg 16/600 columns (GE Lifesciences) following standard protocols. Protein concentrations can be determined on, for example, a NanoDrop system (Thermo Scientific) using calculated extinction coefficients according to predicted protein sequences. Concentration can be adjusted, if needed, by using Vivaspin devices (Sartorius). Finally, purified molecules can be stored in, for example, phosphate-buffered saline at concentrations of about 1 mg/ml at temperatures of 2-8° C.

Quality of purified bispecific antigen binding proteins can be determined by, for example, HPLC-SEC on MabPac SEC-1 columns (5 μm, 7.8×300 mm) running in, for example, 50 mM sodium-phosphate pH 6.8 containing 300 mM NaCl within a Vanquish UHPLC-System.

### Pharmaceutical Compositions

In a sixth aspect, the invention provides a pharmaceutical composition comprising the antigen binding protein of the first aspect of the invention, the nucleic acid or nucleic acids of the second aspect of the invention or the vector or vectors of the third aspect of the invention, and optionally a pharmaceutically acceptable carrier.

Antigen binding proteins of the present invention have been shown to be capable of effecting cytotoxicity against cells presenting the MAGEB2 antigenic peptide. Since this peptide is specifically presented by tumor cells, the antigen binding proteins of the present invention are useful for destroying tumor cells in a patient. An immune response in a patient can be induced by direct administration of the described antigen binding proteins to the patient, ideally in combination with an agent enhancing the immunogenicity (i.e. an adjuvant). The immune response originating from such a therapeutic vaccination can be expected to be highly specific against tumor cells because the peptide GVYDGEEHSV (SEQ ID NO: 1) is not presented or over-presented on normal tissues in comparable copy numbers, preventing the risk of undesired autoimmune reactions against normal tissue cells in the patient.

The invention also relates to a pharmaceutical composition of the invention for use as a medicament (see section “Therapeutic Methods and Uses”).

Such pharmaceutical compositions may comprise a therapeutically effective amount of an antigen binding protein of the invention or an antigen binding protein of the invention further comprising a therapeutic agent, in admixture with a pharmaceutically or physiologically acceptable formulation agent selected for suitability with the mode of administration.

In some embodiments, the antigen binding protein of the present invention will be supplied as part of a sterile pharmaceutical composition, which will normally include at least one pharmaceutically acceptable carrier.

Examples of pharmaceutically acceptable carriers or diluents useful in the present invention include stabilizers such as SPGA, carbohydrates (e.g. sorbitol, mannitol, starch, sucrose, glucose, dextran), proteins such as albumin or casein, protein containing agents such as bovine serum or skimmed milk and buffers (e.g. phosphate buffer).

The form of the pharmaceutical compositions, the route of administration, the dosage and the regimen naturally depend upon the condition to be treated, the severity of the illness, the age, weight, and gender of the patient, etc. This pharmaceutical composition may be in any suitable form (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

Preferably, the pharmaceutical composition is administered by injection, e.g., intravenously. When the pharmaceutical composition comprises a host cell expressing the antigen binding protein of the invention, preferably a TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL® electrolyte solution (Abbott, Chicago, IL), PLASMALYTE A (Baxter, Deerfield, IL), about 5% dextrose in water, or Ringer's lactate. In an embodiment, the pharmaceutically acceptable carrier is supplemented with human serum albumen.

Empirical considerations, such as the biological half-life, generally will contribute to the determination of the dosage. Frequency of administration may be determined and adjusted over the course of therapy and is based on reducing the number of cancer cells, maintaining the reduction of cancer cells, reducing the proliferation of cancer cells, or killing the cancer cells. Alternatively, sustained continuous release formulations of the antigen binding protein may be appropriate. Various formulations and devices for achieving sustained release are known in the art.

In one embodiment, dosages for the antigen binding proteins may be determined empirically in individuals who have been given one or more administration(s). Individuals are given incremental dosages of the antigen binding protein. To assess efficacy of the antigen binding protein, a marker of the cancer cell state can be followed. These include direct measurements of cancer cell proliferation and cell death by FACS, other imaging techniques; an improvement in health as assessed by such measurements, or an increase in quality of life as measured by accepted tests or prolongation of survival. It will be apparent to one of skill in the art that the dosage will vary depending on the individual, the stage of the disease, and the past and concurrent treatments being used.

The doses used for the administration can be adapted as a function of various parameters, and in particular as a function of the mode of administration used, of the relevant pathology, or alternatively of the desired duration of treatment.

Pharmaceutical compositions (including antigen binding proteins, vectors and/or nucleic acids) of the invention may be provided in substantially pure form, for example at least 80%, at least 85%, at least 90%, at least 91%, at least 92%, at least 93%, at least 94%, at least 95%, at least 96%, at least 97%, at least 98%, at least 99% or 100% pure.

In some embodiments, the antigen binding protein is dissolved or dispersed in a pharmaceutically acceptable carrier or aqueous medium. In some embodiments, certain amino acid residues may undergo post-translational modification when the antigen binding protein is dissolved or dispersed in a pharmaceutically acceptable carrier or aqueous medium. In some embodiments, the post-translational modification is an N-terminal modification. In some embodiments, the post-translational N-terminal modification is the cyclization of one or more glutamine (Q) residues of the antigen binding protein. In some embodiments, the post-translational N-terminal modification is the cyclization of one or more glutamate (E) residues of the antigen binding protein. In some embodiments, the post-translational N-terminal modification results in a pyroglutamate residue.

### Therapeutic Methods and Uses

In a seventh aspect, the invention provides the antigen binding protein of the first aspect of the invention, the nucleic acid or nucleic acids of the second aspect of the invention, the vector or vectors of the third aspect of the invention, or the pharmaceutical composition of the sixth aspect of the invention for use in the treatment of a proliferative disease, wherein preferably the proliferative disease is cancer.

The antigen binding proteins of the invention are in particular for use in immune therapy for the treatment of a proliferative disease.

In preferred embodiments, the antigen binding proteins of the first aspect of the invention, the nucleic acid or nucleic acids of the second aspect of the invention, the vector or vectors of the third aspect of the invention, or the pharmaceutical composition of the sixth aspect of the invention are for use in the treatment of a MAGEB2-expressing cancer.

In a similar aspect, the present invention also relates to a method of treatment of a proliferative disease, wherein preferably the proliferative disease is cancer, comprising administering to a subject in need thereof a therapeutically effective amount of the antigen binding protein of the first aspect of the invention, the nucleic acid or nucleic acids of the second aspect of the invention, the vector or vectors of the third aspect of the invention, or the pharmaceutical composition of the sixth aspect of the invention. In one embodiment, the cells of the cancer present GVYDGEEHSV (SEQ ID NO: 1) in a complex with an MHC molecule on the cell surface.

In preferred embodiments of the method of treatment, the cancer is a MAGEB2-expressing cancer.

In some embodiments of the medical use and the method of treatment, the MAGEB2-expressing cancer is a solid cancer.

Beneficial treatment of particular cancers/tumors is shown in the prior art e.g. WO 2016/102272.

Thus and in further embodiments, the medical use and the method of treatment may be selected from the group consisting of liver cancer, lung cancer, chronic lymphocytic leukemia (CLL), colorectal cancer (CRC), gallbladder cancer (GBC), glioblastoma (GBM), gastric cancer (GC), hepatocellular carcinoma (HCC), head and neck cancer, head and neck squamous cell carcinoma (HNSCC), melanoma (MEL), Non-Hodgkin lymphoma (NHL), non-small cell lung cancer adenocarcinoma (NSCLCadeno), non-small cell lung cancer (NSCLC), squamous cell non-small cell lung cancer (NSCLCsquam), ovarian cancer (OC), esophageal cancer (OSCAR), renal cell carcinoma (RCC), small cell lung cancer (SCLC), urinary bladder carcinoma (UBC), and uterine and endometrial cancer (UEC).

In particular embodiments, the medical use and the method of treatment may be selected from the group consisting of hepatocellular carcinoma cancer (HCC), melanoma, non-small cell lung cancer (NSCLC), and non-Hodgkin lymphoma (NHL) and UEC (uterine endometrial cancer).

In a preferred embodiment, the antigen binding protein is or comprises a TCR or (a) functional fragment(s) thereof.

In some embodiments, the antigen binding protein is conjugated to a therapeutically active agent.

Preferably, the therapeutically active agent is selected from the group consisting of a radionuclide, a chemotherapeutic agent and a toxin.

In one embodiment, the treatment further comprises administering at least one chemotherapeutic agent to the subject in need of treatment.

In one embodiment, the treatment further comprises administering radiation therapy to the subject in need of treatment.

In a related aspect, the invention relates to a method of eliciting an immune response in a patient who has a proliferative disease, in particular a cancer that presents MAGEB2 in a complex with an MHC protein, comprising administering to the patient an antigen binding protein of the present disclosure, wherein said cancer is selected from the group of cancers consisting of hepatocellular carcinoma cancer (HCC), melanoma, non-small cell lung cancer (NSCLC), and non-Hodgkin lymphoma (NHL) and UEC (uterine endometrial cancer). In one embodiment, the immune response referred to in said method is a cytotoxic T cell response.

In yet another aspect, the invention relates to the use of an antigen binding protein, the nucleic acid(s), vector(s), or the pharmaceutical composition according to the invention for the manufacture of a medicament for the treatment of a proliferative disease in a subject.

In yet another aspect, the invention relates to the use of the antigen binding protein, the nucleic acid(s) or vector(s), or the pharmaceutical composition according to the invention for treating a disease in a subject.

Among the texts providing guidance for cancer therapy is Cancer, Principles and Practice of Oncology, 4th Edition, DeVita et al, Eds. J. B. Lippincott Co., Philadelphia, Pa. (1993). An appropriate therapeutic approach is chosen according to the particular type of cancer, and other factors such as the general condition of the patient, as is recognized in the pertinent field. An antigen binding protein of the present invention can be used by itself or can be added to a therapy regimen using other anti-neoplastic agents in treating a cancer patient.

Accordingly, in some embodiments, the antigen binding protein can be administered concurrently with, before, or after a variety of drugs and treatments widely employed in cancer treatment such as, for example, chemotherapeutic agents, non-chemotherapeutic, anti-neoplastic agents, and/or radiation.

In one embodiment, efficacy of the treatment with an antigen binding protein of the invention is assayed in vivo, for instance in a mouse model of cancer and by measuring, for example, changes in tumor volume between treated and control groups.

The antigen binding protein of the invention, the nucleic acid of the invention, the vector of the invention or the pharmaceutical composition of the invention can be administered by any feasible method.

In an aspect, the TCR-elicited immune response or T cell response may refer to the proliferation and activation of effector functions induced by a peptide, such as GVYDGEEHSV (SEQ ID NO: 1), in vitro, ex vivo or in vivo. For MHC class I restricted cytotoxic T cells, for example, effector functions may be lysis of peptide-pulsed, peptide-precursor pulsed or naturally peptide-presenting target cells, secretion of cytokines, preferably interferon-gamma, TNF-alpha, or IL-2 induced by peptide, secretion of effector molecules, for example, granzymes or perforins induced by peptide, or degranulation.

For the purposes of the invention, the amount or dose of the antigen binding protein of the first aspect of the invention, the nucleic acid(s) of the second aspect of the invention, the vector(s) of the third aspect of the invention, or the pharmaceutical composition of the fifth aspect of the invention administered may be sufficient to effect, e.g., a therapeutic or prophylactic response, in the subject or animal over a reasonable time frame. For example, the dose of the antigen binding protein, the nucleic acid(s), the vector(s), or the pharmaceutical composition according to the invention should be sufficient to bind to a cancer antigen, or detect, treat or prevent cancer in a period of from about 2 hours or longer, e.g., 12 to 24 or more hours, from the time of administration. In certain embodiments, the time period could be even longer. The dose will be determined by the efficacy of the antigen binding protein, nucleic acid(s), vector(s), or pharmaceutical composition according to the invention and the condition of the animal (e.g., human), as well as the body weight of the animal (e.g., human) to be treated.

### Kits

In an eighth aspect, the invention also provides kits comprising the antigen binding protein of the first aspect of the invention, a nucleic acid or nucleic acids of the second aspect of the invention or a vector or vectors of the third aspect of the invention.

In one embodiment, the kit comprises
a) at least one antigen binding protein of the invention as defined herein above in the section “Antigen binding proteins”, nucleic acid(s) encoding said antigen binding protein, or vector(s) comprising said nucleic acid(s),
b) optionally packaging material, and
c) optionally a label or packaging insert contained within said packaging material indicating that said antigen binding protein is effective for a method of treating cancer or for use in the treatment of cancer.

In preferred embodiments, the kit comprises (a) nucleic acid(s) encoding the antigen binding protein of the invention, or (a) vector(s) comprising said nucleic acid(s).

The kits of the present disclosure may further include any other reagents useful for transfection/transduction for introducing the nucleic acid(s) or expression vector(s) into cells.

Components of the kits may be present in separate containers, or multiple components may be present in a single container. A suitable container includes a single tube, one or more wells of a plate (e.g., a 96-well plate, a 384-well plate, etc.), or the like.

In a related embodiment, the at least one antigen binding proteins of the invention is contained in a single and/or multi-chambered pre-filled syringes (e.g., liquid syringes and lyosyringes).

In one embodiment, the invention encompasses kits for producing a single-dose administration unit.

Accordingly, in one embodiment, the at least one antigen binding protein of the invention as mentioned in a) of the kit of the invention is a dried antigen binding protein of the invention contained in a first container. The kit then further contains a second container having an aqueous formulation.

Accordingly, in one embodiment, the kit comprises
a) a first container comprising at least one dried antigen binding protein of the invention as defined herein above in the section “Antigen binding proteins”,
b) a second container comprising an aqueous formulation;
c) optionally packaging material, and
d) optionally a label or packaging insert contained within said packaging material indicating that said antigen binding protein is effective for a method of treating cancer or for use in the treatment of cancer.

The aqueous formulation is typically an aqueous solution comprising pharmaceutically acceptable carriers as defined herein above.

The definitions of the words or drawing elements described herein are meant to include not only the combination of elements which are literally set forth, but all equivalent structure, material or acts for performing substantially the same function in substantially the same way to obtain substantially the same result. In this sense, it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described herein, what is conceptually equivalent, what can be obviously substituted, and also what incorporates the essential ideas.

### Items

The present invention can also be characterised in terms of the following items.
1. An antigen binding protein specifically binding to a MAGEB2 antigenic peptide that is in a complex with a major histocompatibility complex (MHC) protein, wherein the MAGEB2 antigenic peptide comprises or consists of the amino acid sequence GVYDGEEHSV (SEQ ID NO: 1),
wherein the antigen binding protein comprises complementarity determining regions (CDRs) CDRa1, CDRa2, and CDRa3, and CDRb1, CDRb2, and CDRb3, wherein CDRa1, CDRa2, CDRa3, CDRb1, CDRb2 and CDRb3 form an antigen binding domain A;
preferably wherein the variable domain V_{A} comprises or consists of the amino acid sequence of EDVEQSSFLSVREGDSAVINCTYTDASSTYFYWYKQEPGAGLQLLTYIXSNMDMKQDQRLTVLLNKKD KHLSLRIADTQTGDSAIYFCAVKDNARLXFGDXTQLVVKP (SEQ ID NO: 152), wherein X is any amino acid, or an amino acid sequence having at least 85%, 90%, 95%, 98% or 99% identity to SEQ ID NO: 152, wherein X is any amino acid,
preferably wherein the variable domain V_{B} comprises or consists of the amino acid sequence of AVISQKPSRXIVQRGXSVTIQCQVDRPVTMMXWYRQQPGQSXTLIATAYDXGSATYESGFXIDKFPISRP NLTFSTLTVSNXXPEDSSIYLCSVLGAYGYTFGSGTRLTVV
(SEQ ID NO: 154), wherein X is any amino acid, or an amino acid sequence having at least 85%, 90%, 95%, 98% or 99% identity to SEQ ID NO: 154, wherein X is any amino acid, preferably wherein
CDRa1 comprises an amino acid sequence of SEQ ID NO: 2, CDRa3 comprises an amino acid sequence selected from the group consisting of: SEQ ID NOs: 3 and 4, CDRb1 comprises an amino acid sequence of SEQ ID NO: 5, and CDRb3 comprises an amino acid sequence of SEQ ID NO: 6;
wherein the CDRa1, CDRa3, CDRb1 and/or CDRb3 sequence(s) may comprise one, two or three amino acid mutations.
2. The antigen binding protein of item 1, wherein
CDRa2 comprises an amino acid sequence selected from the group consisting of: SEQ ID NOs: 7 and 8, and CDRb2 comprises an amino acid sequence selected from the group consisting of: SEQ ID NOs: 9 and 10;
wherein the CDRa2, and/or CDRb2 sequence(s) may comprise one, two or three amino acid mutations.
3. An antigen binding protein specifically binding to a MAGEB2 antigenic peptide that is in a complex with a major histocompatibility complex (MHC) protein, wherein the MAGEB2 antigenic peptide comprises or consists of the amino acid sequence GVYDGEEHSV (SEQ ID NO: 1),
wherein the antigen binding protein comprises a first polypeptide comprising a variable domain V_{A} comprising complementarity determining regions (CDRs) CDRa1, CDRa2, and CDRa3, and a second polypeptide comprising a variable domain V_{B} comprising CDRb1, CDRb2, and CDRb3, wherein CDRa1, CDRa2, CDRa3, CDRb1, CDRb2 and CDRb3 form an antigen binding domain A;
preferably wherein the variable domain V_{A} comprises or consists of the amino acid sequence according to SEQ ID NO: 152 or an amino acid sequence having at least 85%, 90%, 95%, 98% or 99% identity to SEQ ID NO: 152, wherein X is any amino acid, particularly preferably wherein the V_{A} comprises an amino acid sequence of EDVEQSSFLSVREGDSAVINCTYTDASSTYFYWYKQEPGAGLQLLTYIX₁SNMDMKQDQRLTVLLNKKD KHLSLRIADTQTGDSAIYFCAVKDNARLX₂FGDX₃TQLVVKP (SEQ ID NO: 153), wherein X₁ is Y or F, X₂ is L or M, and X₃ is V or G, or an amino acid sequence having at least 85%, 90%, 95%, 98% or 99% identity to SEQ ID NO: 153, wherein X₁ is Y or F, X₂ is L or M, and X₃ is V or G, preferably wherein the variable domain V_{B} comprises or consists of the amino acid sequence according to SEQ ID NO: 154, wherein X is any amino acid, or an amino acid sequence having at least 85%, 90%, 95%, 98% or 99% identity to SEQ ID NO: 154, wherein X is any amino acid, particularly preferably wherein the V_{B} comprises an amino acid sequence of AVISQKPSRX₁IVQRGX₂SVTIQCQVDRPVTMMX₃WYRQQPGQSX₄TLIATAYDX₅GSATYESGFX₆IDKFPI SRPNLTFSTLTVSNX₇X₈PEDSSIYLCSVLGAYGYTFGSGTRLTVV (SEQ ID NO: 155), wherein X₁ is V, Y, or D; X₂ is T or N; X₃ is Y or F; X₄ is L or P; X₅ is E or V; X₆ is D or V; X₇ is V or M; and X₈ is E or S;
or an amino acid sequence having at least 85%, 90%, 95%, 98% or 99% identity to SEQ ID NO: 155, wherein X₁ is V, Y, or D; X₂ is T or N; X₃ is Y or F; X₄ is L or P; X₅ is E or V; X₆ is D or V; X₇ is V or M; and X₈ is E or S;
preferably wherein
(1) CDRa1 comprises SEQ ID NO: 2, CDRa3 comprises SEQ ID NO: 3, CDRb1 comprises SEQ ID NO: 5, and CDRb3 comprises SEQ ID NO: 6; or
(2) CDRa1 comprises SEQ ID NO: 2, CDRa3 comprises SEQ ID NO: 4, CDRb1 comprises SEQ ID NO: 5, and CDRb3 comprises SEQ ID NO: 6;
wherein the CDRa1, CDRa3, CDRb1 and/or CDRb3 sequence(s) may comprise one, two or three amino acid mutations.
4. The antigen binding protein of item 3, wherein
   (1) CDRa1 comprises SEQ ID NO: 2, CDRa2 comprises SEQ ID NO: 7, CDRa3 comprises SEQ ID NO: 3, CDRb1 comprises SEQ ID NO: 5, CDRb2 comprises SEQ ID NO: 9, and CDRb3 comprises SEQ ID NO: 6;
   (2) CDRa1 comprises SEQ ID NO: 2, CDRa2 comprises SEQ ID NO: 8, CDRa3 comprises SEQ ID NO: 4, CDRb1 comprises SEQ ID NO: 5, CDRb2 comprises SEQ ID NO: 10, and CDRb3 comprises SEQ ID NO: 6;
   (3) CDRa1 comprises SEQ ID NO: 2, CDRa2 comprises SEQ ID NO: 7, CDRa3 comprises SEQ ID NO: 3, CDRb1 comprises SEQ ID NO: 5, CDRb2 comprises SEQ ID NO: 10, and CDRb3 comprises SEQ ID NO: 6; or
   (4) CDRa1 comprises SEQ ID NO: 2, CDRa2 comprises SEQ ID NO: 8, CDRa3 comprises SEQ ID NO: 4, CDRb1 comprises SEQ ID NO: 5, CDRb2 comprises SEQ ID NO: 9, and CDRb3 comprises SEQ ID NO: 6;
   wherein the CDRa1, CDRa2, CDRa3, CDRb1, CDRb2 and/or CDRb3 sequence(s) may comprise one, two or three amino acid mutations.
5. The antigen binding protein of any one of items 1 to 4,
   wherein the V_{A} comprises an amino acid sequence according to SEQ ID NO: 153, wherein X₁ is Y or F, X₂ is L or M, and X₃ is V or G, or an amino acid sequence having at least 85%, 90%, 95%, 98% or 99% identity to SEQ ID NO: 153, wherein X₁ is Y or F, X₂ is L or M, and X₃ is V or G, or
   wherein the V_{A} comprises an amino acid sequence selected from the group consisting of SEQ ID NOs: 11, 12, 13, and 14, or
   an amino acid sequence having at least 85%, 90%, 95%, 98% or 99% identity to SEQ ID NO: 11, and comprising the CDRa1, CDRa2, and CDRa3 of SEQ ID NOs: 2, 7, and 3, respectively,
   an amino acid sequence having at least 85%, 90%, 95%, 98% or 99% identity to SEQ ID NO: 12, and comprising the CDRa1, CDRa2, and CDRa3 of SEQ ID NOs: 2, 8, and 4, respectively,
   an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, or
   an amino acid sequence having at least 85%, 90%, 95%, 98% or 99% identity to SEQ ID NO: 14, and comprising the CDRa1, CDRa2, and CDRa3 of SEQ ID NOs: 2, 8, and 4, respectively; and
   wherein the V_{B} comprises an amino acid sequence according to SEQ ID NO: 155, wherein X₁ is V, Y, or D; X₂ is T or N; X₃ is Y or F; X₄ is L or P; X₅ is E or V; X₆ is D or V; X₇ is V or M; and X₈ is E or S; or an amino acid sequence having at least 85%, 90%, 95%, 98% or 99% identity to SEQ ID NO: 155, wherein X₁ is V, Y, or D; X₂ is T or N; X₃ is Y or F; X₄ is L or P; X₅ is E or V; X₆ is D or V; X₇ is V or M; and X₈ is E or S; or
   wherein the V_{B} comprises an amino acid sequence selected from the group consisting of SEQ ID NOs: 15, 16, 17, 18, 19, 20, 21, 22, and 23, or
   an amino acid sequence having at least 85%, 90%, 95%, 98% or 99% identity to SEQ ID NO: 15, and comprising the CDRb1, CDRb2, and CDRb3 of SEQ ID NOs: 5, 9, and 6, respectively,
   an amino acid sequence having at least 85%, 90%, 95%, 98% or 99% identity to SEQ ID NO: 16, and comprising the CDRb1, CDRb2, and CDRb3 of SEQ ID NOs: 5, 10, and 6, respectively,
   an amino acid sequence having at least 85%, 90%, 95%, 98% or 99% identity to SEQ ID NO: 17, and comprising the CDRb1, CDRb2, and CDRb3 of SEQ ID NOs: 5, 10, and 6, respectively,
   an amino acid sequence having at least 85%, 90%, 95%, 98% or 99% identity to SEQ ID NO: 18, and comprising the CDRb1, CDRb2, and CDRb3 of SEQ ID NOs: 5, 10, and 6, respectively,
   an amino acid sequence having at least 85%, 90%, 95%, 98% or 99% identity to SEQ ID NO: 19, and comprising the CDRb1, CDRb2, and CDRb3 of SEQ ID NOs: 5, 10, and 6, respectively,
   an amino acid sequence having at least 85%, 90%, 95%, 98% or 99% identity to SEQ ID NO: 20, and comprising the CDRb1, CDRb2, and CDRb3 of SEQ ID NOs: 5, 10, and 6, respectively,
   an amino acid sequence having at least 85%, 90%, 95%, 98% or 99% identity to SEQ ID NO: 21, and comprising the CDRb1, CDRb2, and CDRb3 of SEQ ID NOs: 5, 10, and 6, respectively,
   an amino acid sequence having at least 85%, 90%, 95%, 98% or 99% identity to SEQ ID NO: 22, and comprising the CDRb1, CDRb2, and CDRb3 of SEQ ID NOs: 5, 9, and 6, respectively, or
   an amino acid sequence having at least 85%, 90%, 95%, 98% or 99% identity to SEQ ID NO: 23, and comprising the CDRb1, CDRb2, and CDRb3 of SEQ ID NOs: 5, 9, and 6, respectively,
   wherein optionally the CDRa1, CDRa2, CDRa3, CDRb1, CDRb2, and/or CDRb3 sequences may comprise one, two or three amino acid mutations, preferably amino acid substitutions.
6. The antigen binding protein of any one of items 1 to 5,
   wherein the V_{A} comprises an amino acid sequence of SEQ ID NO: 11 or an amino acid sequence having at least 85%, 90%, 95%, 98% or 99% identity to SEQ ID NO: 11, and comprising the CDRa1, CDRa2, and CDRa3 of SEQ ID NOs: 2, 7, and 3, respectively, and
   wherein the V_{B} comprises an amino acid sequence of SEQ ID NO: 15 or an amino acid sequence having at least 85%, 90%, 95%, 98% or 99% identity to SEQ ID NO: 15, and comprising the CDRb1, CDRb2, and CDRb3 of SEQ ID NOs: 5, 9, and 6, respectively;
   wherein the V_{A} comprises an amino acid sequence of SEQ ID NO: 12 or an amino acid sequence having at least 85%, 90%, 95%, 98% or 99% identity to SEQ ID NO: 12, and comprising the CDRa1, CDRa2, and CDRa3 of SEQ ID NOS: 2, 8, and 4, respectively, and
   wherein the V_{B} comprises an amino acid sequence of SEQ ID NO: 16 or an amino acid sequence having at least 85%, 90%, 95%, 98% or 99% identity to SEQ ID NO: 16, and comprising the CDRb1, CDRb2, and CDRb3 of SEQ ID NOs: 5, 10, and 6, respectively;
   wherein the V_{A} comprises an amino acid sequence of SEQ ID NO: 13 or an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, and
   wherein the V_{B} comprises an amino acid sequence of SEQ ID NO: 17 or an amino acid sequence having at least 85%, 90%, 95%, 98% or 99% identity to SEQ ID NO: 17, and comprising the CDRb1, CDRb2, and CDRb3 of SEQ ID NOs: 5, 10, and 6, respectively;
   wherein the V_{A} comprises an amino acid sequence of SEQ ID NO: 14 or an amino acid sequence having at least 85%, 90%, 95%, 98% or 99% identity to SEQ ID NO: 14, and comprising the CDRa1, CDRa2, and CDRa3 of SEQ ID NOS: 2, 8, and 4, respectively, and
   wherein the V_{B} comprises an amino acid sequence of SEQ ID NO: 18 or an amino acid sequence having at least 85%, 90%, 95%, 98% or 99% identity to SEQ ID NO: 18, and comprising the CDRb1, CDRb2, and CDRb3 of SEQ ID NOs: 5, 10, and 6, respectively;
   wherein the V_{A} comprises an amino acid sequence of SEQ ID NO: 13 or an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, and
   wherein the V_{B} comprises an amino acid sequence of SEQ ID NO: 19 or an amino acid sequence having at least 85%, 90%, 95%, 98% or 99% identity to SEQ ID NO: 19, and comprising the CDRb1, CDRb2, and CDRb3 of SEQ ID NOs: 5, 10, and 6, respectively;
   wherein the V_{A} comprises an amino acid sequence of SEQ ID NO: 11 or an amino acid sequence having at least 85%, 90%, 95%, 98% or 99% identity to SEQ ID NO: 11, and comprising the CDRa1, CDRa2, and CDRa3 of SEQ ID NOs: 2, 7, and 3, respectively, and
   wherein the V_{B} comprises an amino acid sequence of SEQ ID NO: 19 or an amino acid sequence having at least 85%, 90%, 95%, 98% or 99% identity to SEQ ID NO: 19, and comprising the CDRb1, CDRb2, and CDRb3 of SEQ ID NOs: 5, 10, and 6, respectively;
   wherein the V_{A} comprises an amino acid sequence of SEQ ID NO: 14 or an amino acid sequence having at least 85%, 90%, 95%, 98% or 99% identity to SEQ ID NO: 14, and comprising the CDRa1, CDRa2, and CDRa3 of SEQ ID NOs: 2, 8, and 4, respectively, and
   wherein the V_{B} comprises an amino acid sequence of SEQ ID NO: 20 or an amino acid sequence having at least 85%, 90%, 95%, 98% or 99% identity to SEQ ID NO: 20, and comprising the CDRb1, CDRb2, and CDRb3 of SEQ ID NOs: 5, 10, and 6, respectively;
   wherein the V_{A} comprises an amino acid sequence of SEQ ID NO: 13 or an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, and
   wherein the V_{B} comprises an amino acid sequence of SEQ ID NO: 21 or an amino acid sequence having at least 85%, 90%, 95%, 98% or 99% identity to SEQ ID NO: 21, and comprising the CDRb1, CDRb2, and CDRb3 of SEQ ID NOs: 5, 10, and 6, respectively;
   wherein the V_{A} comprises an amino acid sequence of SEQ ID NO: 11 or an amino acid sequence having at least 85%, 90%, 95%, 98% or 99% identity to SEQ ID NO: 11, and comprising the CDRa1, CDRa2, and CDRa3 of SEQ ID NOs: 2, 7, and 3, respectively, and
   wherein the V_{B} comprises an amino acid sequence of SEQ ID NO: 21 or an amino acid sequence having at least 85%, 90%, 95%, 98% or 99% identity to SEQ ID NO: 21, and comprising the CDRb1, CDRb2, and CDRb3 of SEQ ID NOs: 5, 10, and 6, respectively;
   wherein the V_{A} comprises an amino acid sequence of SEQ ID NO: 12 or an amino acid sequence having at least 85%, 90%, 95%, 98% or 99% identity to SEQ ID NO: 12, and comprising the CDRa1, CDRa2, and CDRa3 of SEQ ID NOs: 2, 8, and 4, respectively, and
   wherein the V_{B} comprises an amino acid sequence of SEQ ID NO: 18 or an amino acid sequence having at least 85%, 90%, 95%, 98% or 99% identity to SEQ ID NO: 18, and comprising the CDRb1, CDRb2, and CDRb3 of SEQ ID NOs: 5, 10, and 6, respectively;
   wherein the V_{A} comprises an amino acid sequence of SEQ ID NO: 13 or an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, and
   wherein the V_{B} comprises an amino acid sequence of SEQ ID NO: 22 or an amino acid sequence having at least 85%, 90%, 95%, 98% or 99% identity to SEQ ID NO: 22, and comprising the CDRb1, CDRb2, and CDRb3 of SEQ ID NOs: 5, 9, and 6, respectively; or
   wherein the V_{A} comprises an amino acid sequence of SEQ ID NO: 14 or an amino acid sequence having at least 85%, 90%, 95%, 98% or 99% identity to SEQ ID NO: 14, and comprising the CDRa1, CDRa2, and CDRa3 of SEQ ID NOS: 2, 8, and 4, respectively, and
   wherein the V_{B} comprises an amino acid sequence of SEQ ID NO: 23 or an amino acid sequence having at least 85%, 90%, 95%, 98% or 99% identity to SEQ ID NO: 23, and comprising the CDRb1, CDRb2, and CDRb3 of SEQ ID NOs: 5, 9, and 6, respectively;
   wherein optionally said CDRa1, CDRa2, CDRa3, CDRb1, CDRb2, and/or CDRb3 sequences may comprise one, two or three amino acid mutations, preferably amino acid substitutions.
7. The antigen binding protein of any one of items 1 to 6,
   wherein the V_{A} comprises an amino acid sequence selected from the group consisting of SEQ ID NOs: 11, 12, 13, and 14; and
   wherein the V_{B} comprises an amino acid sequence selected from the group consisting of SEQ ID NOs: 15, 16, 17, 18, 19, 20, 21, 22, and 23.
8. The antigen binding protein of any one of items 1 to 7,
   wherein the V_{A} comprises an amino acid sequence of SEQ ID NO: 11 and the Vb comprises an amino acid sequence of SEQ ID NO: 15;
   wherein the V_{A} comprises an amino acid sequence of SEQ ID NO: 12 and the Vb comprises an amino acid sequence of SEQ ID NO: 16;
   wherein the V_{A} comprises an amino acid sequence of SEQ ID NO: 13 and the Vb comprises an amino acid sequence of SEQ ID NO: 17;
   wherein the V_{A} comprises an amino acid sequence of SEQ ID NO: 14 and the Vb comprises an amino acid sequence of SEQ ID NO: 18;
   wherein the V_{A} comprises an amino acid sequence of SEQ ID NO: 13 and the Vb comprises an amino acid sequence of SEQ ID NO: 19;
   wherein the V_{A} comprises an amino acid sequence of SEQ ID NO: 11 and the Vb comprises an amino acid sequence of SEQ ID NO: 19;
   wherein the V_{A} comprises an amino acid sequence of SEQ ID NO: 14 and the Vb comprises an amino acid sequence of SEQ ID NO: 20;
   wherein the V_{A} comprises an amino acid sequence of SEQ ID NO: 13 and the Vb comprises an amino acid sequence of SEQ ID NO: 21;
   wherein the V_{A} comprises an amino acid sequence of SEQ ID NO: 11 and the Vb comprises an amino acid sequence of SEQ ID NO: 21;
   wherein the V_{A} comprises an amino acid sequence of SEQ ID NO: 12 and the Vb comprises an amino acid sequence of SEQ ID NO: 18;
   wherein the V_{A} comprises an amino acid sequence of SEQ ID NO: 13 and the Vb comprises an amino acid sequence of SEQ ID NO: 22; or
   wherein the V_{A} comprises an amino acid sequence of SEQ ID NO: 14 and the Vb comprises an amino acid sequence of SEQ ID NO: 23;
   optionally wherein the framework region(s) comprise(s) at least one amino acid substitution selected from the group of amino acid substitutions consisting of:
      an amino acid substitution at position 19 in FR1-a, wherein said amino acid substitution is S19A, S191, S19L, or S19V, preferably S19A or S19V, more preferably S19A,
      an amino acid substitution at position 39 in FR2-a, wherein said amino acid substitution is L39F, L39I, or L39V, most preferably L39F,
      an amino acid substitution at position 13 in FR1-b, wherein said amino acid substitution is C13V, C13T, C13S, or C13A, preferably C13V,
      amino acid substitutions at positions 94 and/or 95 in FR3-b, wherein said amino acid substitution at position 94 is M94V, and/or said amino acid substitution at position 95 is S95E, or S95D, preferably S95E, respectively;
   and wherein the positions of the substitutions are given according to the IMGT nomenclature.
9. The antigen binding protein of any one of items 1 to 8, further comprising:
   a constant domain, wherein the constant domain comprises an amino acid sequence selected from the group consisting of SEQ ID NOs: 24 and 25, or an amino acid sequence having at least 85%, 90%, 95%, 98% or 99% identity to SEQ ID NOs: 24 and 25;
   and/or a linker, wherein the linker comprises an amino acid sequence of SEQ ID NO: 26, or an amino acid sequence having at least 85%, 90%, 95%, 98% or 99% identity to SEQ ID NO: 26.
10. The antigen binding protein of any one of items 1 to 9,
   wherein the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively;
   wherein the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively;
   wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively;
   wherein the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively;
   wherein the first polypeptide comprises an amino acid sequence of SEQ ID NO: 31, or an amino acid sequence having at least 85%, 90%, 95%, 98% or 99% identity to SEQ ID NO: 31 and comprising the CDRa1, CDRa2, and CDRa3 of SEQ ID NO: 2, 8, and 4, respectively;
   wherein the first polypeptide comprises an amino acid sequence of SEQ ID NO: 32, or an amino acid sequence having at least 85%, 90%, 95%, 98% or 99% identity to SEQ ID NO: 32 and comprising the CDRa1, CDRa2, and CDRa3 of SEQ ID NO: 2, 8, and 4, respectively; or
   wherein the first polypeptide comprises an amino acid sequence of SEQ ID NO: 33, or an amino acid sequence having at least 85%, 90%, 95%, 98% or 99% identity to SEQ ID NO: 33 and comprising the CDRa1, CDRa2, and CDRa3 of SEQ ID NO: 2, 8, and 4, respectively; and
   the second polypeptide comprises an amino acid sequence of SEQ ID NO: 34, or an amino acid sequence having at least 85%, 90%, 95%, 98% or 99% identity to SEQ ID NO: 34 and comprising the CDRb1, CDRb2, and CDRb3 of SEQ ID NO: 5, 9, and 6, respectively;
   the second polypeptide comprises an amino acid sequence of SEQ ID NO: 35, or an amino acid sequence having at least 85%, 90%, 95%, 98% or 99% identity to SEQ ID NO: 35 and comprising the CDRb1, CDRb2, and CDRb3 of SEQ ID NO: 5, 10, and 6, respectively;
   the second polypeptide comprises an amino acid sequence of SEQ ID NO: 36, or an amino acid sequence having at least 85%, 90%, 95%, 98% or 99% identity to SEQ ID NO: 36 and comprising the CDRb1, CDRb2, and CDRb3 of SEQ ID NO: 5, 10, and 6, respectively;
   the second polypeptide comprises an amino acid sequence of SEQ ID NO: 37, or an amino acid sequence having at least 85%, 90%, 95%, 98% or 99% identity to SEQ ID NO: 37 and comprising the CDRb1, CDRb2, and CDRb3 of SEQ ID NO: 5, 10, and 6, respectively;
   the second polypeptide comprises an amino acid sequence of SEQ ID NO: 38, or an amino acid sequence having at least 85%, 90%, 95%, 98% or 99% identity to SEQ ID NO: 38 and comprising the CDRb1, CDRb2, and CDRb3 of SEQ ID NO: 5, 10, and 6, respectively;
   the second polypeptide comprises an amino acid sequence of SEQ ID NO: 39, or an amino acid sequence having at least 85%, 90%, 95%, 98% or 99% identity to SEQ ID NO: 39 and comprising the CDRb1, CDRb2, and CDRb3 of SEQ ID NO: 5, 10, and 6, respectively;
   the second polypeptide comprises an amino acid sequence of SEQ ID NO: 40, or an amino acid sequence having at least 85%, 90%, 95%, 98% or 99% identity to SEQ ID NO: 40 and comprising the CDRb1, CDRb2, and CDRb3 of SEQ ID NO: 5, 10, and 6, respectively;
   the second polypeptide comprises an amino acid sequence of SEQ ID NO: 41, or an amino acid sequence having at least 85%, 90%, 95%, 98% or 99% identity to SEQ ID NO: 41 and comprising the CDRb1, CDRb2, and CDRb3 of SEQ ID NO: 5, 9, and 6, respectively;
   the second polypeptide comprises an amino acid sequence of SEQ ID NO: 42, or an amino acid sequence having at least 85%, 90%, 95%, 98% or 99% identity to SEQ ID NO: 42 and comprising the CDRb1, CDRb2, and CDRb3 of SEQ ID NO: 5, 9, and 6, respectively;
   the second polypeptide comprises an amino acid sequence of SEQ ID NO: 43, or an amino acid sequence having at least 85%, 90%, 95%, 98% or 99% identity to SEQ ID NO: 43 and comprising the CDRb1, CDRb2, and CDRb3 of SEQ ID NO: 5, 9, and 6, respectively;
   the second polypeptide comprises an amino acid sequence of SEQ ID NO: 44, or an amino acid sequence having at least 85%, 90%, 95%, 98% or 99% identity to SEQ ID NO: 44 and comprising the CDRb1, CDRb2, and CDRb3 of SEQ ID NO: 5, 9, and 6, respectively; or
   the second polypeptide comprises an amino acid sequence of SEQ ID NO: 45, or an amino acid sequence having at least 85%, 90%, 95%, 98% or 99% identity to SEQ ID NO: 45 and comprising the CDRb1, CDRb2, and CDRb3 of SEQ ID NO: 5, 9, and 6, respectively;
   wherein optionally said CDRa1, CDRa2, CDRa3, CDRb1, CDRb2 and/or CDRb3 sequences may comprise one, two or three amino acid mutations, preferably amino acid substitutions.
11. The antigen binding protein of any one of items 1 to 10,
   wherein the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
   the second polypeptide comprises an amino acid sequence of SEQ ID NO: 34, or an amino acid sequence having at least 85%, 90%, 95%, 98% or 99% identity to SEQ ID NO: 34 and comprising the CDRb1, CDRb2, and CDRb3 of SEQ ID NO: 5, 9, and 6, respectively;
   wherein the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively, and
   the second polypeptide comprises an amino acid sequence of SEQ ID NO: 35, or an amino acid sequence having at least 85%, 90%, 95%, 98% or 99% identity to SEQ ID NO: 35 and comprising the CDRb1, CDRb2, and CDRb3 of SEQ ID NO: 5, 10, and 6, respectively;
   wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
   the second polypeptide comprises an amino acid sequence of SEQ ID NO: 36, or an amino acid sequence having at least 85%, 90%, 95%, 98% or 99% identity to SEQ ID NO: 36 and comprising the CDRb1, CDRb2, and CDRb3 of SEQ ID NO: 5, 10, and 6, respectively;
   wherein the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
   the second polypeptide comprises an amino acid sequence of SEQ ID NO: 37, or an amino acid sequence having at least 85%, 90%, 95%, 98% or 99% identity to SEQ ID NO: 37 and comprising the CDRb1, CDRb2, and CDRb3 of SEQ ID NO: 5, 10, and 6, respectively;
   wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
   the second polypeptide comprises an amino acid sequence of SEQ ID NO: 38, or an amino acid sequence having at least 85%, 90%, 95%, 98% or 99% identity to SEQ ID NO: 38 and comprising the CDRb1, CDRb2, and CDRb3 of SEQ ID NO: 5, 10, and 6, respectively;
   wherein the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
   the second polypeptide comprises an amino acid sequence of SEQ ID NO: 38, or an amino acid sequence having at least 85%, 90%, 95%, 98% or 99% identity to SEQ ID NO: 38 and comprising the CDRb1, CDRb2, and CDRb3 of SEQ ID NO: 5, 10, and 6, respectively;
   wherein the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
   the second polypeptide comprises an amino acid sequence of SEQ ID NO: 39, or an amino acid sequence having at least 85%, 90%, 95%, 98% or 99% identity to SEQ ID NO: 39 and comprising the CDRb1, CDRb2, and CDRb3 of SEQ ID NO: 5, 10, and 6, respectively;
   wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
   the second polypeptide comprises an amino acid sequence of SEQ ID NO: 40, or an amino acid sequence having at least 85%, 90%, 95%, 98% or 99% identity to SEQ ID NO: 40 and comprising the CDRb1, CDRb2, and CDRb3 of SEQ ID NO: 5, 10, and 6, respectively;
   wherein the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
   the second polypeptide comprises an amino acid sequence of SEQ ID NO: 40, or an amino acid sequence having at least 85%, 90%, 95%, 98% or 99% identity to SEQ ID NO: 40 and comprising the CDRb1, CDRb2, and CDRb3 of SEQ ID NO: 5, 10, and 6, respectively;
   wherein the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively, and
   the second polypeptide comprises an amino acid sequence of SEQ ID NO: 37, or an amino acid sequence having at least 85%, 90%, 95%, 98% or 99% identity to SEQ ID NO: 37 and comprising the CDRb1, CDRb2, and CDRb3 of SEQ ID NO: 5, 10, and 6, respectively;
   wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
   the second polypeptide comprises an amino acid sequence of SEQ ID NO: 41, or an amino acid sequence having at least 85%, 90%, 95%, 98% or 99% identity to SEQ ID NO: 41 and comprising the CDRb1, CDRb2, and CDRb3 of SEQ ID NO: 5, 9, and 6, respectively;
   wherein the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
   the second polypeptide comprises an amino acid sequence of SEQ ID NO: 42, or an amino acid sequence having at least 85%, 90%, 95%, 98% or 99% identity to SEQ ID NO: 42 and comprising the CDRb1, CDRb2, and CDRb3 of SEQ ID NO: 5, 9, and 6, respectively;
   wherein the first polypeptide comprises an amino acid sequence of SEQ ID NO: 31, or an amino acid sequence having at least 85%, 90%, 95%, 98% or 99% identity to SEQ ID NO: 31 and comprising the CDRa1, CDRa2, and CDRa3 of SEQ ID NO: 2, 8, and 4, respectively, and
   the second polypeptide comprises an amino acid sequence of SEQ ID NO: 43, or an amino acid sequence having at least 85%, 90%, 95%, 98% or 99% identity to SEQ ID NO: 43 and comprising the CDRb1, CDRb2, and CDRb3 of SEQ ID NO: 5, 9, and 6, respectively;
   wherein the first polypeptide comprises an amino acid sequence of SEQ ID NO: 32, or an amino acid sequence having at least 85%, 90%, 95%, 98% or 99% identity to SEQ ID NO: 32 and comprising the CDRa1, CDRa2, and CDRa3 of SEQ ID NO: 2, 8, and 4, respectively, and
   the second polypeptide comprises an amino acid sequence of SEQ ID NO: 44, or an amino acid sequence having at least 85%, 90%, 95%, 98% or 99% identity to SEQ ID NO: 44 and comprising the CDRb1, CDRb2, and CDRb3 of SEQ ID NO: 5, 9, and 6, respectively; or
   wherein the first polypeptide comprises an amino acid sequence of SEQ ID NO: 33, or an amino acid sequence having at least 85%, 90%, 95%, 98% or 99% identity to SEQ ID NO: 33 and comprising the CDRa1, CDRa2, and CDRa3 of SEQ ID NO: 2, 8, and 4, respectively, and
   the second polypeptide comprises an amino acid sequence of SEQ ID NO: 45, or an amino acid sequence having at least 85%, 90%, 95%, 98% or 99% identity to SEQ ID NO: 45 and comprising the CDRb1, CDRb2, and CDRb3 of SEQ ID NO: 5, 9, and 6, respectively;
   wherein optionally said CDRa1, CDRa2, CDRa3, CDRb1, CDRb2 and/or CDRb3 sequences may comprise one, two or three amino acid mutations, preferably amino acid substitutions.
12. The antigen binding protein of any one of items 1 to 11,
   wherein the first polypeptide comprises an amino acid sequence of SEQ ID NO: 27 and the second polypeptide comprises an amino acid sequence of SEQ ID NO: 34;
   wherein the first polypeptide comprises an amino acid sequence of SEQ ID NO: 28 and the second polypeptide comprises an amino acid sequence of SEQ ID NO: 35;
   wherein the first polypeptide comprises an amino acid sequence of SEQ ID NO: 29 and the second polypeptide comprises an amino acid sequence of SEQ ID NO: 36;
   wherein the first polypeptide comprises an amino acid sequence of SEQ ID NO: 30 and the second polypeptide comprises an amino acid sequence of SEQ ID NO: 37;
   wherein the first polypeptide comprises an amino acid sequence of SEQ ID NO: 29 and the second polypeptide comprises an amino acid sequence of SEQ ID NO: 38;
   wherein the first polypeptide comprises an amino acid sequence of SEQ ID NO: 27 and the second polypeptide comprises an amino acid sequence of SEQ ID NO: 38;
   wherein the first polypeptide comprises an amino acid sequence of SEQ ID NO: 30 and the second polypeptide comprises an amino acid sequence of SEQ ID NO: 39;
   wherein the first polypeptide comprises an amino acid sequence of SEQ ID NO: 29 and the second polypeptide comprises an amino acid sequence of SEQ ID NO: 40;
   wherein the first polypeptide comprises an amino acid sequence of SEQ ID NO: 27 and the second polypeptide comprises an amino acid sequence of SEQ ID NO: 40;
   wherein the first polypeptide comprises an amino acid sequence of SEQ ID NO: 28 and the second polypeptide comprises an amino acid sequence of SEQ ID NO: 37;
   wherein the first polypeptide comprises an amino acid sequence of SEQ ID NO: 29 and the second polypeptide comprises an amino acid sequence of SEQ ID NO: 41;
   wherein the first polypeptide comprises an amino acid sequence of SEQ ID NO: 30 and the second polypeptide comprises an amino acid sequence of SEQ ID NO: 42;
   wherein the first polypeptide comprises an amino acid sequence of SEQ ID NO: 31 and the second polypeptide comprises an amino acid sequence of SEQ ID NO: 43;
   wherein the first polypeptide comprises an amino acid sequence of SEQ ID NO: 32 and the second polypeptide comprises an amino acid sequence of SEQ ID NO: 44; or
   wherein the first polypeptide comprises an amino acid sequence of SEQ ID NO: 33 and the second polypeptide comprises an amino acid sequence of SEQ ID NO: 45.
13. The antigen binding protein of any one of items 1 to 12, wherein antigen binding domain A is derived from a TCR, or is a TCR or fragment(s) thereof.
14. The antigen binding protein of item 13, wherein the TCR is selected from the group consisting of an α/β TCR, a γ/δ TCR, functional fragments of a TCR, and a fusion protein or chimeric protein comprising (a) functional fragments of a TCR.
15. The antigen binding protein of item 13 or item 14, wherein V_{A} and V_{B} are TCRα and TCRβ domains, respectively.
16. The antigen binding protein of any one of items 1 to 15, wherein the MHC protein is an HLA protein, preferably HLA-A, more preferably HLA-A*02.
17. The antigen binding protein of any one of items 1 to 16, wherein the antigen binding protein does not significantly bind to at least 1, at least 2, at least 3, at least 4, at least 5, or all similar peptides selected from the group consisting of SEQ ID NOs: 59 to 67, 106, and 133 to 151.
18. The antigen binding protein of any one of items 1 to 17, wherein the antigen binding protein is a soluble protein.
19. The antigen binding protein of any one of items 1 to 18, wherein the antigen binding protein is a multispecific antigen binding protein, in particular a bispecific antigen binding protein.
20. The antigen binding protein of any one of items 1 to 19, wherein the antigen binding protein comprises a further antigen binding domain B.
21. The antigen binding protein of any one of items 1 to 19, wherein the antigen binding protein further comprises a variable domain VH comprising a CDRH1, a CDRH2 and a CDRH3, and a VL comprising a CDRL1, a CDRL2, and a CDRL3, wherein CDRH1, CDRH2, CDRH3, CDRL1, CDRL2 and CDRL3 form an antigen binding domain B.
22. The antigen binding protein of item 20 or item 21, wherein the antigen binding domain B specifically binds to immune cells, preferably T cells or natural killer (NK) cells.
23. The antigen binding protein of item 22, wherein the antigen binding domain B specifically binds to an antigen selected from the group consisting of CD2, CD3, in particular CD3γ, CD3δ, and/or CD3ε, CD4, CD5, CD7, CD8, CD10, CD11b, CD11c, CD14, CD16, CD18, CD22, CD25, CD28, CD32a, CD32b, CD33, CD41, CD41b, CD42a, CD42b, CD44, CD45RA, CD49, CD55, CD56, CD61, CD64, CD68, CD69, CD89, CD90, CD94, CD95, CD117, CD123, CD125, CD134, CD137, CD152, CD163, CD193, CD203c, CD235a, CD278, CD279, CD287, Ly-6.2C, Mel14, Nkp46, NKG2D, GITR, F_{C}εRI, TCRα/β, TCRγ/δ, HLA-DR and 4-1 BB, or combinations thereof.
24. The antigen binding protein of item 22 or item 23, wherein the antigen binding domain B specifically binds to an α/β T cell receptor (TCR)/CD3 complex.
25. The antigen binding protein of any one of items 21 to 24, wherein the CDRH3 comprises the amino acid sequence of SEQ ID NO: 50 [GSYYDYEGFVY] or SEQ ID NO: 58 [GSYYDYDGFVY], and optionally wherein the CDRH3 comprises up to three amino acid mutations, preferably amino acid substitutions.
26. The antigen binding protein of any one of items 21 to 25, wherein the VH comprises
   (a) a CDRH1 comprising the amino acid sequence of X₁YVMH, wherein X₁ is S, R, K or H [SEQ ID NO: 111];
   (b) a CDRH2 comprising the amino acid sequence of YINPX₁X₂DVTKYX₃X₄KFX₅G,
   wherein
   X₁ is Y, R, K, or H, preferably Y or R;
   X₂ is N, R, or K, preferably N or R;
   X₃ is A or N;
   X₄ is E or Q, preferably E; and
   X₅ is Q or K [SEQ ID NO: 112], and
   (c) a CDRH3 comprising the amino acid sequence of GSYYDYEGFVY [SEQ ID NO: 50] or GSYYDYDGFVY [SEQ ID NO: 58], and wherein the VL comprises
   (a) a CDRL1 comprising the amino acid sequence of SATSSVXYMH, wherein X is S, R, or K [SEQ ID NO: 113],
   (b) a CDRL2 comprising the amino acid sequence of DTSKLAX, wherein X is S, R or K [SEQ ID NO: 114], and
   (c) a CDRL3 comprising the amino acid sequence of QQWSSNPLT [SEQ ID NO: 53];
and optionally wherein each of the CDRH1, the CDRH2 and the CDRH3, and the CDRL1, CDRL2, and CDRL3 comprises up to three amino acid substitutions.
27. The antigen binding protein of any one of items 21 to 26, wherein the VH comprises:
   (a) a CDRH1 comprising the amino acid sequence of SYVMH [SEQ ID NO: 48],
   (b) a CDRH2 comprising the amino acid sequence of YINPRNDVTKYAEKFQG [SEQ ID NO: 49] or YINPYNDVTKYAEKFQG [SEQ ID NO: 57], and
   (c) a CDRH3 comprising the amino acid sequence of GSYYDYEGFVY [SEQ ID NO: 50] or GSYYDYDGFVY [SEQ ID NO: 58], and
   wherein the VL comprises:
   (a) a CDRL1 comprising the amino acid sequence of SATSSVSYMH [SEQ ID NO: 51],
   (b) a CDRL2 comprising the amino acid sequence of DTSKLAS [SEQ ID NO: 52], and
   (c) a CDRL3 comprising the amino acid sequence of QQWSSNPLT [SEQ ID NO: 53];
and optionally wherein each of the CDRH1, the CDRH2 and the CDRH3, and the CDRL1, CDRL2, and CDRL3 comprises up to three amino acid substitutions.
28. The antigen binding protein of any one of items 21 to 27,
   wherein the VH comprises an amino acid sequence selected from the group consisting of SEQ ID NOs: 46, 54, 55 and 56, or an amino acid sequence having at least 85%, 90%, 95%, 98% or 99% identity to SEQ ID NOs: 46, 54, 55 or 56 and preferably comprising the CDRH1, CDRH2, and CDRH3 according to any one of the previous items; and
   wherein the VL comprises an amino acid sequence of SEQ ID NO: 47, or an amino acid sequence having at least 85%, 90%, 95%, 98% or 99% identity to SEQ ID NO: 47, and preferably comprising the CDRL1, CDRL2, and CDRL3 according to any one of the previous items;
   and optionally wherein each of said CDRH1, CDRH2 and CDRH3, and said CDRL1, CDRL2 and CDRL3 comprises up to three amino acid substitutions.
29. The antigen binding protein of any one of items 1 to 28, wherein the antigen binding site B comprises
   (i) the V_{H} that comprises an amino acid sequence selected from the group consisting of SEQ ID NOs: 46, 54, 55 and 56, or (an) antigen binding fragment(s) thereof, and
   (ii) the V_{L} that comprises an amino acid sequence of SEQ ID NO: 47, or (an) antigen binding fragment(s) thereof.
30. The antigen binding protein of any one of items 1 to 29, wherein the first polypeptide and the second polypeptide are covalently or non-covalently linked together.
31. The antigen binding protein of any one of items 1 to 30, wherein the binding domains A and/or B are in the format selected from the group consisting of an antibody, a single chain fragment variable (scFv), disulfide-stabilized Fv (dsFv), Fab, Fab′, F(ab′)2, a nanobody, a DARPin, a Knottin, a diabody or oligomers thereof.
32. The antigen binding protein of any one of items 21 to 31, wherein the binding domains A and B are in the format of an scFv.
33. The antigen binding protein of any one of items 21 to 31, wherein said antigen binding protein comprises a single chain TCR (scTCR) and/or a single-chain bispecific antibody.
34. The antigen binding protein of any one of items 21 to 33, wherein the antigen binding protein comprises two antigen binding sites A and B, wherein a first polypeptide chain comprises a structure represented by formula:

V1-L1-V2 [I]
wherein V1 is a first variable domain; V2 is a second variable domain; L₁ is a linker domain; and wherein a second polypeptide chain comprises a structure represented by formula:

V3-L3-V4 [II]
wherein V3 is a third variable domain; V4 is a fourth variable domain; L₃ is a linker; optionally wherein:
   V1 and V2 form the antigen binding site A and V3 and V4 form the antigen binding site B, or
   V1 and V2 form the antigen binding site B and V3 and V4 form the antigen binding site A, or
   V1 and V3 form the antigen binding site A and V2 and V4 form the antigen binding site B, or
   V1 and V3 form the antigen binding site B and V2 and V4 form the antigen binding site A, or
   V1 and V4 form the antigen binding site A and V2 and V3 form the antigen binding site B, or
   V1 and V4 form the antigen binding site B and V2 and V3 form the antigen binding site A, and
   optionally, wherein the first polypeptide and the second polypeptide are covalently or non-covalently linked together.
35. The antigen binding protein of any one of items 21 to 34, wherein the antigen binding protein comprises two antigen binding sites A and B, wherein a first polypeptide chain comprises a structure represented by formula:

V1-L1-V2-L2-C1 [III]
wherein V1 is a first variable domain; V2 is a second variable domain; L1 and L2 are linkers; L2 may be present or absent; C1 is a constant domain or a dimerizing portion thereof and present or absent, preferably wherein C1 is CL a light chain constant domain or a dimerizing portion thereof;
and wherein a second polypeptide chain comprises a structure represented by formula:

V3-L3-V4-L4-C2 [IV]
wherein V3 is a third variable domain; V4 is a fourth variable domain; L₃ and L₄ are linkers; L₄ may be present or absent; C2 is a constant domain or a dimerizing portion thereof and is present or absent, preferably wherein C2 is CH a heavy chain constant domain or a dimerizing portion thereof; and wherein preferably
V1 and V2 form the antigen binding site A and V3 and V4 form the antigen binding site B, or
V1 and V2 form the antigen binding site B and V3 and V4 form the antigen binding site A, or
V1 and V3 form the antigen binding site A and V2 and V4 form the antigen binding site B, or
V1 and V3 form the antigen binding site B and V2 and V4 form the antigen binding site A, or
V1 and V4 form the antigen binding site A and V2 and V3 form the antigen binding site B, or
V1 and V4 form the antigen binding site B and V2 and V3 form the antigen binding site A, and
optionally, wherein the first polypeptide and the second polypeptide are covalently or non-covalently linked together.
36. The antigen binding protein of item 35, wherein the first polypeptide chain has a structure represented by the formula [V]:

V1-L1-V2-L2-C1-L5-FC1 [V]
and the second polypeptide chain has a structure represented by the formula [VI]:

V3-L3-V4-L4-C2-L6-FC2 [VI]
wherein V1 to V4, L1 to L4, C1 and C2 are as defined in item 35 and wherein L5 and L6 are linkers and present or absent and FC1, and FC2 are F_{C}-domains and wherein FC1 and FC2 are the same or different.
37. The antigen binding protein of item 36, wherein F_{C1} and/or F_{C2} comprises or consists of
   the amino acid sequence SEQ ID NO: 24 [Fc knob] and/or SEQ ID NO: 25 [F_{C} hole]; and/or
   wherein L₁ and L₃ comprise or consist of the amino acid sequence GGGSGGGG [SEQ ID NO: 26].
38. The antigen binding protein of item 36, wherein
   V1 comprises CDRa1, CDRa2 and CDRa3 as defined in the preceding items,
   V2 comprises CDRH1, CDRH2, and CDRH3 as defined in the preceding items,
   V3 comprises CDRL1, CDRL2, and CDRL3 as defined in the preceding items,
   V4 comprises CDRb1, CDRb2 and/or CDRb3 as defined in the preceding items; optionally
   wherein L₁ and L₃ comprise or consist of the amino acid sequence GGGSGGGG of SEQ ID NO: 26, and wherein L₂, L₄, L₅ and L₆ are absent, and optionally
   wherein F_{C1} comprises or consists of the amino acid sequence SEQ ID NO: 25 [F_{C} hole], and, F_{C2} comprises or consists of the amino acid sequence SEQ ID NO: 24 [Fc knob], or
   wherein F_{C1} comprises or consists of the amino acid sequence SEQ ID NO: 24 [Fc knob], and F_{C2} comprises or consists of the amino acid sequence SEQ ID NO: 25 [F_{C} hole].
39. The antigen binding protein of any one of items 1 to 38, wherein the amino acid sequences of the binding domains A and B are chimeric, humanized or human.
40. The antigen binding protein of any one of items 1 to 39, wherein the N-terminal or the C-terminal amino acid(s) of the polypeptide chains of the antigen binding protein comprises (a) modification(s).
41. The antigen binding protein of any one of items 1 to 39, wherein the N-terminal amino acid(s) of the polypeptide chains of the antigen binding protein comprise a modification.
42. The antigen binding protein of any one of items 18 to 41, wherein the antigen binding protein is capable of activating:
   a CD4+ T cell, preferably a CD4+CD8-T cell, and/or
   a CD8+ T cell, preferably a CD8+CD4-T cell.
43. The antigen binding protein of any one of items 18 to 42, wherein the antigen binding protein binds the MAGEB2 antigenic peptide of SEQ ID NO: 1 with a K_{D} of less than 50 nM, preferably less than 20 nM, more preferably less than 5 nM, as measured by biolayer interferometry.
44. The antigen binding protein of any one of items 18 to 43, wherein the antigen binding protein is capable of killing MAGEB2-expressing tumor cells in an in vitro cytotoxicity assay, wherein the MAGEB2-expressing tumor cells have a MAGEB2 copy number per cell of less than 200.
45. The antigen binding protein of item 44, wherein the MAGEB2-expressing tumor cells have a MAGEB2 copy number per cell of less than 100, preferably less than 50.
46. A nucleic acid or nucleic acids encoding the antigen binding protein as defined in any one of items 1 to 45.
47. A vector or vectors comprising the nucleic acid or nucleic acids of item 46.
48. A host cell comprising the nucleic acid or nucleic acids of item 46 or the vector(s) of item 47.
49. The host cell of item 48, wherein the host cell is a cell for recombinant expression, such as a Chinese Hamster Ovary (CHO) cell or a yeast cell.
50. A method of making the antigen binding protein of any one of items 1 to 45, comprising culture the host cell of item 48 or item 49 under suitable conditions and optionally isolating the antigen binding protein produced by the host cells.
51. An antigen binding protein as produced by the method of item 50.
52. A pharmaceutical composition comprising an antigen binding protein of any one of items 1 to 45 or 51, (a) nucleic acid(s) of item 46, or (a) vector(s) of item 47, and optionally a pharmaceutically acceptable carrier.
53. A pharmaceutical composition of item 52, wherein the antigen binding protein is dissolved or dispersed in a pharmaceutically acceptable carrier or aqueous medium.
54. The antigen binding protein of any one of items 1 to 45 or 51, the nucleic acid(s) of item 46, the vector(s) of item 47, or the pharmaceutical composition of items 52 or 53, for use in the treatment of cancer.
55. The antigen binding protein of any one of items 1 to 45 or 51, the nucleic acid(s) of item 46, the vector(s) of item 47, or the pharmaceutical composition of item 52 or 53, for use in the treatment of a MAGEB2-expressing cancer.
56. A method of treatment of a MAGEB2-expressing cancer, comprising administering to a subject in need thereof a therapeutically effective amount of the antigen binding protein of any one of items 1 to 45 or 51, the nucleic acid(s) of item 46, the vector(s) of item 47, or the pharmaceutical composition of 52 or 53.
57. The antigen binding protein for the use of item 54 or item 55, or the method of treatment of item 56, wherein the MAGEB2-expressing cancer is selected from the group consisting of hepatocellular carcinoma cancer (HCC), melanoma, non-small cell lung cancer (NSCLC), and non-Hodgkin lymphoma (NHL) and UEC (uterine endometrial cancer).
58. The antigen binding protein, nucleic acid(s), vector(s), or pharmaceutical composition for the use of item 54, 55 or 57, or the method of treatment of item 56 or item 57, wherein the antigen binding protein is conjugated to a therapeutically active agent.
59. The antigen binding protein, nucleic acid(s), vector(s), or pharmaceutical composition for the use of item 58, or the method of treatment of item 58, wherein the therapeutically active agent is selected from the group consisting of a radionuclide, a chemotherapeutic agent and a toxin.
60. The antigen binding protein, nucleic acid(s), vector(s), or pharmaceutical composition for the use of any one of items 54, 55, or 57 to 59, or the method of treatment of any one of items 56 to 59, wherein the treatment further comprises administering at least one chemotherapeutic agent to the subject in need of treatment.
61. The antigen binding protein, nucleic acid(s), vector(s), or pharmaceutical composition for the use of any one of items 54, 55, or 57 to 60, or the method of treatment of any one of items 56 to 60, wherein the treatment further comprises administering radiation therapy to the subject in need of treatment.
62. A kit comprising an antigen binding protein of any one of items 1 to 45 or 51, (a) nucleic acid(s) of item 46, and/or (a) vector(s) of item 47.
63. A composition comprising or consisting of the antigen binding molecule of any one of items 1 to 45 or 51, (a) nucleic acid(s) of item 46, and/or (a) vector(s) of item 47.
64. A method of treating cancer in a subject in need thereof, comprising administering to the subject the antigen binding protein of any one of items 1 to 45 or 51 and/or the nucleic acid or nucleic acids of item 46, wherein the cells of the cancer present GVYDGEEHSV (SEQ ID NO: 1) in a complex with an MHC molecule on the cell surface.
65. An antigen binding protein comprising
   a first polypeptide comprising a variable domain V_{A} comprising complementarity determining regions (CDRs) CDRa1, CDRa2, and CDRa3, and a second polypeptide comprising a variable domain V_{B} comprising CDRb1, CDRb2, and CDRb3, wherein CDRa1, CDRa2, CDRa3, CDRb1, CDRb2 and CDRb3 form an antigen binding domain A;
   wherein
   (1) CDRa1 comprises SEQ ID NO: 2, CDRa3 comprises SEQ ID NO: 3, CDRb1 comprises SEQ ID NO: 5, and CDRb3 comprises SEQ ID NO: 6; or
   (2) CDRa1 comprises SEQ ID NO: 2, CDRa3 comprises SEQ ID NO: 4, CDRb1 comprises SEQ ID NO: 5, and CDRb3 comprises SEQ ID NO: 6;
   wherein the CDRa1, CDRa3, CDRb1 and/or CDRb3 sequence(s) may comprise one, two or three amino acid mutations.
66. The antigen binding protein of item 65 wherein the one, two, or three amino acid mutations are conservative substitutions.
67. The antigen binding protein of item 65 comprising a first polypeptide comprising a variable domain V_{A} comprising complementarity determining regions (CDRs) CDRa1, CDRa2, and CDRa3, and a second polypeptide comprising a variable domain V_{B} comprising CDRb1, CDRb2, and CDRb3, wherein CDRa1, CDRa2, CDRa3, CDRb1, CDRb2 and CDRb3 form an antigen binding domain A; wherein
(1) CDRa1 comprises SEQ ID NO: 2, CDRa3 comprises SEQ ID NO: 3, CDRb1 comprises SEQ ID NO: 5, and CDRb3 comprises SEQ ID NO: 6; or
(2) CDRa1 comprises SEQ ID NO: 2, CDRa3 comprises SEQ ID NO: 4, CDRb1 comprises SEQ ID NO: 5, and CDRb3 comprises SEQ ID NO: 6.

### EXAMPLES

### Example 1: Generation of Stable scTCR

For the present invention, the TCR R76P1E1 (SEQ ID NOs: 76 and 77) was converted into a single chain TCR construct (scTCR R76P1E1, SEQ ID NO: 89) using the variable alpha (SEQ ID NO: 78) and beta (SEQ ID NO: 79) domains and a glycine-serine linker sequence (SEQ ID NO: 85). For TCR maturation via yeast surface display, the DNA of the corresponding sequence was synthesized and transformed into Saccharomyces cerevisiae EBY100 (MATa AGA1:GAL1¬AGA1:URA3 ura3¬52 trp1 leu2¬delta200 his3¬delta200 pep4:HIS3 prbd1.6R can1 GAL) (ATCC® MYA¬4941™) together with a yeast display vector based on pCT302 (Boder and Wittrup, Methods Enzymol. 2000; 328:430-44). The resulting fusion protein after homologous recombination in the yeast (SEQ ID NO: 88) contains a leader peptide at the N-terminus of the Aga2p protein (SEQ ID NO: 84) (Boder and Wittrup, Nat Biotechnol. 1997 June; 15 (6): 553-7), the protein of interest, namely the scTCR R76P1E1 (SEQ ID NO: 89) or its variants and additional peptide tags (FLAG and Myc (SEQ ID NOs: 86 and 87)) to determine the expression level of the fusion protein. Libraries of scTCR variants were generated via PCR using degenerate primers and the transformation of yeast cells was performed as described in WO 2018/091396 and resulted in up to 109 yeast clones per library.

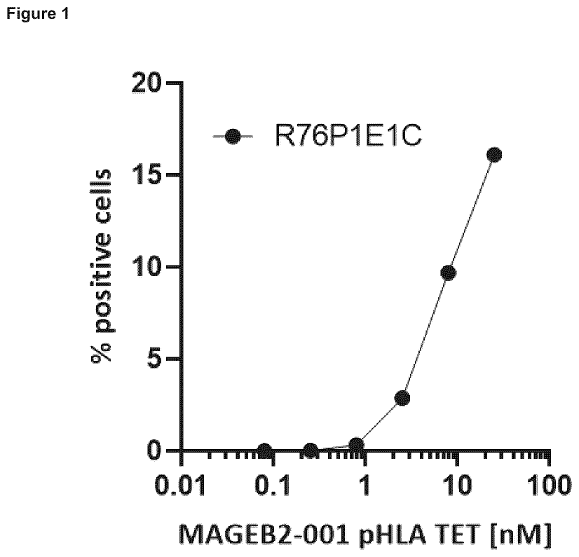

The selection process for the yeast clones bearing mutant scTCR variants with improved binding to MAGEB2-001 in the context of HLA-A*02 was performed with prior art methods. Functional binding via HLA-A*02/MAGEB2-001 tetramer staining was applied to select for most promising candidates. As a starting point for the scTCR conversion, the variant scTCR R76P1E1_bC13V (SEQ ID NO: 90) was chosen. The scTCR conversion by yeast surface display revealed six framework mutations in combination with two single point CDR mutations (SEQ ID NO: 4 and 92), resulting in the further stabilized scTCR R76P1E1C (SEQ ID NO: 91) showing expression as well as HLA-A*02/MAGEB2-001 tetramer binding (FIG. 1).

### Example 2: Affinity Maturation of Stabilized scTCR Improved the Binding to HLA-A*02/MAGEB2-001 and Provided Highly Specific Binding

To generate scTCR molecules with higher binding affinity towards HLA-A*02/MAGEB2-001, all CDRs were matured individually, using a cysteine-remediated version (scTCR R76P1E1S (SEQ ID NO: 93)) of the previously identified stabilized scTCR R76P1E1C (SEQ ID NO: 91). The CDR residues were randomized by using degenerate DNA oligo primers and the resulting DNA libraries were transformed as described in Example 1.

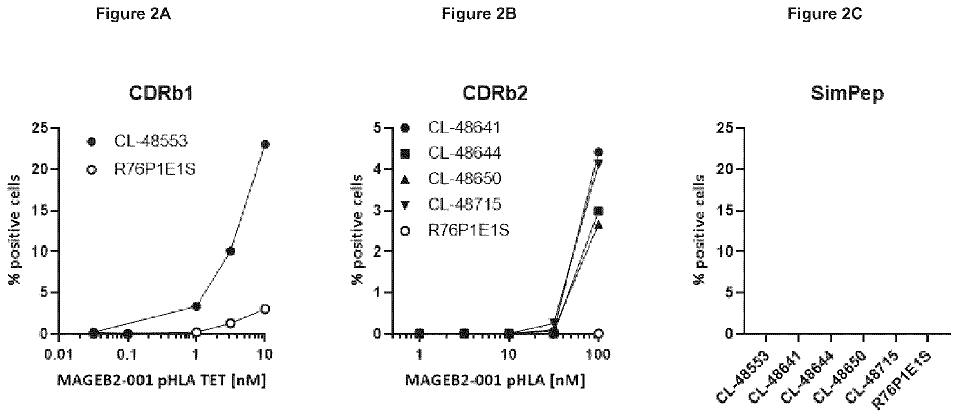

For the selection of affinity enhanced and specific R76P1E1S scTCR variants, decreasing concentrations of HLA-A*02/MAGEB2-001 tetramer or monomer were used for each selection round. After four selection rounds, single scTCR clones were isolated and sequenced, resulting in a multitude of affinity matured CDR sequences. As shown, a strong improvement in binding of HLA-A*02/MAGEB2-001 tetramers or monomers could be demonstrated for scTCR with matured CDRb1 and CDRb2 (SEQ ID NOs: 5 and 107-110, Table 2). The selectivity of HLA-A*02/MAGEB2-001 binding was retained during maturation as confirmed by the low binding of the scTCR to a mix of HLA-A*02 tetramers containing peptides (similar peptides or SimPeps) with high degree of sequence similarity to MAGEB2-001 peptide (SEQ ID NO: 1). While all selected CDRb2 scTCR maturation variants showed substantial staining with HLA-A*02/MAGEB2-001 monomers, the non-matured stabilized scTCR R76P1E1S as reference did not show staining (FIG. 2B and Table 2). Selected CDRb1 variants showed improved binding to HLA-A*02/MAGEB2-001 tetramers, compared to the reference. Furthermore, binding of matured scTCR to a mix of similar peptides, applied in a high avidity format of HLA-A*02 tetramers at a concentration of 25 nM, could not be detected, which confirms the capability of the scTCR maturation variants to bind the MAGEB2-001 target peptide in a highly specific manner.

TABLE 2
Binding data of yeast bearing scTCRs with mutant CDR2s¹

| | | Yeast cells stained | | | | Yeast cells stained | |
|---|---|---|---|---|---|---|---|
| | | positive with | | | | positive with | |
| | | | HLA- | | | | HLA- |
| CDRb1 | | HLA-A*02/ | A*02/Sim | CDRb2 | | HLA-A*02/ | A*02/ |
| | SEQ | MAGEB2- | Pep, | | SEQ | MAGEB2- | SimPep, |
| Se- | ID | 001, | tetramer | | ID | 001, | tetramer |
| quence | NO | tetramer | mix | Sequence | NO | monomer | mix, |
| RPVTM | 5 | 23.0% | 0.0% | AYDNNRT | 107 | 4.4% | 0.0% |
| SQVTM* | 82 | 3.6% | 0.0% | AFDDNTT | 108 | 3.0% | 0.0% |
| | | | | AYDDGSA | 109 | 2.7% | 0.0% |
| | | | | AYSDGTA | 110 | 4.1% | 0.0% |
| | | | | ANQGSEA* | 83 | 0.0% | 0.0% |

¹Stabilized scTCR comprising non-modified and matured CDR1 beta and CDR2 beta were stained with 10 nM HLA-A*02/MAGEB2-001 tetramer or 100 nM HLA-A*02/MAGEB2-001 monomer, respectively, and counterstained with a mix of HLA-A*02 tetramers, each applied at a concentration of 25 nM, containing peptides (similar peptides or SimPeps, SEQ ID NOs: 59-67) with high sequence similarity to MAGEB2-001 (SEQ ID NO: 1).
*corresponding CDR from R76P1E1S scTCR (SEQ ID NO: 93).

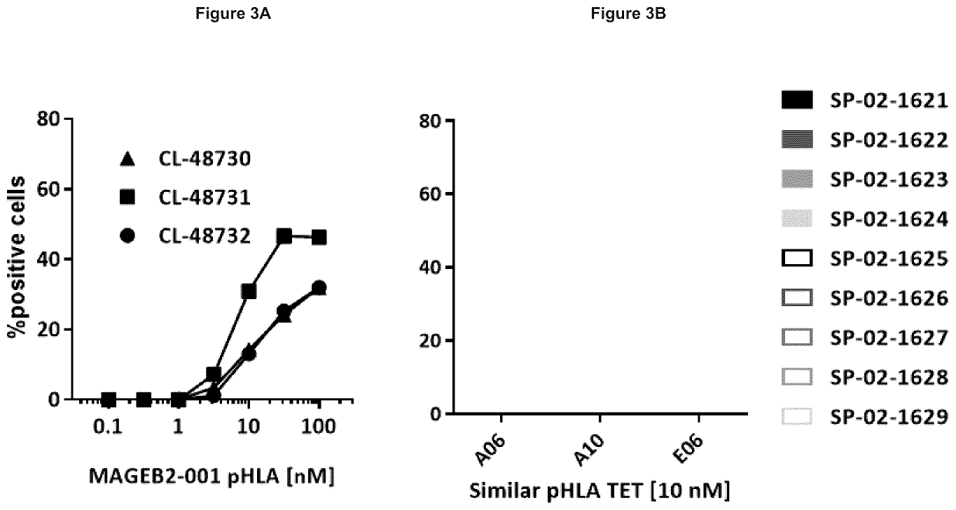

To further increase the affinity of scTCR clones, matured CDRs identified in above-described CDR libraries were systematically combined in one DNA library and transformed into Saccharomyces cerevisiae EBY100 as described in example 1. This library was selected using HLA-A*02/MAGEB2-001 monomer and scTCR from single yeast clones were sequenced and analyzed regarding their binding towards HLA-A*02 monomers containing either the MAGEB2-001 target peptide (SEQ ID NO: 1) or one peptide derived from the group of 9 peptides (similar peptides) sharing sequence similarities with MAGEB2-001 (SEQ ID NOs: 59-67). All the selected high affinity scTCR variants, CL-48730, CL-48731, and CL-48732 (SEQ ID NOS: 94-96, respectively) bound strongly to HLA-A*02/MAGEB2001 monomer. None of the scTCR variants exhibited binding above background levels to any of the similar peptides (SEQ ID NOs: 59-67) in the context of HLA-A*02 tetramers applied at a concentration of 10 nM (FIG. 3B). The presented data confirm the high binding specificity of the scTCR variants with combined CDR mutations.

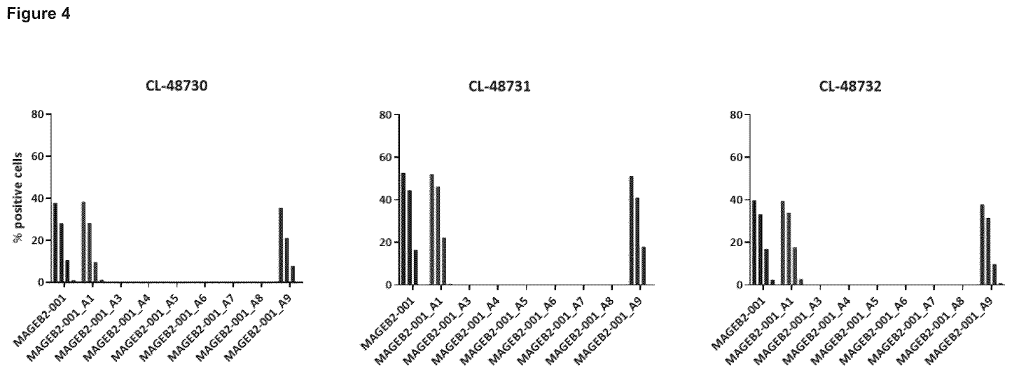

These selected high affinity scTCRs from yeast surface display were further examined regarding their functional epitope on the target peptide in context of the HLA-A*02 presentation, called binding motif. This was addressed by single substitutions of positions 1, 3, 4, 5, 6, 7, 8 and 9 of the MAGEB2-001 target peptide (SEQ ID NOs: 68-75) and assessment of binding of scTCR-bearing yeast cells to the respective MAGEB2-001 peptide variants in context of HLA-A*02. Four concentrations (31.6 nM, 10 nM, 3.2 nM, 1 nM) of HLA-A*02 monomers with MAGEB2-001 or the respective substituted peptides were used to stain the high affinity scTCR-bearing yeast cells and revealed a broad binding motif for all scTCR variants with strong recognition of positions 3 to 8 (FIG. 4).

### Example 3: Biooptimization of scTCRs Resulted in Low Nanomolar to Subnanomolar Binding EC₅₀ Values

For further optimization of the scTCRs, CL-48730 (SEQ ID NO: 94) was selected as starting point. Two potential isomerization sites, one in CDRa1, the other in CDRb2, were remediated, resulting in CL-21753 (SEQ ID NO: 97). Subsequently, two yeast surface display libraries were generated as described in Example 1, one error-prone PCR-based, the other with defined amino acid substitutions to further optimize the properties of the binders.

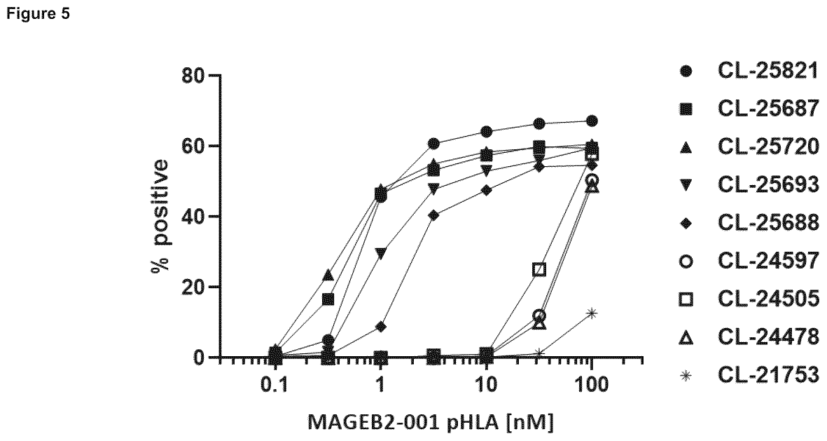

After selection with decreasing concentrations of HLA-A*02/MAGEB2-001 monomer and continuous counterselection with HLA-A*02/similar peptide tetramers, eight clones were further investigated and compared to the parental scTCR CL-21753. All of these clones showed a strong improvement in HLA-A*02/MAGEB2-001 binding, compared to their parental clone CL-21753 (FIG. 5), resulting in low nanomolar to subnanomolar EC₅₀ values (Table 3).

TABLE 3
Binding data of yeast-bearing scTCR molecules and respective variable chain sequences.²

| | | | | | | | | | | EC₅₀ | |
|---|---|---|---|---|---|---|---|---|---|---|---|
| | | FRa | | | | FRb | | | | for | |
| | | muta- | | | | muta- | | | | HLA- | |
| | | tions | | | | tions | | | | A*02/ | Simi- |
| | | (com- | | | | (com- | | | | MAGEB2- | lar |
| | | pared | | | | pared | | | | 001 | pep- |
| Mole- | SEQ | to pa- | | | | to | | | | bind- | tide |
| cule | ID | rental | | | | parental | | | | ing | bind- |
| name | NOs | TCR) | CDRa1 | CDRa2 | CDRa3 | TCR) | CDRb1 | CDRb2 | CDRb3 | [nM] | ing |
| CL- | 97 | L9S, | DASSTY | IFSNMDM | AVKDNA | C13V, T17N, | RPVTM | AYDEGSA | SVLGAYGYT | n.d. | 0 |
| 21753 | | S19A, | | | RLM | L19V | | | | | |
| | | L39F | | | | | | | | | |
| CL- | 102 | L9S, | DASSTY | IFSNMDM | AVKDNA | D11V, | RPVTM | AYDEGSA | SVLGAYGYT | 0.7 | 0 |
| 25821 | | S19A, | | | RLM | C13V, T17N, | | | | | |
| | | L39F, | | | | L19V, F40Y, | | | | | |
| | | G121V | | | | M94V | | | | | |
| CL- | 101 | L9S, | DASSTY | IFSNMDM | AVKDNA | D11Y, C13V, | RPVTM | AYDVGSA | SVLGAYGYT | 0.4 | 0 |
| 25720 | | S19A, | | | RLM | T17N, L19V, | | | | | |
| | | L39F | | | | L50P | | | | | |
| CL- | 100 | L9S, | DASSTY | IFSNMDM | AVKDNA | C13V, T17N, | RPVTM | AYDEGSA | SVLGAYGYT | 1.0 | 0 |
| 25693 | | S19A, | | | RLM | L19V, F40Y, | | | | | |
| | | L39F | | | | V72D, L103F | | | | | |
| CL- | 98 | L9S, | DDSSTY | IFSNMDM | AVKDNA | C13V, T17N, | RPVTM | AYDEGSA | CVLGAYGYT | 2.0 | 0 |
| 25688 | | S19A, | | | RLM | L19V, L50P | | | | | |
| | | L39F | | | | | | | | | |
| CL- | 99 | L9S, | DASSTY | IYSNMDM | AVKDNA | C13V, T17N, | RPVTM | AYDEGSA | SVLGAYGYT | 0.5 | 0 |
| 25687 | | S19A, | | | RLL | L19V, V72D | | | | | |
| | | L39F | | | | | | | | | |
| CL- | 103 | L9S, | DSSSTY | IFSNMDM | AVKDNA | C13V, L19V, | RPVTM | AYDEGSA | SVLGAYGYT | 51 | 0 |
| 24597 | | S19A, | | | RLM | M94L, S95E | | | | | |
| | | L39F | | | | | | | | | |
| CL- | 104 | L9S, | DASSTY | IFSNMDM | AVKDNA | C13V, L19V, | RPVTM | AYDEGSA | SVLGAYGYT | 35 | 0 |
| 24505 | | S19A, | | | RLM | S95E | | | | | |
| | | L39F | | | | | | | | | |
| CL- | 105 | L9S, | DASSTY | IFSNMDM | AVKDNA | C13V, L19V, | RPVTM | AYDEGSA | SVLGAYGYT | 50 | 0 |
| 24478 | | S19A, | | | RLM | N93S, S95E | | | | | |
| | | L39F | | | | | | | | | |

²For scTCR-bearing yeast cells, binding towards HLA-A*02/MAGEB2-001 monomers is presented as EC₅₀ values and binding towards 9 similar peptides (SEQ ID NOs: 59-67) in context of 10 nM HLA-A*02 tetramer is presented as % positive cells.
FR: Framework region;
n.d.: not determined

### Example 4: Bispecific Variants can be Produced as Soluble Molecules and Show Good Stability

TCR variants containing distinct mutations were included into an exemplary bispecific TCR format denoted as TCER molecules (cf. WO 2019/012138) containing the humanized recruiting domain “BMhanced” (VH and VL: SEQ ID NOs: 46 and 47, respectively) and human IgG1 F_{C} domains. DNA coding for the polypeptide chains were synthesized and cloned into mono-cistronic, controlled by HCMV-derived promoter elements, pUC19-derivatives. Plasmid DNA was amplified in E. coli according to standard culture methods and subsequently purified using commercial-available kits (Macherey & Nagel). Purified plasmid DNA was used for chemical-mediated transient transfection of CHO-S cells. Transfected CHO-cells were cultured 10-12 days at 32° C. to 37° C. and received one to three feeds of Cellboost 7a and 7b (GE Healthcare™) solution.

Conditioned cell supernatant was cleared by filtration (0.22 μm) utilizing Sartoclear Dynamics® Lab Filter Aid (Sartorius). Bispecific antigen binding proteins were purified using an Äkta Pure 25 L FPLC system (GE Lifesciences) equipped to perform affinity and size-exclusion chromatography in line. Affinity chromatography was performed on MAbSelect SuRE or protein L columns (GE Lifesciences) following standard affinity chromatographic protocols. Size exclusion chromatography was performed directly after elution (pH 2.8) from the affinity column to obtain highly pure monomeric protein using, Superdex 200 μg 26/600 columns (GE Lifesciences) following standard protocols. Protein concentrations were determined on a NanoDrop system (Thermo Scientific) using calculated extinction coefficients according to predicted protein sequences. Concentration was adjusted, if needed, by using Vivaspin devices (Sartorius). Finally, purified molecules were stored in phosphate-buffered saline at concentrations of about 1 mg/ml at temperatures of 2-8° C.

Quality of purified bispecific antigen binding proteins was determined by HPLC-SEC on MabPac SEC-1 columns (5 μm, 4×300 mm) or XBridge Premier SEC (dimensions 4.6×150 mm, particle size 2.5 μm) running in 50 mM sodium-phosphate pH 6.8 containing 300 mM NaCl within a Vanquish uHPLC-System. Detector wavelength was set to 214 nm.

Starting with an orientation run for comparison of different variable domain orders, the TCER molecules with identical variable domain sequences (TPP-4017 to TPP-4020) were produced successfully and provided acceptable heat-stress stability (Table 4). TPP-4020 was selected as base molecule for further engineering.

All further variants showed production yields exceeding 10 mg/L. Also, compared to the precursor molecule TPP-4020 all subsequent molecules showed significant increase in productivity. Heat-stress testing of the molecules at 40° C. revealed very good to acceptable stabilities for all produced bispecific molecules showing less than 13% high-molecular-weight species (HMWS) formation upon 14 days of heat-stress. Again, compared to the precursor molecule TPP-4020, all subsequent molecules exhibited significantly higher monomer content upon heat stress indicating increased stability of the generated variants.

TABLE 4
Productivity and quality of purified TCER molecules

| | | Monomer after | | Monomer |
|---|---|---|---|---|
| | | acidic elution | Final | after |
| | Yield | from capture | Monomer | 14 days at |
| TCER Variant | [mg/L] | column [%] | [%] | 40° C. [%] |
| TPP-4017 | 15 | 65 | 97.12 | 84.86 |
| TPP-4018 | 12 | 65 | 91.88 | 47.87 |
| TPP-4019 | 5 | 41 | 95.78 | 66.55 |
| TPP-4020 | 11 | 59 | 97.59 | 87.36 |
| TPP-4606 | 40 | 75 | 98.38 | 91.76 |
| TPP-4784 | 35 | 78 | 98.55 | 94.16 |
| TPP-4787 | 46 | 83 | 98.38 | 95.97 |
| TPP-4788 | 31 | 82 | 98.49 | 92.48 |
| TPP-4794 | 31 | 87 | 98.49 | 93.98 |
| TPP-4798 | 28 | 64 | 96.42 | 91.85 |
| TPP-4799 | 83 | 83 | 98.49 | 96.75 |
| TPP-4805 | 43 | 85 | 98.52 | 94.58 |
| TPP-4810 | 26 | 77 | 97.93 | 95.20 |
| TPP-4812 | 36 | 85 | 98.32 | 94.75 |
| TPP-4813 | 59 | 85 | 98.25 | 93.48 |

### Example 5: The Bispecific TCR Variants Show Strong Binding to HLA-A*02/MAGEB2-001

Maturated R76P1E1S TCR variants expressed as soluble bispecific TCR molecules (e.g. TCER molecules) were analyzed, in various orientations, for their binding affinity towards HLA-A*02/MAGBE2-001 monomers via biolayer interferometry. Measurements were performed on an Octet RED384 or HTX system using settings recommended by the manufacturer. Briefly, binding kinetics were measured at 30° C. and 1000 rpm shake speed using PBS, 0.05% Tween, 0.1% BSA as buffer. HLA-A*02/MAGEB2-001 were immobilized on HIS1K-sensors (at a concentration of 10 μg/ml for 120 s) prior to analyzing serial dilutions of bispecific variants. Binding of the bispecific TCR variants towards HLA-A*02/MAGBE2-001 monomers was demonstrated for all orientations with KD values in the range of 1.61 nM to 13.7 nM (Table 5). The orientation of the TCER molecule did not influence the binding properties as exemplified with TPP-4017 to TPP-4020 having a comparable affinity with KDs of 9.4 to 13.7 nM.

TABLE 5
Binding affinities of different bispecific variants were
analyzed for their binding affinity towards HLA-A*02/MAGEB2-
001 monomers via biolayer interferometry.

| bispecific | KD |
|---|---|
| Variant | [M] |
| TPP-4017 | 1.29E−08 |
| TPP-4018 | 1.05E−08 |
| TPP-4019 | 9.40E−09 |
| TPP-4020 | 1.37E−08 |
| TPP-4606 | 3.54E−09 |
| TPP-4784 | 2.95E−09 |
| TPP-4787 | 2.43E−09 |
| TPP-4788 | 2.54E−09 |
| TPP-4794 | 3.20E−09 |
| TPP-4798 | 1.61E−09 |
| TPP-4799 | 2.72E−09 |
| TPP-4805 | 2.78E−09 |
| TPP-4810 | 3.08E−09 |
| TPP-4812 | 2.96E−09 |
| TPP-4813 | 2.47E−09 |

### Example 6: The Bispecific TCR Molecules Show High Selectivity

For identification of potential off-target peptides, exemplary bispecific TCR molecules (e.g. TCER molecules) incorporating the TCR variants of R76P1E1S were analyzed for binding towards MAGEB2-001 and potential off-target peptides. The human proteome was searched for peptides which display a high sequence similarity to the target peptide and have been confirmed as naturally occurring HLA ligands on normal tissues using the XPRESIDENT® MS technology as for example disclosed in PCT/EP03/050383. A number of 28 most relevant potential off target peptides was identified and screened for bispecific TCR binding using SP-01-0010 as a negative control. MAGEB2-001 and off-target peptide binding were measured by biolayer interferometry and off-target binding signals of 20% or lower compared to MAGEB2 binding signals were considered as non-relevant. Measurements were performed on an Octet RED384 or HTX system using settings recommended by the manufacturer. Briefly, binding kinetics were measured at 30° C. and 1000 rpm shake speed using PBS, 0.05% Tween, 0.1% BSA as buffer. HLA-A*02/MAGEB2-001 were immobilized on HIS1K-sensors (at a concentration of 10 μg/ml for 120 s) prior to analyzing TCR variants.

No relative response signal higher than 20% in comparison to MAGEB2-001 was observed for any of the 29 analyzed peptides demonstrating high selectivity of the bispecific TCR molecules-see Table 6.

TABLE 6
MAGEB2-001 and off-target peptide recognition
were measured by biolayer interferometry.³

| | TPP- | TPP- | TPP- | TPP- | TPP- | TPP- |
|---|---|---|---|---|---|---|
| | 4787 | 4794 | 4799 | 4810 | 4812 | 4813 |
| SP-02-1621 | 0 | 1 | 0 | 1 | 1 | 1 |
| SP-02-1622 | 1 | 2 | 1 | 3 | 2 | 1 |
| SP-02-1623 | 1 | 0 | 1 | 2 | 1 | 1 |
| SP-02-1624 | 0 | 0 | 0 | 0 | 0 | 0 |
| SP-02-1625 | 0 | 0 | 0 | 0 | 0 | 0 |
| SP-02-1626 | 0 | 0 | 0 | 0 | 0 | 0 |
| SP-02-1627 | 0 | 0 | 0 | 0 | 0 | 0 |
| SP-02-1628 | 0 | 0 | 0 | 0 | 0 | 0 |
| SP-02-1629 | 0 | 0 | 0 | 0 | 0 | 0 |
| SP-01-0010 | 0 | 0 | 0 | 0 | 0 | 0 |
| SP-02-1631 | 11 | 15 | 11 | 13 | 14 | 18 |
| SP-02-1635 | 10 | 11 | 8 | 11 | 11 | 15 |
| SP-02-1640 | 9 | 9 | 8 | 10 | 10 | 12 |
| SP-02-1641 | 12 | 15 | 10 | 15 | 15 | 18 |
| SP-02-1642 | 13 | 16 | 12 | 15 | 13 | 19 |
| SP-02-1643 | 7 | 10 | 8 | 7 | 10 | 11 |
| SP-02-1644 | 15 | 16 | 13 | 17 | 17 | 19 |
| SP-02-1647 | 13 | 15 | 13 | 15 | 15 | 20 |
| SP-02-1648 | 14 | 16 | 14 | 15 | 15 | 19 |
| SP-02-1650 | 13 | 15 | 12 | 15 | 17 | 18 |
| SP-02-1651 | 13 | 16 | 17 | 16 | 16 | 20 |
| SP-02-1652 | 11 | 11 | 9 | 9 | 14 | 14 |
| SP-02-1653 | 12 | 13 | 10 | 13 | 15 | 14 |
| SP-02-1655 | 15 | 14 | 11 | 14 | 15 | 15 |
| SP-02-1656 | 13 | 13 | 10 | 13 | 12 | 18 |
| SP-02-1664 | 10 | 14 | 10 | 13 | 15 | 17 |
| SP-02-1673 | 9 | 12 | 10 | 12 | 13 | 15 |
| SP-02-1722 | 12 | 19 | 12 | 19 | 14 | 17 |
| SP-02-1724 | 8 | 15 | 18 | 17 | 15 | 17 |

³Bispecific TCR molecules were analyzed for binding towards MAGEB2-001 and 28 potential off-target peptides and SP-01-0010 as negative control. The response signals for the off-target peptides are depicted in % response signal as measured for the MAGEB2-001 target peptide.

### Example 7: The Bispecific TCR Molecules Efficiently Mediated T Cell-Dependent Tumor Cell Lysis

The activity of MAGEB2-001-targeting bispecific TCR molecules (e.g. TCER molecules) regarding the induction of tumor cell lysis was evaluated by assessing human PBMC-mediated lysis of the human cancer cell lines SKMEL-5, RPMI7951, and SCC25 presenting different copy numbers of MAGEB2-001 peptide in the context of HLA-A*02 on the tumor cell surface (SKMEL-5-194, RPMI7951-23 and SCC25-16 MAGEB2-001 copies per cell, as determined by quantitative M/S analysis) as determined by LDH-release assay. The MAGEB2-001-targeting bispecific TCR molecules constructs induced a concentration-dependent lysis of MAGEB2-001 positive tumor cell lines (FIGS. 6A-6C). These results demonstrate that the MAGEB2-001 directed bispecific TCR molecules are able to efficiently mediate T cell-dependent tumor cell lysis even on tumor cells expressing as little as 20 MAGEB2-001 copy numbers per tumor cell.

### Example 8: Bispecific TCR Molecules Show Beneficial Safety Profile

All bispecific TCR molecules induced a dose-dependent lysis of the target-positive cell line SKMEL5 (194 cpc) while no cytotoxicity against any of the tested primary or iPSC-derived cell types was observed at bispecific doses up to 100 nM (FIGS. 7A-7C).

### Example 9: In Vivo Efficacy in Tumor Xenograft Bearing NOG Mice

A pharmacodynamic study was performed in the hyper immunodeficient NOG mouse strain to test the ability of a TCER molecule in recruiting the activity of human immune T cells by specific binding to a T cell antigen and by specific binding to a human tumor-specific HLA-peptide complex on human cancer cells (FIG. 9). The NOG mouse strain hosting the subcutaneously injected human tumor cell line BCPAP (including 450 to 500 of MAGEB2-001 peptide copies in the context of HLA-A*02) were intravenously injected with human peripheral blood mononuclear (PBMC) cell xenografts. The human PBMC (1×10⁷ cells/mouse) were transplanted within 24 hours when individual BCPAP tumor volume reached >120 mm³ as calculated based on caliper measurements. Treatment was initiated within one hour after transplantation of human blood cells. Six female mice per group (stratified randomization according to tumor size) received intravenous bolus injections (5 ml/kg body weight, once weekly dosing, three doses) into the tail vein. The injected dose of the TCER molecule was 0.25 mg/kg body weight per injection (group 2), PBS was used in the vehicle control group (group 1). As shown in FIG. 9 for individual mice, the treatment with the TCER molecule induced pronounced anti-tumor responses in all mice of group 2. Treatment with TCER TPP-4799 targeting HLA-A*02/MAGEB2-001 peptide complexes strongly inhibited tumor growth as indicated by reduction of individual tumor volume in comparison to the increase observed in the control group 1. Treatment with TCER TPP-4799 induced complete remission of the BCPAP tumor in three of six mice starting at Day 18, remaining tumor volumes ranged between 9 and 14 mm³ at Day 21 compared to volumes ranging between 742 and 1286 mm³ in the vehicle treated mice of group 1.

### SEQUENCES

TABLE 7
Exemplary amino acid sequences referred to herein

| | | | Descrip- | |
|---|---|---|---|---|
| SEQ | Descrip- | Descrip- | tion/ | |
| ID | tion/ | tion/ | Region/ | |
| NO | TCRs | Chain | Domain | Sequence |
| 1 | | | MAGEB2 | GVYDGEEHSV |
| | | | target | |
| | | | peptide | |
| 2 | TPP-4799, | alpha | CDR1 | DASSTY |
| | TPP-4787, | | | |
| | TPP-4810, | | | |
| | TPP-4794, | | | |
| | TPP-4812, | | | |
| | TPP-4813, | | | |
| | TPP-4606, | | | |
| | TPP-4784, | | | |
| | TPP-4788, | | | |
| | TPP-4798, | | | |
| | TPP-4805, | | | |
| | TPP-4020, | | | |
| | TPP-4017, | | | |
| | TPP-4018, | | | |
| | TPP-4019 | | | |
| 3 | TPP-4799, | alpha | CDR3 | AVKDNARLL |
| | TPP-4810, | | | |
| | TPP-4812, | | | |
| | TPP-4813, | | | |
| | TPP-4784, | | | |
| | TPP-4788, | | | |
| | TPP-4805 | | | |
| 4 | TPP-4787, | alpha | CDR3 | AVKDNARLM |
| | TPP-4794, | | | |
| | TPP-4606, | | | |
| | TPP-4798, | | | |
| | TPP-4020, | | | |
| | TPP-4017, | | | |
| | TPP-4018, | | | |
| | TPP-4019 | | | |
| 5 | TPP-4799, | beta | CDR1 | RPVTM |
| | TPP-4787, | | | |
| | TPP-4810, | | | |
| | TPP-4794, | | | |
| | TPP-4812, | | | |
| | TPP-4813, | | | |
| | TPP-4606, | | | |
| | TPP-4784, | | | |
| | TPP-4788, | | | |
| | TPP-4798, | | | |
| | TPP-4805, | | | |
| | TPP-4020, | | | |
| | TPP-4017, | | | |
| | TPP-4018, | | | |
| | TPP-4019, | | | |
| | CL-48553 | | | |
| 6 | TPP-4799, | beta | CDR3 | SVLGAYGYT |
| | TPP-4787, | | | |
| | TPP-4810, | | | |
| | TPP-4794, | | | |
| | TPP-4812, | | | |
| | TPP-4813, | | | |
| | TPP-4606, | | | |
| | TPP-4784, | | | |
| | TPP-4788, | | | |
| | TPP-4798, | | | |
| | TPP-4805, | | | |
| | TPP-4020, | | | |
| | TPP-4017, | | | |
| | TPP-4018, | | | |
| | TPP-4019 | | | |
| 7 | TPP-4799, | alpha | CDR2 | IYSNMDM |
| | TPP-4810, | | | |
| | TPP-4812, | | | |
| | TPP-4813, | | | |
| | TPP-4784, | | | |
| | TPP-4788, | | | |
| | TPP-4805 | | | |
| 8 | TPP-4787, | alpha | CDR2 | IFSNMDM |
| | TPP-4794, | | | |
| | TPP-4606, | | | |
| | TPP-4798, | | | |
| | TPP-4020, | | | |
| | TPP-4017, | | | |
| | TPP-4018, | | | |
| | TPP-4019 | | | |
| 9 | TPP-4799, | beta | CDR2 | AYDEGSA |
| | TPP-4805, | | | |
| | TPP-4020, | | | |
| | TPP-4017, | | | |
| | TPP-4018, | | | |
| | TPP-4019 | | | |
| 10 | TPP-4787, | beta | CDR2 | AYDVGSA |
| | TPP-4810, | | | |
| | TPP-4794, | | | |
| | TPP-4812, | | | |
| | TPP-4813, | | | |
| | TPP-4606, | | | |
| | TPP-4784, | | | |
| | TPP-4788, | | | |
| | TPP-4798 | | | |
| 11 | TPP-4799, | alpha | variable | EDVEQSSFLSVREGDSAVINCTYTDASSTYFYWYKQ |
| | TPP-4813, | | domain | EPGAGLQLLTYIYSNMDMKQDQRLTVLLNKKDKHLS |
| | TPP-4788 | | | LRIADTQTGDSAIYFCAVKDNARLLFGDVTQLVVKP |
| 12 | TPP-4787, | alpha | variable | EDVEQSSFLSVREGDSAVINCTYTDASSTYFYWYKQ |
| | TPP-4798 | | domain | EPGAGLQLLTYIFSNMDMKQDQRLTVLLNKKDKHLS |
| | | | | LRIADTQTGDSAIYFCAVKDNARLMFGDVTQLVVKP |
| 13 | TPP-4810, | alpha | variable | EDVEQSSFLSVREGDSAVINCTYTDASSTYFYWYKQ |
| | TPP-4812, | | domain | EPGAGLQLLTYIYSNMDMKQDQRLTVLLNKKDKHLS |
| | TPP-4784, | | | LRIADTQTGDSAIYFCAVKDNARLLFGDGTQLVVKP |
| | TPP-4805 | | | |
| 14 | TPP-4794, | alpha | variable | EDVEQSSFLSVREGDSAVINCTYTDASSTYFYWYKQ |
| | TPP-4606, | | domain | EPGAGLQLLTYIFSNMDMKQDQRLTVLLNKKDKHLS |
| | TPP-4020, | | | LRIADTQTGDSAIYFCAVKDNARLMFGDGTQLVVKP |
| | TPP-4017, | | | |
| | TPP-4018, | | | |
| | TPP-4019 | | | |
| 15 | TPP-4799 | beta | variable | AVISQKPSRVIVQRGTSVTIQCQVDRPVTMMYWYRQ |
| | | | domain | QPGQSLTLIATAYDEGSATYESGFDIDKFPISRPNLTF |
| | | | | STLTVSNVEPEDSSIYLCSVLGAYGYTFGSGTRLTVV |
| 16 | TPP-4787 | beta | variable | AVISQKPSRYIVQRGTSVTIQCQVDRPVTMMYWYRQ |
| | | | domain | QPGQSLTLIATAYDVGSATYESGFVIDKFPISRPNLTF |
| | | | | STLTVSNVEPEDSSIYLCSVLGAYGYTFGSGTRLTVV |
| 17 | TPP-4810 | beta | variable | AVISQKPSRYIVQRGTSVTIQCQVDRPVTMMYWYRQ |
| | | | domain | QPGQSLTLIATAYDVGSATYESGFDIDKFPISRPNLTF |
| | | | | STLTVSNMEPEDSSIYLCSVLGAYGYTFGSGTRLTVV |
| 18 | TPP-4794, | beta | variable | AVISQKPSRVIVQRGTSVTIQCQVDRPVTMMYWYRQ |
| | TPP-4798 | | domain | QPGQSLTLIATAYDVGSATYESGFVIDKFPISRPNLTF |
| | | | | STLTVSNVEPEDSSIYLCSVLGAYGYTFGSGTRLTVV |
| 19 | TPP-4812, | beta | variable | AVISQKPSRYIVQRGTSVTIQCQVDRPVTMMYWYRQ |
| | TPP-4813 | | domain | QPGQSLTLIATAYDVGSATYESGFDIDKFPISRPNLTF |
| | | | | STLTVSNVEPEDSSIYLCSVLGAYGYTFGSGTRLTVV |
| 20 | TPP-4606 | beta | variable | AVISQKPSRYIVQRGNSVTIQCQVDRPVTMMFWYR |
| | | | domain | QQPGQSPTLIATAYDVGSATYESGFVIDKFPISRPNLT |
| | | | | FSTLTVSNMEPEDSSIYLCSVLGAYGYTFGSGTRLTV |
| | | | | V |
| 21 | TPP-4784, | beta | variable | AVISQKPSRYIVQRGTSVTIQCQVDRPVTMMFWYRQ |
| | TPP-4788 | | domain | QPGQSLTLIATAYDVGSATYESGFDIDKFPISRPNLTF |
| | | | | STLTVSNMEPEDSSIYLCSVLGAYGYTFGSGTRLTVV |
| 22 | TPP-4805 | beta | variable | AVISQKPSRDIVQRGTSVTIQCQVDRPVTMMYWYRQ |
| | | | domain | QPGQSLTLIATAYDEGSATYESGFDIDKFPISRPNLTF |
| | | | | STLTVSNMEPEDSSIYLCSVLGAYGYTFGSGTRLTVV |
| 23 | TPP-4020, | beta | variable | AVISQKPSRDIVQRGNSVTIQCQVDRPVTMMFWYR |
| | TPP-4017, | | domain | QQPGQSLTLIATAYDEGSATYESGFVIDKFPISRPNLT |
| | TPP-4018, | | | FSTLTVSNMSPEDSSIYLCSVLGAYGYTFGSGTRLTV |
| | TPP-4019 | | | V |
| 24 | TPP-4799, | | fc knob | EPKSSDKTHTCPPCPAPPVAGPSVFLFPPKPKDTLMI |
| | TPP-4787, | | | SRTPEVTCVVVDVSHEDPEVKFNWYVDGVEVHNAK |
| | TPP-4810, | | | TKPREEQYQSTYRVVSVLTVLHQDWLNGKEYKCKVS |
| | TPP-4794, | | | NKALPASIEKTISKAKGQPREPQVYTLPPCRDELTKN |
| | TPP-4812, | | | QVSLWCLVKGFYPSDIAVEWESNGQPENNYKTTPP |
| | TPP-4813, | | | VLDSDGSFFLYSKLTVDKSRWQQGNVFSCSVMHEA |
| | TPP-4606, | | | LHNHYTQKSLSLSP |
| | TPP-4784, | | | |
| | TPP-4788, | | | |
| | TPP-4798, | | | |
| | TPP-4805, | | | |
| | TPP-4020, | | | |
| | TPP-4017, | | | |
| | TPP-4018, | | | |
| | TPP-4019 | | | |
| 25 | TPP-4799, | | fc hole | EPKSSDKTHTCPPCPAPPVAGPSVFLFPPKPKDTLMI |
| | TPP-4787, | | | SRTPEVTCVVVDVSHEDPEVKFNWYVDGVEVHNAK |
| | TPP-4810, | | | TKPREEQYQSTYRVVSVLTVLHQDWLNGKEYKCKVS |
| | TPP-4794, | | | NKALPASIEKTISKAKGQPREPQVCTLPPSRDELTKN |
| | TPP-4812, | | | QVSLSCAVKGFYPSDIAVEWESNGQPENNYKTTPPV |
| | TPP-4813, | | | LDSDGSFFLVSKLTVDKSRWQQGNVFSCSVMHEAL |
| | TPP-4606, | | | HNHYTQKSLSLSP |
| | TPP-4784, | | | |
| | TPP-4788, | | | |
| | TPP-4798, | | | |
| | TPP-4805, | | | |
| | TPP-4020, | | | |
| | TPP-4017, | | | |
| | TPP-4018, | | | |
| | TPP-4019 | | | |
| 26 | TPP-4799, | | TCER | GGGSGGGG |
| | TPP-4787, | | linker | |
| | TPP-4810, | | | |
| | TPP-4794, | | | |
| | TPP-4812, | | | |
| | TPP-4813, | | | |
| | TPP-4606, | | | |
| | TPP-4784, | | | |
| | TPP-4788, | | | |
| | TPP-4798, | | | |
| | TPP-4805, | | | |
| | TPP-4020, | | | |
| | TPP-4017, | | | |
| | TPP-4018, | | | |
| | TPP-4019 | | | |
| 27 | TPP-4799, | | first | EDVEQSSFLSVREGDSAVINCTYTDASSTYFYWYKQ |
| | TPP-4813, | | polypep- | EPGAGLQLLTYIYSNMDMKQDQRLTVLLNKKDKHLS |
| | TPP-4788 | | tide | LRIADTQTGDSAIYFCAVKDNARLLFGDVTQLVVKPG |
| | | | | GGSGGGGEVQLVQSGAEVKKPGASVKVSCKASGY |
| | | | | KFTSYVMHWVRQAPGQGLEWMGYINPRNDVTKYA |
| | | | | EKFQGRVTLTSDTSTSTAYMELSSLRSEDTAVYYCAR |
| | | | | GSYYDYEGFVYWGQGTLVTVSSEPKSSDKTHTCPP |
| | | | | CPAPPVAGPSVFLFPPKPKDTLMISRTPEVTCVVVDV |
| | | | | SHEDPEVKFNWYVDGVEVHNAKTKPREEQYQSTYR |
| | | | | VVSVLTVLHQDWLNGKEYKCKVSNKALPASIEKTISK |
| | | | | AKGQPREPQVCTLPPSRDELTKNQVSLSCAVKGFYP |
| | | | | SDIAVEWESNGQPENNYKTTPPVLDSDGSFFLVSKL |
| | | | | TVDKSRWQQGNVFSCSVMHEALHNHYTQKSLSLS |
| | | | | P |
| 28 | TPP-4787, | | first | EDVEQSSFLSVREGDSAVINCTYTDASSTYFYWYKQ |
| | TPP-4798 | | polypep- | EPGAGLQLLTYIFSNMDMKQDQRLTVLLNKKDKHLS |
| | | | tide | LRIADTQTGDSAIYFCAVKDNARLMFGDVTQLVVKP |
| | | | | GGGSGGGGEVQLVQSGAEVKKPGASVKVSCKASG |
| | | | | YKFTSYVMHWVRQAPGQGLEWMGYINPRNDVTKY |
| | | | | AEKFQGRVTLTSDTSTSTAYMELSSLRSEDTAVYYCA |
| | | | | RGSYYDYEGFVYWGQGTLVTVSSEPKSSDKTHTCPP |
| | | | | CPAPPVAGPSVFLFPPKPKDTLMISRTPEVTCVVVDV |
| | | | | SHEDPEVKFNWYVDGVEVHNAKTKPREEQYQSTYR |
| | | | | VVSVLTVLHQDWLNGKEYKCKVSNKALPASIEKTISK |
| | | | | AKGQPREPQVCTLPPSRDELTKNQVSLSCAVKGFYP |
| | | | | SDIAVEWESNGQPENNYKTTPPVLDSDGSFFLVSKL |
| | | | | TVDKSRWQQGNVFSCSVMHEALHNHYTQKSLSLS |
| | | | | P |
| 29 | TPP-4810, | | first | EDVEQSSFLSVREGDSAVINCTYTDASSTYFYWYKQ |
| | TPP-4812, | | polypep- | EPGAGLQLLTYIYSNMDMKQDQRLTVLLNKKDKHLS |
| | TPP-4784, | | tide | LRIADTQTGDSAIYFCAVKDNARLLFGDGTQLVVKP |
| | TPP-4805 | | | GGGSGGGGEVQLVQSGAEVKKPGASVKVSCKASG |
| | | | | YKFTSYVMHWVRQAPGQGLEWMGYINPRNDVTKY |
| | | | | AEKFQGRVTLTSDTSTSTAYMELSSLRSEDTAVYYCA |
| | | | | RGSYYDYEGFVYWGQGTLVTVSSEPKSSDKTHTCPP |
| | | | | CPAPPVAGPSVFLFPPKPKDTLMISRTPEVTCVVVDV |
| | | | | SHEDPEVKFNWYVDGVEVHNAKTKPREEQYQSTYR |
| | | | | VVSVLTVLHQDWLNGKEYKCKVSNKALPASIEKTISK |
| | | | | AKGQPREPQVCTLPPSRDELTKNQVSLSCAVKGFYP |
| | | | | SDIAVEWESNGQPENNYKTTPPVLDSDGSFFLVSKL |
| | | | | TVDKSRWQQGNVFSCSVMHEALHNHYTQKSLSLS |
| | | | | P |
| 30 | TPP-4794, | | first | EDVEQSSFLSVREGDSAVINCTYTDASSTYFYWYKQ |
| | TPP-4606, | | polypep- | EPGAGLQLLTYIFSNMDMKQDQRLTVLLNKKDKHLS |
| | TPP-4020 | | tide | LRIADTQTGDSAIYFCAVKDNARLMFGDGTQLVVKP |
| | | | | GGGSGGGGEVQLVQSGAEVKKPGASVKVSCKASG |
| | | | | YKFTSYVMHWVRQAPGQGLEWMGYINPRNDVTKY |
| | | | | AEKFQGRVTLTSDTSTSTAYMELSSLRSEDTAVYYCA |
| | | | | RGSYYDYEGFVYWGQGTLVTVSSEPKSSDKTHTCPP |
| | | | | CPAPPVAGPSVFLFPPKPKDTLMISRTPEVTCVVVDV |
| | | | | SHEDPEVKFNWYVDGVEVHNAKTKPREEQYQSTYR |
| | | | | VVSVLTVLHQDWLNGKEYKCKVSNKALPASIEKTISK |
| | | | | AKGQPREPQVCTLPPSRDELTKNQVSLSCAVKGFYP |
| | | | | SDIAVEWESNGQPENNYKTTPPVLDSDGSFFLVSKL |
| | | | | TVDKSRWQQGNVFSCSVMHEALHNHYTQKSLSLS |
| | | | | P |
| 31 | TPP-4017 | | first | EDVEQSSFLSVREGDSAVINCTYTDASSTYFYWYKQ |
| | | | polypep- | EPGAGLQLLTYIFSNMDMKQDQRLTVLLNKKDKHLS |
| | | | tide | LRIADTQTGDSAIYFCAVKDNARLMFGDGTQLVVKP |
| | | | | GGGSGGGGQIQMTQSPSSLSASVGDRVTITCSATS |
| | | | | SVSYMHWYQQKPGKAPKRWIYDTSKLASGVPSRFS |
| | | | | GSGSGTDYTLTISSLQPEDAATYYCQQWSSNPLTFG |
| | | | | GGTKVEIKEPKSSDKTHTCPPCPAPPVAGPSVFLFPP |
| | | | | KPKDTLMISRTPEVTCVVVDVSHEDPEVKFNWYVDG |
| | | | | VEVHNAKTKPREEQYQSTYRVVSVLTVLHQDWLNG |
| | | | | KEYKCKVSNKALPASIEKTISKAKGQPREPQVYTLPP |
| | | | | CRDELTKNQVSLWCLVKGFYPSDIAVEWESNGQPE |
| | | | | NNYKTTPPVLDSDGSFFLYSKLTVDKSRWQQGNVF |
| | | | | SCSVMHEALHNHYTQKSLSLSP |
| 32 | TPP-4018 | | first | QIQMTQSPSSLSASVGDRVTITCSATSSVSYMHWYQ |
| | | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGEDVEQSSFLSVREGDSAVINCTYTDASSTY |
| | | | | FYWYKQEPGAGLQLLTYIFSNMDMKQDQRLTVLLN |
| | | | | KKDKHLSLRIADTQTGDSAIYFCAVKDNARLMFGDG |
| | | | | TQLVVKPEPKSSDKTHTCPPCPAPPVAGPSVFLFPPK |
| | | | | PKDTLMISRTPEVTCVVVDVSHEDPEVKFNWYVDGV |
| | | | | EVHNAKTKPREEQYQSTYRVVSVLTVLHQDWLNGK |
| | | | | EYKCKVSNKALPASIEKTISKAKGQPREPQVYTLPPC |
| | | | | RDELTKNQVSLWCLVKGFYPSDIAVEWESNGQPEN |
| | | | | NYKTTPPVLDSDGSFFLYSKLTVDKSRWQQGNVFS |
| | | | | CSVMHEALHNHYTQKSLSLSP |
| 33 | TPP-4019 | | first | EVQLVQSGAEVKKPGASVKVSCKASGYKFTSYVMH |
| | | | polypep- | WVRQAPGQGLEWMGYINPRNDVTKYAEKFQGRVT |
| | | | tide | LTSDTSTSTAYMELSSLRSEDTAVYYCARGSYYDYEG |
| | | | | FVYWGQGTLVTVSSGGGSGGGGEDVEQSSFLSVRE |
| | | | | GDSAVINCTYTDASSTYFYWYKQEPGAGLQLLTYIFS |
| | | | | NMDMKQDQRLTVLLNKKDKHLSLRIADTQTGDSAIY |
| | | | | FCAVKDNARLMFGDGTQLVVKPEPKSSDKTHTCPP |
| | | | | CPAPPVAGPSVFLFPPKPKDTLMISRTPEVTCVVVDV |
| | | | | SHEDPEVKFNWYVDGVEVHNAKTKPREEQYQSTYR |
| | | | | VVSVLTVLHQDWLNGKEYKCKVSNKALPASIEKTISK |
| | | | | AKGQPREPQVCTLPPSRDELTKNQVSLSCAVKGFYP |
| | | | | SDIAVEWESNGQPENNYKTTPPVLDSDGSFFLVSKL |
| | | | | TVDKSRWQQGNVFSCSVMHEALHNHYTQKSLSLS |
| | | | | P |
| 34 | TPP-4799 | | second | QIQMTQSPSSLSASVGDRVTITCSATSSVSYMHWYQ |
| | | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRVIVQRGTSVTIQCQVDRPVTM |
| | | | | MYWYRQQPGQSLTLIATAYDEGSATYESGFDIDKFPI |
| | | | | SRPNLTFSTLTVSNVEPEDSSIYLCSVLGAYGYTFGS |
| | | | | GTRLTVVEPKSSDKTHTCPPCPAPPVAGPSVFLFPPK |
| | | | | PKDTLMISRTPEVTCVVVDVSHEDPEVKFNWYVDGV |
| | | | | EVHNAKTKPREEQYQSTYRVVSVLTVLHQDWLNGK |
| | | | | EYKCKVSNKALPASIEKTISKAKGQPREPQVYTLPPC |
| | | | | RDELTKNQVSLWCLVKGFYPSDIAVEWESNGQPEN |
| | | | | NYKTTPPVLDSDGSFFLYSKLTVDKSRWQQGNVFS |
| | | | | CSVMHEALHNHYTQKSLSLSP |
| 35 | TPP-4787 | | second | QIQMTQSPSSLSASVGDRVTITCSATSSVSYMHWYQ |
| | | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRYIVQRGTSVTIQCQVDRPVTM |
| | | | | MYWYRQQPGQSLTLIATAYDVGSATYESGFVIDKFPI |
| | | | | SRPNLTFSTLTVSNVEPEDSSIYLCSVLGAYGYTFGS |
| | | | | GTRLTVVEPKSSDKTHTCPPCPAPPVAGPSVFLFPPK |
| | | | | PKDTLMISRTPEVTCVVVDVSHEDPEVKFNWYVDGV |
| | | | | EVHNAKTKPREEQYQSTYRVVSVLTVLHQDWLNGK |
| | | | | EYKCKVSNKALPASIEKTISKAKGQPREPQVYTLPPC |
| | | | | RDELTKNQVSLWCLVKGFYPSDIAVEWESNGQPEN |
| | | | | NYKTTPPVLDSDGSFFLYSKLTVDKSRWQQGNVFS |
| | | | | CSVMHEALHNHYTQKSLSLSP |
| 36 | TPP-4810 | | second | QIQMTQSPSSLSASVGDRVTITCSATSSVSYMHWYQ |
| | | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRYIVQRGTSVTIQCQVDRPVTM |
| | | | | MYWYRQQPGQSLTLIATAYDVGSATYESGFDIDKFPI |
| | | | | SRPNLTFSTLTVSNMEPEDSSIYLCSVLGAYGYTFGS |
| | | | | GTRLTVVEPKSSDKTHTCPPCPAPPVAGPSVFLFPPK |
| | | | | PKDTLMISRTPEVTCVVVDVSHEDPEVKFNWYVDGV |
| | | | | EVHNAKTKPREEQYQSTYRVVSVLTVLHQDWLNGK |
| | | | | EYKCKVSNKALPASIEKTISKAKGQPREPQVYTLPPC |
| | | | | RDELTKNQVSLWCLVKGFYPSDIAVEWESNGQPEN |
| | | | | NYKTTPPVLDSDGSFFLYSKLTVDKSRWQQGNVFS |
| | | | | CSVMHEALHNHYTQKSLSLSP |
| 37 | TPP-4794, | | second | QIQMTQSPSSLSASVGDRVTITCSATSSVSYMHWYQ |
| | TPP-4798 | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRVIVQRGTSVTIQCQVDRPVTM |
| | | | | MYWYRQQPGQSLTLIATAYDVGSATYESGFVIDKFPI |
| | | | | SRPNLTFSTLTVSNVEPEDSSIYLCSVLGAYGYTFGS |
| | | | | GTRLTVVEPKSSDKTHTCPPCPAPPVAGPSVFLFPPK |
| | | | | PKDTLMISRTPEVTCVVVDVSHEDPEVKFNWYVDGV |
| | | | | EVHNAKTKPREEQYQSTYRVVSVLTVLHQDWLNGK |
| | | | | EYKCKVSNKALPASIEKTISKAKGQPREPQVYTLPPC |
| | | | | RDELTKNQVSLWCLVKGFYPSDIAVEWESNGQPEN |
| | | | | NYKTTPPVLDSDGSFFLYSKLTVDKSRWQQGNVFS |
| | | | | CSVMHEALHNHYTQKSLSLSP |
| 38 | TPP-4812, | | second | QIQMTQSPSSLSASVGDRVTITCSATSSVSYMHWYQ |
| | TPP-4813 | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRYIVQRGTSVTIQCQVDRPVTM |
| | | | | MYWYRQQPGQSLTLIATAYDVGSATYESGFDIDKFPI |
| | | | | SRPNLTFSTLTVSNVEPEDSSIYLCSVLGAYGYTFGS |
| | | | | GTRLTVVEPKSSDKTHTCPPCPAPPVAGPSVFLFPPK |
| | | | | PKDTLMISRTPEVTCVVVDVSHEDPEVKFNWYVDGV |
| | | | | EVHNAKTKPREEQYQSTYRVVSVLTVLHQDWLNGK |
| | | | | EYKCKVSNKALPASIEKTISKAKGQPREPQVYTLPPC |
| | | | | RDELTKNQVSLWCLVKGFYPSDIAVEWESNGQPEN |
| | | | | NYKTTPPVLDSDGSFFLYSKLTVDKSRWQQGNVFS |
| | | | | CSVMHEALHNHYTQKSLSLSP |
| 39 | TPP-4606 | | second | QIQMTQSPSSLSASVGDRVTITCSATSSVSYMHWYQ |
| | | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRYIVQRGNSVTIQCQVDRPVT |
| | | | | MMFWYRQQPGQSPTLIATAYDVGSATYESGFVIDKF |
| | | | | PISRPNLTFSTLTVSNMEPEDSSIYLCSVLGAYGYTFG |
| | | | | SGTRLTVVEPKSSDKTHTCPPCPAPPVAGPSVFLFPP |
| | | | | KPKDTLMISRTPEVTCVVVDVSHEDPEVKFNWYVDG |
| | | | | VEVHNAKTKPREEQYQSTYRVVSVLTVLHQDWLNG |
| | | | | KEYKCKVSNKALPASIEKTISKAKGQPREPQVYTLPP |
| | | | | CRDELTKNQVSLWCLVKGFYPSDIAVEWESNGQPE |
| | | | | NNYKTTPPVLDSDGSFFLYSKLTVDKSRWQQGNVF |
| | | | | SCSVMHEALHNHYTQKSLSLSP |
| 40 | TPP-4784, | | second | QIQMTQSPSSLSASVGDRVTITCSATSSVSYMHWYQ |
| | TPP-4788 | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRYIVQRGTSVTIQCQVDRPVTM |
| | | | | MFWYRQQPGQSLTLIATAYDVGSATYESGFDIDKFPI |
| | | | | SRPNLTFSTLTVSNMEPEDSSIYLCSVLGAYGYTFGS |
| | | | | GTRLTVVEPKSSDKTHTCPPCPAPPVAGPSVFLFPPK |
| | | | | PKDTLMISRTPEVTCVVVDVSHEDPEVKFNWYVDGV |
| | | | | EVHNAKTKPREEQYQSTYRVVSVLTVLHQDWLNGK |
| | | | | EYKCKVSNKALPASIEKTISKAKGQPREPQVYTLPPC |
| | | | | RDELTKNQVSLWCLVKGFYPSDIAVEWESNGQPEN |
| | | | | NYKTTPPVLDSDGSFFLYSKLTVDKSRWQQGNVFS |
| | | | | CSVMHEALHNHYTQKSLSLSP |
| 41 | TPP-4805 | | second | QIQMTQSPSSLSASVGDRVTITCSATSSVSYMHWYQ |
| | | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRDIVQRGTSVTIQCQVDRPVT |
| | | | | MMYWYRQQPGQSLTLIATAYDEGSATYESGFDIDKF |
| | | | | PISRPNLTFSTLTVSNMEPEDSSIYLCSVLGAYGYTFG |
| | | | | SGTRLTVVEPKSSDKTHTCPPCPAPPVAGPSVFLFPP |
| | | | | KPKDTLMISRTPEVTCVVVDVSHEDPEVKFNWYVDG |
| | | | | VEVHNAKTKPREEQYQSTYRVVSVLTVLHQDWLNG |
| | | | | KEYKCKVSNKALPASIEKTISKAKGQPREPQVYTLPP |
| | | | | CRDELTKNQVSLWCLVKGFYPSDIAVEWESNGQPE |
| | | | | NNYKTTPPVLDSDGSFFLYSKLTVDKSRWQQGNVF |
| | | | | SCSVMHEALHNHYTQKSLSLSP |
| 42 | TPP-4020 | | second | QIQMTQSPSSLSASVGDRVTITCSATSSVSYMHWYQ |
| | | | polypep- | QKPGKAPKRWIYDTSKLASGVPSRFSGSGSGTDYTL |
| | | | tide | TISSLQPEDAATYYCQQWSSNPLTFGGGTKVEIKGG |
| | | | | GSGGGGAVISQKPSRDIVQRGNSVTIQCQVDRPVT |
| | | | | MMFWYRQQPGQSLTLIATAYDEGSATYESGFVIDKF |
| | | | | PISRPNLTFSTLTVSNMSPEDSSIYLCSVLGAYGYTFG |
| | | | | SGTRLTVVEPKSSDKTHTCPPCPAPPVAGPSVFLFPP |
| | | | | KPKDTLMISRTPEVTCVVVDVSHEDPEVKFNWYVDG |
| | | | | VEVHNAKTKPREEQYQSTYRVVSVLTVLHQDWLNG |
| | | | | KEYKCKVSNKALPASIEKTISKAKGQPREPQVYTLPP |
| | | | | CRDELTKNQVSLWCLVKGFYPSDIAVEWESNGQPE |
| | | | | NNYKTTPPVLDSDGSFFLYSKLTVDKSRWQQGNVF |
| | | | | SCSVMHEALHNHYTQKSLSLSP |
| 43 | TPP-4017 | | second | EVQLVQSGAEVKKPGASVKVSCKASGYKFTSYVMH |
| | | | polypep- | WVRQAPGQGLEWMGYINPRNDVTKYAEKFQGRVT |
| | | | tide | LTSDTSTSTAYMELSSLRSEDTAVYYCARGSYYDYEG |
| | | | | FVYWGQGTLVTVSSGGGSGGGGAVISQKPSRDIVQ |
| | | | | RGNSVTIQCQVDRPVTMMFWYRQQPGQSLTLIATA |
| | | | | YDEGSATYESGFVIDKFPISRPNLTFSTLTVSNMSPED |
| | | | | SSIYLCSVLGAYGYTFGSGTRLTVVEPKSSDKTHTCP |
| | | | | PCPAPPVAGPSVFLFPPKPKDTLMISRTPEVTCVVVD |
| | | | | VSHEDPEVKFNWYVDGVEVHNAKTKPREEQYQSTY |
| | | | | RVVSVLTVLHQDWLNGKEYKCKVSNKALPASIEKTIS |
| | | | | KAKGQPREPQVCTLPPSRDELTKNQVSLSCAVKGFY |
| | | | | PSDIAVEWESNGQPENNYKTTPPVLDSDGSFFLVSK |
| | | | | LTVDKSRWQQGNVFSCSVMHEALHNHYTQKSLSLS |
| | | | | P |
| 44 | TPP-4018 | | second | AVISQKPSRDIVQRGNSVTIQCQVDRPVTMMFWYR |
| | | | polypep- | QQPGQSLTLIATAYDEGSATYESGFVIDKFPISRPNLT |
| | | | tide | FSTLTVSNMSPEDSSIYLCSVLGAYGYTFGSGTRLTV |
| | | | | VGGGSGGGGEVQLVQSGAEVKKPGASVKVSCKAS |
| | | | | GYKFTSYVMHWVRQAPGQGLEWMGYINPRNDVTK |
| | | | | YAEKFQGRVTLTSDTSTSTAYMELSSLRSEDTAVYYC |
| | | | | ARGSYYDYEGFVYWGQGTLVTVSSEPKSSDKTHTCP |
| | | | | PCPAPPVAGPSVFLFPPKPKDTLMISRTPEVTCVVVD |
| | | | | VSHEDPEVKFNWYVDGVEVHNAKTKPREEQYQSTY |
| | | | | RVVSVLTVLHQDWLNGKEYKCKVSNKALPASIEKTIS |
| | | | | KAKGQPREPQVCTLPPSRDELTKNQVSLSCAVKGFY |
| | | | | PSDIAVEWESNGQPENNYKTTPPVLDSDGSFFLVSK |
| | | | | LTVDKSRWQQGNVFSCSVMHEALHNHYTQKSLSLS |
| | | | | P |
| 45 | TPP-4019 | | second | AVISQKPSRDIVQRGNSVTIQCQVDRPVTMMFWYR |
| | | | polypep- | QQPGQSLTLIATAYDEGSATYESGFVIDKFPISRPNLT |
| | | | tide | FSTLTVSNMSPEDSSIYLCSVLGAYGYTFGSGTRLTV |
| | | | | VGGGSGGGGQIQMTQSPSSLSASVGDRVTITCSAT |
| | | | | SSVSYMHWYQQKPGKAPKRWIYDTSKLASGVPSRF |
| | | | | SGSGSGTDYTLTISSLQPEDAATYYCQQWSSNPLTF |
| | | | | GGGTKVEIKEPKSSDKTHTCPPCPAPPVAGPSVFLFP |
| | | | | PKPKDTLMISRTPEVTCVVVDVSHEDPEVKFNWYVD |
| | | | | GVEVHNAKTKPREEQYQSTYRVVSVLTVLHQDWLN |
| | | | | GKEYKCKVSNKALPASIEKTISKAKGQPREPQVYTLP |
| | | | | PCRDELTKNQVSLWCLVKGFYPSDIAVEWESNGQP |
| | | | | ENNYKTTPPVLDSDGSFFLYSKLTVDKSRWQQGNV |
| | | | | FSCSVMHEALHNHYTQKSLSLSP |
| 46 | BMhanced | | T cell | EVQLVQSGAEVKKPGASVKVSCKASGYKFTSYVMH |
| | VH | | Recruiter | WVRQAPGQGLEWMGYINPRNDVTKYAEKFQGRVT |
| | | | VH | LTSDTSTSTAYMELSSLRSEDTAVYYCARGSYYDYEG |
| | | | | FVYWGQGTLVTVSSEPKSS |
| 47 | BMhanced | | T cell | QIQMTQSPSSLSASVGDRVTITCSATSSVSYMHWYQ |
| | VL | | Recruiter | QKPGKAPKRWIYDTSKLASGVPSRFSGSGSGTDYTL |
| | | | VL | TISSLQPEDAATYYCQQWSSNPLTFGGGTKVEIK |
| 48 | BMhanced | | Recruiter | SYVMH |
| | HCDR1 | | HCDR1 | |
| | | | (Kabat) | |
| 49 | BMhanced | | Recruiter | YINPRNDVTKYAEKFQG |
| | HCDR2 | | HCDR2 | |
| | | | (Kabat) | |
| 50 | BMhanced | | Recruiter | GSYYDYEGFVY |
| | HCDR3 | | HCDR3 | |
| | | | (Kabat) | |
| 51 | BMhanced | | Recruiter | SATSSVSYMH |
| | LCDR1 | | LCDR1 | |
| | | | (Kabat) | |
| 52 | BMhanced | | Recruiter | DTSKLAS |
| | LCDR2 | | LCDR2 | |
| | | | (Kabat) | |
| 53 | BMhanced | | Recruiter | QQWSSNPLT |
| | LCDR3 | | LCDR3 | |
| | | | (Kabat) | |
| 54 | BMA031- | | VH for | EVQLVQSGAEVKKPGASVKVSCKASGYKFTSYVMH |
| | V10 VH | | alternative | WVRQAPGQGLEWMGYINPYNDVTKYAEKFQGRVT |
| | | | BM | LTSDTSTSTAYMELSSLRSEDTAVHYCARGSYYDYD |
| | | | recruiter | GFVYWGQGTLVTVSS |
| 55 | BMA031- | | VH for | EVQLVQSGAEVKKPGASVKVSCKASGYKFTSYVMH |
| | V36 VH | | alternative | WVRQAPGQGLEWMGYINPYNDVTKYAEKFQGRVT |
| | | | BM | LTSDTSTSTAYMELSSLRSEDTAVHYCARGSYYDYEG |
| | | | recruiter | FVYWGQGTLVTVSS |
| 56 | BMA031 | | VH for | EVQLVQSGAEVKKPGASVKVSCKASGYKFTSYVMH |
| | D01 VH | | alternative | WVRQAPGQGLEWMGYINPRNDVTKYAEKFQGRVT |
| | | | BM | LTSDTSTSTAYMELSSLRSEDTAVHYCARGSYYDYEG |
| | | | recruiter | FVYWGQGTLVTVSS |
| 57 | BMA | | | YINPYNDVTKYAEKFQG |
| | HCDR2 | | | |
| | variant | | | |
| 58 | BMA | | | GSYYDYDGFVY |
| | HCDR3 | | | |
| | variant | | | |
| 59 | SP-02- | TPP- | Seq- | FIVDGVEVSV |
| | 1621 | 142 | SimPep | |
| 60 | SP-02- | TPP- | Seq- | FVYDEPGHAV |
| | 1622 | 143 | SimPep | |
| 61 | SP-02- | TPP- | Seq- | GLSDGEWQLV |
| | 1623 | 144 | SimPep | |
| 62 | SP-02- | TPP- | Seq- | GLYDGPVHEV |
| | 1624 | 145 | SimPep | |
| 63 | SP-02- | TPP- | Seq- | GVIDGHIYAV |
| | 1625 | 146 | SimPep | |
| 64 | SP-02- | TPP- | Seq- | GVMAGDIYSV |
| | 1626 | 147 | SimPep | |
| 65 | SP-02- | TPP- | Seq- | GVYGGSVHEA |
| | 1627 | 148 | SimPep | |
| 66 | SP-02- | TPP- | Seq- | TVYGGYLCSV |
| | 1628 | 149 | SimPep | |
| 67 | SP-02- | TPP- | Seq- | YTYDEAIHSV |
| | 1629 | 150 | SimPep | |
| 68 | MAGEB2- | TPP- | ScanPep | AVYDGEEHSV |
| | 001_A1 | 151 | | |
| 69 | MAGEB2- | TPP- | ScanPep | GVADGEEHSV |
| | 001_A3 | 153 | | |
| 70 | MAGEB2- | TPP- | ScanPep | GVYAGEEHSV |
| | 001_A4 | 154 | | |
| 71 | MAGEB2- | TPP- | ScanPep | GVYDAEEHSV |
| | 001_A5 | 155 | | |
| 72 | MAGEB2- | TPP- | ScanPep | GVYDGAEHSV |
| | 001_A6 | 156 | | |
| 73 | MAGEB2- | TPP- | ScanPep | GVYDGEAHSV |
| | 001_A7 | 157 | | |
| 74 | MAGEB2- | TPP- | ScanPep | GVYDGEEASV |
| | 001_A8 | 158 | | |
| 75 | MAGEB2- | TPP- | ScanPep | GVYDGEEHAV |
| | 001_A9 | 159 | | |
| 76 | R76P1E1 | CL- | Parent | EDVEQSLFLSVREGDSSVINCTYTDSSSTYLYWYKQE |
| | alpha | 6120 | TCR alpha | PGAGLQLLTYIFSNMDMKQDQRLTVLLNKKDKHLSL |
| | chain | | chain | RIADTQTGDSAIYFCAEKDNARLMFGDGTQLVVKPN |
| | | | (TRAV5*01_ | IQNPDPAVYQLRDSKSSDKSVCLFTDFDSQTNVSQS |
| | | | TRAJ31*0 | KDSDVYITDKTVLDMRSMDFKSNSAVAWSNKSDFA |
| | | | 1_TRAC) | CANAFNNSIIPEDTFFPSPESSCDVKLVEKSFETDTN |
| | | | | LNFQNLSVIGFRILLLKVAGFNLLMTLRLWSS |
| 77 | R76P1E1 | CL- | Parent | AVISQKPSRDICQRGTSLTIQCQVDSQVTMMFWYR |
| | beta | 6120 | TCR beta | QQPGQSLTLIATANQGSEATYESGFVIDKFPISRPNL |
| | chain | | chain | TFSTLTVSNMSPEDSSIYLCSVLGAYGYTFGSGTRLTV |
| | | | (TRBV29- | VEDLNKVFPPEVAVFEPSEAEISHTQKATLVCLATGF |
| | | | 1*01_TRBJ | FPDHVELSWWVNGKEVHSGVSTDPQPLKEQPALN |
| | | | 1- | DSRYCLSSRLRVSATFWQNPRNHFRCQVQFYGLSE |
| | | | 2*01_TRB | NDEWTQDRAKPVTQIVSAEAWGRADCGFTSVSYQ |
| | | | C1*01) | QGVLSATILYEILLGKATLYAVLVSALVLMAMVKRKDF |
| 78 | R76P1E1 | CL- | Parent | EDVEQSLFLSVREGDSSVINCTYTDSSSTYLYWYKQE |
| | Valpha | 6120 | TCR | PGAGLQLLTYIFSNMDMKQDQRLTVLLNKKDKHLSL |
| | | | Valpha | RIADTQTGDSAIYFCAEKDNARLMFGDGTQLVVKP |
| 79 | R76P1E1 | CL- | Parent | AVISQKPSRDICQRGTSLTIQCQVDSQVTMMFWYR |
| | Vbeta | 6120 | TCR Vbeta | QQPGQSLTLIATANQGSEATYESGFVIDKFPISRPNL |
| | | | | TFSTLTVSNMSPEDSSIYLCSVLGAYGYTFGSGTRLTV |
| | | | | V |
| 80 | R76P1E1 | CL- | R76P1E1 | DSSSTY |
| | CDRa1 | 6120 | CDRa1 | |
| 81 | R76P1E1 | CL- | R76P1E1 | AEKDNARLM |
| | CDRa3 | 6120 | CDRa3 | |
| 82 | R76P1E1 | CL- | R76P1E1 | SQVTM |
| | CDRb1 | 6120 | CDRb1 | |
| 83 | R76P1E1 | CL- | R76P1E1 | ANQGSEA |
| | CDRb2 | 6120 | CDRb2 | |
| 84 | Aga2p + | | | MQLLRCFSIFSVIASVLAQELTTICEQIPSPTLESTPYS |
| | leader | | | LSTTTILANGKAMQGVFEYYKSVTFVSNCGSHPSTTS |
| | sequence | | | KGSPINTQYVF |
| 85 | Display | | | GGGGSGGGGSGGGGGGGGSGGGGS |
| | linker | | | |
| 86 | FLAG tag | | | DYKDDDDK |
| 87 | Myc tag | | | EQKLISEEDL |
| 88 | Fusion | | | MQLLRCFSIFSVIASVLAQELTTICEQIPSPTLESTPYS |
| | protein | | | LSTTTILANGKAMQGVFEYYKSVTFVSNCGSHPSTTS |
| | scTCR | | | KGSPINTQYVFGGGGSDYKDDDDKGGGASEDVEQ |
| | R76P1E1 | | | SLFLSVREGDSAVINCTYTDSSSTYLYWYKQEPGAGL |
| | | | | QLLTYIFSNMDMKQDQRLTVLLNKKDKHLSLRIADT |
| | | | | QTGDSAIYFCAEKDNARLMFGDGTQLVVKPGGGGS |
| | | | | GGGGSGGGGSGGGGSGGGGSGAVISQKPSRDIC |
| | | | | QRGTSLTIQCQVDSQVTMMFWYRQQPGQSLTLIAT |
| | | | | ANQGSEATYESGFVIDKFPISRPNLTFSTLTVSNMSP |
| | | | | EDSSIYLCSVLGAYGYTFGSGTRLTVVAAAGGSGGE |
| | | | | QKLISEEDL |
| 89 | scTCR | CL- | scTv | EDVEQSLFLSVREGDSSVINCTYTDSSSTYLYWYKQE |
| | R76P1E1 | 19871 | without | PGAGLQLLTYIFSNMDMKQDQRLTVLLNKKDKHLSL |
| | | | Aga2p or | RIADTQTGDSAIYFCAEKDNARLMFGDGTQLVVKPG |
| | | | preceding | GGGSGGGGSGGGGSGGGGSGGGGSGAVISQKPS |
| | | | or | RDICQRGTSLTIQCQVDSQVTMMFWYRQQPGQSLT |
| | | | following | LIATANQGSEATYESGFVIDKFPISRPNLTFSTLTVSN |
| | | | linkers/ | MSPEDSSIYLCSVLGAYGYTFGSGTRLTVV |
| | | | tags | |
| 90 | scTCR | CL- | Parent | EDVEQSLFLSVREGDSAVINCTYTDSSSTYLYWYKQE |
| | R76P1E1_ | 19872 | clone for | PGAGLQLLTYIFSNMDMKQDQRLTVLLNKKDKHLSL |
| | bC13V | | scTv | RIADTQTGDSAIYFCAEKDNARLMFGDGTQLVVKPG |
| | | | conversion | GGGSGGGGSGGGGSGGGGSGGGGSGAVISQKPS |
| | | | (aS19A + | RDIVQRGTSLTIQCQVDSQVTMMFWYRQQPGQSLT |
| | | | bC13V) | LIATANQGSEATYESGFVIDKFPISRPNLTFSTLTVSN |
| | | | | MSPEDSSIYLCSVLGAYGYTFGSGTRLTVV |
| 91 | R76P1E1C | CL- | Stabilized | EDVEQSSFLSVREGDSAVINCTYTDSSSTYFYWYKQ |
| | | 48396 | clone from | EPGAGLQLLTYIFSNMDMKQDQRLTVLLNKKDKHLS |
| | | | scTv | LRIADTQTGDSAIYFCAVKDNARLMFGDGTQLVVKP |
| | | | conversion | GGGGSGGGGSGGGGSGGGGSGGGGSGAVISQK |
| | | | with free | PSRDIVQRGNSVTIQCQVDSQVTMMFWYRQQPGQ |
| | | | Cys | SLTLIATANQGSEATYESGFVIDKFPISRPNLTFSTLTV |
| | | | | SNMSPEDSSIYLCCVLGAYGYTFGSGTRLTVV |
| 92 | R76P1E1C | CL- | | CVLGAYGYT |
| | CDRb3 | 48396 | | |
| 93 | scTCR | CL- | Cys free | EDVEQSSFLSVREGDSAVINCTYTDSSSTYFYWYKQ |
| | R76P1E1S | 48541 | version of | EPGAGLQLLTYIFSNMDMKQDQRLTVLLNKKDKHLS |
| | | | R76P1E1C, | LRIADTQTGDSAIYFCAVKDNARLMFGDGTQLVVKP |
| | | | used as | GGGGSGGGGSGGGGSGGGGSGGGGSGAVISQK |
| | | | basis for | PSRDIVQRGNSVTIQCQVDSQVTMMFWYRQQPGQ |
| | | | affinity | SLTLIATANQGSEATYESGFVIDKFPISRPNLTFSTLTV |
| | | | maturation | SNMSPEDSSIYLCSVLGAYGYTFGSGTRLTVV |
| 94 | CL-48730 | CL- | Selection | EDVEQSSFLSVREGDSAVINCTYTDSSSTYFYWYKQ |
| | | 48730 | from CDR | EPGAGLQLLTYIFSNMDMKQDQRLTVLLNKKDKHLS |
| | | | combina- | LRIADTQTGDSAIYFCAVKDNARLMFGDGTQLVVKP |
| | | | torial | GGGGSGGGGSGGGGSGGGGSGGGGSGAVISQK |
| | | | library | PSRDIVQRGNSVTIQCQVDRPVTMMFWYRQQPGQ |
| | | | | SLTLIATAYDDGSATYESGFVIDKFPISRPNLTFSTLTV |
| | | | | SNMSPEDSSIYLCSVLGAYGYTFGSGTRLTVV |
| 95 | CL-48731 | CL- | Selection | EDVEQSSFLSVREGDSAVINCTYTDSSSTYFYWYKQ |
| | | 48731 | from CDR | EPGAGLQLLTYIFSNMDMKQDQRLTVLLNKKDKHLS |
| | | | combina- | LRIADTQTGDSAIYFCAVKDNARLMFGDGTQLVVKP |
| | | | torial | GGGGSGGGGSGGGGSGGGGSGGGGSGAVISQK |
| | | | library | PSRDIVQRGNSVTIQCQVDRPVTMMFWYRQQPGQ |
| | | | | SLTLIATAYSDGTATYESGFVIDKFPISRPNLTFSTLTVS |
| | | | | NMSPEDSSIYLCSVLGAYGYTFGSGTRLTVV |
| 96 | CL-48732 | CL- | Selection | EDVEQSSFLSVREGDSAVINCTYTDSSSTYFYWYKQ |
| | | 48732 | from CDR | EPGAGLQLLTYIFSNMDMKQDQRLTVLLNKKDKHLS |
| | | | combina- | LRIADTQTGDSAIYFCAVKDNARLMFGDGTQLVVKP |
| | | | torial | GGGGSGGGGSGGGGSGGGGSGGGGSGAVISQK |
| | | | library | PSRDIVQRGNSVTIQCQVDRPVTMMFWYRQQPGQ |
| | | | | SLTLIATAYDDNTTTYESGFVIDKFPISRPNLTFSTLTVS |
| | | | | NMSPEDSSIYLCSVLGAYGYTFGSGTRLTVV |
| 97 | CL-21753 | CL- | SeqLiab | EDVEQSSFLSVREGDSAVINCTYTDASSTYFYWYKQ |
| | | 21753 | remediated | EPGAGLQLLTYIFSNMDMKQDQRLTVLLNKKDKHLS |
| | | | clone for | LRIADTQTGDSAIYFCAVKDNARLMFGDGTQLVVKP |
| | | | Biooptimi- | GGGGSGGGGSGGGGSGGGGSGGGGSAVISQKPS |
| | | | zation | RDIVQRGNSVTIQCQVDRPVTMMFWYRQQPGQSL |
| | | | (One G | TLIATAYDEGSATYESGFVIDKFPISRPNLTFSTLTVSN |
| | | | less in | MSPEDSSIYLCSVLGAYGYTFGSGTRLTVV |
| | | | linker), | |
| | | | Clone for | |
| | | | TCER | |
| | | | orientation | |
| | | | run | |
| 98 | CL-25688 | CL- | Clone | EDVEQSSFLSVREGDSAVINCTYTDDSSTYFYWYKQ |
| | | 25688 | from | EPGAGLQLLTYIFSNMDMKQDQRLTVLLNKKDKHLS |
| | | | Biooptimi- | LRIADTQTGDSAIYFCAVKDNARLMFGDGTQLVVKP |
| | | | zation | GGGGSGGGGSGGGGSGGGGSGGGGSAVISQKPS |
| | | | | RDIVQRGNSVTIQCQVDRPVTMMFWYRQQPGQSP |
| | | | | TLIATAYDEGSATYESGFVIDKFPISRPNLTFSTLTVSN |
| | | | | MSPEDSSIYLCCVLGAYGYTFGSGTRLTVV |
| 99 | CL-25687 | CL- | Clone | EDVEQSSFLSVREGDSAVINCTYTDASSTYFYWYKQ |
| | | 25687 | from | EPGAGLQLLTYIYSNMDMKQDQRLTVLLNKKDKHLS |
| | | | Biooptimi- | LRIADTQTGDSAIYFCAVKDNARLLFGDGTQLVVKP |
| | | | zation\|2 | GGGGSRGGGSGGGGSGGGGSGGGDSAVISQKPS |
| | | | mutations | RDIVQRGNSVTIQCQVDRPVTMMFWYRQQPGQSL |
| | | | in linker | TLIATAYDEGSATYESGFDIDKFPISRPNLTFSTLTVSN |
| | | | | MSPEDSSIYLCSVLGAYGYTFGSGTRLTVV |
| 100 | CL-25693 | CL- | Clone | EDVEQSSFLSVREGDSAVINCTYTDASSTYFYWYKQ |
| | | 25693 | from | EPGAGLQLLTYIFSNMDMKQDQRLTVLLNKKDKHLS |
| | | | Biooptimi- | LRIADTQTGDSAIYFCAVKDNARLMFGDGTQLVVKP |
| | | | zation | GGGGSGGGGGGGGSGGGGSGGGGSAVISQKPS |
| | | | | RDIVQRGNSVTIQCQVDRPVTMMYWYRQQPGQSL |
| | | | | TLIATAYDEGSATYESGFDIDKFPISRPNLTFSTLTVSN |
| | | | | MSPEDSSIYFCSVLGAYGYTFGSGTRLTVV |
| 101 | CL-25720 | CL- | Clone | EDVEQSSFLSVREGDSAVINCTYTDASSTYFYWYKQ |
| | | 25720 | from | EPGAGLQLLTYIFSNMDMKQDQRLTVLLNKKDKHLS |
| | | | Biooptimi- | LRIADTQTGDSAIYFCAVKDNARLMFGDGTQLVVKP |
| | | | zation | GGGGSGGGGSGGGGSGGGGSGGGGSAVISQKPS |
| | | | | RYIVQRGNSVTIQCQVDRPVTMMFWYRQQPGQSPT |
| | | | | LIATAYDVGSATYESGFVIDKFPISRPNLTFSTLTVSNM |
| | | | | SPEDSSIYLCSVLGAYGYTFGSGTRLTVV |
| 102 | CL-25821 | CL- | Clone | EDVEQSSFLSVREGDSAVINCTYTDASSTYFYWYKQ |
| | | 25821 | from | EPGAGLQLLTYIFSNMDMKQDQRLTVLLNKKDKHLS |
| | | | Biooptimi- | LRIADTQTGDSAIYFCAVKDNARLMFGDVTQLVVKP |
| | | | zation | GGGGSGGGGSGGGGSGGGGSGGGGSAVISQKPS |
| | | | | RVIVQRGNSVTIQCQVDRPVTMMYWYRQQPGQSLT |
| | | | | LIATAYDEGSATYESGFVIDKFPISRPNLTFSTLTVSNV |
| | | | | SPEDSSIYLCSVLGAYGYTFGSGTRLTVV |
| 103 | CL-24597 | CL- | Clone | EDVEQSSFLSVREGDSAVINCTYTDSSSTYFYWYKQ |
| | | 24597 | from | EPGAGLQLLTYIFSNMDMKQDQRLTVLLNKKDKHLS |
| | | | Biooptimi- | LRIADTQTGDSAIYFCAVKDNARLMFGDGTQLVVKP |
| | | | zation | GGGGSGGGGSGGGGSGGGGSGGGGSAVISQKPS |
| | | | | RDIVQRGTSVTIQCQVDRPVTMMFWYRQQPGQSLT |
| | | | | LIATAYDEGSATYESGFVIDKFPISRPNLTFSTLTVSNL |
| | | | | EPEDSSIYLCSVLGAYGYTFGSGTRLTVV |
| 104 | CL-24505 | CL- | Clone | EDVEQSSFLSVREGDSAVINCTYTDASSTYFYWYKQ |
| | | 24505 | from | EPGAGLQLLTYIFSNMDMKQDQRLTVLLNKKDKHLS |
| | | | Biooptimi- | LRIADTQTGDSAIYFCAVKDNARLMFGDGTQLVVKP |
| | | | zation | GGGGSGGGGSGGGGSGGGGSGGGGSAVISQKPS |
| | | | | RDIVQRGTSVTIQCQVDRPVTMMFWYRQQPGQSLT |
| | | | | LIATAYDEGSATYESGFVIDKFPISRPNLTFSTLTVSNM |
| | | | | EPEDSSIYLCSVLGAYGYTFGSGTRLTVV |
| 105 | CL-24478 | CL- | Clone | EDVEQSSFLSVREGDSAVINCTYTDASSTYFYWYKQ |
| | | 24478 | from | EPGAGLQLLTYIFSNMDMKQDQRLTVLLNKKDKHLS |
| | | | Biooptimi- | LRIADTQTGDSAIYFCAVKDNARLMFGDGTQLVVKP |
| | | | zation | GGGGSGGGGSGGGGSGGGGSGGGGSAVISQKPS |
| | | | | RDIVQRGTSVTIQCQVDRPVTMMFWYRQQPGQSLT |
| | | | | LIATAYDEGSATYESGFVIDKFPISRPNLTFSTLTVSSM |
| | | | | EPEDSSIYLCSVLGAYGYTFGSGTRLTVV |
| 106 | SP-02- | | Seq- | RLYDGLFKVI |
| | 1640 | | SimPep | |
| 107 | CL-48641 | beta | CDR2 | AYDNNRT |
| 108 | CL-48644 | beta | CDR2 | AFDDNTT |
| 109 | CL-48650 | beta | CDR2 | AYDDGSA |
| 110 | CL-48715 | beta | CDR2 | AYSDGTA |
| 111 | BMhanced | | | X₁YVMH, where X₁ is S, R, K or H |
| | CDRH1 | | | |
| | consensus | | | |
| 112 | BMhanced | | | YINPX₁X₂DVTKYX₃X₄KFX₅G, where |
| | CDRH2 | | | X₁ is Y, R, K, or H, preferably Y or R; |
| | consensus | | | X₂ is N, R, or K, preferably N or R; |
| | | | | X₃ is A or N; |
| | | | | X₄ is E or Q, preferably E; and |
| | | | | X₅ is Q or K |
| 113 | BMhanced | | | SATSSVXYMH, where X is S, R, or K |
| | CDRL1 | | | |
| | consensus | | | |
| 114 | BMhanced | | | DTSKLAX, where X is S, R or K |
| | CDRL2 | | | |
| | consensus | | | |
| 115 | 20G6 - | | VH | QVQLVESGGGVVQPGRSLRLSCAASGFTFTKAWM |
| | alterna- | | | HWVRQAPGKQLEWVAQIKDKSNSYATYYADSVKGR |
| | tive T cell | | | FTISRDDSKNTLYLQMNSLRAEDTAVYYCRGVYYALS |
| | recruiter | | | PFDYWGQGTLVTVSS |
| | (anti- | | | |
| | CD3) | | | |
| 116 | 20G6 - | | VL | DIVMTQTPLSLSVTPGQPASISCKSSQSLVHNNANT |
| | alterna- | | | YLSWYLQKPGQSPQSLIYKVSNRFSGVPDRFSGSGS |
| | tive T cell | | | GTDFTLKISRVEAEDVGVYYCGQGTQYPFTFGSGTK |
| | recruiter | | | VEIK |
| | (anti- | | | |
| | CD3) | | | |
| 117 | 2B4 - | | VH | EVQLVESGGGLVQPGGSLRLSCAASGFTFSDYYMT |
| | alterna- | | | WVRQAPGKGLEWVAFIRNRARGYTSDHNPSVKGRF |
| | tive T cell | | | TISRDNAKNSLYLQMNSLRAEDTAVYYCARDRPSYY |
| | recruiter | | | VLDYWGQGTTVTVSS |
| | (anti- | | | |
| | CD3) | | | |
| 118 | 2B4 - | | VL | DIVMTQSPDSLAVSLGERATINCKSSQSLFNVRSRK |
| | alterna- | | | NYLAWYQQKPGQPPKLLISWASTRESGVPDRFSGS |
| | tive T cell | | | GSGTDFTLTISSLQAEDVAVYYCKQSYDLFTFGSGTK |
| | recruiter | | | LEIK |
| | (anti- | | | |
| | CD3) | | | |
| 119 | 38E4v - | | VH | EVQLVQSGAEVKKPGASVKVSCKASGFTFTSYYIHW |
| | alterna- | | | VRQAPGQGLEWIGWIYPENDNTKYNEKFKDRVTITA |
| | tive T cell | | | DTSTSTAYLELSSLRSEDTAVYYCARDGYSRYYFDYW |
| | recruiter | | | GQGTLVTVSS |
| | (anti- | | | |
| | CD3) | | | |
| 120 | 38E4v - | | VL | DIVMTQSPDSLAVSLGERATINCKSSQSLLNSRTRKN |
| | alterna- | | | YLAWYQQKPGQSPKLLIYWTSTRKSGVPDRFSGSG |
| | tive T cell | | | SGTDFTLTISSLQAEDVAVYYCKQSFILRTFGQGTKVE |
| | recruiter | | | IK |
| | (anti- | | | |
| | CD3) | | | |
| 121 | CE115 - | | VH | QVQLVESGGGVVQPGGSLRLSCAASGFTFSNAWM |
| | alterna- | | | HWVRQAPGKGLEWVAQIKDKSQNYATYVAESVKGR |
| | tive T cell | | | FTISRADSKNSIYLQMNSLKTEDTAVYYCRYVHYAAG |
| | recruiter | | | YGVDIWGQGTTVTVSS |
| | (anti- | | | |
| | CD3) | | | |
| 122 | CE115 - | | VL | DIVMTQSPLSLPVTPGEPASISCRSSQPLVHSNRNTY |
| | alterna- | | | LHWYQQKPGQAPRLLIYKVSNRFSGVPDRFSGSGS |
| | tive T cell | | | GTDFTLKISRVEAEDVGVYYCGQGTQVPYTFGQGTK |
| | recruiter | | | LEIK |
| | (anti- | | | |
| | CD3) | | | |
| 123 | CRIS7 - | | VH | QVQLVQSGPEVKKPGSSVKVSCKASGYTFSRSTMH |
| | alterna- | | | WVRQAPGQGLEWIGYINPSSAYTNYNQKFKDRVTIT |
| | tive T cell | | | ADKSTSTAYMELSSLRSEDTAVYYCARPQVHYDYNG |
| | recruiter | | | FPYWGQGTLVTVSS |
| | (anti- | | | |
| | CD3) | | | |
| 124 | CRIS7 - | | VL | DIQMTQSPSTLSASVGDRVTMTCSASSSVSYMNWY |
| | alterna- | | | QQKPGKAPKRWIYDSSKLASGVPSRFSGSGSGTDYT |
| | tive T cell | | | LTISSLQPDDFATYYCQQWSRNPPTFGGGTKVEIK |
| | recruiter | | | |
| | (anti- | | | |
| | CD3) | | | |
| 125 | F2B - | | VH | EVQLVESGGGLVQPGRSLRLSCAASGFTFDDYAMH |
| | alterna- | | | WVRQAPGKGLEWVSGISWNSGSIGYADSVKGRFTI |
| | tive T cell | | | SRDNAKNSLYLQMNSLRAEDTALYYCAKDSRGYGD |
| | recruiter | | | YRLGGAYWGQGTLVTVSS |
| | (anti- | | | |
| | CD3) | | | |
| 126 | F2B - | | VL | EIVMTQSPATLSVSPGERATLSCRASQSVSSNLAWY |
| | alterna- | | | QQKPGQAPRLLIYGASTRATGIPARFSGSGSGTEFTL |
| | tive T cell | | | TISSLQSEDFAVYYCQQYNNWPWTFGQGTKVEIK |
| | recruiter | | | |
| | (anti- | | | |
| | CD3) | | | |
| 127 | H2M2691 | | VH | EVQLVESGGGLVQPGRSLRLSCAASGFTFDDYTMH |
| | N - | | | WVRQAPGKGLEWVSGISWNSGSIGYADSVKGRFTI |
| | alterna- | | | SRDNAKKSLYLQMNSLRAEDTALYYCAKDNSGYGH |
| | tive T cell | | | YYYGMDVWGQGTTVTVAS |
| | recruiter | | | |
| | (anti- | | | |
| | CD3) | | | |
| 128 | H2M2691 | | VL | EIVMTQSPATLSVSPGERATLSCRASQSVSSNLAWY |
| | N - | | | QQKPGQAPRLLIYGASTRATGIPARFSGSGSGTEFTL |
| | alterna- | | | TISSLQSEDFAVYYCQHYINWPLTFGGGTKVEIK |
| | tive T cell | | | |
| | recruiter | | | |
| | (anti- | | | |
| | CD3) | | | |
| 129 | MT301 - | | VH | QVQLVQSGAEVKKPGASVKVSCKASGYTFTKYVIHW |
| | alterna- | | | VRQAPGQGLEWMGYINPYNDVSKYNEKFRGRVTMT |
| | tive T cell | | | TDTSTSTAYMELRSLRSDDTAVYYCARNKDYYPMDY |
| | recruiter | | | WGQGTTVTVSS |
| | (anti- | | | |
| | CD3) | | | |
| 130 | MT301 - | | VL | EIVLTQSPATLSLSPGERATLSCSASSSVSYMHWYQ |
| | alterna- | | | QKPGQAPRLLIYDTSKLASGIPARFSGSGSGTDFTLTI |
| | tive T cell | | | SSLEPEDFAVYYCQQWSSNPLTFGGGTKVEIK |
| | recruiter | | | |
| | (anti- | | | |
| | CD3) | | | |
| 131 | alterna- | | VH | QVQLVQSGGGVVQPGRSLRLSCVASGFTFSSYGM |
| | tive T cell | | | HWVRQAPGKGLEWVAAIWYNARKQDYADSVKGRF |
| | recruiter | | | TISRDNSKNTLYLQMNSLRAEDTAVYYCTRGTGYNW |
| | (anti- | | | FDPWGQGTLVTVSS |
| | CD3) | | | |
| 132 | alterna- | | VL | DIQMTQSPSSLSASVGDRVTITCRASQSISSYLNWY |
| | tive T cell | | | QQKPGKAPKLLIYAASSLQSGVPSRFSGSGSGTDFT |
| | recruiter | | | LTISSLQPEDFATYYCQQSYSTPPTFGQGTKVEIK |
| | (anti- | | | |
| | CD3) | | | |
| 133 | SP-01- | | Seq- | YLLPAIVHI |
| | 0010 | | SimPep | |
| 134 | SP-02- | | Seq- | GMYDGPVYEV |
| | 1631 | | SimPep | |
| 135 | SP-02- | | Seq- | FIYDNTEFTL |
| | 1635 | | SimPep | |
| 136 | SP-02- | | Seq- | NQWDGTQHGV |
| | 1641 | | SimPep | |
| 137 | SP-02- | | Seq- | SQYSGQLHEV |
| | 1642 | | SimPep | |
| 138 | SP-02- | | Seq- | VLYEGPVRGL |
| | 1643 | | SimPep | |
| 139 | SP-02- | | Seq- | GLYEGEFTLA |
| | 1644 | | SimPep | |
| 140 | SP-02- | | Seq- | GMYDGPVFDL |
| | 1647 | | SimPep | |
| 141 | SP-02- | | Seq- | GLYDGPVCEV |
| | 1648 | | SimPep | |
| 142 | SP-02- | | Seq- | GLYEGLDWLA |
| | 1650 | | SimPep | |
| 143 | SP-02- | | Seq- | GLYEGLDWLS |
| | 1651 | | SimPep | |
| 144 | SP-02- | | Seq- | FLHDGQEYTL |
| | 1652 | | SimPep | |
| 145 | SP-02- | | Seq- | ALYPGTDYTV |
| | 1653 | | SimPep | |
| 146 | SP-02- | | Seq- | SLYPFQIHSI |
| | 1655 | | SimPep | |
| 147 | SP-02- | | Seq- | RMYPFQIHSI |
| | 1656 | | SimPep | |
| 148 | SP-02- | | Seq- | LLYDGKLSSA |
| | 1664 | | SimPep | |
| 149 | SP-02- | | Seq- | ALYPGTNYTV |
| | 1673 | | SimPep | |
| 150 | SP-02- | | Seq- | ALENSEEHSA |
| | 1722 | | SimPep | |
| 151 | SP-02- | | Seq- | KFYDGVVQTV |
| | 1724 | | SimPep | |
| 152 | V_{A} | | | EDVEQSSFLSVREGDSAVINCTYTDASSTYFYWYKQ |
| | consensus | | | EPGAGLQLLTYIXSNMDMKQDQRLTVLLNKKDKHLS |
| | | | | LRIADTQTGDSAIYFCAVKDNARLXFGDXTQLVVKP |
| 153 | V_{A} | | | EDVEQSSFLSVREGDSAVINCTYTDASSTYFYWYKQ |
| | consensus | | | EPGAGLQLLTYIX₁SNMDMKQDQRLTVLLNKKDKHL |
| | | | | SLRIADTQTGDSAIYFCAVKDNARLX₂FGDX₃TQLVVK |
| | | | | P, wherein |
| | | | | X₁ is Y or F; |
| | | | | X₂ is L or M; |
| | | | | X₃ is V or G |
| 154 | V_{B} | | | AVISQKPSRXIVQRGXSVTIQCQVDRPVTMMXWYRQ |
| | consensus | | | QPGQSXTLIATAYDXGSATYESGFXIDKFPISRPNLTF |
| | | | | STLTVSNXXPEDSSIYLCSVLGAYGYTFGSGTRLTVV |
| 155 | V_{B} | | | AVISQKPSRX₁IVQRGX₂SVTIQCQVDRPVTMMX₃WY |
| | consensus | | | RQQPGQSX₄TLIATAYDX₅GSATYESGFX₆IDKFPISRP |
| | | | | NLTFSTLTVSNX₇X₈PEDSSIYLCSVLGAYGYTFGSGT |
| | | | | RLTVV, |
| | | | | wherein |
| | | | | X₁ is V or Y or D; |
| | | | | X₂ is T or N; |
| | | | | X₃ is Y or F; |
| | | | | X₄ is L or P; |
| | | | | X₅ is E or V; |
| | | | | X₆ is D or V; |
| | | | | X₇ is V or M; |
| | | | | X₈ is E or S |

1. An antigen binding protein specifically binding to a MAGEB2 antigenic peptide that is in a complex with a major histocompatibility complex (MHC) protein, wherein the MAGEB2 antigenic peptide comprises or consists of the amino acid sequence GVYDGEEHSV (SEQ ID NO: 1),
wherein the antigen binding protein comprises a first polypeptide comprising a variable domain V_{A} comprising complementarity determining regions (CDRs) CDRa1, CDRa2, and CDRa3, and a second polypeptide comprising a variable domain V_{B} comprising CDRb1, CDRb2, and CDRb3, wherein CDRa1, CDRa2, CDRa3, CDRb1, CDRb2 and CDRb3 form an antigen binding domain A;
wherein
(1) CDRa1 comprises DASSTY (SEQ ID NO: 2), CDRa3 comprises AVKDNARLL (SEQ ID NO: 3), CDRb1 comprises RPVTM (SEQ ID NO: 5), and CDRb3 comprises SVLGAYGYT (SEQ ID NO: 6); or
(2) CDRa1 comprises SEQ ID NO: 2, CDRa3 comprises AVKDNARLM (SEQ ID NO: 4), CDRb1 comprises SEQ ID NO: 5, and CDRb3 comprises SEQ ID NO: 6;
wherein the CDRa1, CDRa3, CDRb1 and/or CDRb3 sequence(s) may comprise one, two or three amino acid mutations.

2. The antigen binding protein of claim 1, wherein
(1) CDRa1 comprises SEQ ID NO: 2, CDRa2 comprises IYSNMDM (SEQ ID NO: 7), CDRa3 comprises SEQ ID NO: 3, CDRb1 comprises SEQ ID NO: 5, CDRb2 comprises AYDEGSA (SEQ ID NO: 9), and CDRb3 comprises SEQ ID NO: 6;
(2) CDRa1 comprises SEQ ID NO: 2, CDRa2 comprises IFSNMDM (SEQ ID NO: 8), CDRa3 comprises SEQ ID NO: 4, CDRb1 comprises SEQ ID NO: 5, CDRb2 comprises AYDVGSA (SEQ ID NO: 10), and CDRb3 comprises SEQ ID NO: 6;
(3) CDRa1 comprises SEQ ID NO: 2, CDRa2 comprises SEQ ID NO: 7, CDRa3 comprises SEQ ID NO: 3, CDRb1 comprises SEQ ID NO: 5, CDRb2 comprises SEQ ID NO: 10, and CDRb3 comprises SEQ ID NO: 6; or
(4) CDRa1 comprises SEQ ID NO: 2, CDRa2 comprises SEQ ID NO: 8, CDRa3 comprises SEQ ID NO: 4, CDRb1 comprises SEQ ID NO: 5, CDRb2 comprises SEQ ID NO: 9, and CDRb3 comprises SEQ ID NO: 6;
wherein the CDRa1, CDRa2, CDRa3, CDRb1, CDRb2 and/or CDRb3 sequence(s) may comprise one, two or three amino acid mutations.

3. The antigen binding protein of claim 1,
wherein the V_{A} comprises an amino acid sequence of EDVEQSSFLSVREGDSAVINCTYTDASSTYFYWYKQEPGAGLQLLTYIX₁SNMDMKQDQRLTVLLNKKD KHLSLRIADTQTGDSAIYFCAVKDNARLX₂FGDX₃TQLVVKP (SEQ ID NO: 153), wherein X₁ is Y or F, X₂ is L or M, and X₃ is V or G, or an amino acid sequence having at least 85%, 90%, 95%, 98% or 99% identity to SEQ ID NO: 153, wherein X₁ is Y or F, X₂ is L or M, and X₃ is V or G, and
wherein the V_{B} comprises an amino acid sequence of AVISQKPSRX₁IVQRGX₂SVTIQCQVDRPVTMMX₃WYRQQPGQSX₄TLIATAYDX₅GSATYESGFX₆IDKFPI SRPNLTFSTLTVSNX₇X₈PEDSSIYLCSVLGAYGYTFGSGTRLTWV,
(SEQ ID NO: 155), wherein X₁ is V, Y, or D; X₂ is T or N; X₃ is Y or F; X₄ is L or P; X₅ is E or V; X₆ is D or V; X₇ is V or M; and X₈ is E or S; or an amino acid sequence having at least 85%, 90%, 95%, 98% or 99% identity to SEQ ID NO: 155, wherein X₁ is V, Y, or D; X₂ is T or N; X₃ is Y or F; X₄ is L or P; X₅ is E or V; X₆ is D or V; X₇ is V or M; and X₈ is E or S.

4. The antigen binding protein of claim 3,
wherein the V_{A} comprises an amino acid sequence selected from the group consisting of SEQ ID NOs: 11, 12, 13, and 14, or
an amino acid sequence having at least 85%, 90%, 95%, 98% or 99% identity to SEQ ID NO: 11, and comprising the CDRa1, CDRa2, and CDRa3 of SEQ ID NOs: 2, 7, and 3, respectively, or
an amino acid sequence having at least 85%, 90%, 95%, 98% or 99% identity to SEQ ID NO: 12, and comprising the CDRa1, CDRa2, and CDRa3 of SEQ ID NOs: 2, 8, and 4, respectively, or
an amino acid sequence having at least 85%, 90%, 95%, 98% or 99% identity to SEQ ID NO: 13, and comprising the CDRa1, CDRa2, and CDRa3 of SEQ ID NOs: 2, 7, and 3, respectively, or
an amino acid sequence having at least 85%, 90%, 95%, 98% or 99% identity to SEQ ID NO: 14, and comprising the CDRa1, CDRa2, and CDRa3 of SEQ ID NOS: 2, 8, and 4, respectively; and
wherein the V_{B} comprises an amino acid sequence selected from the group consisting of SEQ ID NOs: 15, 16, 17, 18, 19, 20, 21, 22, and 23, or
an amino acid sequence having at least 85%, 90%, 95%, 98% or 99% identity to SEQ ID NO: 15, and comprising the CDRb1, CDRb2, and CDRb3 of SEQ ID NOs: 5, 9, and 6, respectively, or
an amino acid sequence having at least 85%, 90%, 95%, 98% or 99% identity to SEQ ID NO: 16, and comprising the CDRb1, CDRb2, and CDRb3 of SEQ ID NOs: 5, 10, and 6, respectively, or
an amino acid sequence having at least 85%, 90%, 95%, 98% or 99% identity to SEQ ID NO: 17, and comprising the CDRb1, CDRb2, and CDRb3 of SEQ ID NOs: 5, 10, and 6, respectively, or
an amino acid sequence having at least 85%, 90%, 95%, 98% or 99% identity to SEQ ID NO: 18, and comprising the CDRb1, CDRb2, and CDRb3 of SEQ ID NOs: 5, 10, and 6, respectively, or
an amino acid sequence having at least 85%, 90%, 95%, 98% or 99% identity to SEQ ID NO: 19, and comprising the CDRb1, CDRb2, and CDRb3 of SEQ ID NOs: 5, 10, and 6, respectively, or
an amino acid sequence having at least 85%, 90%, 95%, 98% or 99% identity to SEQ ID NO: 20, and comprising the CDRb1, CDRb2, and CDRb3 of SEQ ID NOs: 5, 10, and 6, respectively, or
an amino acid sequence having at least 85%, 90%, 95%, 98% or 99% identity to SEQ ID NO: 21, and comprising the CDRb1, CDRb2, and CDRb3 of SEQ ID NOs: 5, 10, and 6, respectively, or
an amino acid sequence having at least 85%, 90%, 95%, 98% or 99% identity to SEQ ID NO: 22, and comprising the CDRb1, CDRb2, and CDRb3 of SEQ ID NOs: 5, 9, and 6, respectively, or
an amino acid sequence having at least 85%, 90%, 95%, 98% or 99% identity to SEQ ID NO: 23, and comprising the CDRb1, CDRb2, and CDRb3 of SEQ ID NOs: 5, 9, and 6, respectively,
wherein optionally the CDRa1, CDRa2, CDRa3, CDRb1, CDRb2, and/or CDRb3 sequences may comprise one, two or three amino acid mutations, preferably amino acid substitutions.

5. The antigen binding protein of claim 1,
wherein the V_{A} comprises an amino acid sequence of SEQ ID NO: 11 and the Vb comprises an amino acid sequence of SEQ ID NO: 15; or
wherein the V_{A} comprises an amino acid sequence of SEQ ID NO: 12 and the Vb comprises an amino acid sequence of SEQ ID NO: 16; or
wherein the V_{A} comprises an amino acid sequence of SEQ ID NO: 13 and the Vb comprises an amino acid sequence of SEQ ID NO: 17; or
wherein the V_{A} comprises an amino acid sequence of SEQ ID NO: 14 and the Vb comprises an amino acid sequence of SEQ ID NO: 18; or
wherein the V_{A} comprises an amino acid sequence of SEQ ID NO: 13 and the Vb comprises an amino acid sequence of SEQ ID NO: 19; or
wherein the V_{A} comprises an amino acid sequence of SEQ ID NO: 11 and the Vb comprises an amino acid sequence of SEQ ID NO: 19; or
wherein the V_{A} comprises an amino acid sequence of SEQ ID NO: 14 and the Vb comprises an amino acid sequence of SEQ ID NO: 20; or
wherein the V_{A} comprises an amino acid sequence of SEQ ID NO: 13 and the Vb comprises an amino acid sequence of SEQ ID NO: 21; or
wherein the V_{A} comprises an amino acid sequence of SEQ ID NO: 11 and the Vb comprises an amino acid sequence of SEQ ID NO: 21; or
wherein the V_{A} comprises an amino acid sequence of SEQ ID NO: 12 and the Vb comprises an amino acid sequence of SEQ ID NO: 18; or
wherein the V_{A} comprises an amino acid sequence of SEQ ID NO: 13 and the Vb comprises an amino acid sequence of SEQ ID NO: 22; or
wherein the V_{A} comprises an amino acid sequence of SEQ ID NO: 14 and the Vb comprises an amino acid sequence of SEQ ID NO: 23.

6. The antigen binding protein of claim 1,
wherein the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
the second polypeptide comprises an amino acid sequence of SEQ ID NO: 34, or an amino acid sequence having at least 85%, 90%, 95%, 98% or 99% identity to SEQ ID NO: 34 and comprising the CDRb1, CDRb2, and CDRb3 of SEQ ID NO: 5, 9, and 6, respectively; or
wherein the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively, and
the second polypeptide comprises an amino acid sequence of SEQ ID NO: 35, or an amino acid sequence having at least 85%, 90%, 95%, 98% or 99% identity to SEQ ID NO: 35 and comprising the CDRb1, CDRb2, and CDRb3 of SEQ ID NO: 5, 10, and 6, respectively; or
wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
the second polypeptide comprises an amino acid sequence of SEQ ID NO: 36, or an amino acid sequence having at least 85%, 90%, 95%, 98% or 99% identity to SEQ ID NO: 36 and comprising the CDRb1, CDRb2, and CDRb3 of SEQ ID NO: 5, 10, and 6, respectively; or
wherein the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
the second polypeptide comprises an amino acid sequence of SEQ ID NO: 37, or an amino acid sequence having at least 85%, 90%, 95%, 98% or 99% identity to SEQ ID NO: 37 and comprising the CDRb1, CDRb2, and CDRb3 of SEQ ID NO: 5, 10, and 6, respectively; or
wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
the second polypeptide comprises an amino acid sequence of SEQ ID NO: 38, or an amino acid sequence having at least 85%, 90%, 95%, 98% or 99% identity to SEQ ID NO: 38 and comprising the CDRb1, CDRb2, and CDRb3 of SEQ ID NO: 5, 10, and 6, respectively; or
wherein the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
the second polypeptide comprises an amino acid sequence of SEQ ID NO: 38, or an amino acid sequence having at least 85%, 90%, 95%, 98% or 99% identity to SEQ ID NO: 38 and comprising the CDRb1, CDRb2, and CDRb3 of SEQ ID NO: 5, 10, and 6, respectively; or
wherein the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
the second polypeptide comprises an amino acid sequence of SEQ ID NO: 39, or an amino acid sequence having at least 85%, 90%, 95%, 98% or 99% identity to SEQ ID NO: 39 and comprising the CDRb1, CDRb2, and CDRb3 of SEQ ID NO: 5, 10, and 6, respectively; or
wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
the second polypeptide comprises an amino acid sequence of SEQ ID NO: 40, or an amino acid sequence having at least 85%, 90%, 95%, 98% or 99% identity to SEQ ID NO: 40 and comprising the CDRb1, CDRb2, and CDRb3 of SEQ ID NO: 5, 10, and 6, respectively; or
wherein the first polypeptide comprises an amino acid sequence of SEQ ID NO: 27, or an amino acid sequence having at least 85%, 90%, 95%, 98% or 99% identity to SEQ ID NO: 27 and comprising the CDRa1, CDRa2, and CDRa3 of SEQ ID NO: 2, 7, and 3, respectively, and
the second polypeptide comprises an amino acid sequence of SEQ ID NO: 40, or an amino acid sequence having at least 85%, 90%, 95%, 98% or 99% identity to SEQ ID NO: 40 and comprising the CDRb1, CDRb2, and CDRb3 of SEQ ID NO: 5, 10, and 6, respectively; or
wherein the first polypeptide comprises an amino acid sequence of SEQ ID NO: 28, or an amino acid sequence having at least 85%, 90%, 95%, 98% or 99% identity to SEQ ID NO: 28 and comprising the CDRa1, CDRa2, and CDRa3 of SEQ ID NO: 2, 8, and 4, respectively, and
the second polypeptide comprises an amino acid sequence of SEQ ID NO: 37, or an amino acid sequence having at least 85%, 90%, 95%, 98% or 99% identity to SEQ ID NO: 37 and comprising the CDRb1, CDRb2, and CDRb3 of SEQ ID NO: 5, 10, and 6, respectively; or
wherein the first polypeptide comprises an amino acid sequence of SEQ ID NO: 29, or an amino acid sequence having at least 85%, 90%, 95%, 98% or 99% identity to SEQ ID NO: 29 and comprising the CDRa1, CDRa2, and CDRa3 of SEQ ID NO: 2, 7, and 3, respectively, and
the second polypeptide comprises an amino acid sequence of SEQ ID NO: 41, or an amino acid sequence having at least 85%, 90%, 95%, 98% or 99% identity to SEQ ID NO: 41 and comprising the CDRb1, CDRb2, and CDRb3 of SEQ ID NO: 5, 9, and 6, respectively; or
wherein the first polypeptide comprises an amino acid sequence of SEQ ID NO: 30, or an amino acid sequence having at least 85%, 90%, 95%, 98% or 99% identity to SEQ ID NO: 30 and comprising the CDRa1, CDRa2, and CDRa3 of SEQ ID NO: 2, 8, and 4, respectively, and
the second polypeptide comprises an amino acid sequence of SEQ ID NO: 42, or an amino acid sequence having at least 85%, 90%, 95%, 98% or 99% identity to SEQ ID NO: 42 and comprising the CDRb1, CDRb2, and CDRb3 of SEQ ID NO: 5, 9, and 6, respectively; or
wherein the first polypeptide comprises an amino acid sequence of SEQ ID NO: 31, or an amino acid sequence having at least 85%, 90%, 95%, 98% or 99% identity to SEQ ID NO: 31 and comprising the CDRa1, CDRa2, and CDRa3 of SEQ ID NO: 2, 8, and 4, respectively, and
the second polypeptide comprises an amino acid sequence of SEQ ID NO: 43, or an amino acid sequence having at least 85%, 90%, 95%, 98% or 99% identity to SEQ ID NO: 43 and comprising the CDRb1, CDRb2, and CDRb3 of SEQ ID NO: 5, 9, and 6, respectively; or
wherein the first polypeptide comprises an amino acid sequence of SEQ ID NO: 32, or an amino acid sequence having at least 85%, 90%, 95%, 98% or 99% identity to SEQ ID NO: 32 and comprising the CDRa1, CDRa2, and CDRa3 of SEQ ID NO: 2, 8, and 4, respectively, and
the second polypeptide comprises an amino acid sequence of SEQ ID NO: 44, or an amino acid sequence having at least 85%, 90%, 95%, 98% or 99% identity to SEQ ID NO: 44 and comprising the CDRb1, CDRb2, and CDRb3 of SEQ ID NO: 5, 9, and 6, respectively; or
wherein the first polypeptide comprises an amino acid sequence of SEQ ID NO: 33, or an amino acid sequence having at least 85%, 90%, 95%, 98% or 99% identity to SEQ ID NO: 33 and comprising the CDRa1, CDRa2, and CDRa3 of SEQ ID NO: 2, 8, and 4, respectively, and
the second polypeptide comprises an amino acid sequence of SEQ ID NO: 45, or an amino acid sequence having at least 85%, 90%, 95%, 98% or 99% identity to SEQ ID NO: 45 and comprising the CDRb1, CDRb2, and CDRb3 of SEQ ID NO: 5, 9, and 6, respectively;
wherein optionally said CDRa1, CDRa2, CDRa3, CDRb1, CDRb2 and/or CDRb3 sequences may comprise one, two or three amino acid mutations, preferably amino acid substitutions.

7. The antigen binding protein of claim 1, wherein the antigen binding protein further comprises a variable domain VH comprising a CDRH1, a CDRH2 and a CDRH3, and a VL comprising a CDRL1, a CDRL2, and a CDRL3, wherein CDRH1, CDRH2, CDRH3, CDRL1, CDRL2 and CDRL3 form an antigen binding domain B.

8. The antigen binding protein of claim 7, wherein the antigen binding domain B specifically binds to immune cells, preferably T cells or natural killer (NK) cells.

9. The antigen binding protein of claim 7, wherein the VH comprises:
(a) a CDRH1 comprising the amino acid sequence of SYVMH (SEQ ID NO: 48),
(b) a CDRH2 comprising the amino acid sequence of YINPRNDVTKYAEKFQG (SEQ ID NO: 49) or YINPYNDVTKYAEKFQG (SEQ ID NO: 57), and
(c) a CDRH3 comprising the amino acid sequence of GSYYDYEGFVY (SEQ ID NO: 50) or GSYYDYDGFVY (SEQ ID NO: 58), and
wherein the VL comprises:
(a) a CDRL1 comprising the amino acid sequence of SATSSVSYMH (SEQ ID NO: 51),
(b) a CDRL2 comprising the amino acid sequence of DTSKLAS (SEQ ID NO: 52), and
(c) a CDRL3 comprising the amino acid sequence of QQWSSNPLT (SEQ ID NO: 53);
and optionally wherein each of the CDRH1, the CDRH2 and the CDRH3, and the CDRL1, CDRL2, and CDRL3 comprises up to three amino acid substitutions.

10. The antigen binding protein of claim 7, wherein the antigen binding protein comprises two antigen binding sites A and B, wherein a first polypeptide chain comprises a structure represented by formula:
V1-L1-V2 [I]
wherein V1 is a first variable domain; V2 is a second variable domain; L₁ is a linker domain;
and wherein a second polypeptide chain comprises a structure represented by formula:
V3-L3-V4 [II]
wherein V3 is a third variable domain; V4 is a fourth variable domain; L₃ is a linker;
optionally wherein:
V1 and V2 form the antigen binding site A and V3 and V4 form the antigen binding site B, or
V1 and V2 form the antigen binding site B and V3 and V4 form the antigen binding site A, or
V1 and V3 form the antigen binding site A and V2 and V4 form the antigen binding site B, or
V1 and V3 form the antigen binding site B and V2 and V4 form the antigen binding site A, or
V1 and V4 form the antigen binding site A and V2 and V3 form the antigen binding site B, or
V1 and V4 form the antigen binding site B and V2 and V3 form the antigen binding site A, and
optionally, wherein the first polypeptide and the second polypeptide are covalently or non-covalently linked together.

11. The antigen binding protein of claim 1, wherein the N-terminal or the C-terminal amino acid(s) of the polypeptide chains of the antigen binding protein comprises (a) modification(s).

12. A nucleic acid or nucleic acids encoding the antigen binding protein of claim 1.

13. A vector or vectors comprising the nucleic acid or nucleic acids of claim 12.

14. A host cell comprising the nucleic acid or nucleic acids of claim 12.

15. A host cell comprising the vector(s) of claim 13.

16. A pharmaceutical composition comprising the antigen binding protein of claim 1, and optionally a pharmaceutically acceptable carrier.

17. A pharmaceutical composition comprising the nucleic acid(s) of claim 12, and optionally a pharmaceutically acceptable carrier.

18. A pharmaceutical composition comprising the vector(s) of claim 13, and optionally a pharmaceutically acceptable carrier.

19. The antigen binding protein of claim 1, for use in the treatment of cancer.

20. The pharmaceutical composition of claim 16, for use in the treatment of cancer.
